(19) United States
(12) Patent Application Publication
(10) Pub. No.: US 2012/0054917 A1
(43) Pub. Date: Mar. 1, 2012

(54) METHOD FOR IMPROVING BIOMASS YIELD

(76) Inventors: Steven John Hanley, Harpenden (GB); Angela Karp, Harpenden (GB)

(21) Appl. No.: 13/143,842

(22) PCT Filed: Jan. 11, 2010
(86) PCT No.: PCT/GB2010/000025

§ 371 Date: Nov. 11, 2011

(30) Foreign Application Priority Data
Jan. 9, 2009 (GB) 0900334.4
Jan. 9, 2009 (GB) 0900336.9
Jan. 9, 2009 (GB) 0900338.5
Jan. 9, 2009 (GB) 0900339.3
Jan. 9, 2009 (GB) 0900341.9
Jan. 9, 2009 (GB) 0900342.7
Jan. 9, 2009 (GB) 0900343.5
Jan. 9, 2009 (GB) 0900344.3
Jan. 9, 2009 (GB) 0900345.0
Jan. 9, 2009 (GB) 0900352.6
Jan. 9, 2009 (GB) 0900353.4

(51) Int. Cl. A01H 5/00; C12Q 1/68; C12N 5/10; A01H 1/06; C12N 15/82

(52) U.S. Cl. 800/290; 800/298; 435/320.1; 435/419; 435/6.11

(57) ABSTRACT

A method for predicting harvestable biomass yield in a crop comprising: genotyping a sample obtained from a crop plant for one or more markers genetically linked to a polynucleotide sequence having at least 50, 55, 60, 65, 70, 75, 80, 85, 90, 95, 97, 98, 99 or 100% identity to SEQ BD NO 1, 2, 3, 4, 5, 6, 7, 8, 9, 10, 11, 12, 13, 14, 15, 16, 17, 18, 19, 20, 21, 22, 23, 24 or 25, whereby the markers individually or collectively identify a haplotype associated with yield in a plurality of crop plants and correlating the haplotype with the harvested biomass yield.

### FIELD OF THE INVENTION

The present invention relates to methods for improving harvestable biomass yield in plants

### BACKGROUND TO THE INVENTION

The present invention relates generally to the field of molecular biology and concerns a method for increasing total harvestable biomass yield in field-grown plants. More specifically, the present invention concerns a method for increasing total harvestable biomass yield by transfer, through conventional genetics or transgenesis, of a specific genomic region which confers enhanced harvestable yield in field-grown plants.

The total biomass produced above-ground by a plant can be harvested and used as feedstock for food, forage, bioenergy (including heat and power, transport biofuels and biogas), biomaterials and biorefineries.

Total harvestable biomass yield is calculated according to the plants parts that constitute relevant harvestable product, the most precise being the use of only one part (e.g. grain) and the most generic when the total above ground biomass is used.

In food crops the most important aspect is the yield in terms of harvestable edible portion which ranges from seed, grain and fruits to all types of vegetative parts for vegetable and salad crops (e.g. leaves, roots tubers, modified inflorescences etc). For forage there may be additional parts of the plant that animals can eat or the whole crop may be relevant.

The production of first generation liquid biofuels requires easily accessible sugars, starches or oils. As these are present in harvestable food portions, the relevant total yield can be calculated according to the relevant edible food portions.

In contrast, for many other end-uses, all the above ground parts may be harvested and utilised—e.g biomass for bioenergy, biomass for advanced generation biofuels and biomass for biorefineries. Whether the total plant is harvested with or without leaves and with or without flowers depends on the crop and precise end-use function.

Selective breeding has been employed for centuries to improve, or attempt to improve, phenotypic traits of agronomic and economic interest in plants such as yield. Generally speaking, selective breeding involves the selection of individuals to serve as parents of the next generation on the basis of one or more phenotypic traits of interest. However, such phenotypic selection is frequently complicated by non-genetic factors that can impact the phenotype(s) of interest. Non-genetic factors that can have such effects include, but are not limited to environmental influences such as soil type and quality, rainfall, temperature range, and others.

Variation in agronomic traits falls into two categories: qualitative and quantitative. The term “qualitative trait” is used when variation in the trait falls into discrete categories. Qualitative variation of this kind is normally under the control of one or two genes whose inheritance can be simply monitored in a cross. However, the majority of traits of interest to breeders, including total harvestable biomass yield, are quantitative in nature and are under the control of several genes each of which may have an important but small effect on the trait. The effects of each the genes, which may act independently or interact with each other in different ways, are influenced by the environment. Consequently, harvestable biomass yield is measured as a quantitative character and genomic regions that influence yield are referred to as quantitative trait loci (QTL).

It can be very difficult to map the genetic loci that contribute to the expression of quantitative traits. For QTL analysis the progeny of a given cross may be analysed for the trait and each individual assigned a score depending on the phenotype observed. All the individuals in the mapping population are then screened using molecular markers. Association between markers and the trait scores are searched for using software packages. Because of the environmental influence, the mapping population needs to be as big as possible and large numbers of molecular markers need to be used. Moreover, the mapping population should be grown and assessed at more than one site to ensure that robust QTL have been identified. Because of the nature of QTL, for a given complex trait such as yield, several QTL may be identified in different locations on the genetic map in a single cross. Attention is focussed on the QTL which contribute most to the heritable variation that is observed in the population. If the same QTL come out strongest when the population is grown at another site, confidence of their importance is gained. By nature, QTL mapping is a long term process and very resource intensive.

### SUMMARY

This disclosure concerns markers that define alleles of a gene at a quantitative trait locus (QTL) associated with improved harvestable biomass yield in crop plants. Methods for predicting harvestable biomass yield in a crop plant, for example, by determining a contribution to harvestable biomass yield by the allele, using the disclosed markers is disclosed. Kits for performing such methods also form part of the invention. Transgenic crop plants comprising an exogenous gene associated with harvestable biomass yield are disclosed. Transgenic crop plants expressing a recombinant polypeptide associated with harvestable biomass yield also form part of the invention.

The present invention relates to Xyld1, Xyld2, Xyld3, Xyld4, Xyld5, Xyld6, Xyld7, Xyld8, Xyld9 and Xyld10 polynucleotides and polypeptides and homologues thereof, in particular, to these genes found in Populus and Salix and homologues thereof.

Examples of polynucleotides and polypeptides of Xyld1, Xyld2, Xyld3, Xyld4, Xyld5, Xyld6, Xyld7, Xyld8, Xyld9 and Xyld10 are shown in the Table below:

| | | Salix | Salix | Salix | Salix |
|---|---|---|---|---|---|
| | Populus | Allele A | Allele A | Allele C | Allele C |
| | polynucleotide | polynucleotide | polypeptide | polynucleotide | polypeptide |
| | sequence | sequence | sequence | sequence | sequence |
| Xyld1 | SEQ ID NO. 4 | SEQ ID NO. 5 | SEQ ID NO. 27 | | |
| Xyld2 | SEQ ID NO. 6 | SEQ ID NO. 7 | SEQ ID NO. 28 | | |
| Xyld3 | SEQ ID NO. 8 | SEQ ID NO. 9 | SEQ ID NO. 29 | | |
| Xyld4 | SEQ ID NO. 10 | SEQ ID NO. 11 | SEQ ID NO. 30 | SEQ ID NO. 12 | SEQ ID NO. 31 |
| Xyld5 | SEQ ID NO. 13 | | | | |
| Xyld6 | SEQ ID NO. 14 | SEQ ID NO. 15 | SEQ ID NO. 32 | SEQ ID NO. 16 | SEQ ID NO. 33 |
| Xyld7 | SEQ ID NO. 3 | SEQ ID NO. 2 | | SEQ ID NO. 1 | SEQ ID NO. 26 |
| Xyld8 | SEQ ID NO. 17 | SEQ ID NO. 18 | SEQ ID NO. 34 | SEQ ID NO. 19 | SEQ ID NO. 35 |
| Xyld9 | SEQ ID NO. 20 | SEQ ID NO. 21 | SEQ ID NO. 36 | SEQ ID NO. 22 | SEQ ID NO. 37 |
| Xyld10 | SEQ ID NO. 23 | SEQ ID NO. 24 | SEQ ID NO. 38 | SEQ ID NO. 25 | SEQ ID NO. 39 |

Polynucleotides useful in the invention may comprise nucleotide sequences having at least 50, 55, 60, 65, 70, 75, 80, 85, 90, 95, 97, 98, 99 or 100% identity to the Xyld1, Xyld2, Xyld3, Xyld4, Xyld5, Xyld6, Xyld7, Xyld8, Xyld9 and Xyld10 polynucleotides.

Polypeptides useful in the invention may comprise amino acid sequences having at least 50, 55, 60, 65, 70, 75, 80, 85, 90, 95, 97, 98, 99 or 100% identity to the Xyld1, Xyld2, Xyld3, Xyld4, Xyld5, Xyld6, Xyld7, Xyld8, Xyld9 and Xyld10 polypeptides.

In preferred aspects of the present invention, polynucleotides and polypeptides of the Salix allele C genes are provided for use in the invention.

In preferred aspects of the present invention, polynucleotide and polypeptide sequences of Xyld7, in particular Xyld7 allele C, are provided for use in the invention.

According to a first aspect of of the present invention there is provided Xyld1, Xyld2, Xyld3, Xyld4, Xyld5, Xyld6, Xyld7, Xyld8, Xyld9 and Xyld10 polynucleotides and polypeptides.

According to another aspect of the present invention there is provided a method for predicting harvestable biomass yield in a crop comprising: genotyping a sample obtained from a crop plant for one or more markers genetically linked to a polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, whereby the markers individually or collectively identify a haplotype associated with yield in a plurality of crop plants and correlating the haplotype with the harvested biomass yield.

According to a further aspect of the present invention there is provided a method for predicting harvestable biomass yield in a crop comprising: genotyping a sample obtained from a crop plant for one or more markers genetically linked to a polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, whereby the markers individually or collectively identify a haplotype associated with yield in a plurality of crop plants and correlating the haplotype with the harvested biomass yield.

According to a further aspect of the present invention there is provided a method for determining the contribution of an allele to harvestable biomass yield in a crop, wherein the allele is an allele of a polynucleotide sequence, said polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, the method comprising: genotyping a sample obtained from a crop plant for one or more markers genetically linked to said polynucleotide, which markers individually or collectively identify a haplotype correlated with a contribution to harvestable biomass yield.

According to a further aspect of the present invention there is provided a method for determining the contribution of an allele to harvestable biomass yield in a crop, wherein the allele is an allele of a polynucleotide sequence, said polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, the method comprising: genotyping a sample obtained from a crop plant for one or more markers genetically linked to said polynucleotide, which markers individually or collectively identify a haplotype correlated with a contribution to harvestable biomass yield.

According to a further aspect of the present invention there is provided a method of identifying an allele that is associated with harvestable biomass yield in a crop comprising: obtaining a sample from a crop plant; amplifying DNA present in said sample and detecting the presence of a polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25 in the amplified DNA.

According to a further aspect of the present invention there is provided a method of identifying an allele that is associated with harvestable biomass yield in a crop comprising: obtaining a sample from a crop plant; amplifying DNA present in said sample and detecting the presence of a polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39 in the amplified DNA.

According to a further aspect of the present invention there is provided a method of selecting a crop by marker assisted selection of an allele associated with harvestable biomass yield, wherein said allele is an allele of a polynucleotide sequence, said polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, said method comprising: determining the presence of one or more markers, which markers are genetically linked to said polynucleotide.

According to a further aspect of the present invention there is provided a method of selecting a crop by marker assisted selection of an allele associated with harvestable biomass yield, wherein said allele is an allele of a polynucleotide sequence, said polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, said method comprising: determining the presence of one or more markers, which markers are genetically linked to said polynucleotide.

According to a further aspect of the present invention there is provided an isolated nucleic acid sequence comprising a marker or plurality of markers associated with a QTL associated with harvestable biomass yield in a crop wherein the marker or plurality of markers are genetically linked to a polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25.

According to a further aspect of the present invention there is provided an isolated nucleic acid sequence comprising a marker or plurality of markers associated with a QTL associated with harvestable biomass yield in a crop wherein the marker or plurality of markers are genetically linked to a polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39.

According to a further aspect of the present invention there is provided a method for producing a transgenic crop plant, comprising introducing into an unmodified crop plant an exogenous polynucleotide, wherein said polynucleotide comprises a nucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25.

According to a further aspect of the present invention there is provided a method for producing a transgenic crop plant, comprising introducing into an unmodified crop plant an exogenous polynucleotide, wherein said polynucleotide comprises a nucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39.

According to a further aspect of the present invention there is provided a method for producing a transgenic crop plant that expresses a recombinant polypeptide encoded by a sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, comprising introducing an exogenous polynucleotide comprising a cDNA encoding said recombinant polypeptide into an unmodified crop plant.

According to a further aspect of the present invention there is provided a method for producing a transgenic crop plant that expresses a recombinant polypeptide comprising an amino acid sequence having at least 50, 55, 60, 65, 70, 75, 80, 85, 90, 95, 97, 98, 99 or 100% identity to SEQ ID NO 26, 27, 28, 29, 30, 31, 32, 33, 34, 35, comprising introducing an exogenous polynucleotide comprising a cDNA encoding said recombinant polypeptide into an unmodified crop plant.

According to a further aspect of the present invention there is provided a transgenic crop plant comprising an exogenous gene, wherein said gene comprises a sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25.

According to a further aspect of the present invention there is provided a transgenic crop plant comprising an exogenous gene, wherein said gene comprises a sequence encoding a polypeptide, the polypeptide having at least 50, 55, 60, 65, 70, 75, 80, 85, 90, 95, 97, 98, 99 or 100% identity to the polypeptide of SEQ ID NO 26, 27, 28, 29, 30, 31, 32, 33, 34, 35, 36, 37, 38 or 39.

According to a further aspect of the present invention there is provided a transgenic crop plant expressing a recombinant polypeptide encoded by a sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25.

According to a further aspect of the present invention there is provided a transgenic crop plant expressing a recombinant polypeptide having at least 50, 55, 60, 65, 70, 75, 80, 85, 90, 95, 97, 98, 99 or 100% identity to SEQ ID NO 26, 27, 28, 29, 30, 31, 32, 33, 34, 35, 36, 37, 38 or 39.

According to a further aspect of the present invention there is provided a transgenic crop plant comprising a nucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, wherein said nucleotide sequence is operably linked to a heterologous regulatory element.

According to a further aspect of the present invention there is provided a transgenic crop plant comprising a nucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, wherein said nucleotide sequence is operably linked to a heterologous regulatory element.

According to a further aspect of the present invention there is provided a use of an exogenous polynucleotide comprising a sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA sequence, for improving harvestable biomass yield of a crop plant by transformation of the crop plant with the exogenous polynucleotide.

According to a further aspect of the present invention there is provided a use of an exogenous polynucleotide comprising a sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, for improving harvestable biomass yield of a crop plant by transformation of the crop plant with the exogenous polynucleotide.

According to a further aspect of the present invention there is provided a genetic construct comprising (a) a nucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA sequence, and (b) a promoter sequence capable of directing expression of the protein encoded by the nucleotide sequence in a plant comprising the genetic construct.

According to a further aspect of the present invention there is provided a genetic construct comprising (a) a nucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, and (b) a promoter sequence capable of directing expression of the protein encoded by the nucleotide sequence in a plant comprising the genetic construct.

According to a further aspect of the present invention there is provided a plant transformation vector comprising the genetic construct of the invention.

According to a further aspect of the present invention there is provided a plant or plant cell comprising a transformation vector of the invention.

In one or more embodiments of the invention, the marker is within an interval of less than 45, 40, 35, 30, 25, 20, 15, 10, 5, 4, 3, 2, 1 or 0 centimorgans (cM) from a nucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25.

In one or more embodiments of the invention, the marker is within an interval of less than 45, 40, 35, 30, 25, 20, 15, 10, 5, 4, 3, 2, 1 or 0 centimorgans (cM) from a nucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39.

Plants that are particularly useful in the methods of the invention include in particular monocotyledonous and dicotyledonous fodder crops, forage crops, ornamental crops, fruit crops, food crops, algae, forestry trees, bioenergy crops and biofuel crops including the following species and species hybrids: Acacia spp., Acer spp., Actinidia ssp., Agave spp., Agropyron spp., Agrostis spp., Allium spp., Alnus spp., Alopecurus spp., Amaranthus spp., Ananas spp., Apium spp., Arachis spp., Areca spp., Arundo spp., Arrhenatherum spp., Asparagus spp; Avena spp., Atriplex spp., Attalea spp., Beta spp., Betula spp., Brassica spp., Bromus spp., Bouteloua spp., Camelina spp., Camellia spp., Cannabis spp., Capsicum spp., Carica spp., Carex spp., Carthamus spp., Castanea spp., Carum spp., Cinnamomum spp., Citrus spp., Cocos spp., Coffea spp., Corchorus spp., Cotoneatser spp., Cucurbita spp., Cupressus spp., Cynodon spp., Daucus spp., Dactylis spp., Eucalyptus spp., Elaeis spp., Eleusine spp., Fagus spp., Festuca spp., Ficus spp., Fraxinus spp., Geranium spp., Ginkgo spp., Glycine spp., Gossypium spp., Helianthus spp., Hemerocallis spp., Heracleum spp., Hedysarum spp., Hibiscus spp., Hordeum spp., Indigo spp., Ipomoea spp., Lettuca spp., Jatropha spp., Lotus spp., Lactuca spp., Lathyrus spp., Lens spp., Linum spp., Lolium spp., Lupinus spp., Lezula spp., Lycopersicon spp., Malus spp., Manihot spp., Medicago spp., Melilotus spp., Mentha spp., Miscanthus spp., Musa spp., Nicotiana spp., Olea spp., Onobrychis spp., Ophiopogon spp., Oryza spp., Panicum spp., Papaver spp., Petunia spp., Phaseolus spp., Pennisetum spp., Phalaris spp., Phoenix spp., Phleum spp., Phyllostachys spp., Physalis spp., Panicum spp., Picea spp., Pinus spp., Pistacia spp., Pisum spp., Poa spp., Podocarpus spp., Pogmania spp., Populus spp., Prunus spp., Quercus spp., Ribes spp., Robinia spp., Rosa spp., Raphanus spp., Rheum spp., Ricinus spp., Rubus spp., Salix spp., Sequoia spp., Sesamum spp., Setaria spp., Saccharum spp., Sambucus spp., Secale spp., Sinapis spp., Solanum spp., Sorghum spp., Trifolium spp., Triticum spp., Triticosecale spp., Trisetum spp., Tagetes spp., Theobroma spp., Triadica spp., Vicia spp., Vitis spp., Vigna spp., Viola spp., Watsonia spp., Zea spp. amongst others.

According to another aspect of the present invention there is provided a polypetide having the amino acid sequence od SEQ ID NO:1.

The foregoing and other objects and features of the disclosures will become more apparent from the following detailed description, which proceeds with reference to the accompanying figures.

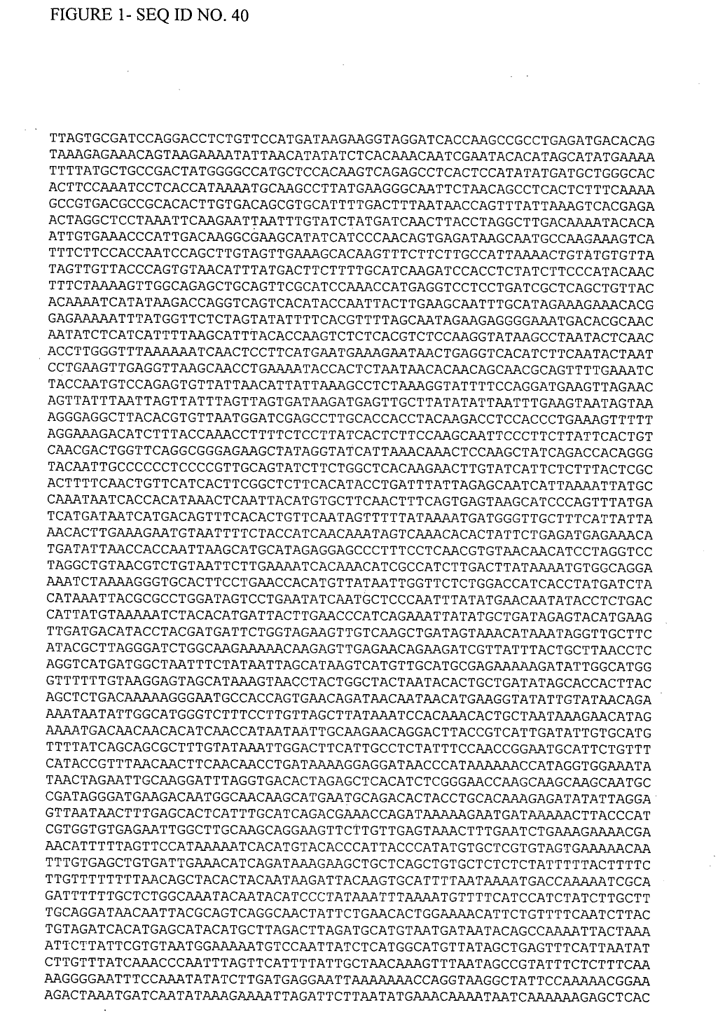

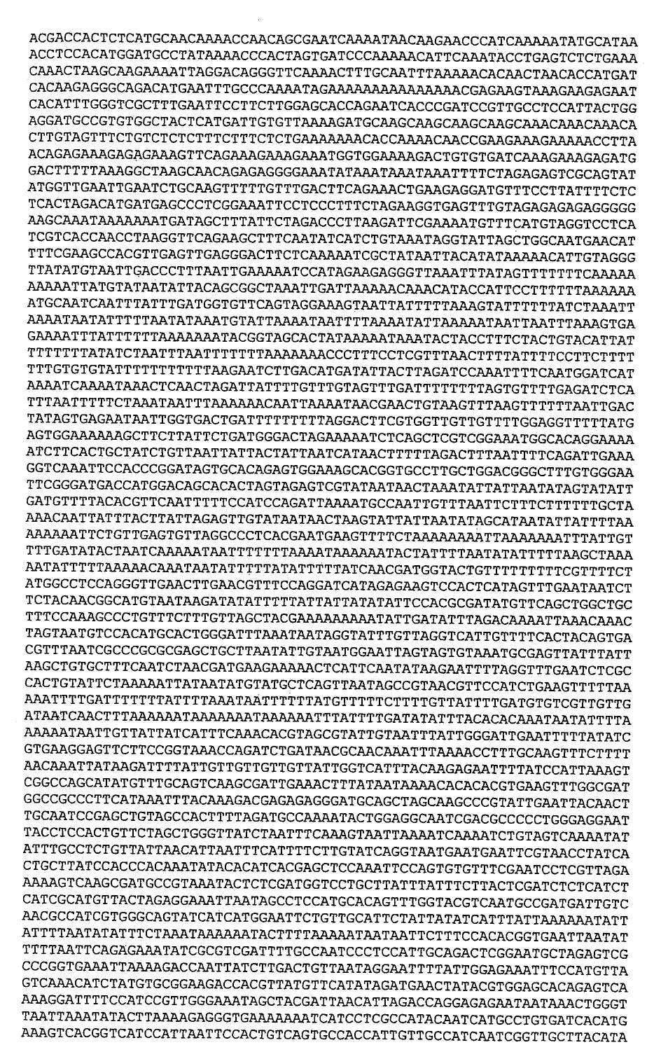

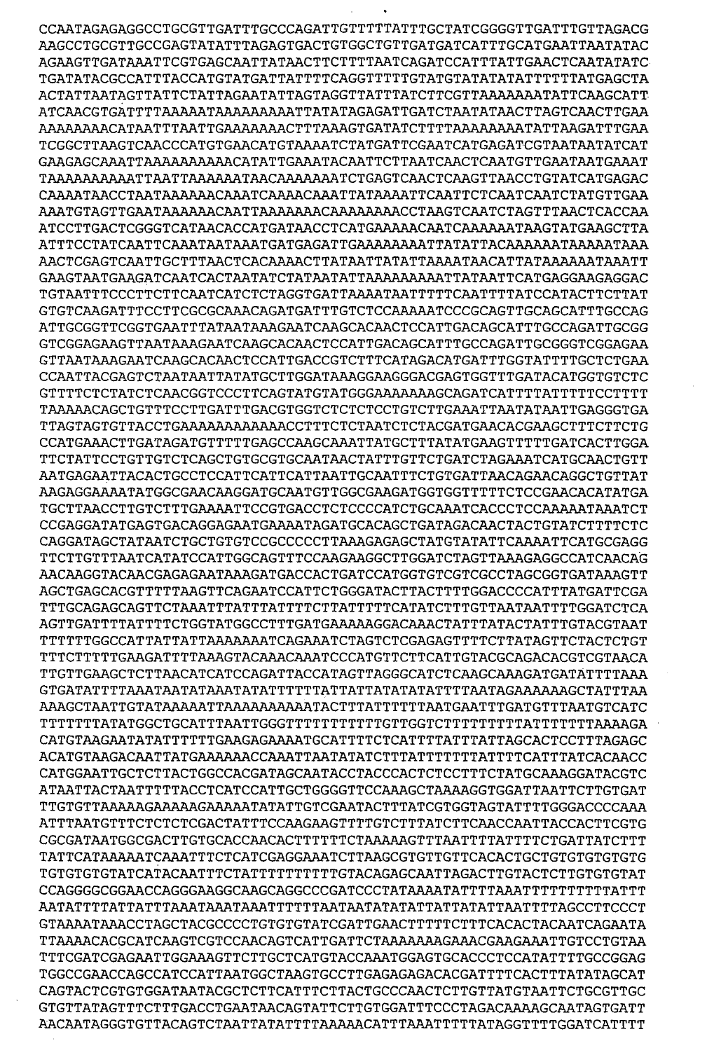

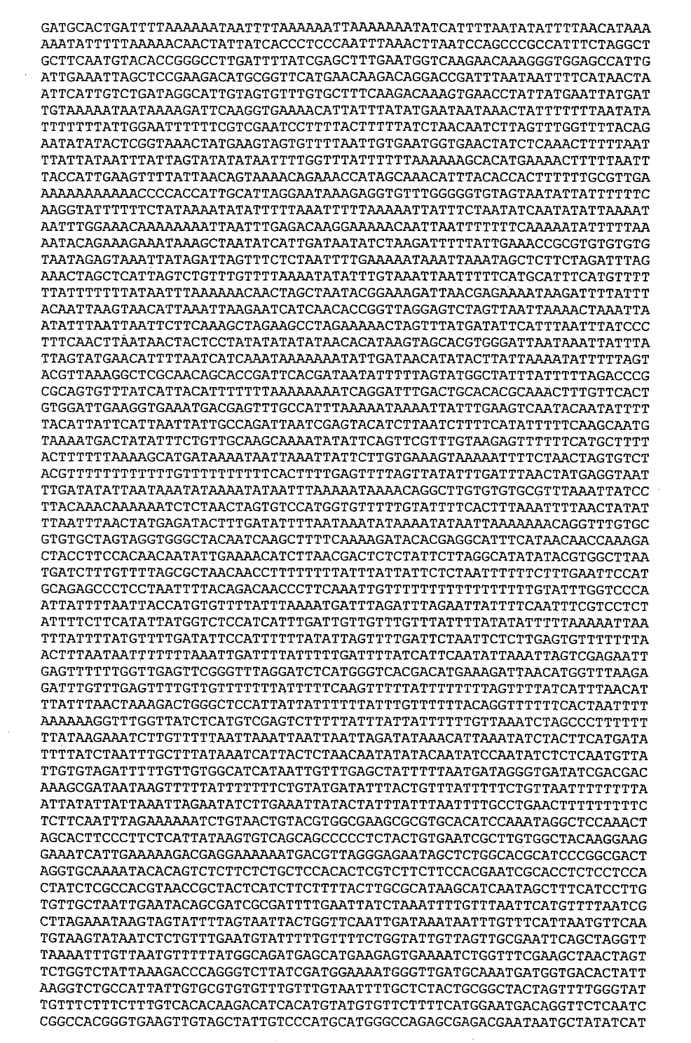

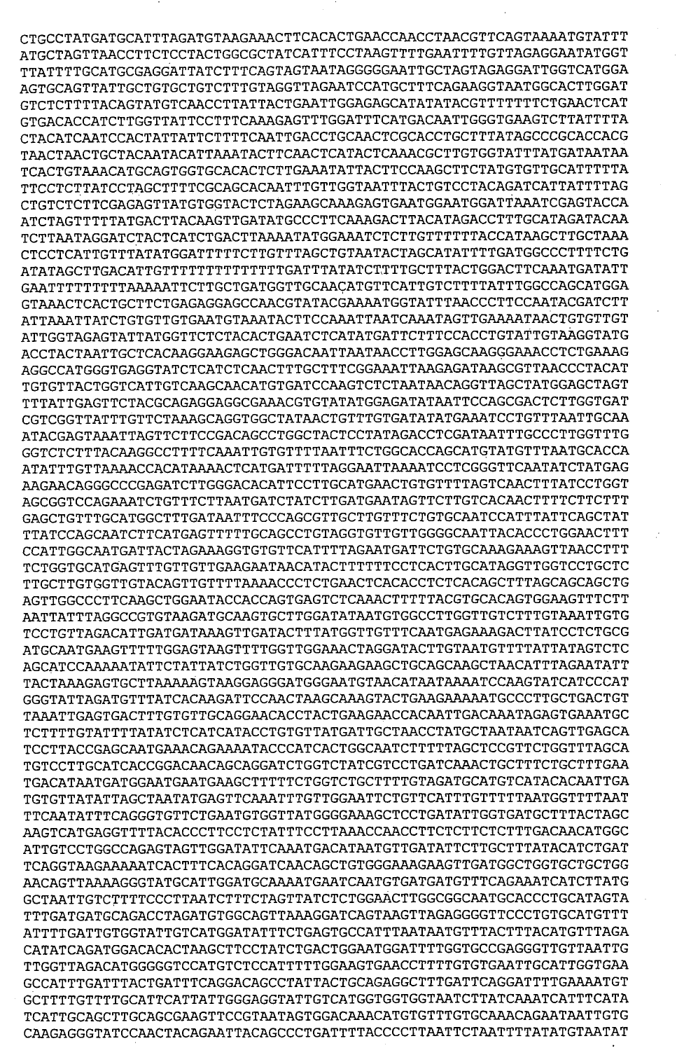

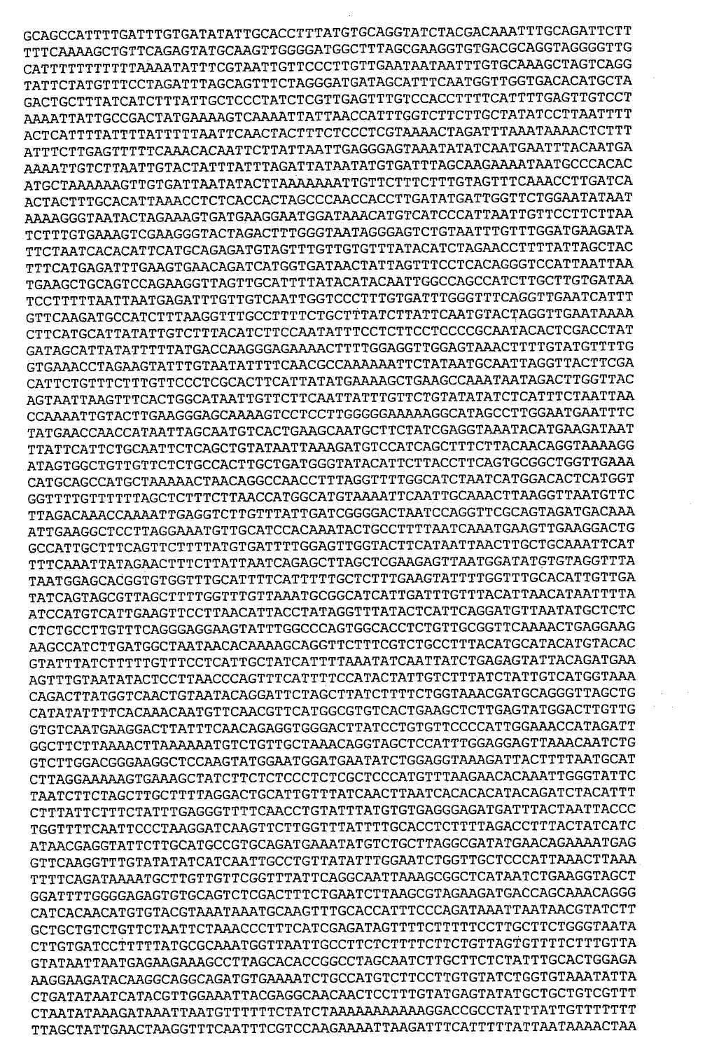

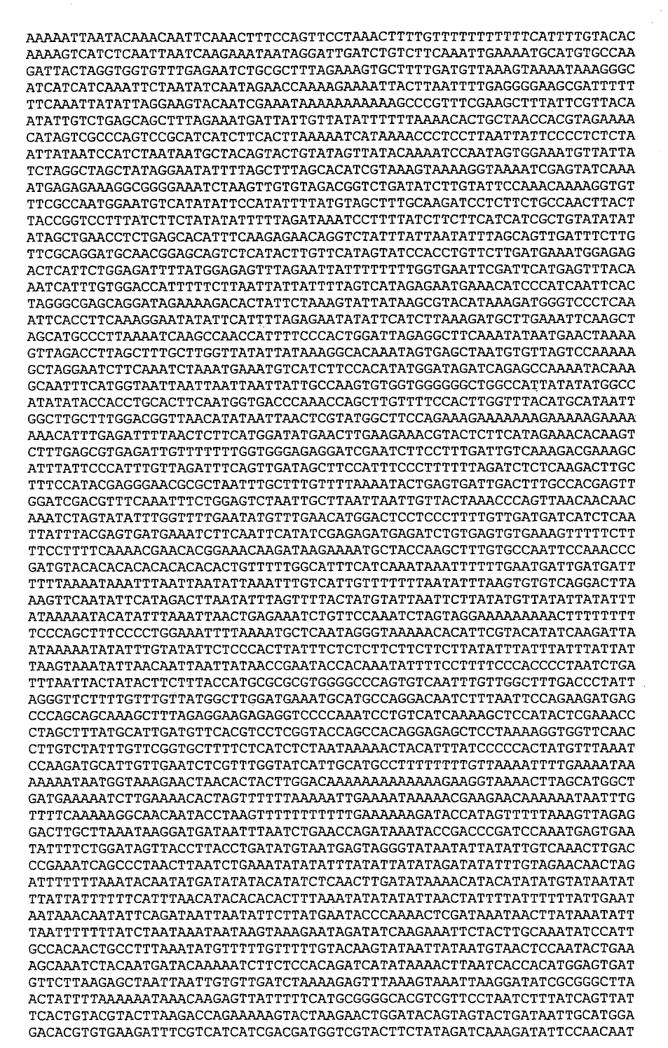

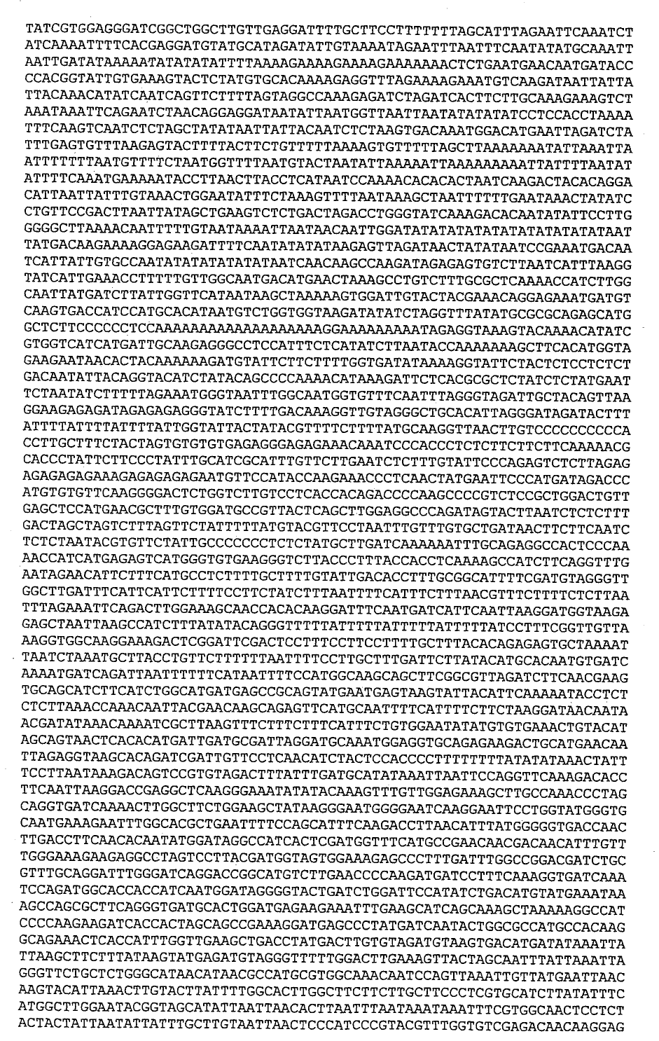

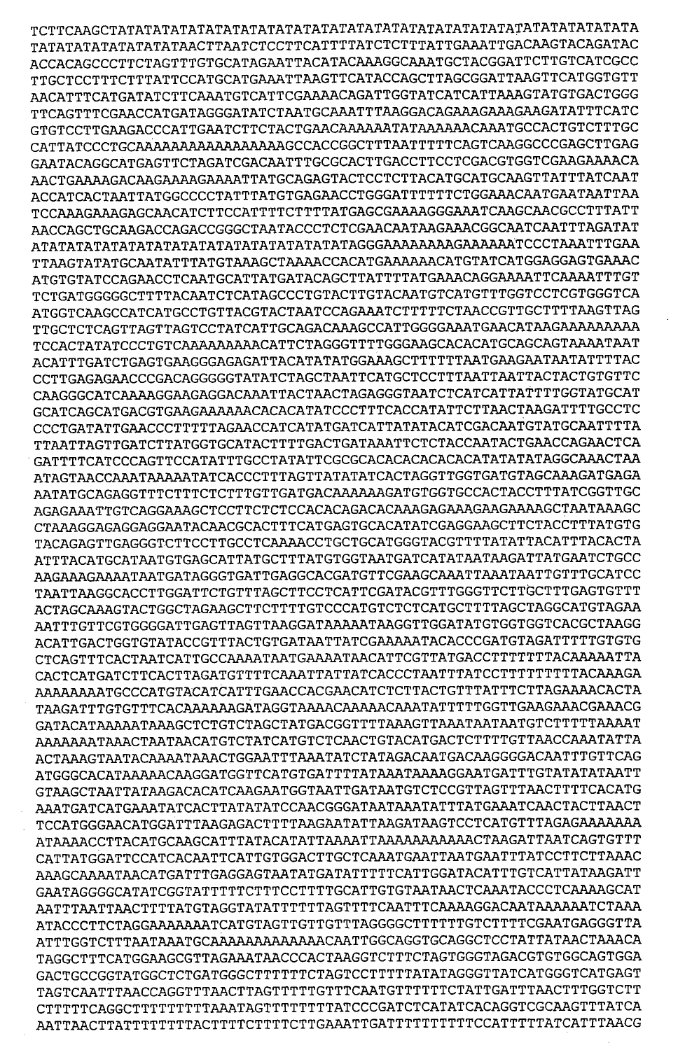

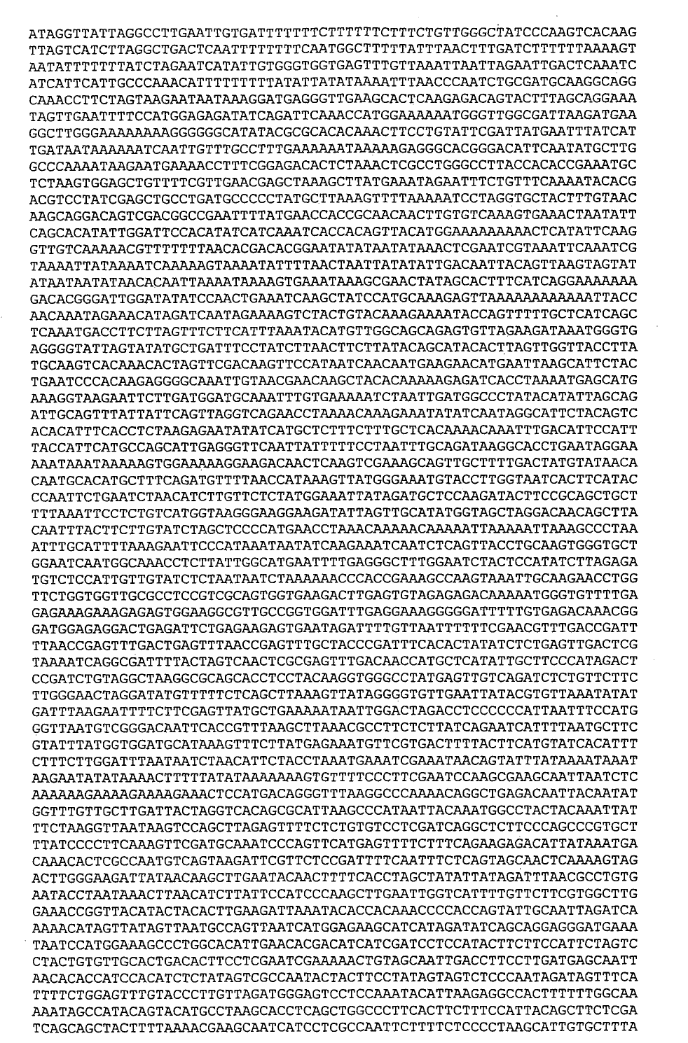

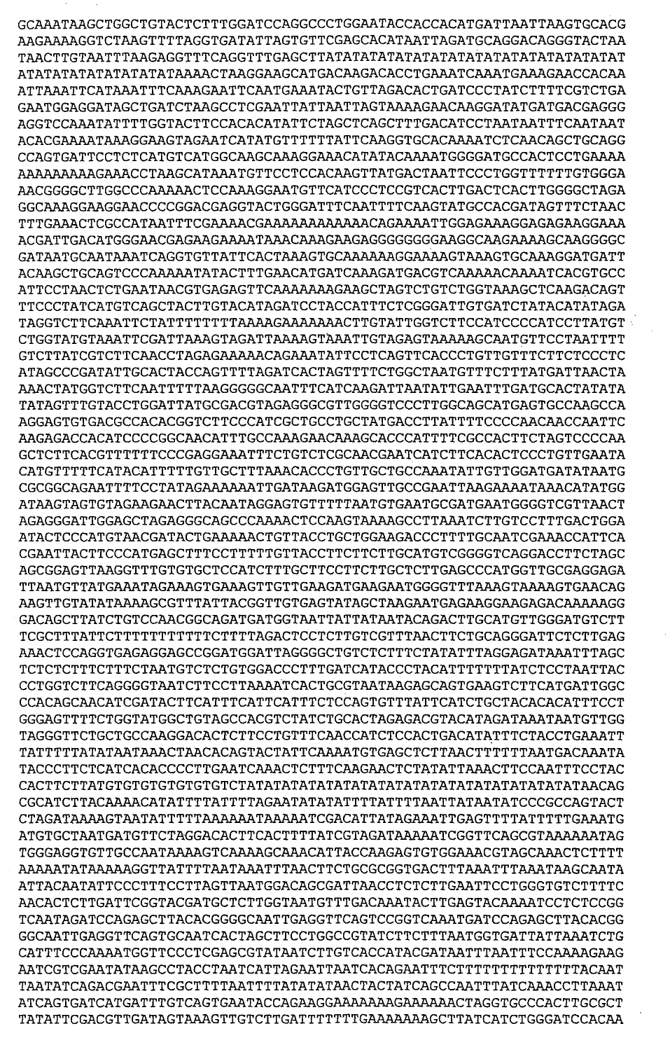

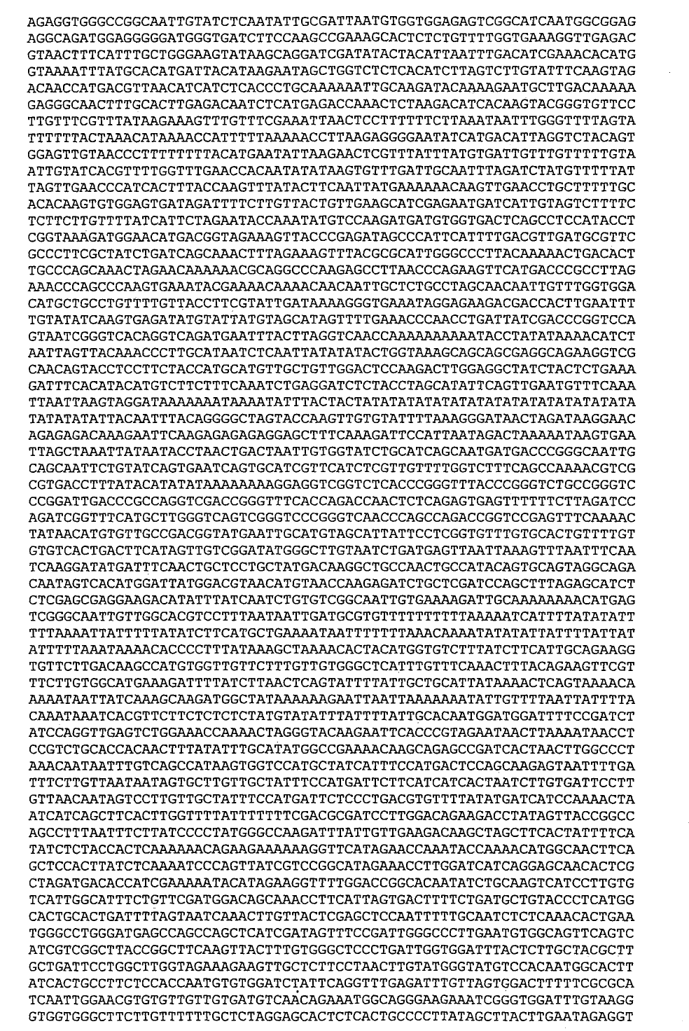

FIG. 1: shows the sequence of a QTL region in Populus associated with improved yield.

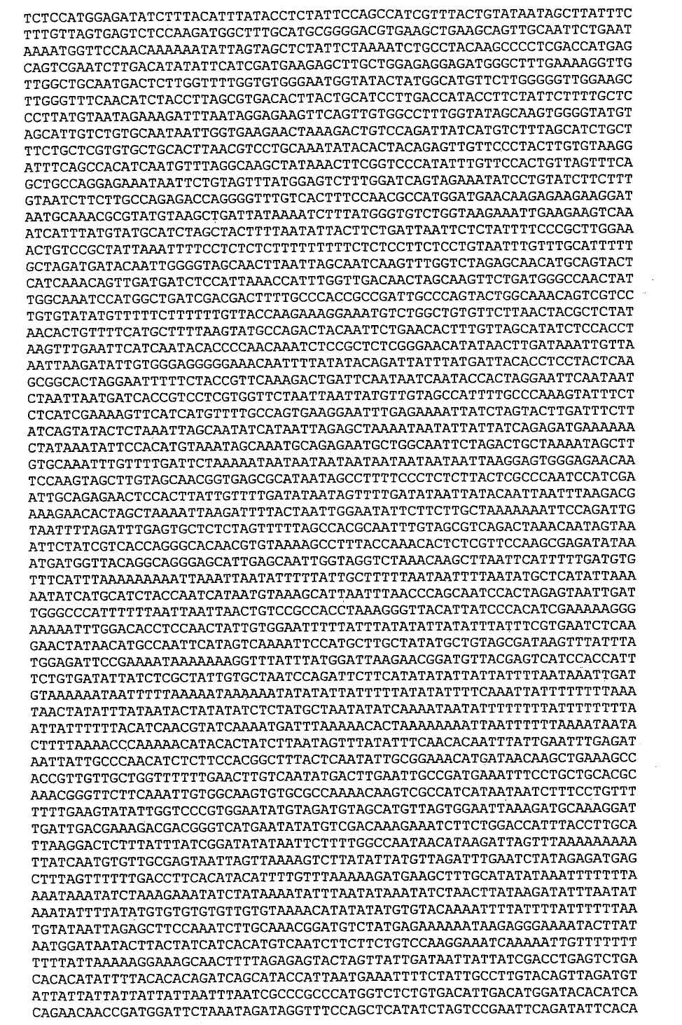

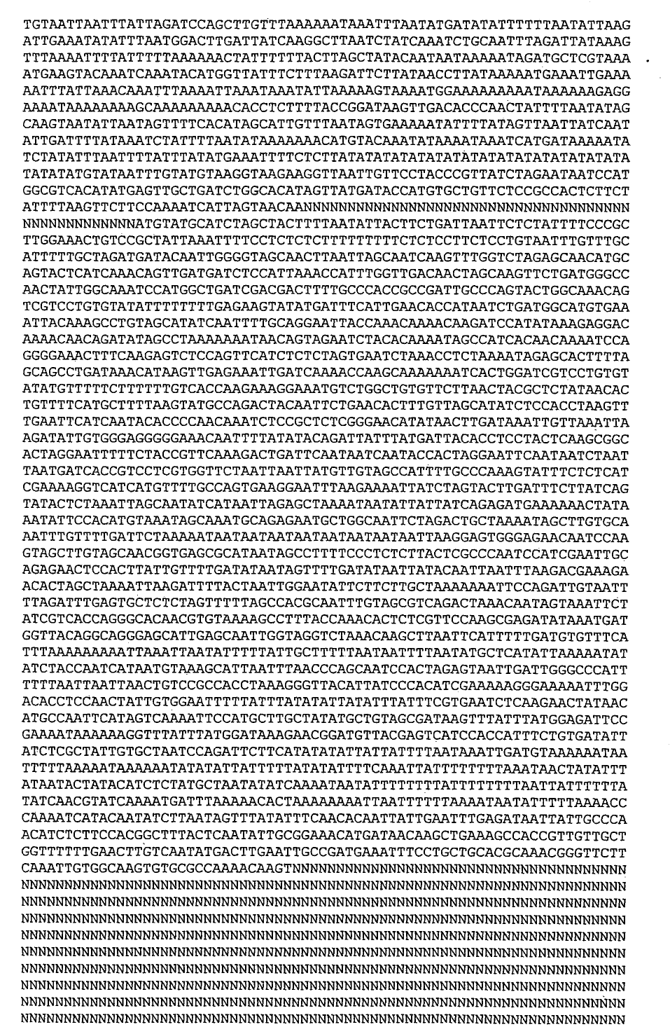

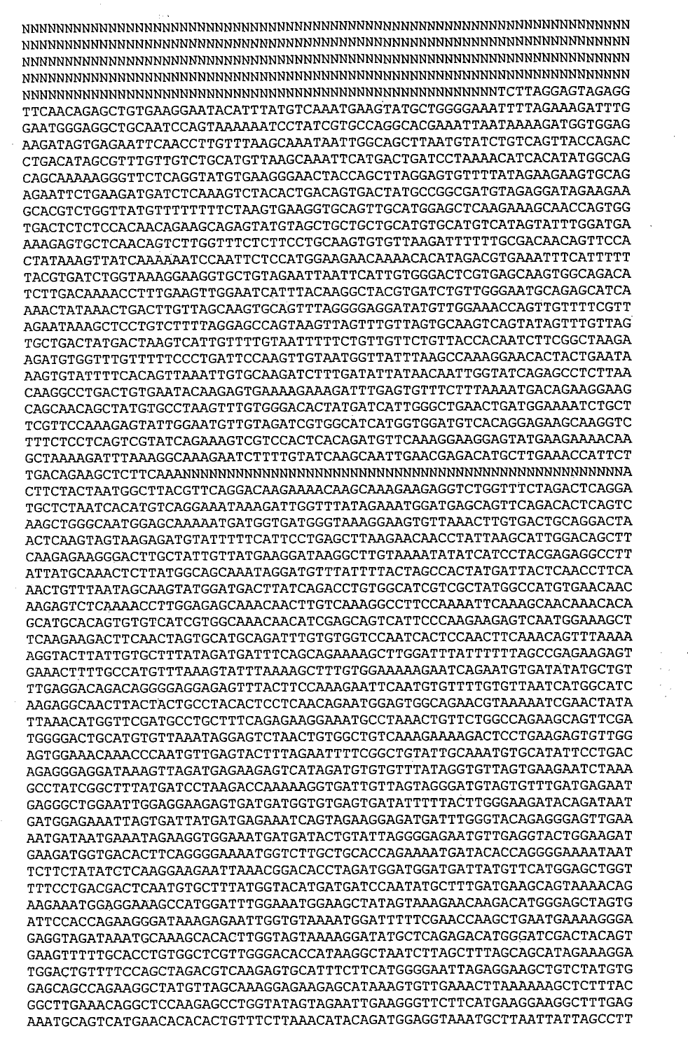

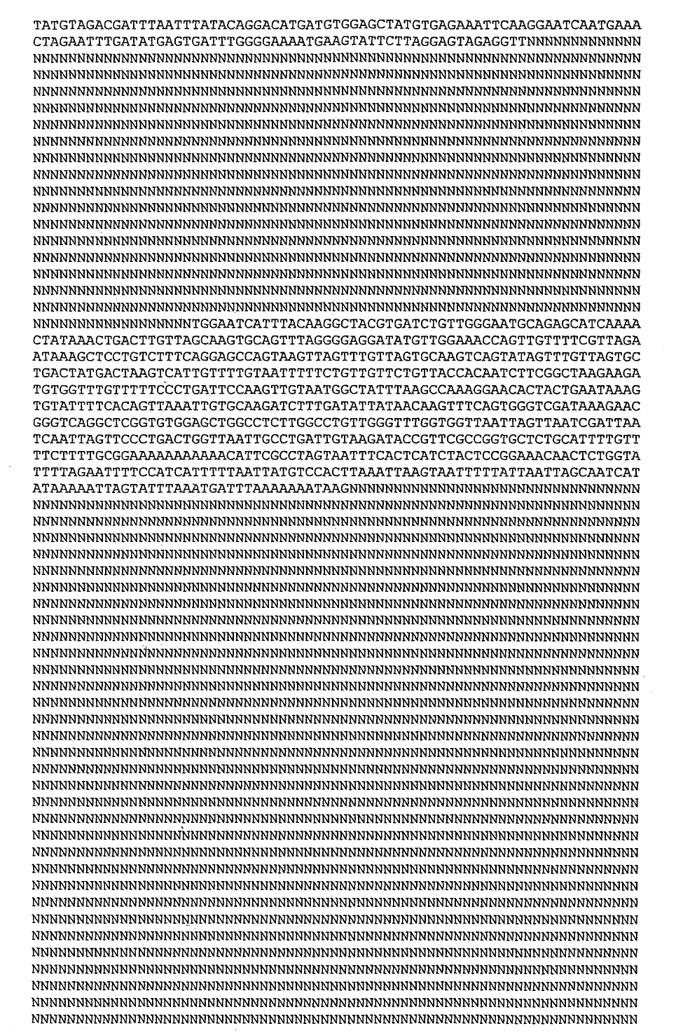

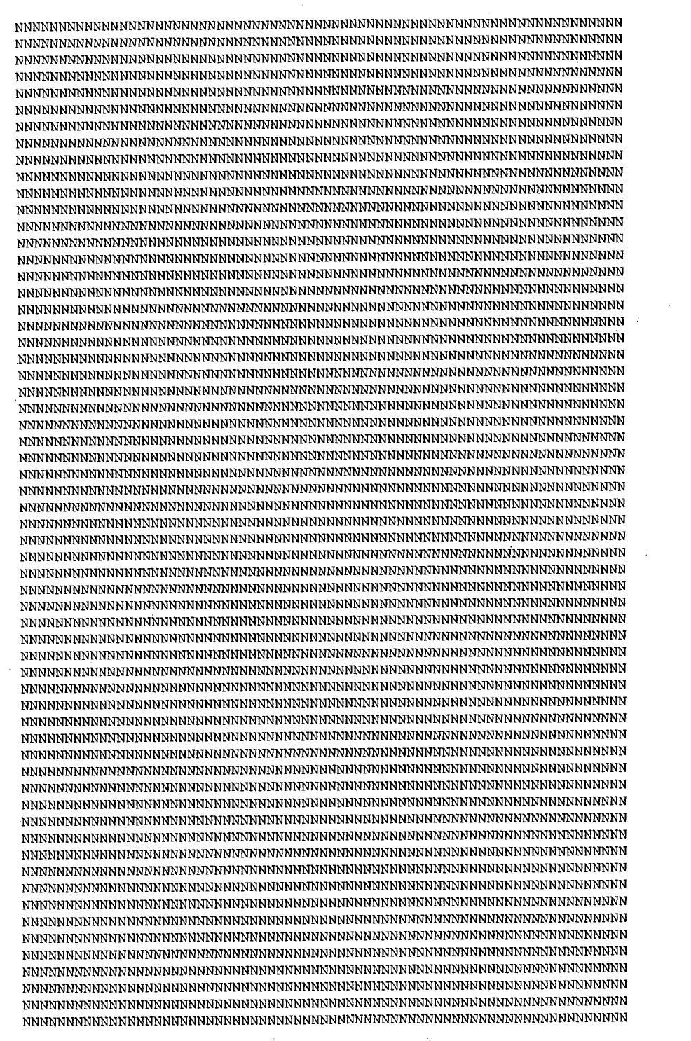

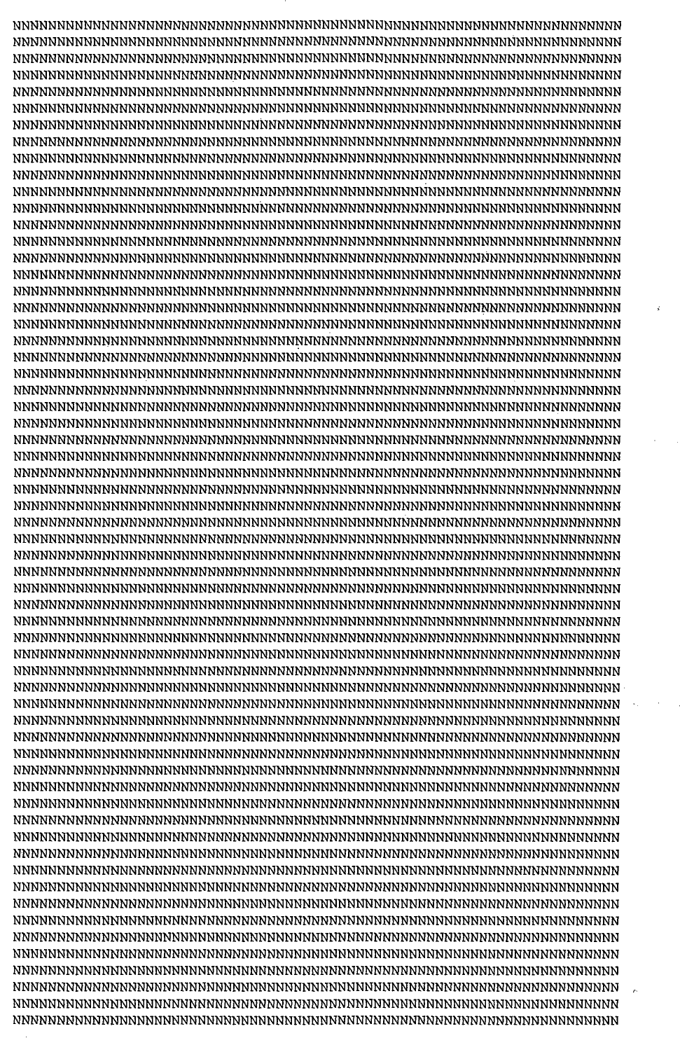

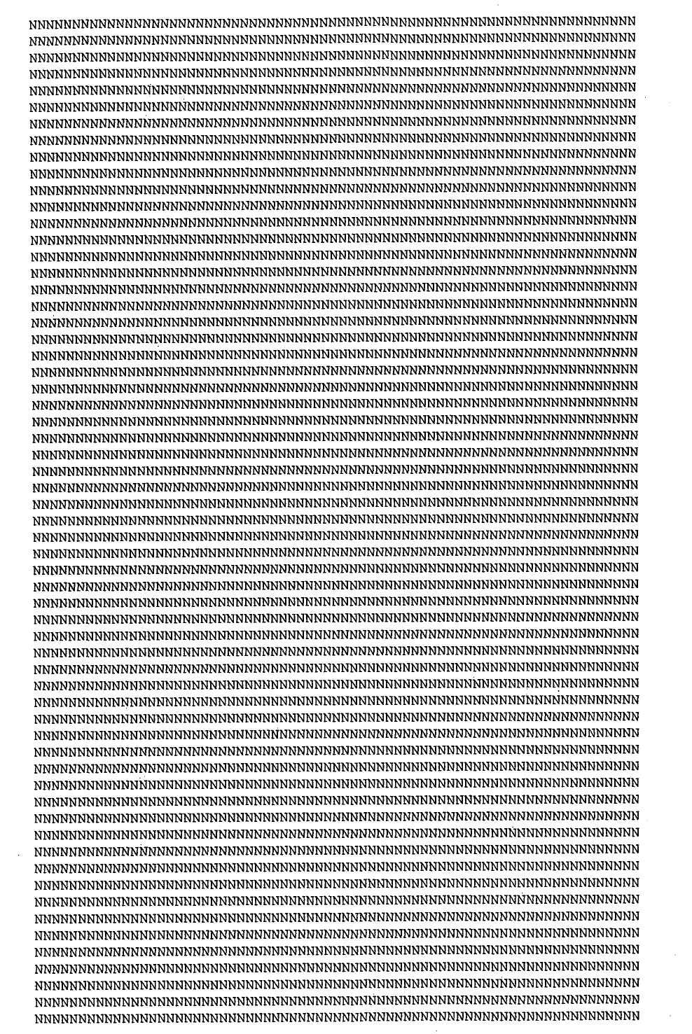

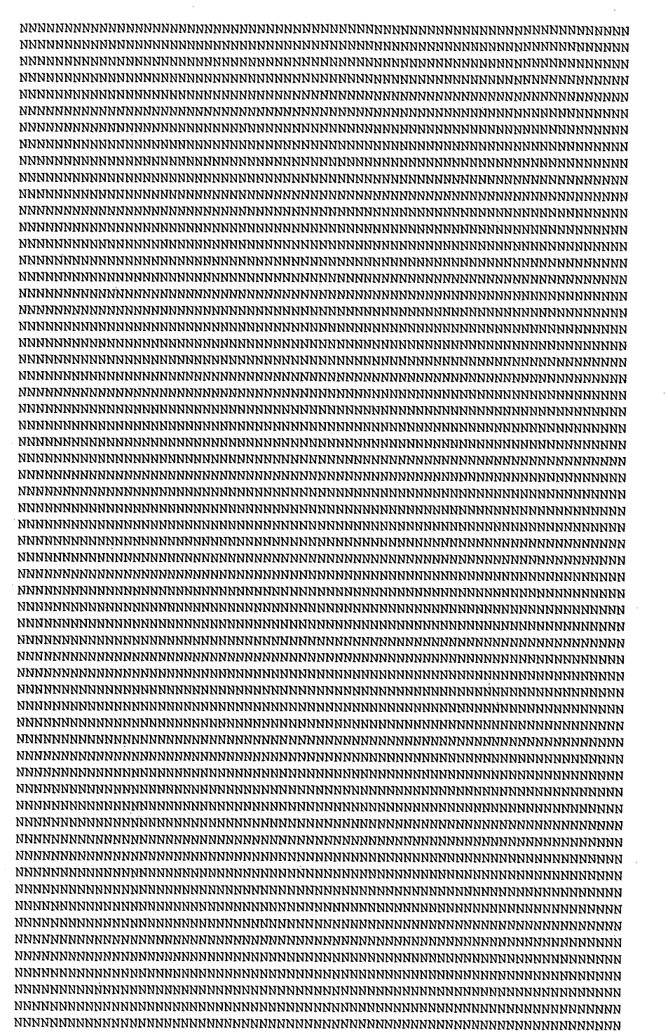

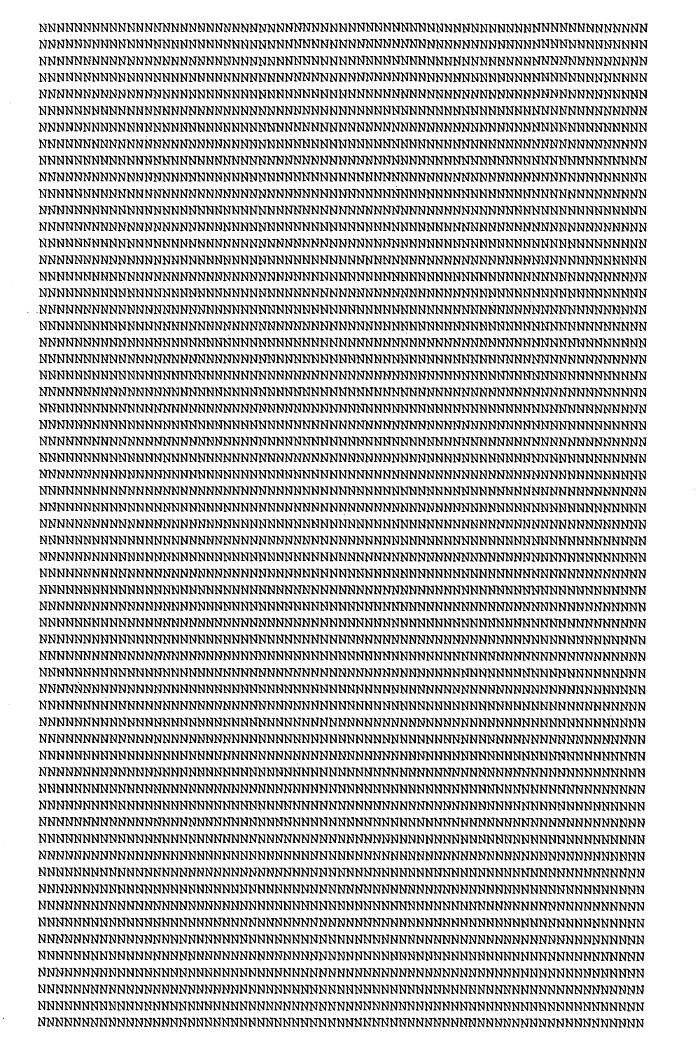

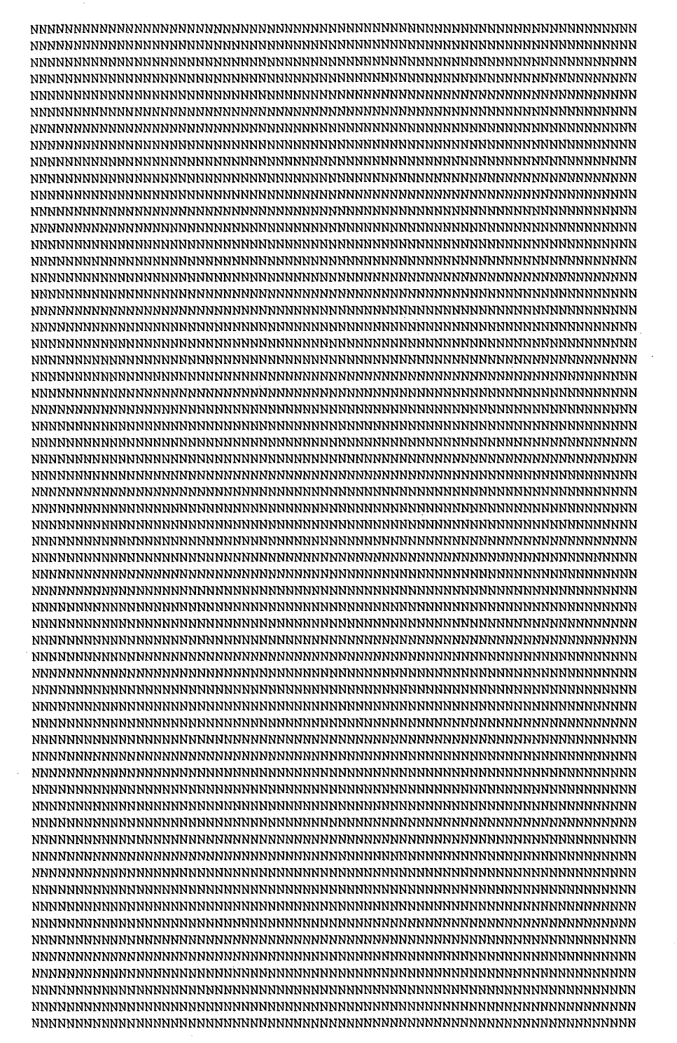

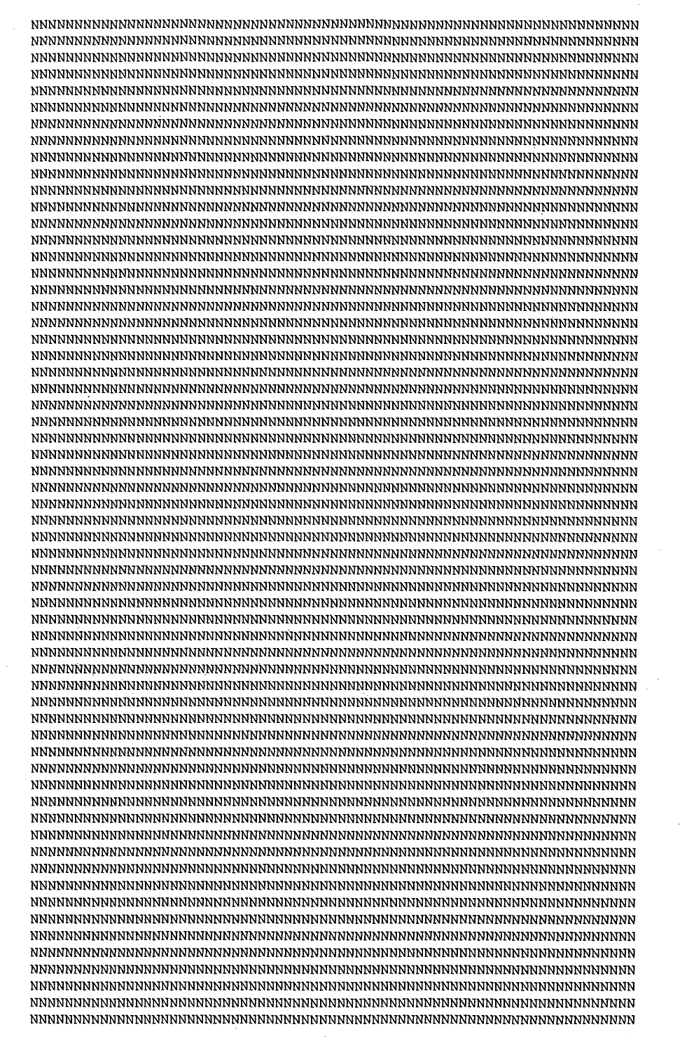

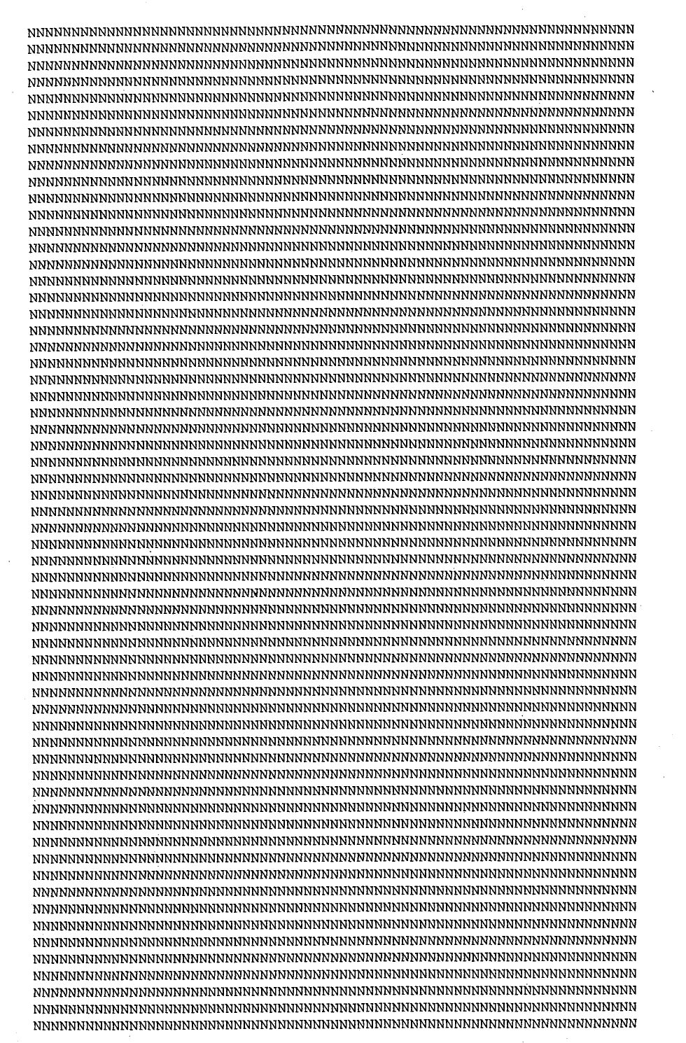

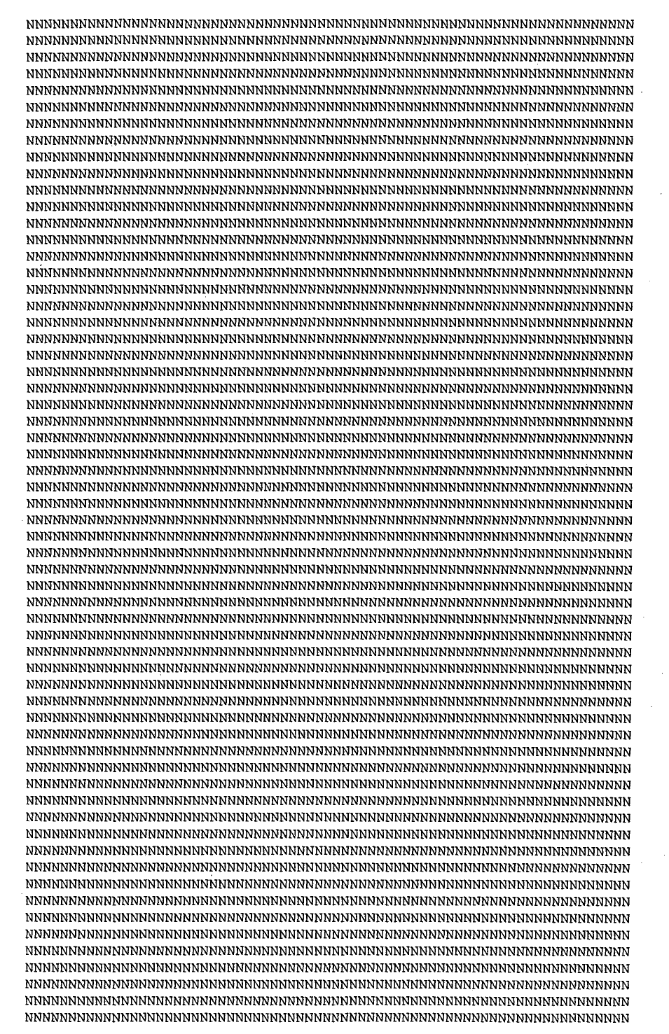

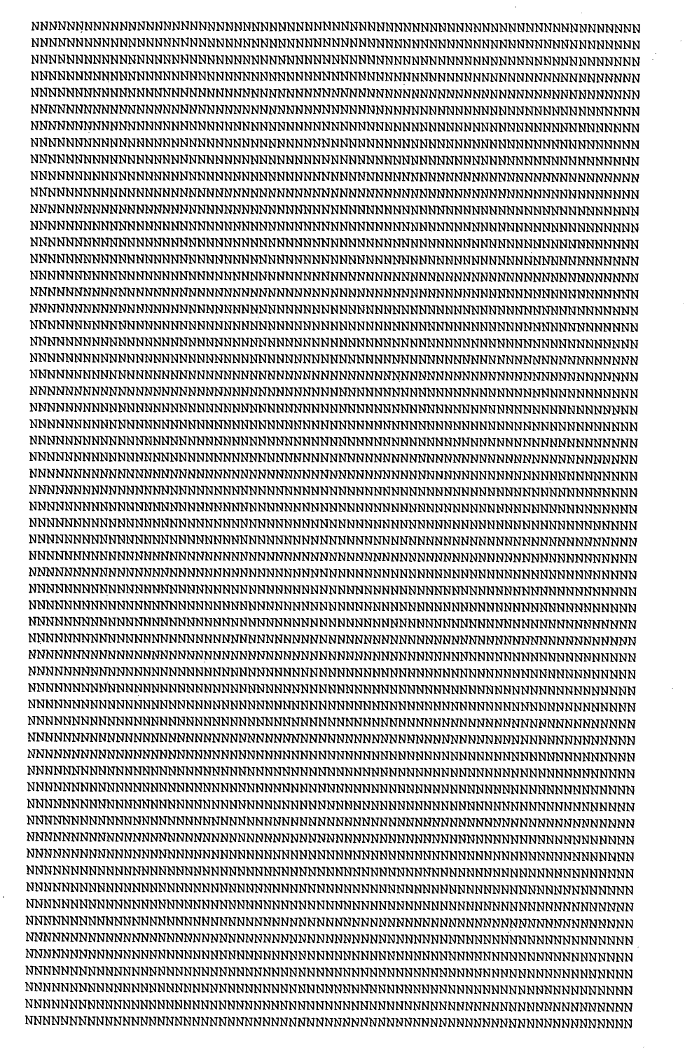

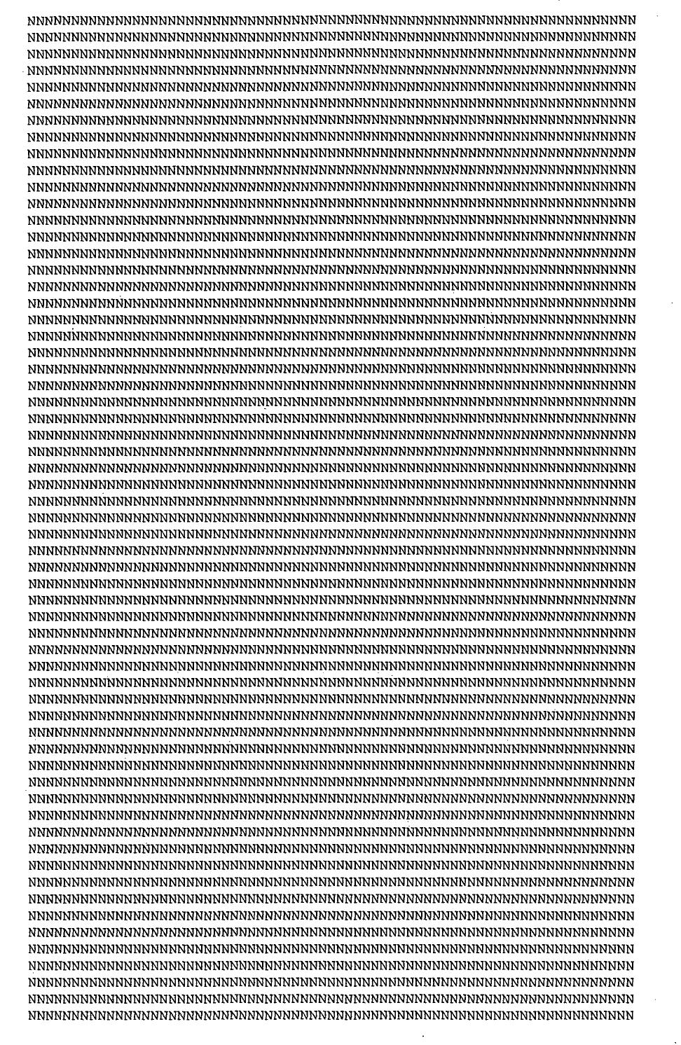

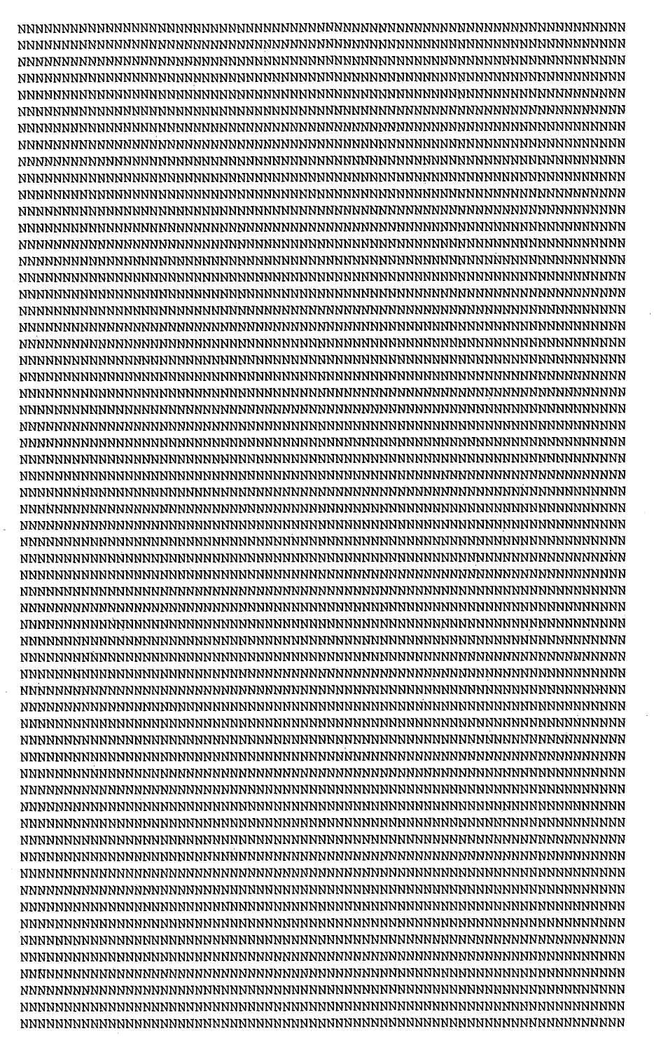

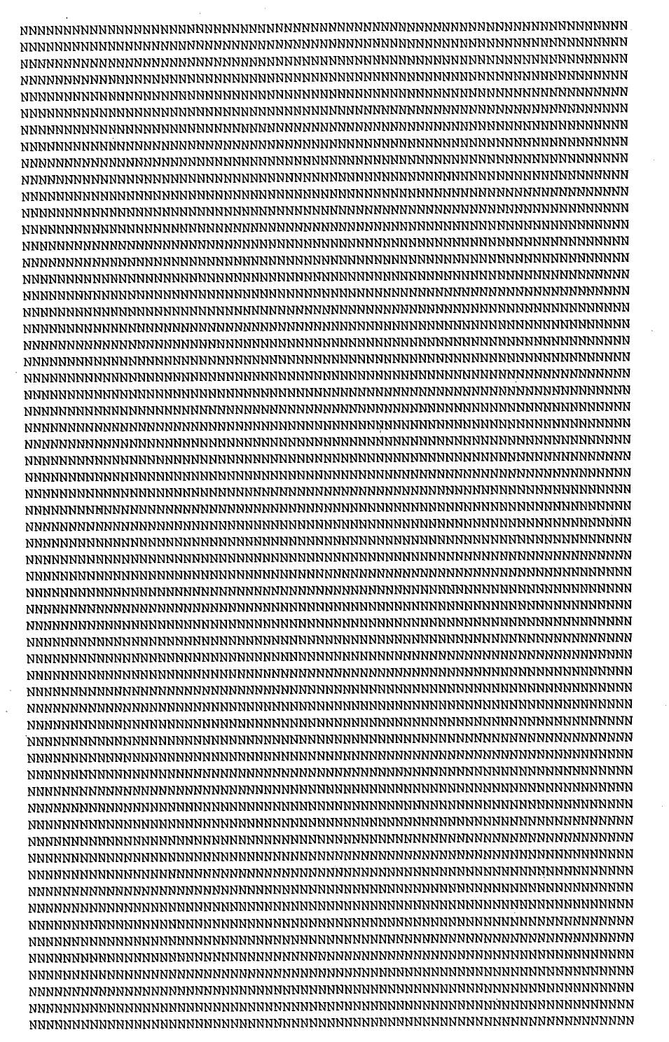

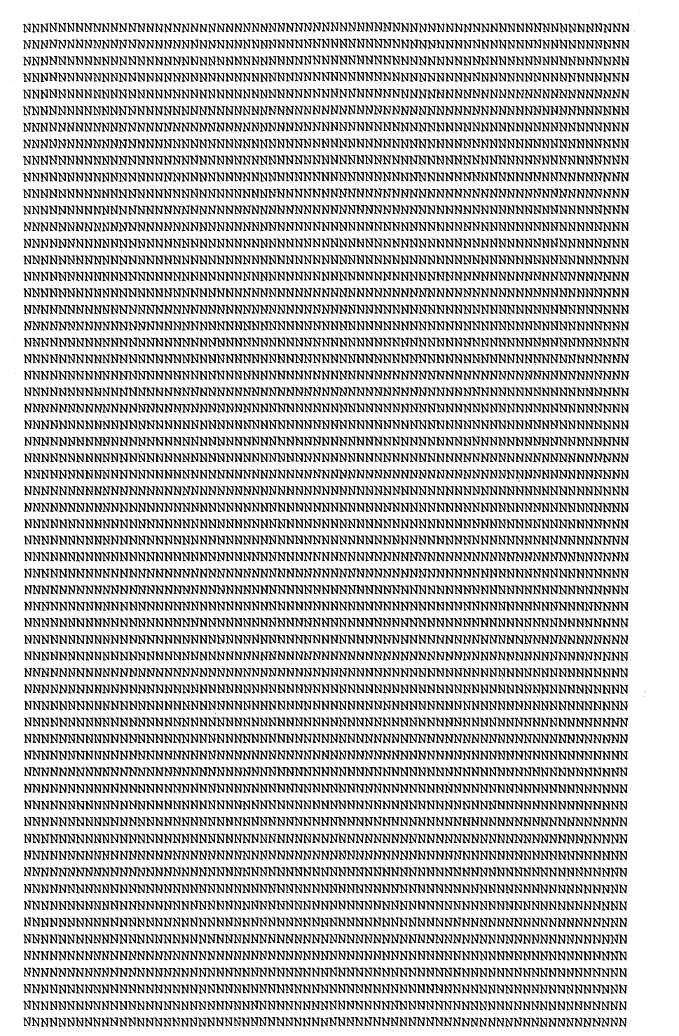

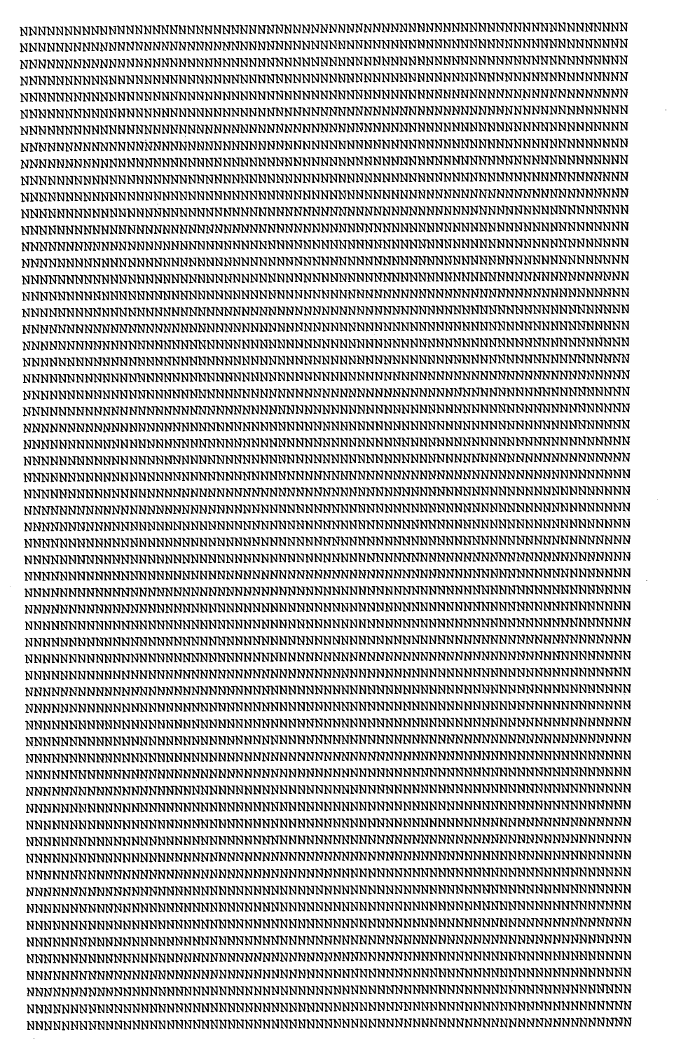

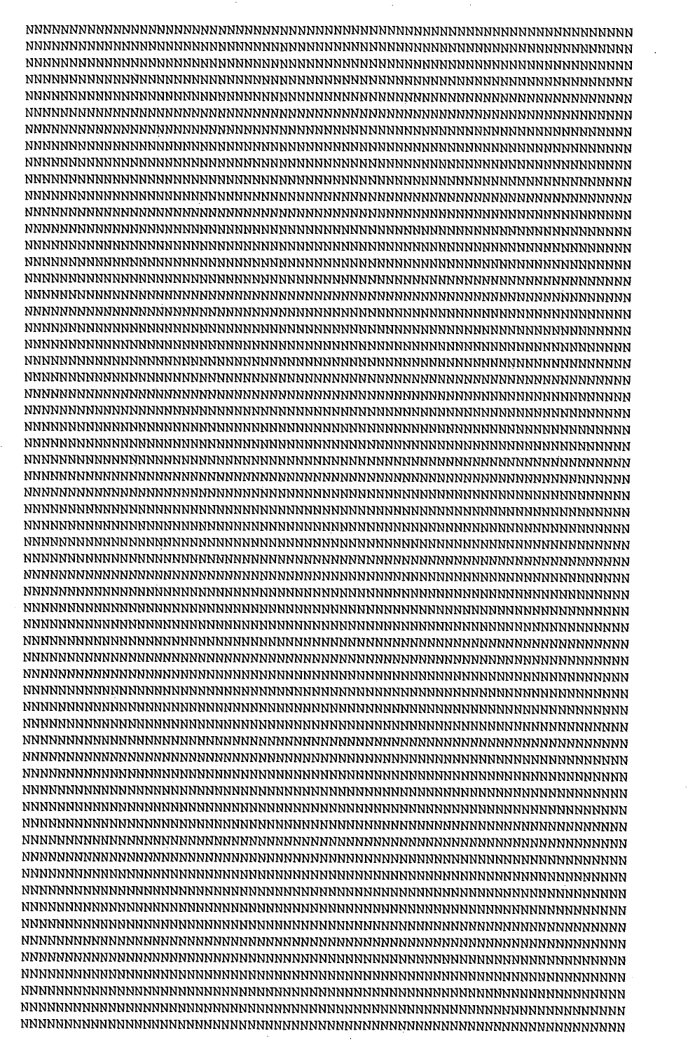

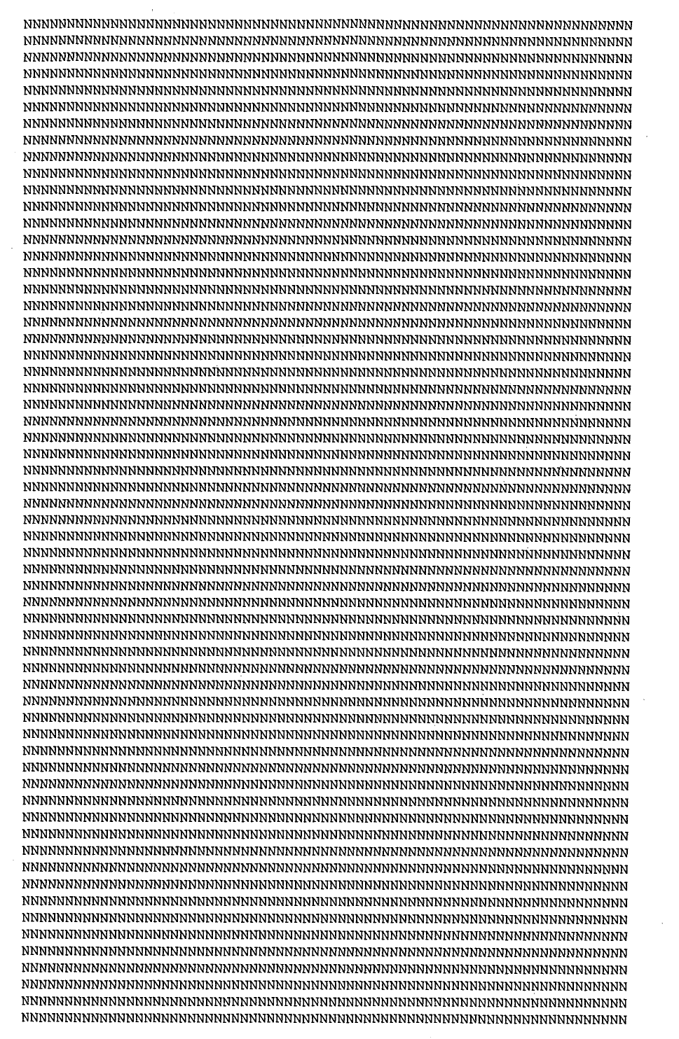

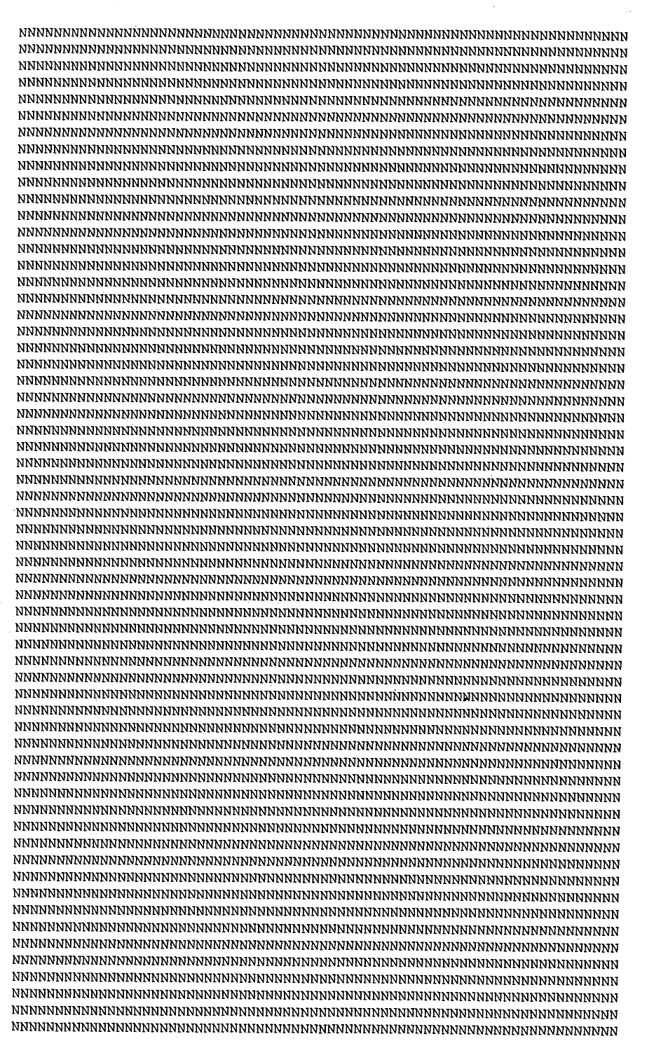

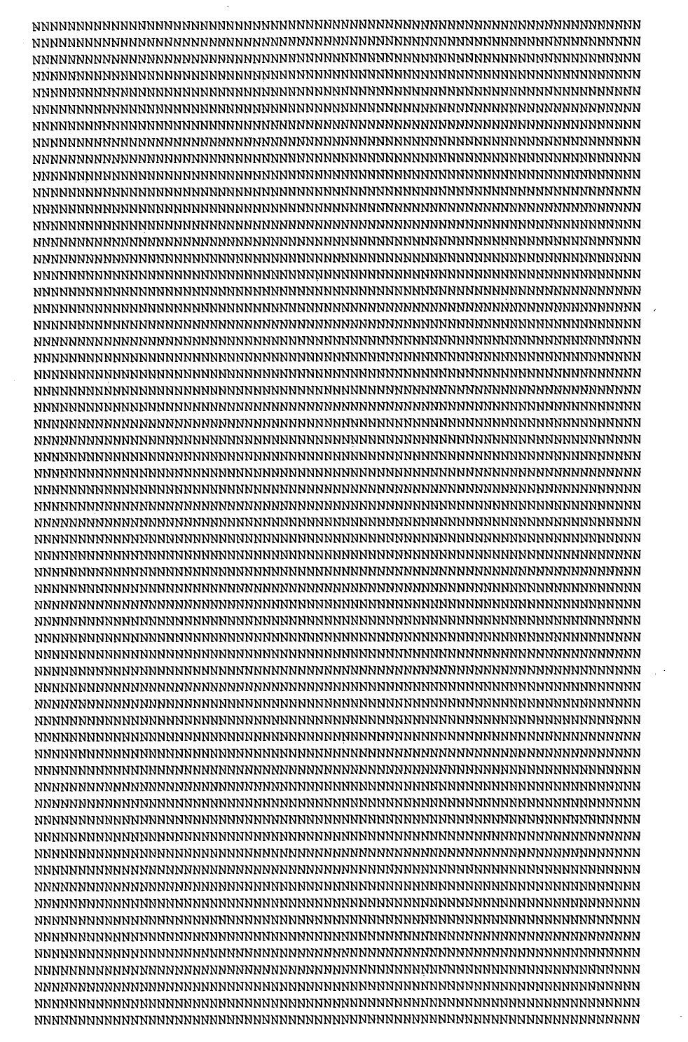

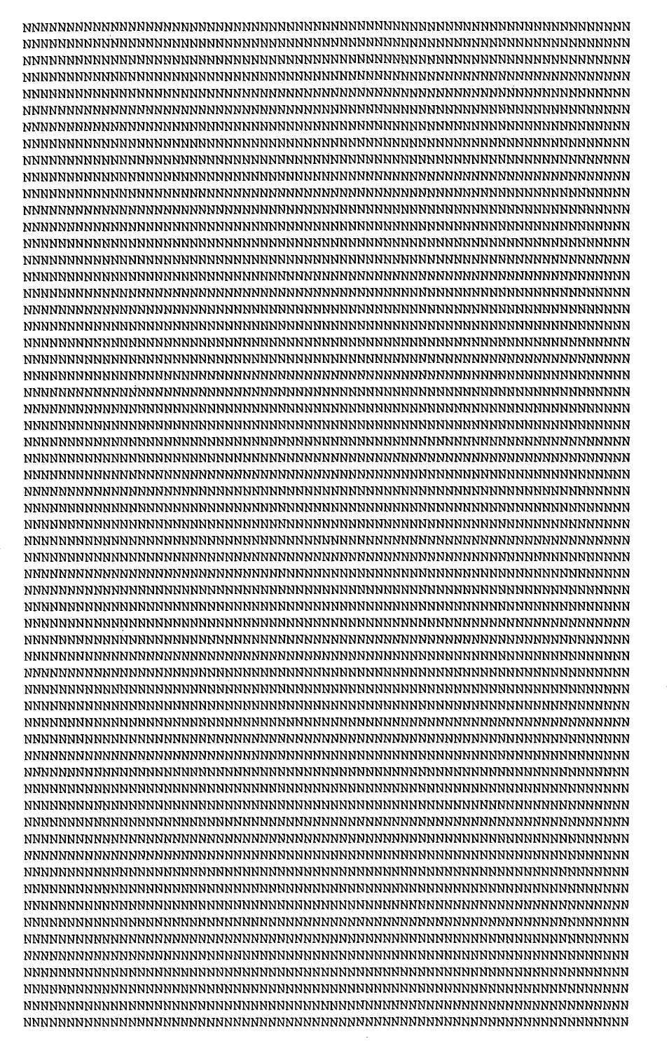

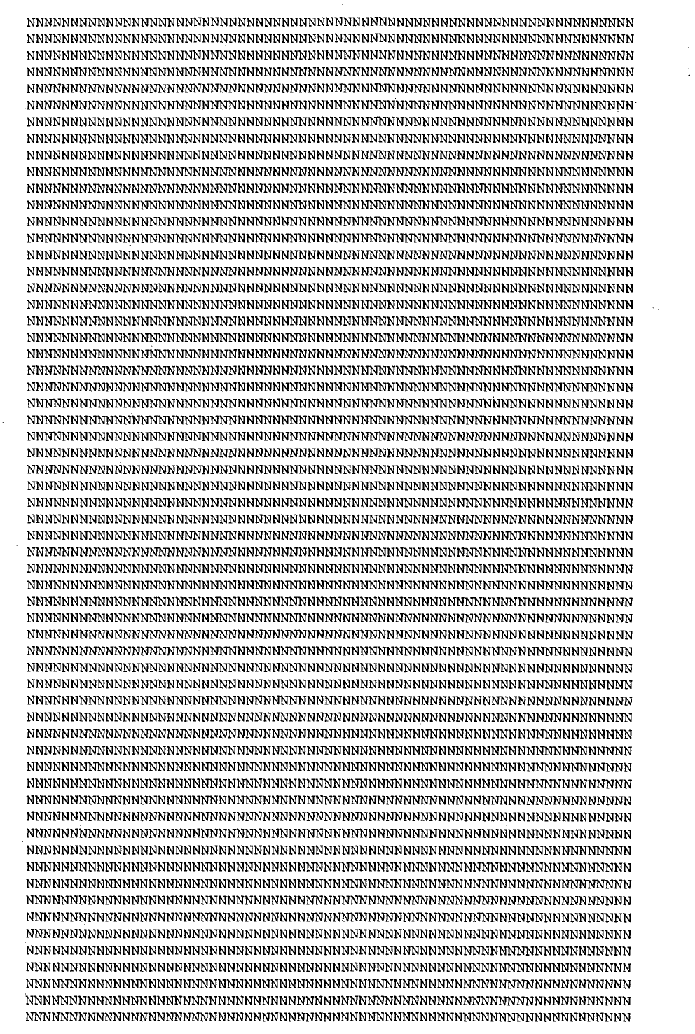

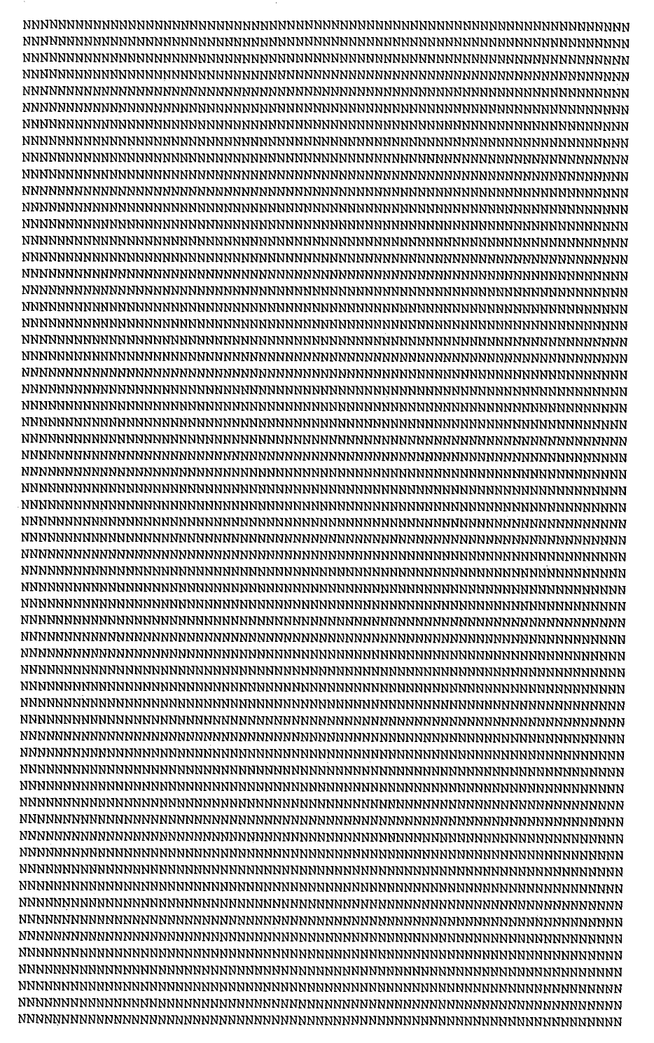

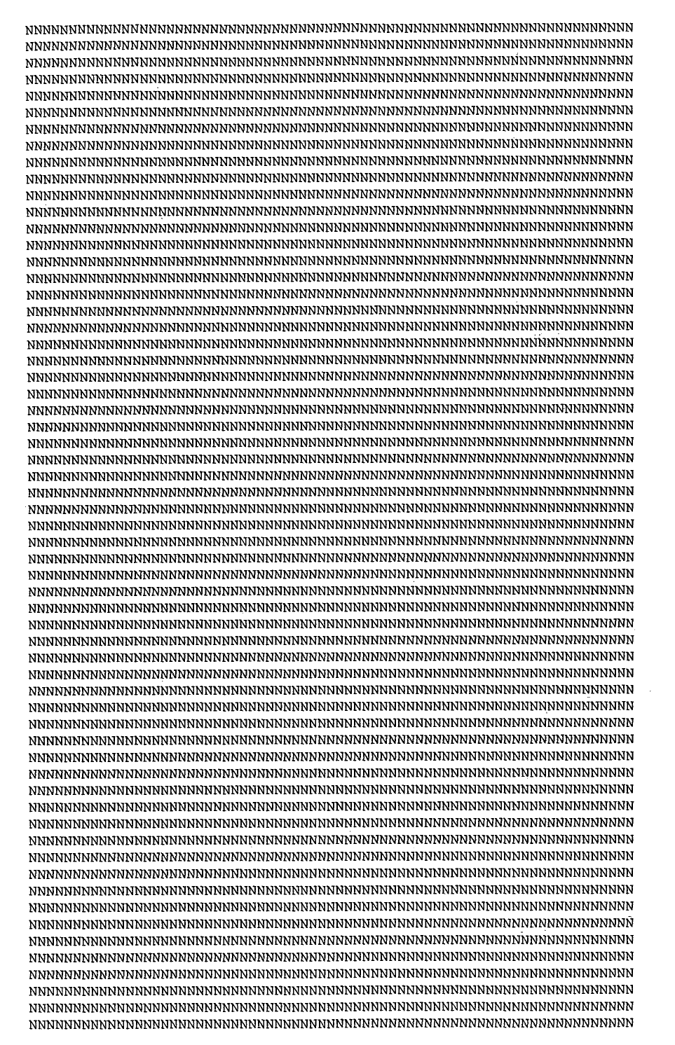

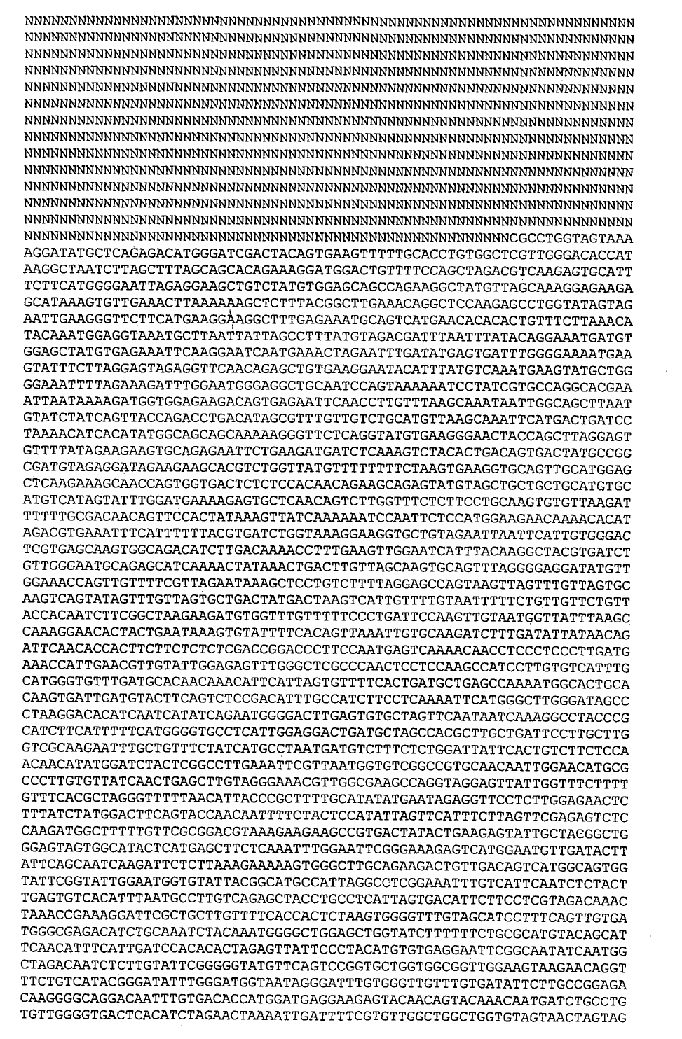

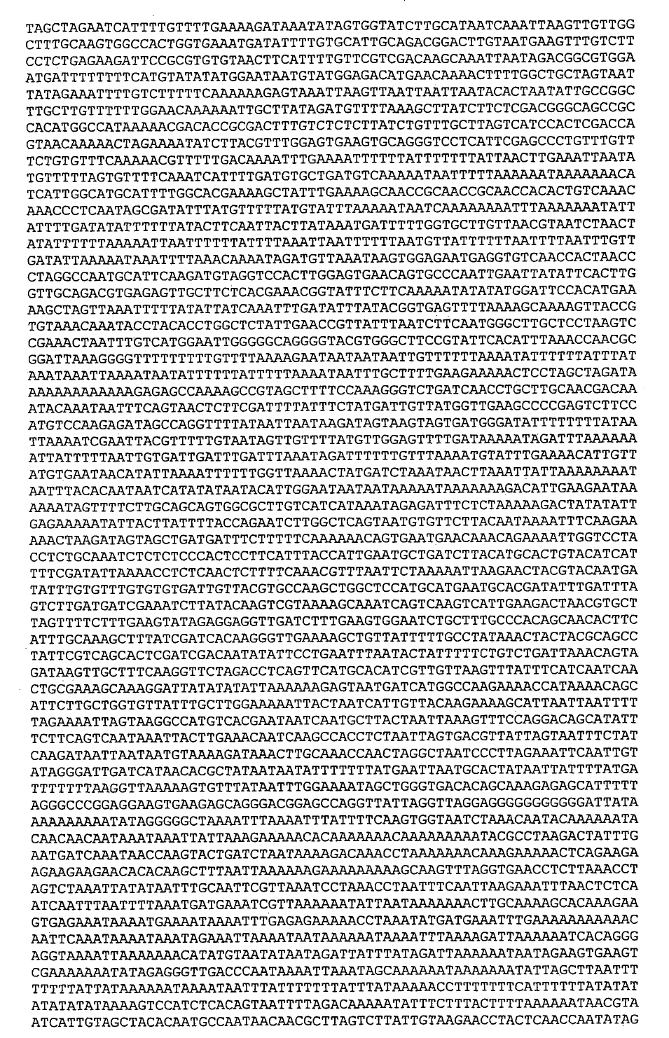

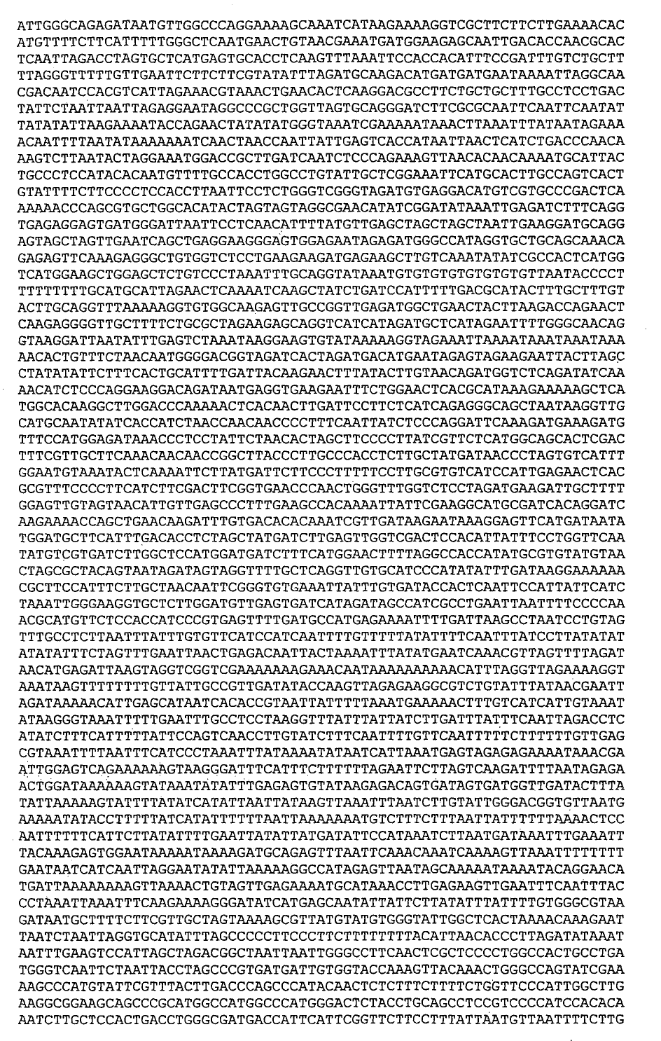

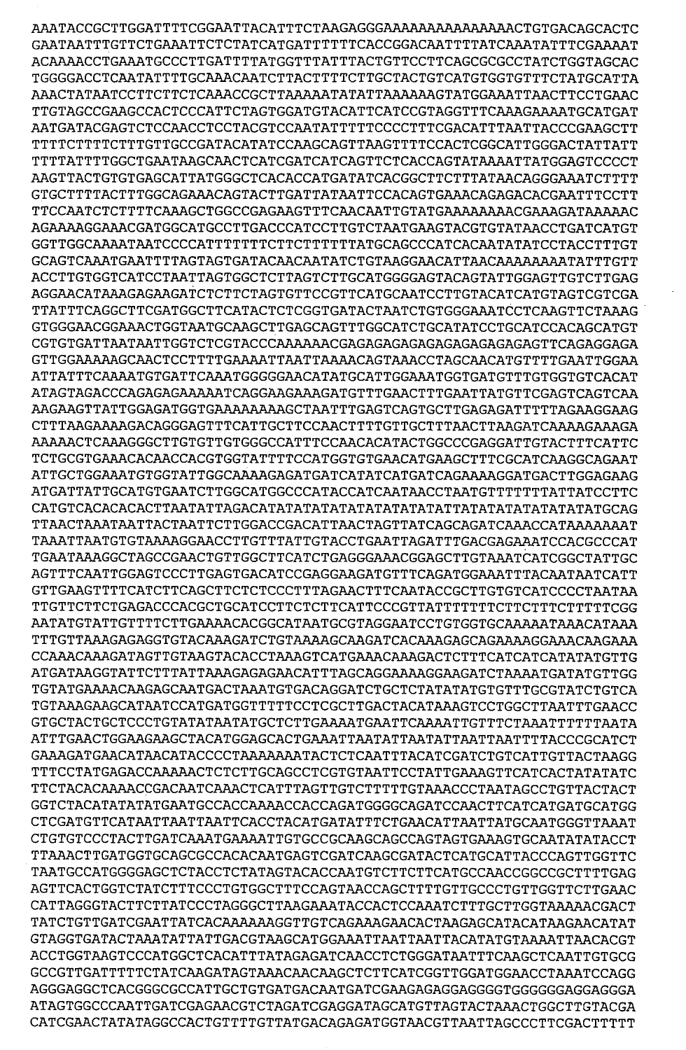

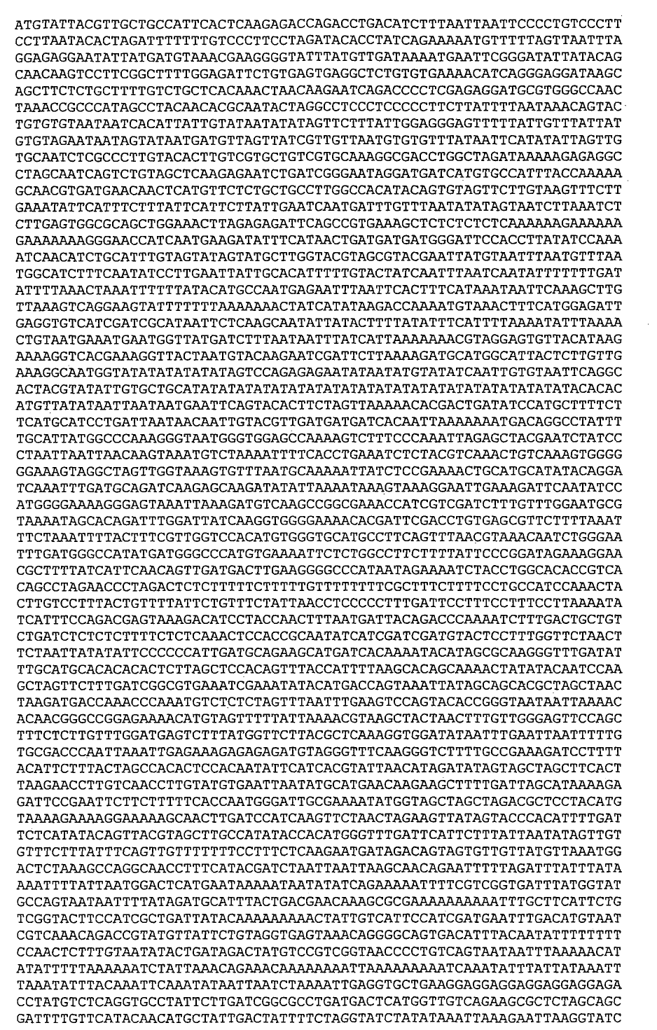

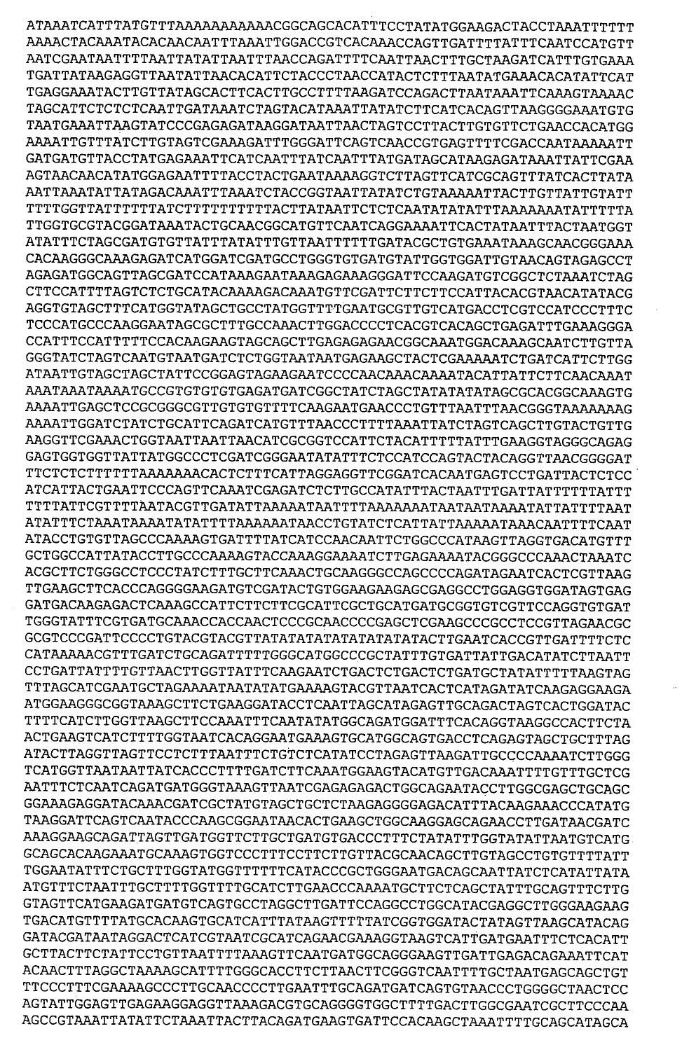

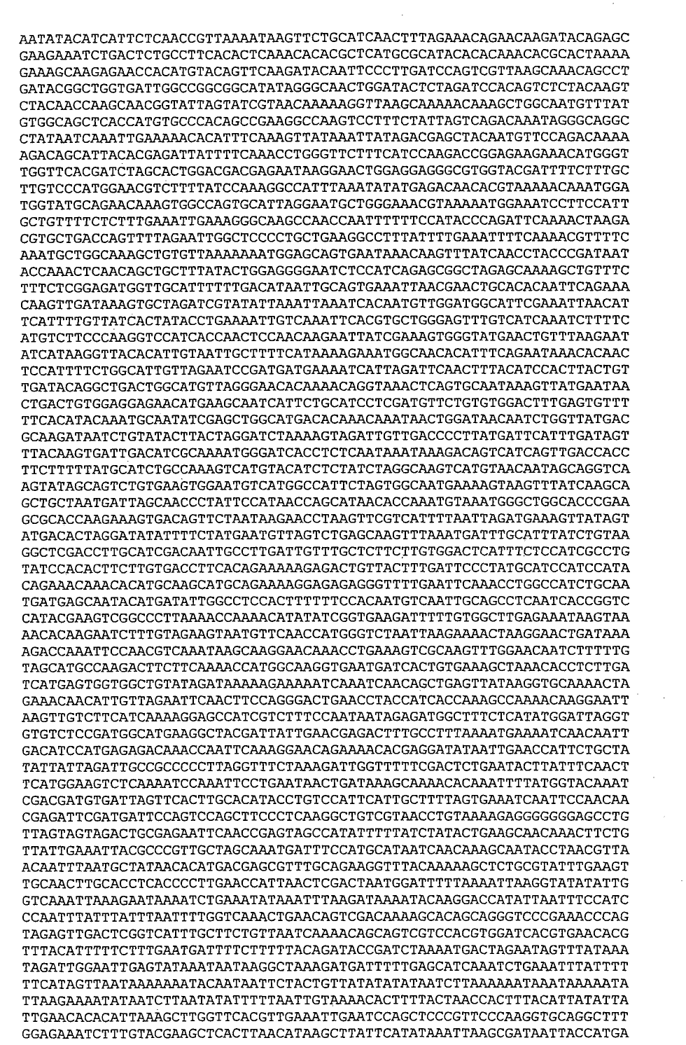

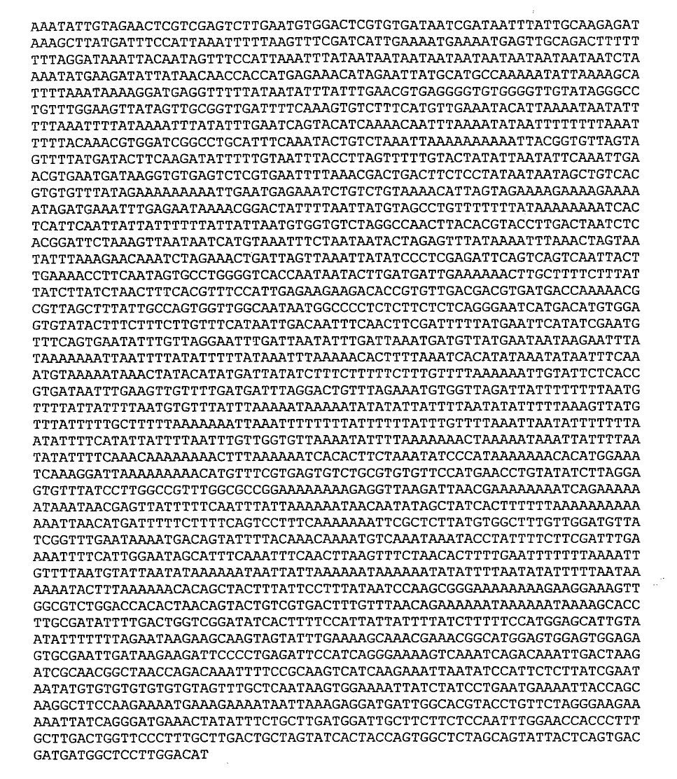

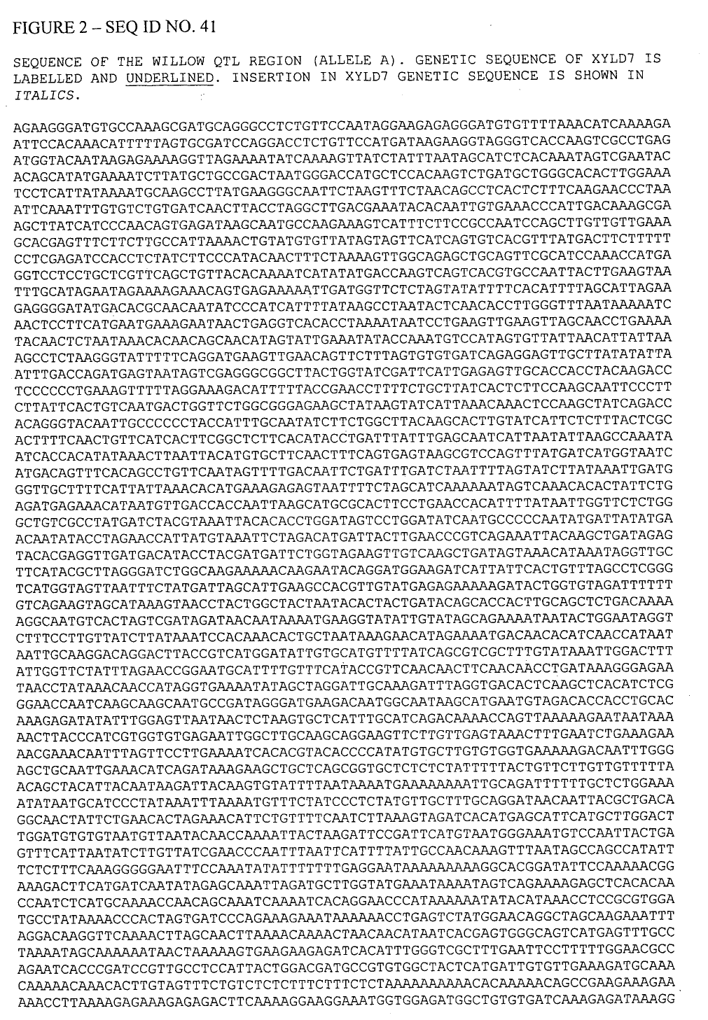

FIG. 2 shows the sequence of a QTL region in Salix associated with improved yield. The sequence is derived from allele A.

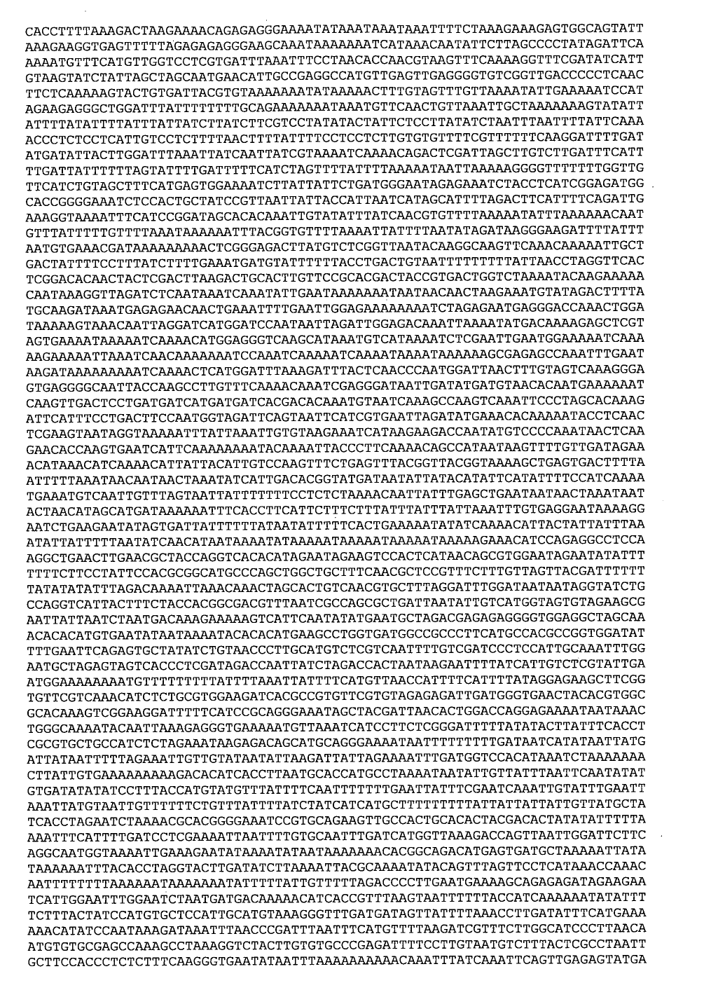

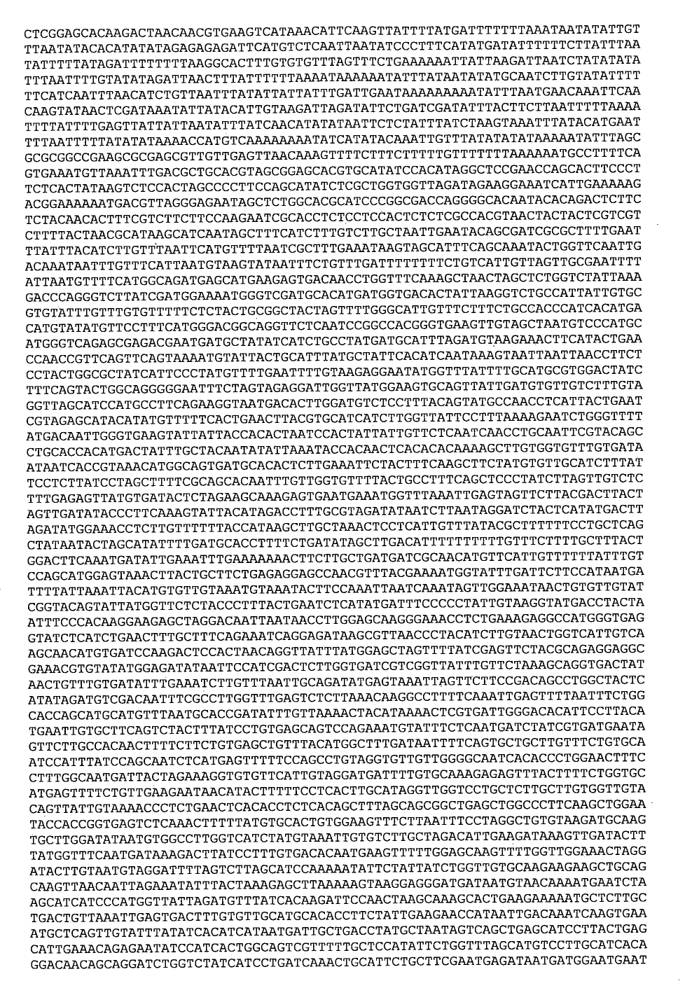

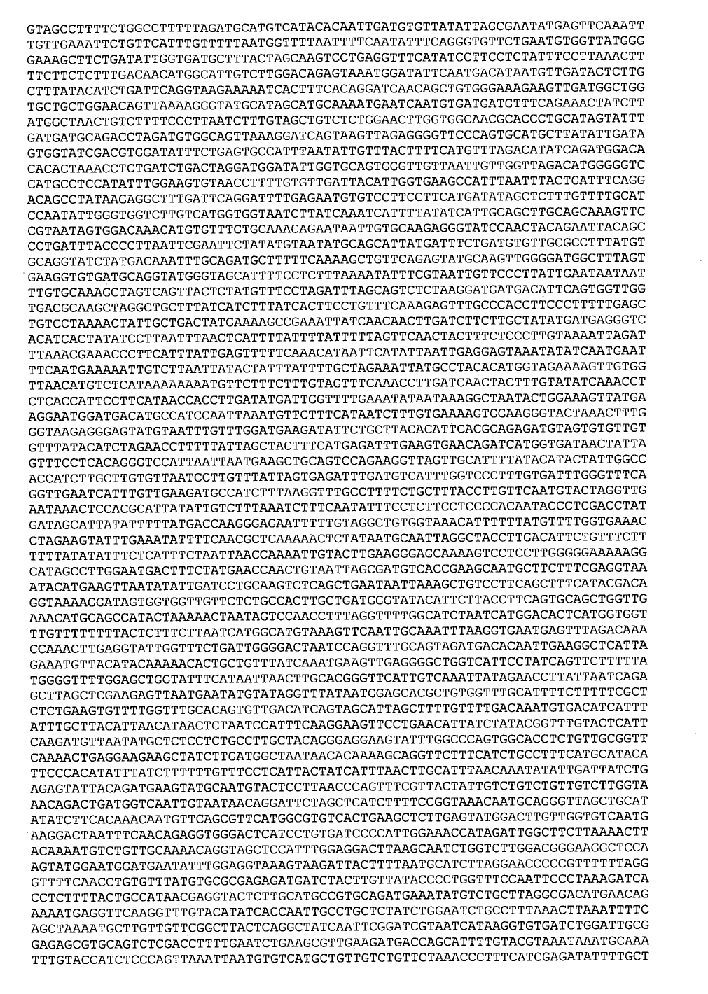

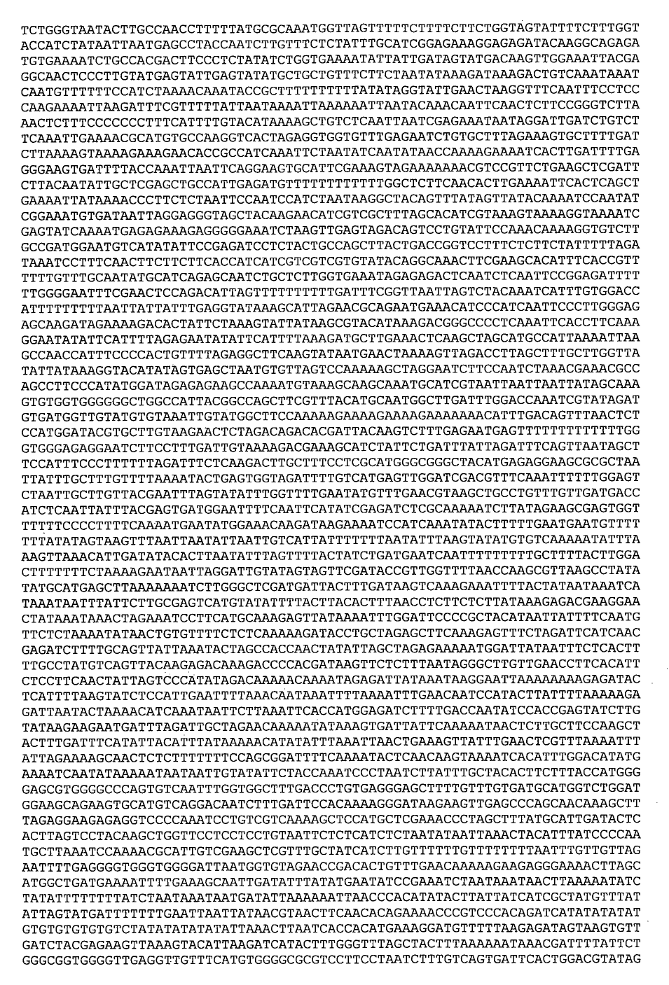

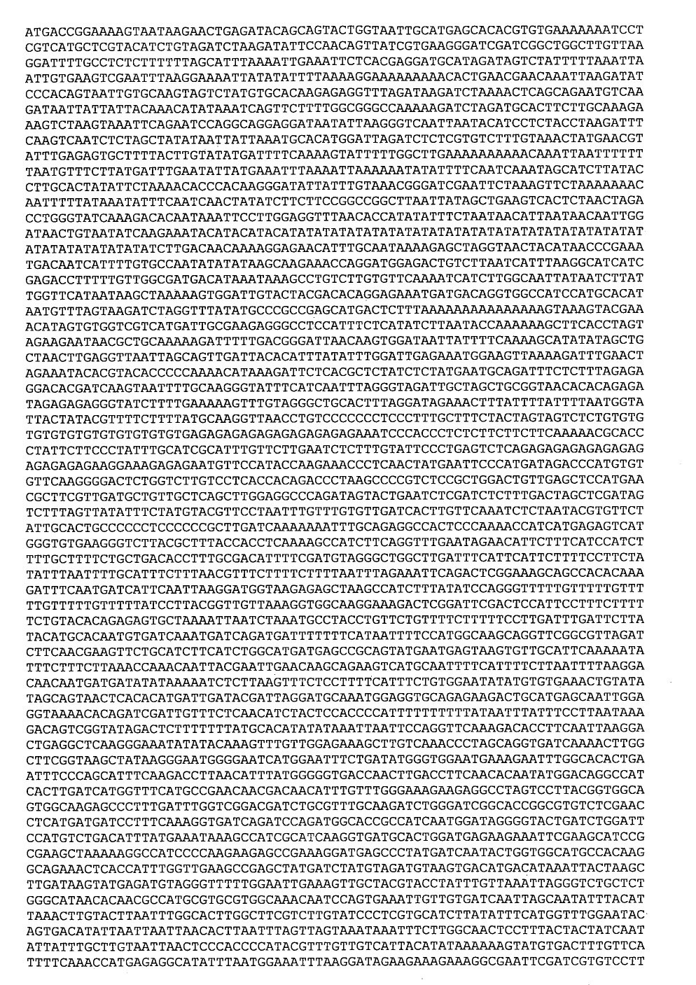

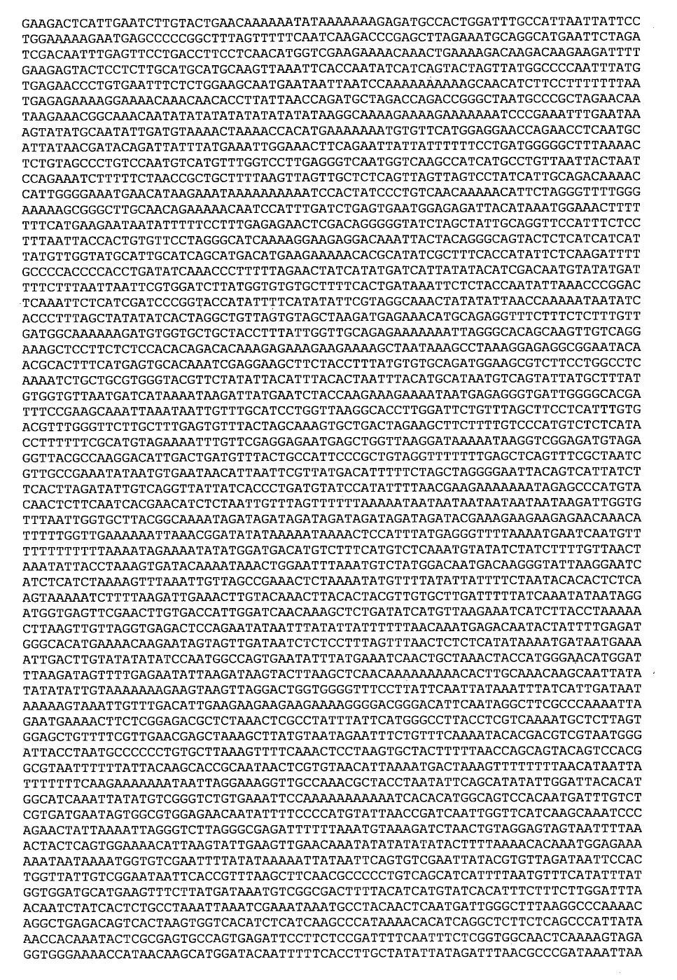

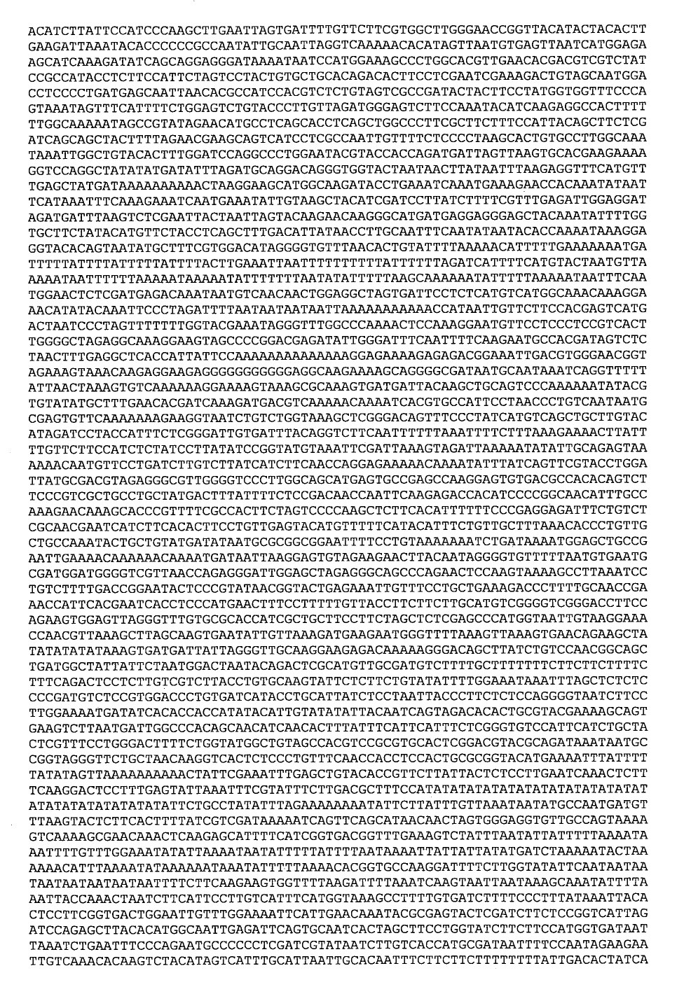

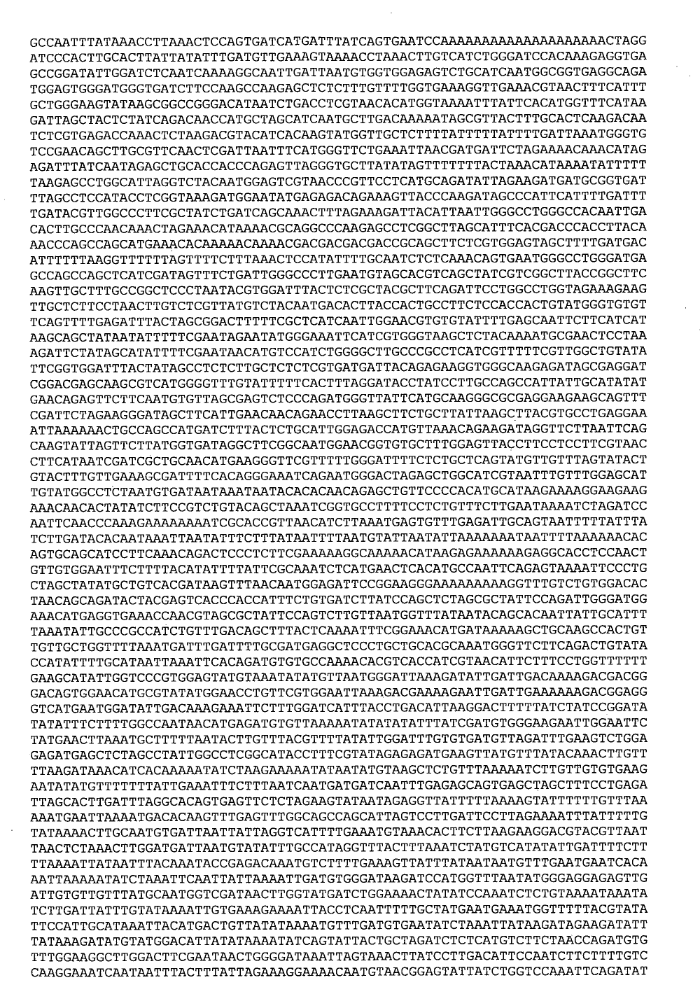

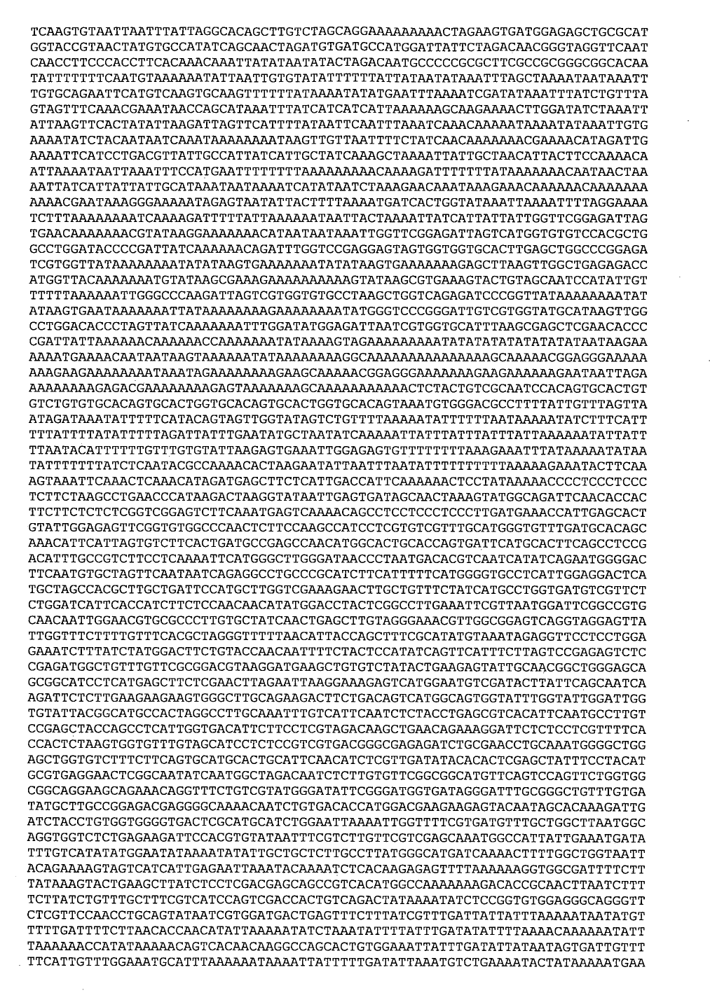

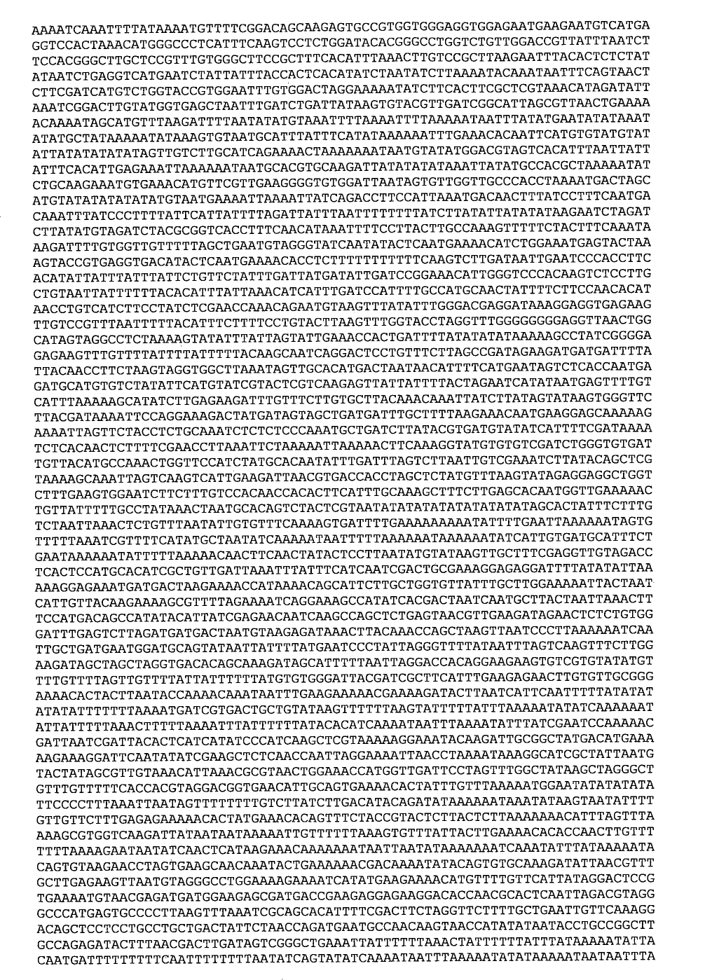

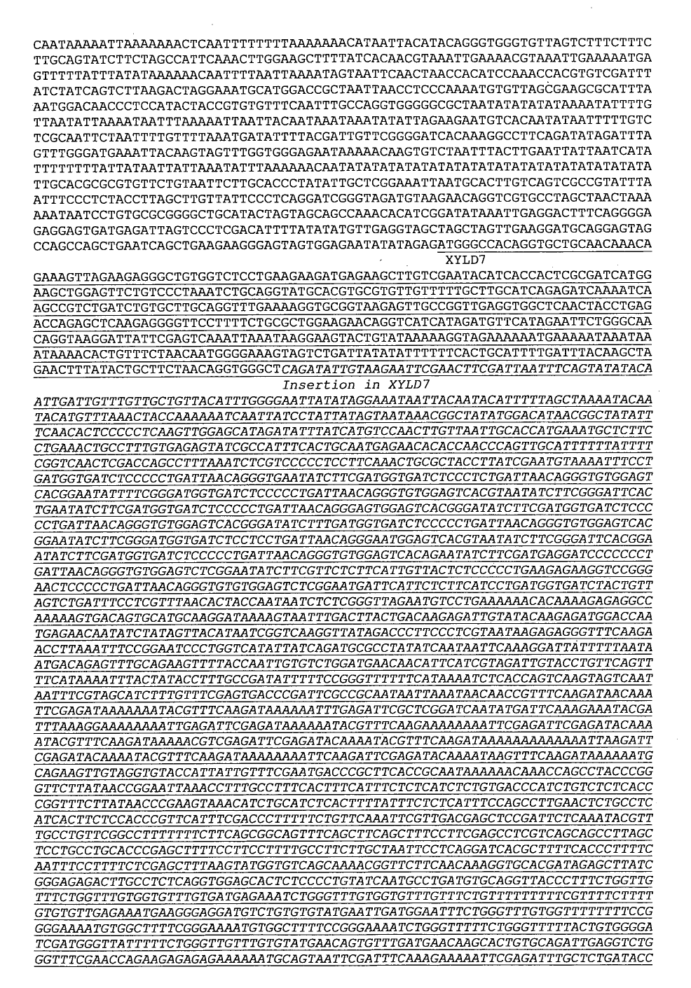

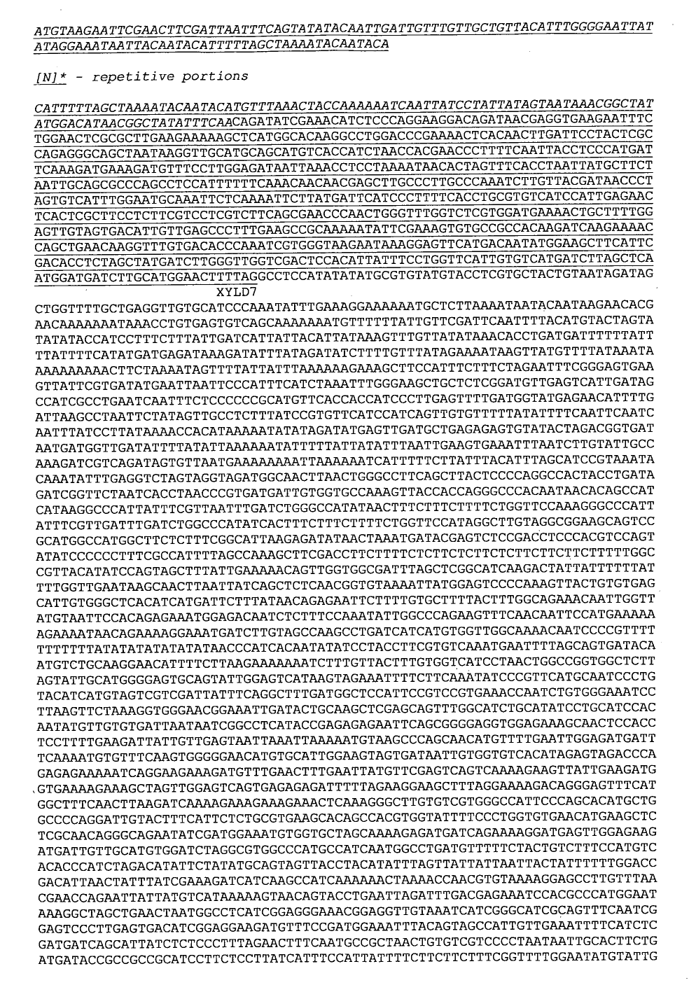

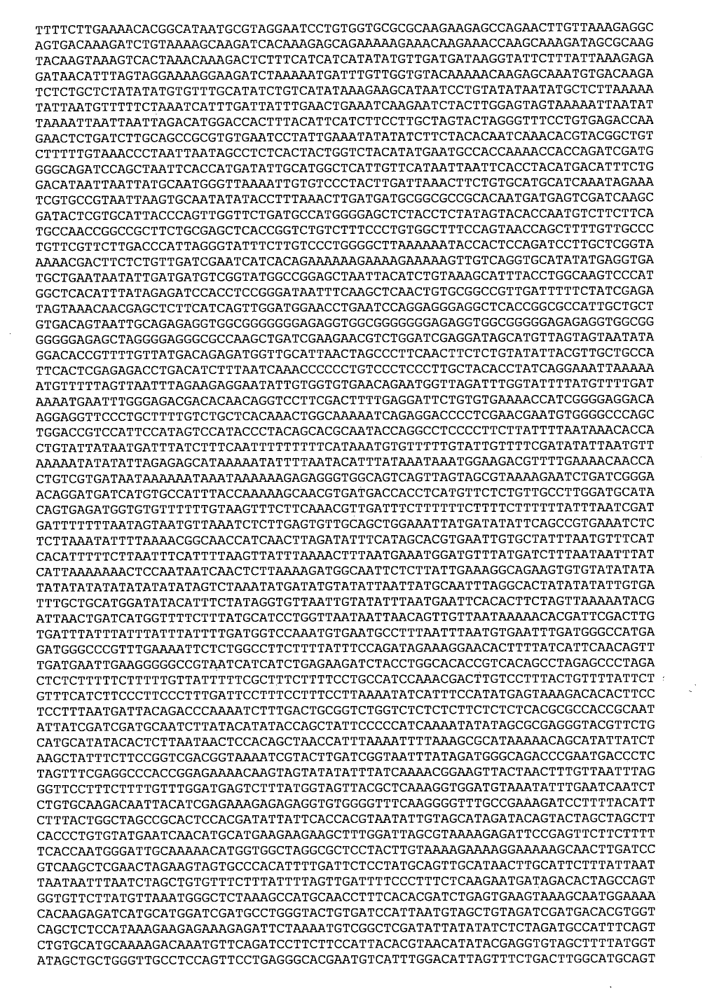

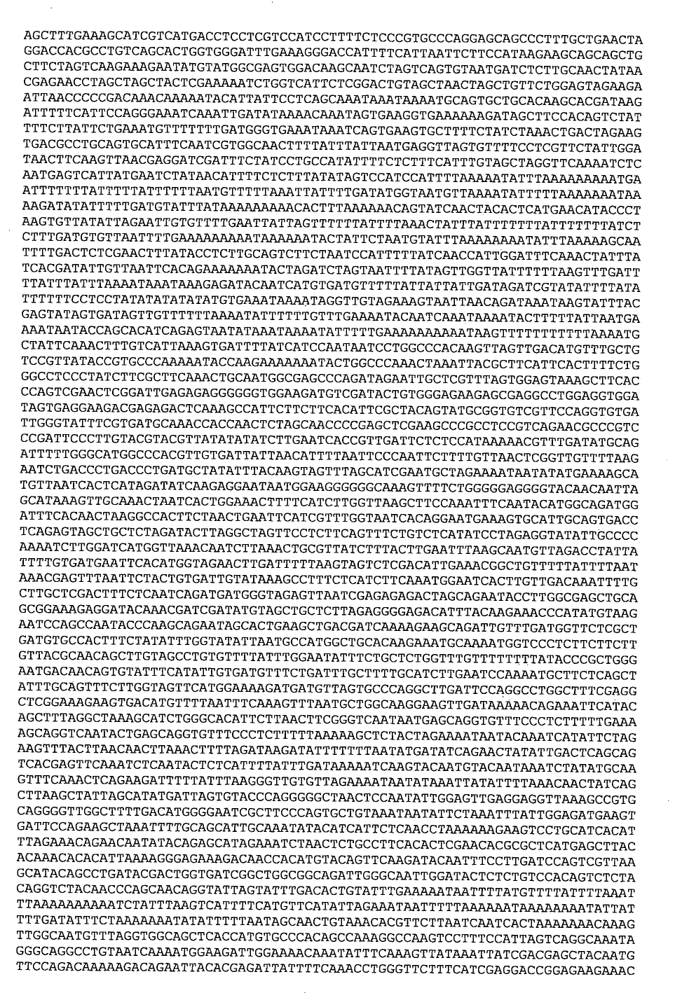

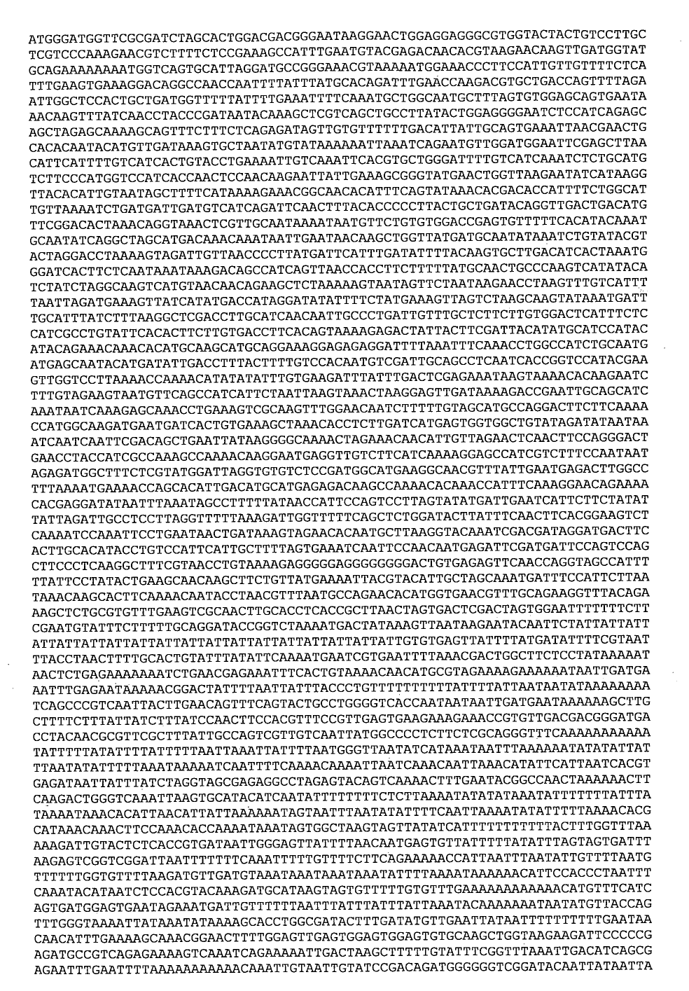

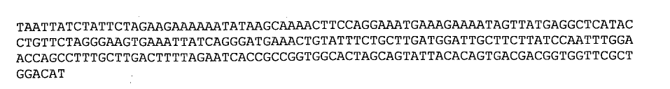

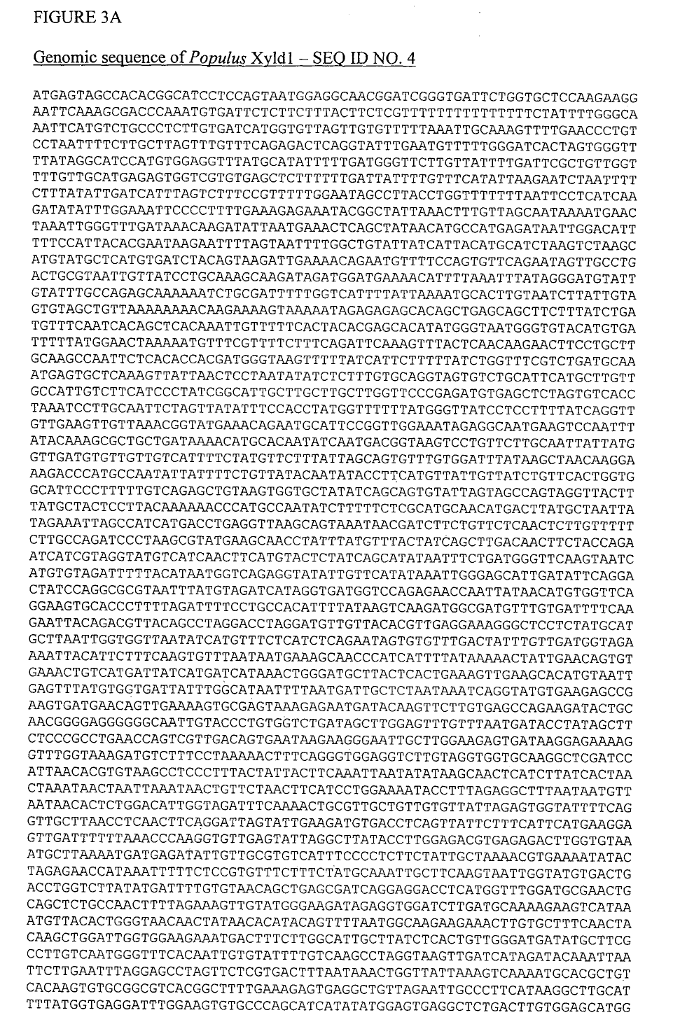

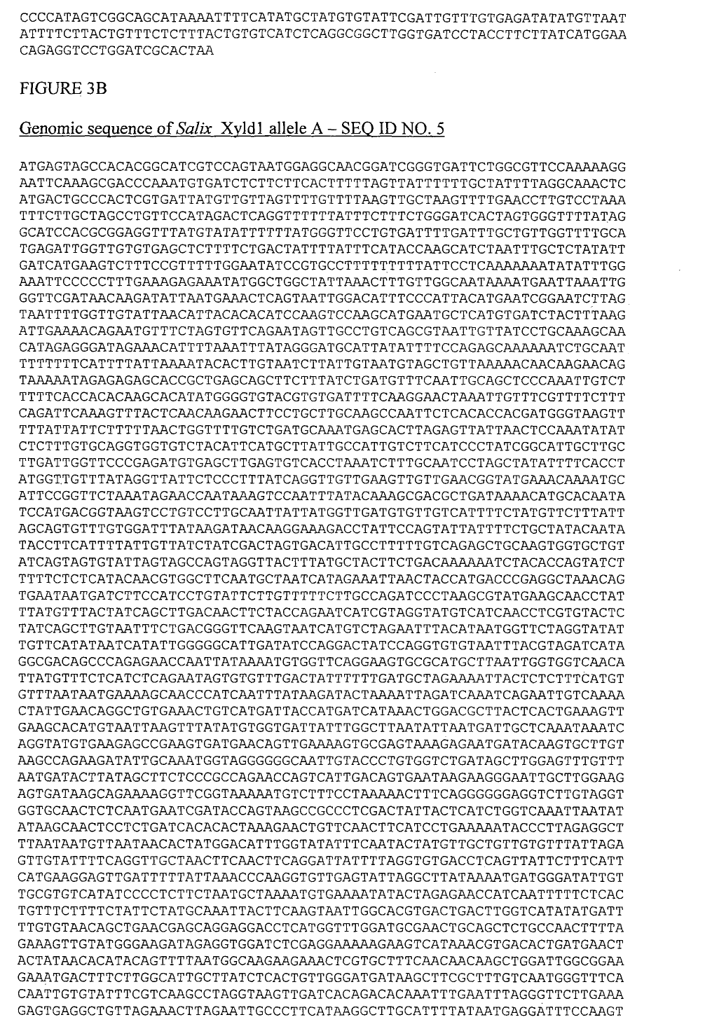

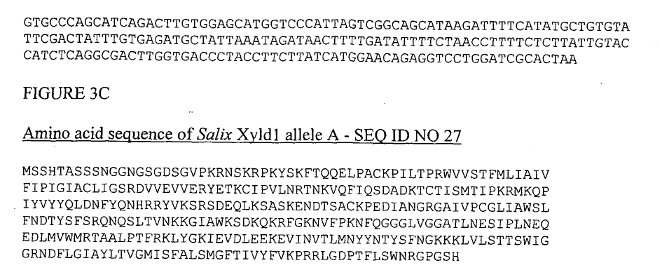

FIG. 3A: shows the nucleotide sequence of the Xyld1 polynucleotide of Populus (SEQ ID NO 4). SEQ ID NO 4 is located within the QTL region shown in FIG. 1.

FIG. 3B: shows the nucleotide sequence of the Xyld1 allele A polynucleotide of Salix (SEQ ID NO 5).

FIG. 3C: shows the amino acid sequence of the Xyld1 allele A polypeptide of Salix (SEQ ID NO 27).

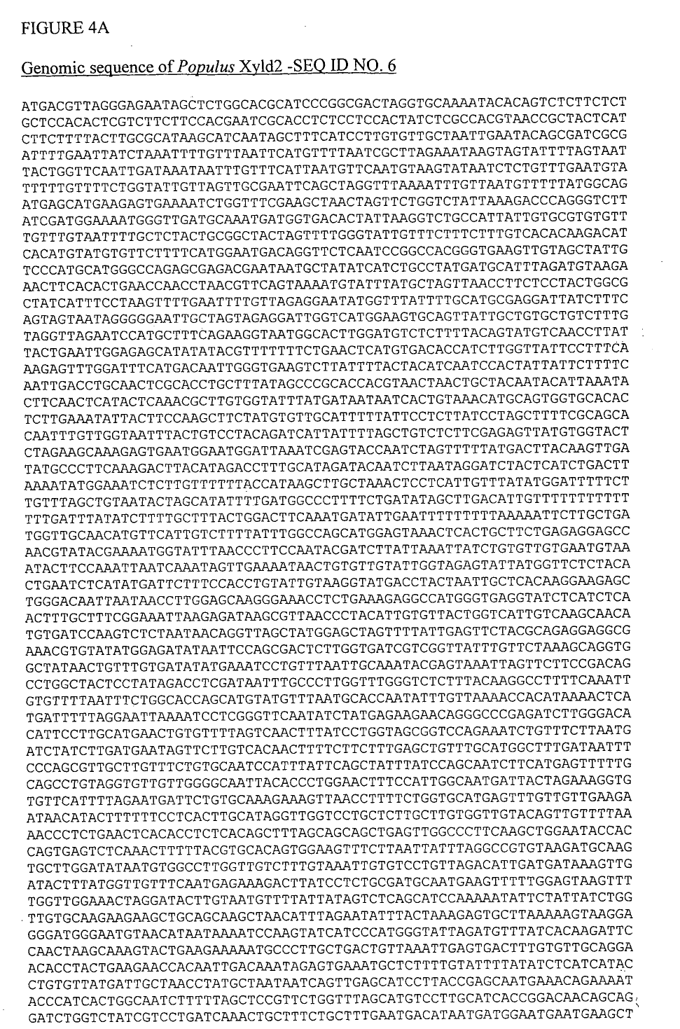

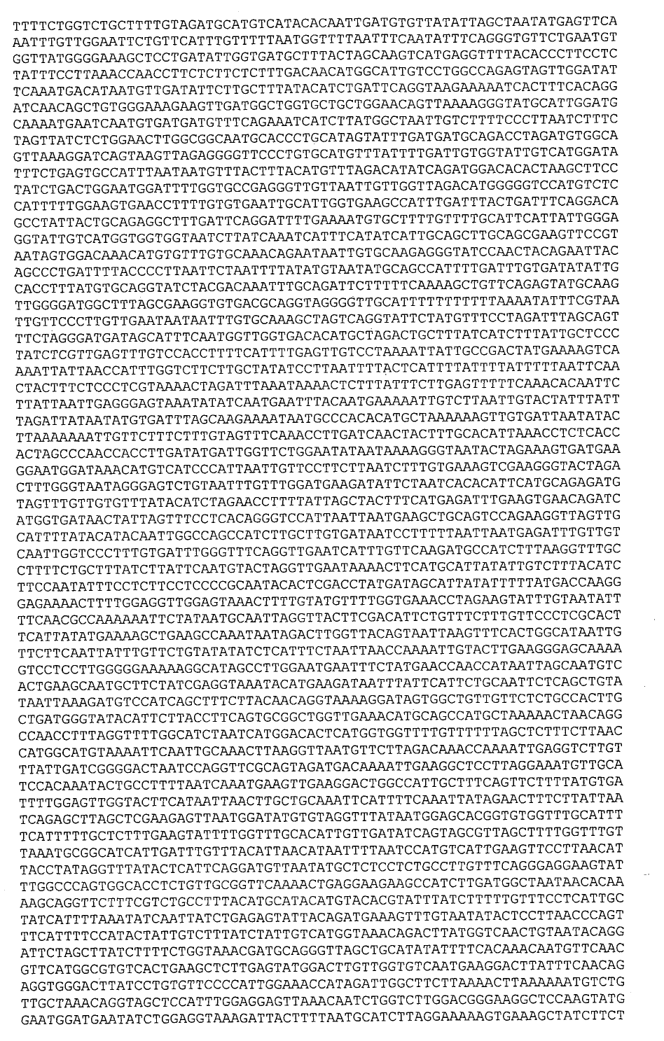

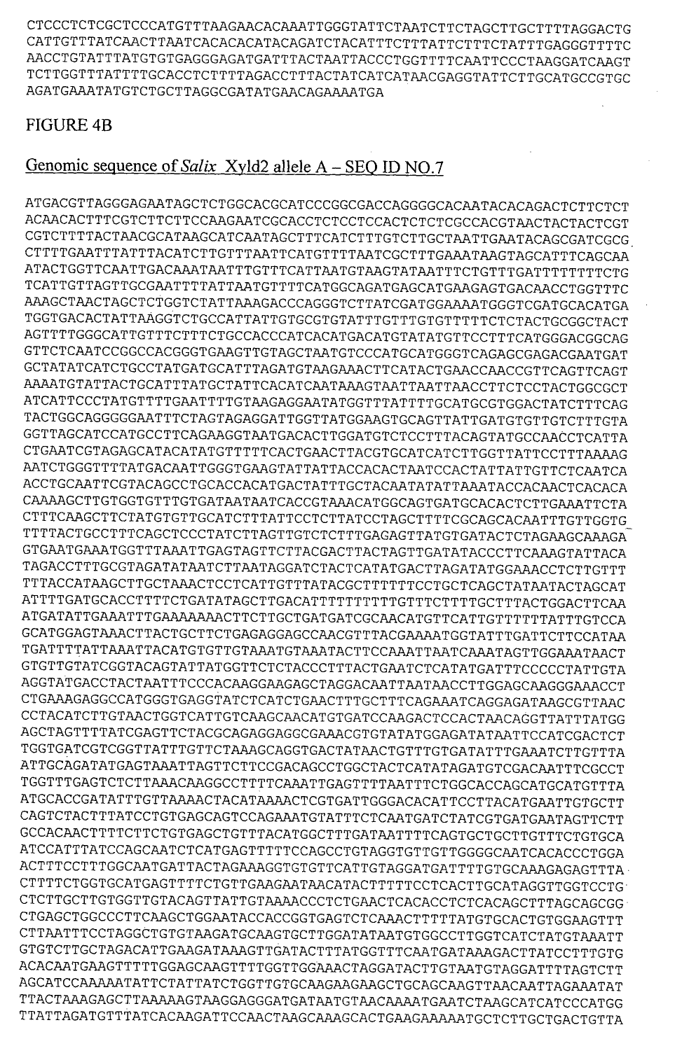

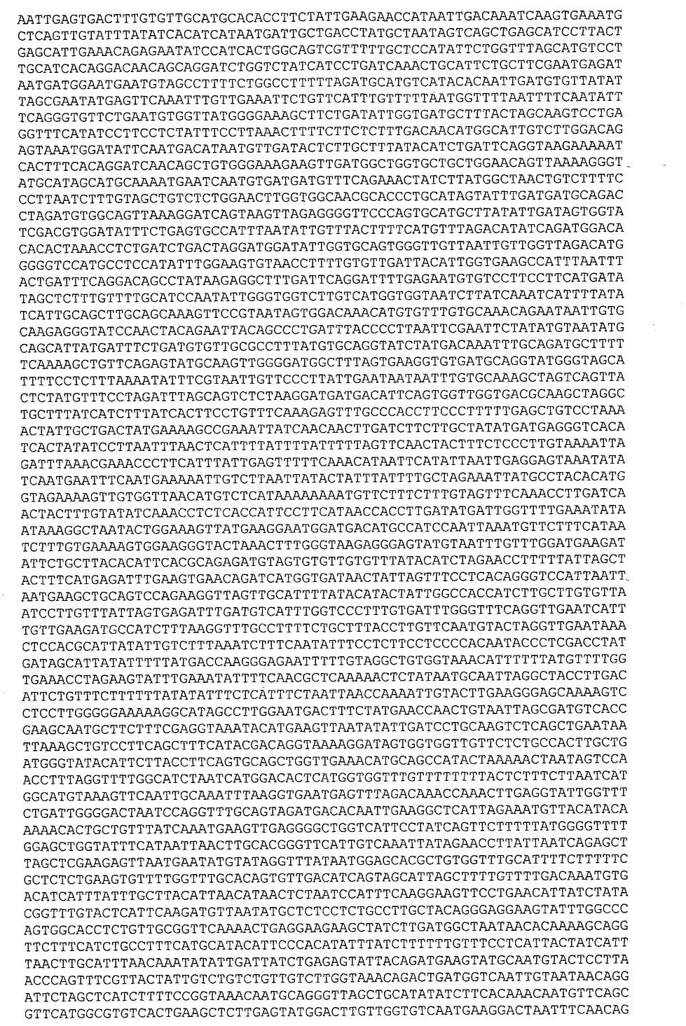

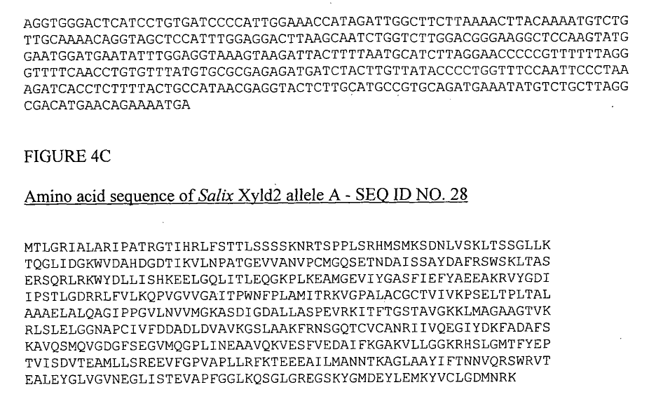

FIG. 4A: shows the nucleotide sequence of the Xyld2 polynucleotide of Populus (SEQ ID NO 6). SEQ ID NO 6 is located within the QTL region shown in FIG. 1.

FIG. 4B: shows the nucleotide sequence of the Xyld2 allele A polynucleotide of Salix (SEQ ID NO 7).

FIG. 4C: shows the amino acid sequence of the Xyld2 allele A polypeptide of Salix (SEQ ID NO 28).

FIG. 5A: shows the nucleotide sequence of the Xyld3 polynucleotide of Populus (SEQ ID NO 8). SEQ ID NO 8 is located within the QTL region shown in FIG. 1.

FIG. 5B: shows the nucleotide sequence of the Xyld3 allele A polynucleotide of Salix (SEQ ID NO 9).

FIG. 5C: shows the amino acid sequence of the Xyld3 allele A polypeptide of Salix (SEQ ID NO 29).

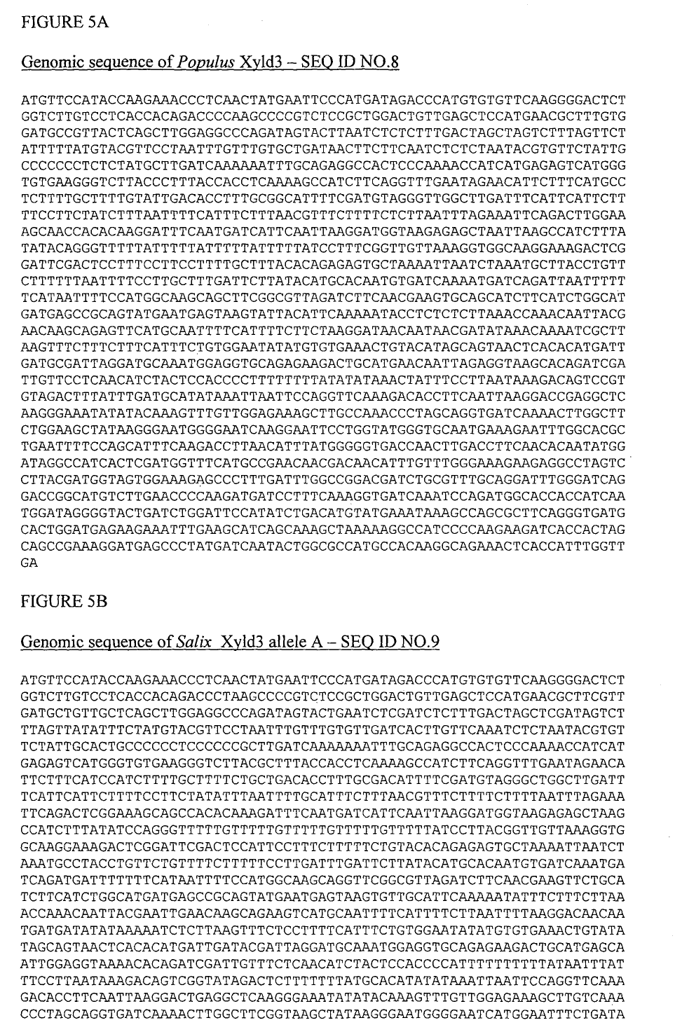

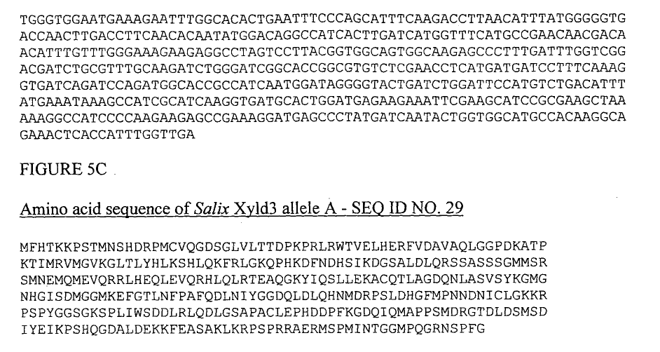

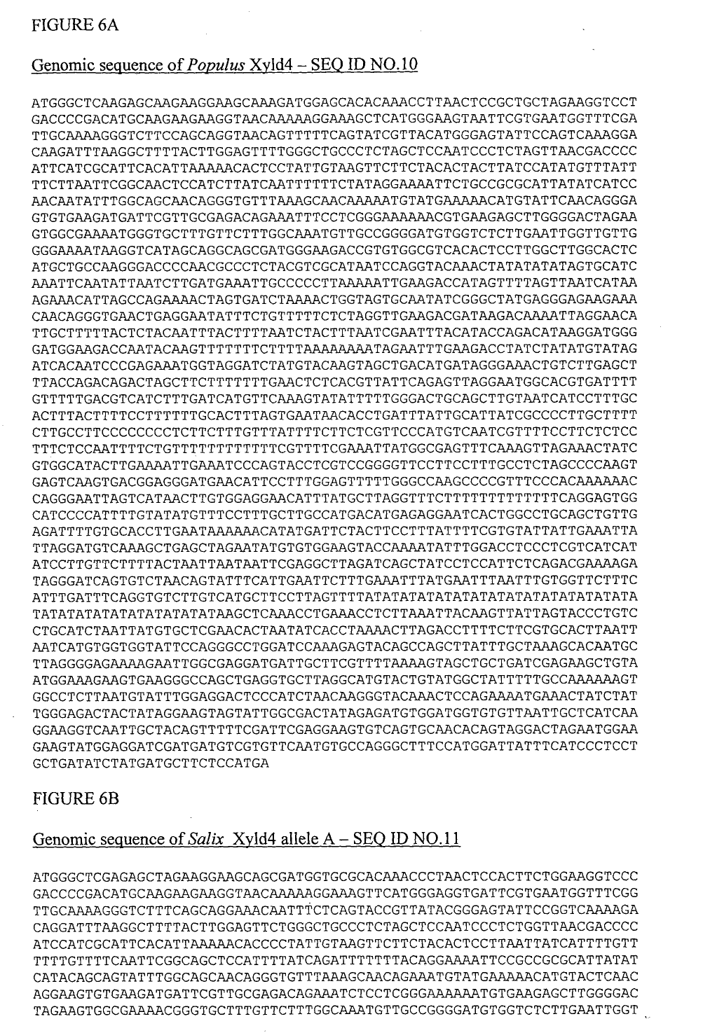

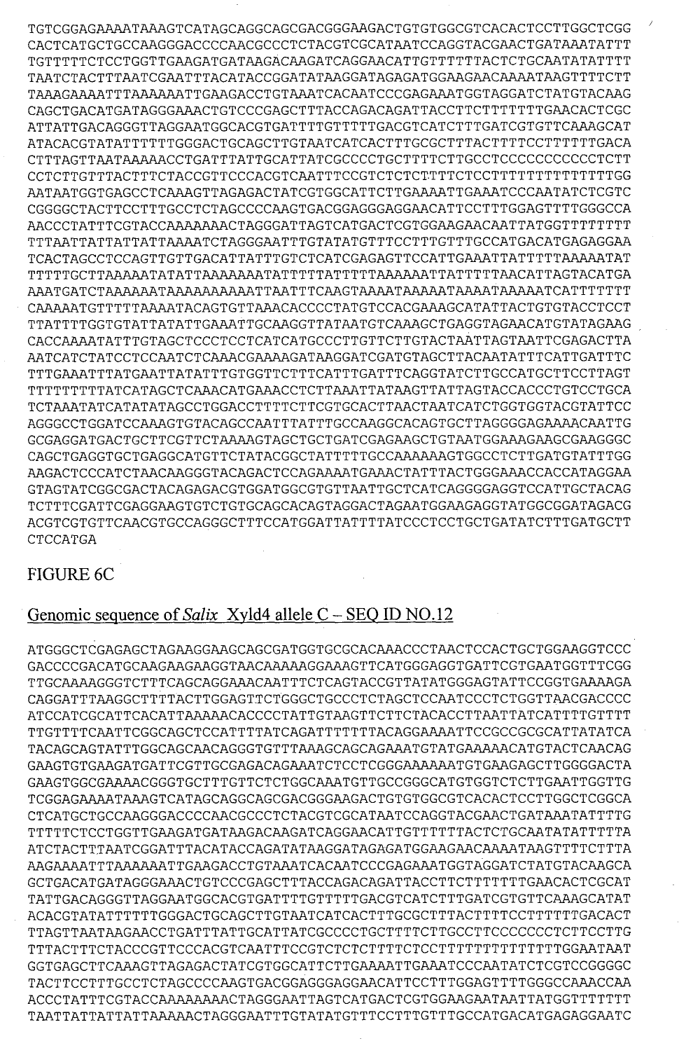

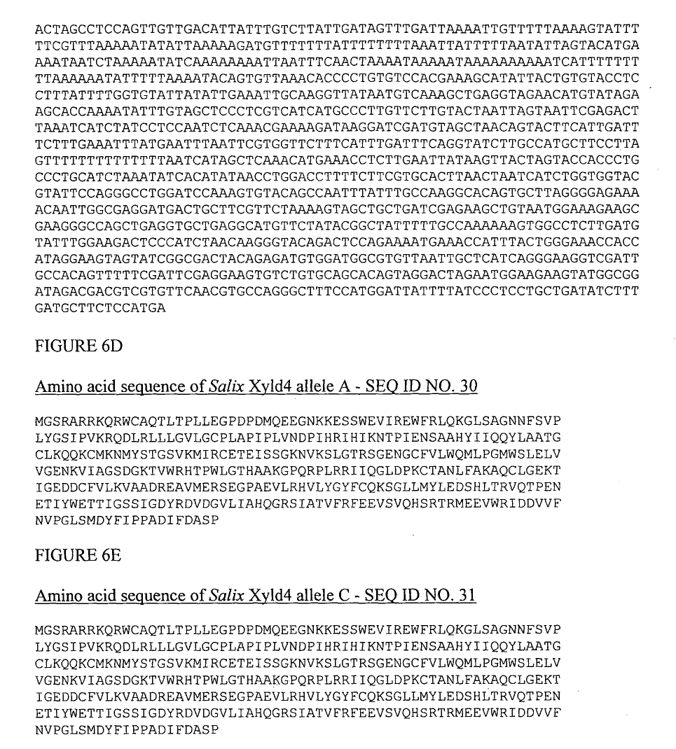

FIG. 6A: shows the nucleotide sequence of the Xyld4 polynucleotide of Populus (SEQ ID NO 10). SEQ ID NO 10 is located within the QTL region shown in FIG. 1.

FIG. 6B: shows the nucleotide sequence of the Xyld4 allele A polynucleotide of Salix (SEQ ID NO 11).

FIG. 6C: shows the nucleotide sequence of the Xyld4 allele C polynucleotide of Salix (SEQ ID NO 12).

FIG. 6D: shows the amino acid sequence of the Xyld4 allele A polypeptide of Salix (SEQ ID NO 30).

FIG. 6E: shows the amino acid sequence of the Xyld4 allele C polypeptide of Salix (SEQ ID NO 31).

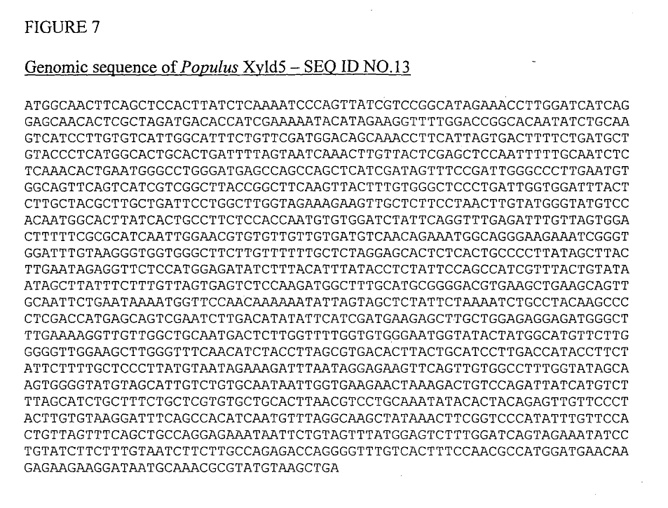

FIG. 7: shows the nucleotide sequence of the Xyld5 polynucleotide of Populus (SEQ ID NO 13). SEQ ID NO 13 is located within the QTL region shown in FIG. 1.

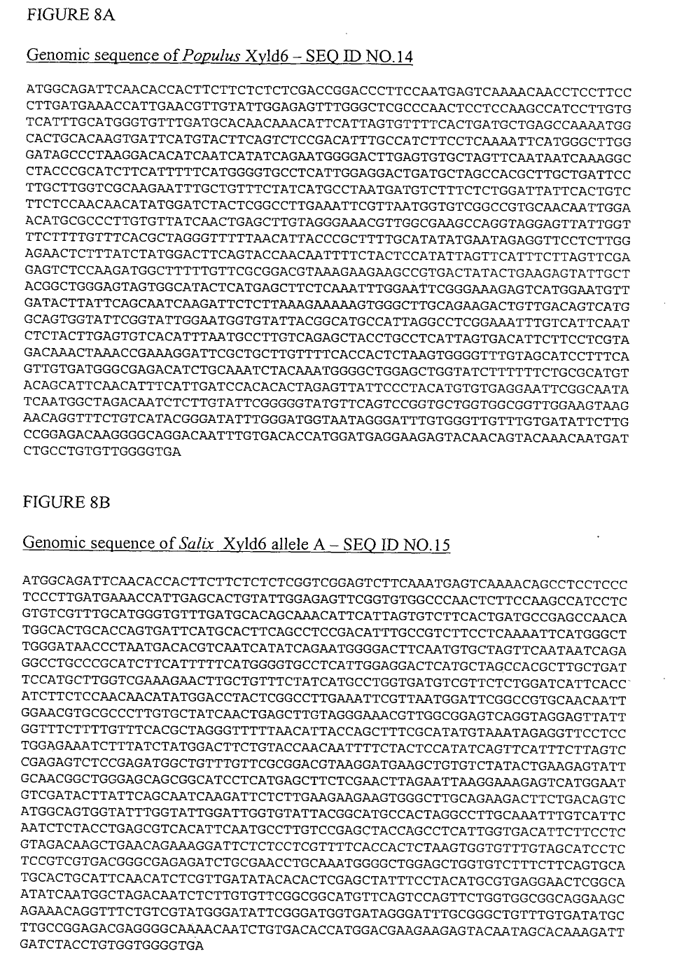

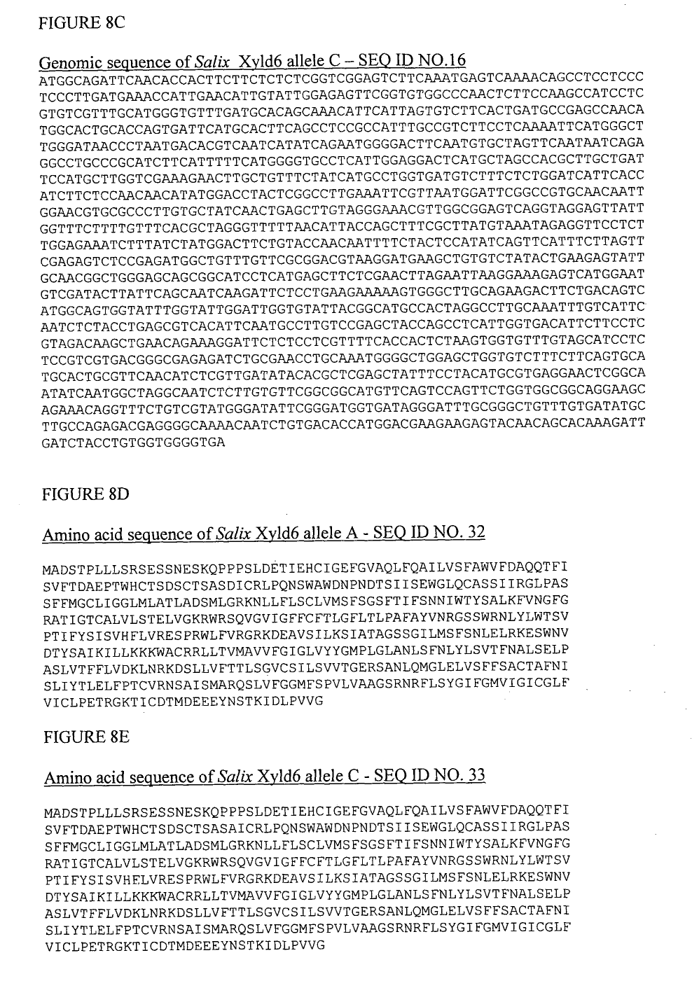

FIG. 8A: shows the nucleotide sequence of the Xyld6 polynucleotide of Populus (SEQ ID NO 14). SEQ ID NO 14 is located within the QTL region shown in FIG. 1.

FIG. 8B: shows the nucleotide sequence of the Xyld6 allele A polynucleotide of Salix (SEQ ID NO 15).

FIG. 8C: shows the nucleotide sequence of the Xyld6 allele C polynucleotide of Salix (SEQ ID NO 16).

FIG. 8D: shows the amino acid sequence of the Xyld6 allele A polypeptide of Salix (SEQ ID NO 32).

FIG. 8E: shows the amino acid sequence of the Xyld6 allele C polypeptide of Salix (SEQ ID NO 33).

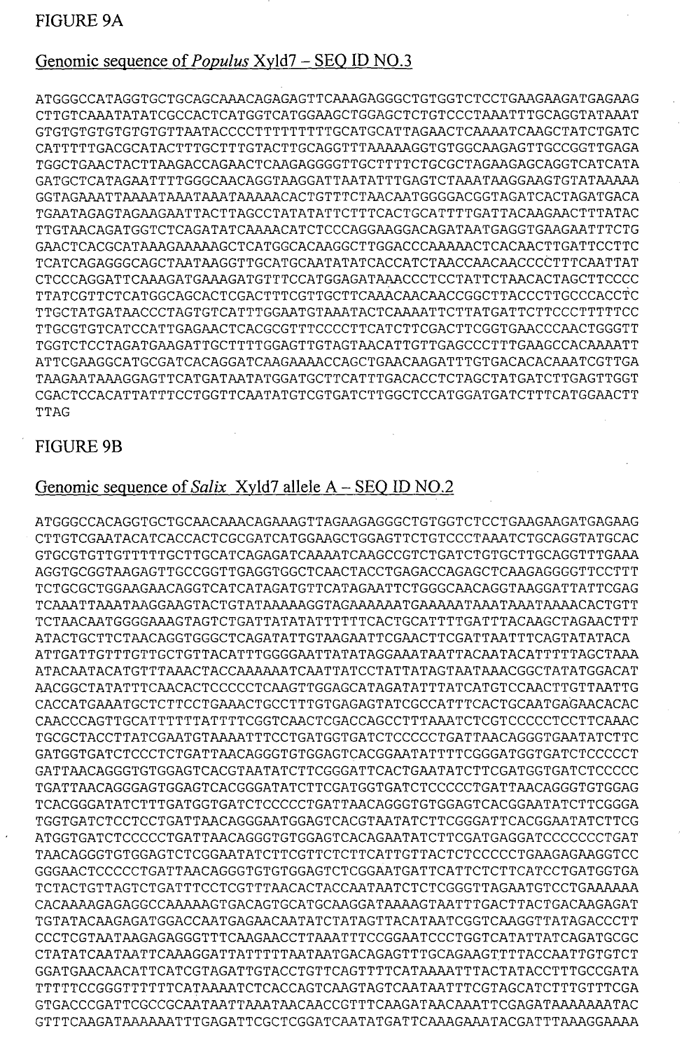

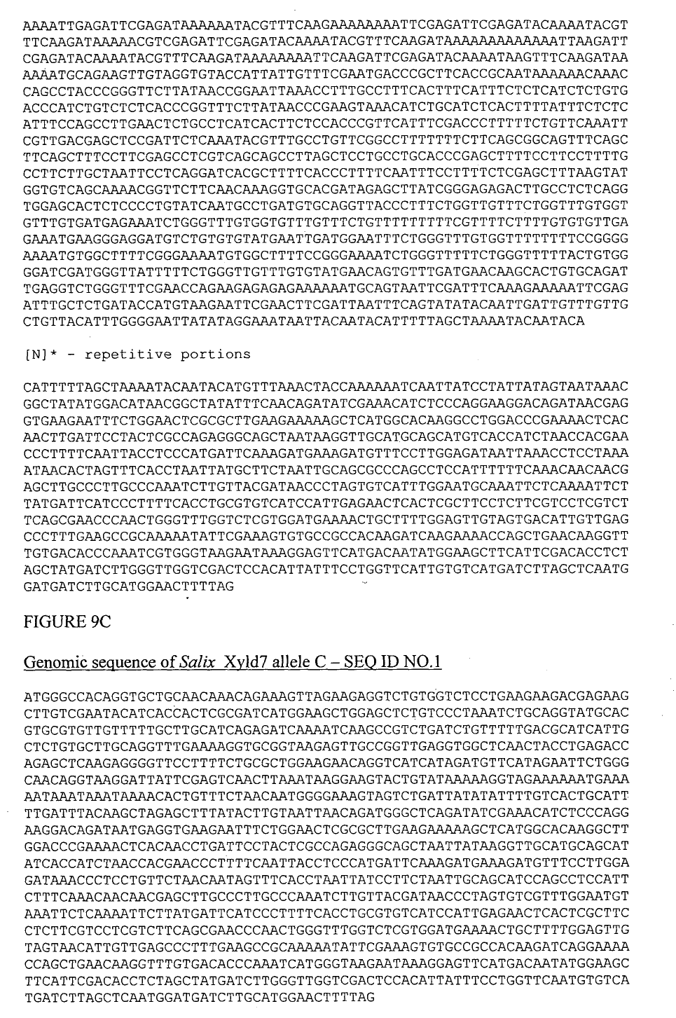

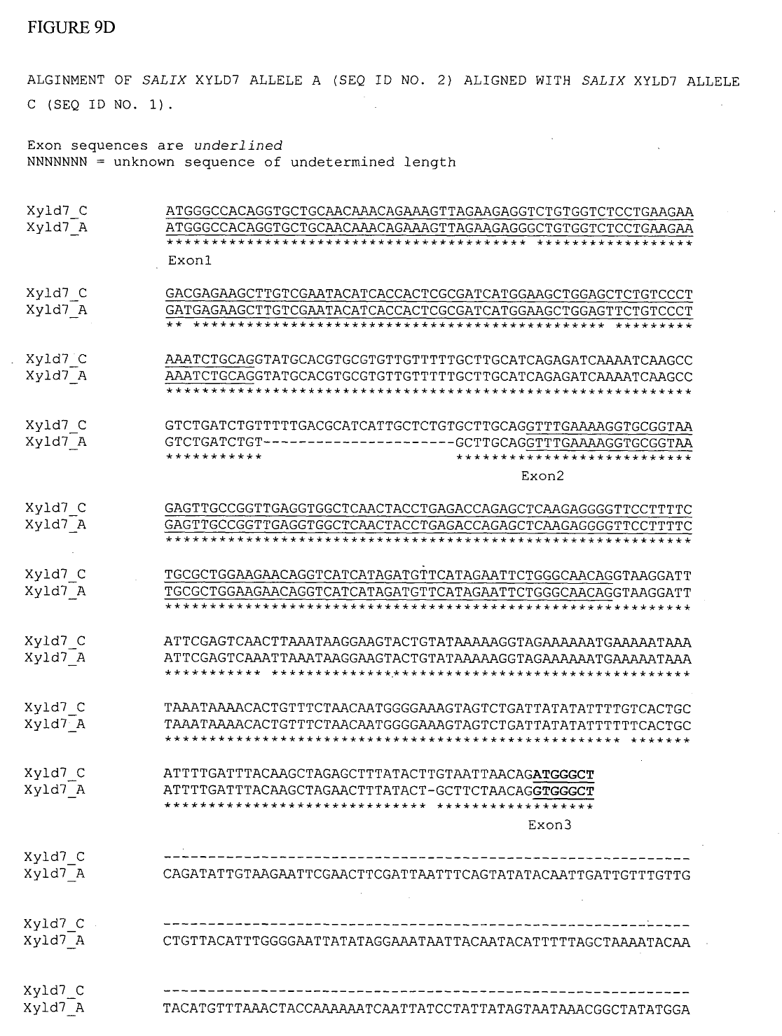

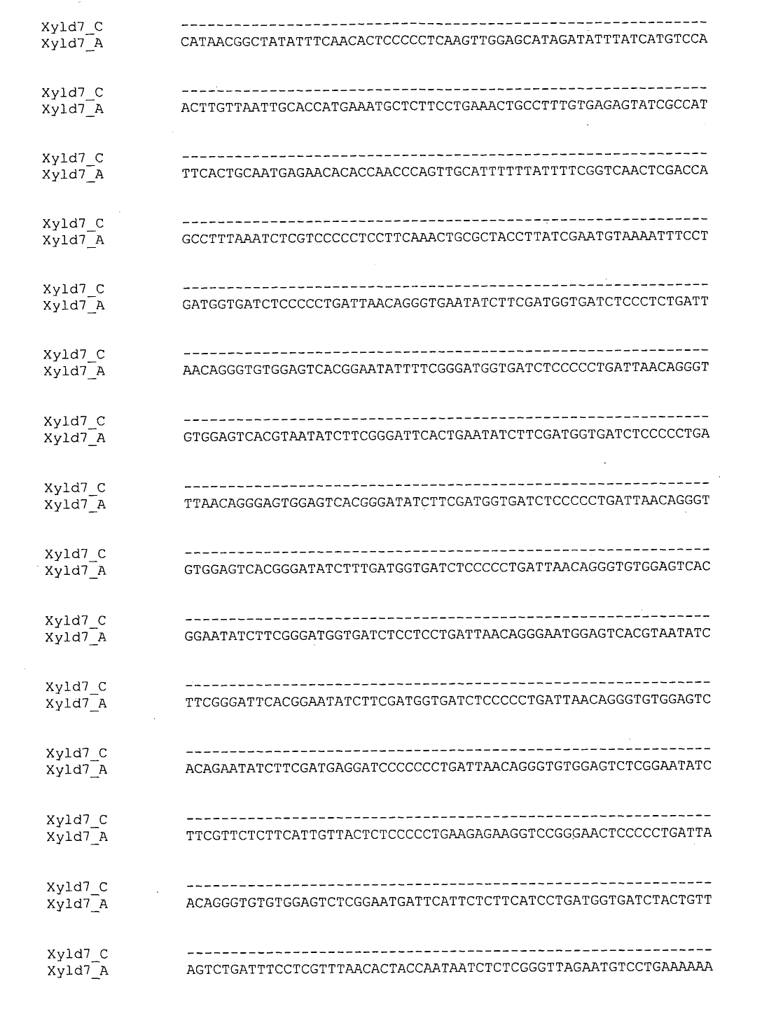

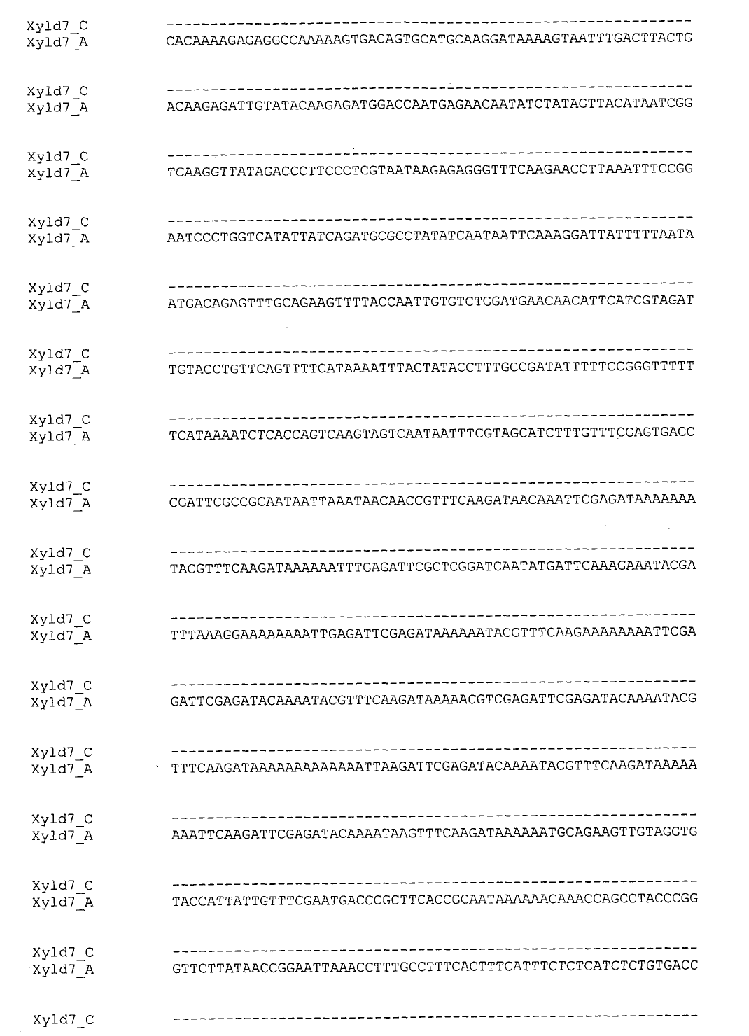

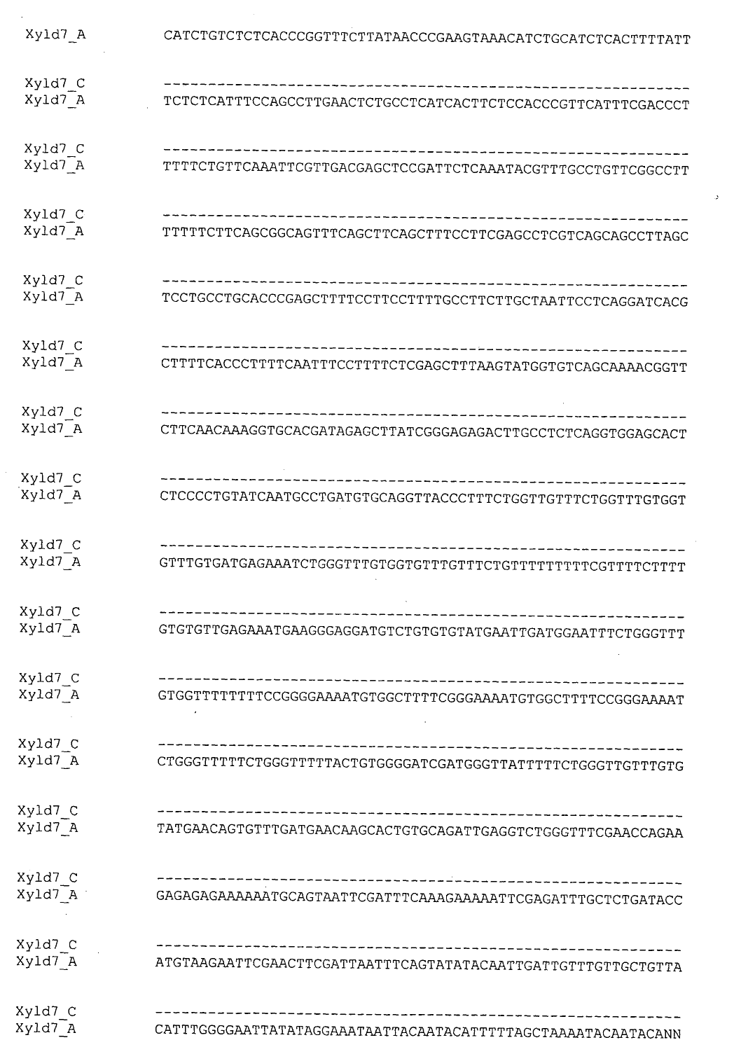

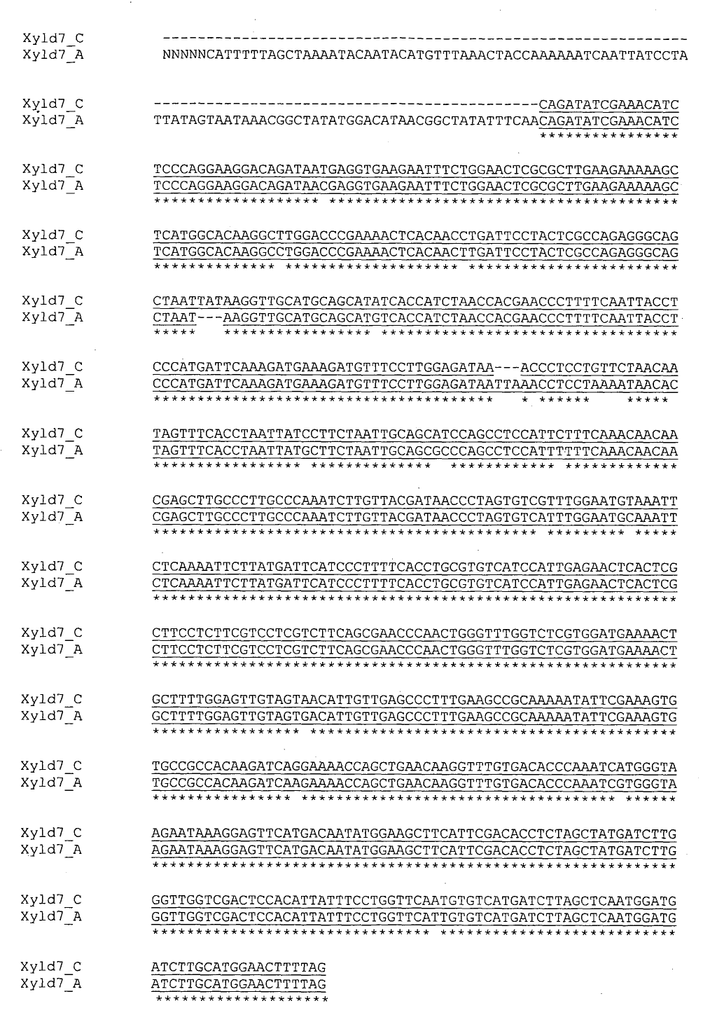

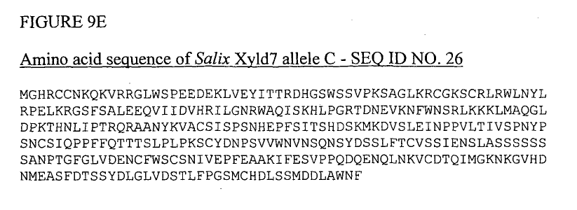

FIG. 9A: shows the nucleotide sequence of the Xyld7 polynucleotide of Populus (SEQ ID NO 3). SEQ ID NO 3 is located within the QTL region shown in FIG. 1.

FIG. 9B: shows the nucleotide sequence of the Xyld7 allele A polynucleotide of Salix (SEQ ID NO 2).

FIG. 9C: shows the nucleotide sequence of the Xyld7 allele C polynucleotide of Salix (SEQ ID NO 1).

FIG. 9D: shows the nucleotide sequence of the Xyld7 allele A polynucleotide of Salix (SEQ ID NO 2) aligned with the Xyld7 allele C polynucleotide of Salix (SEQ ID NO 1) to indicate Gene Xyld7 allele A insertion region.

FIG. 9E: shows the amino acid sequence of the Xyld7 allele C polypeptide in Salix (SEQ ID NO 26).

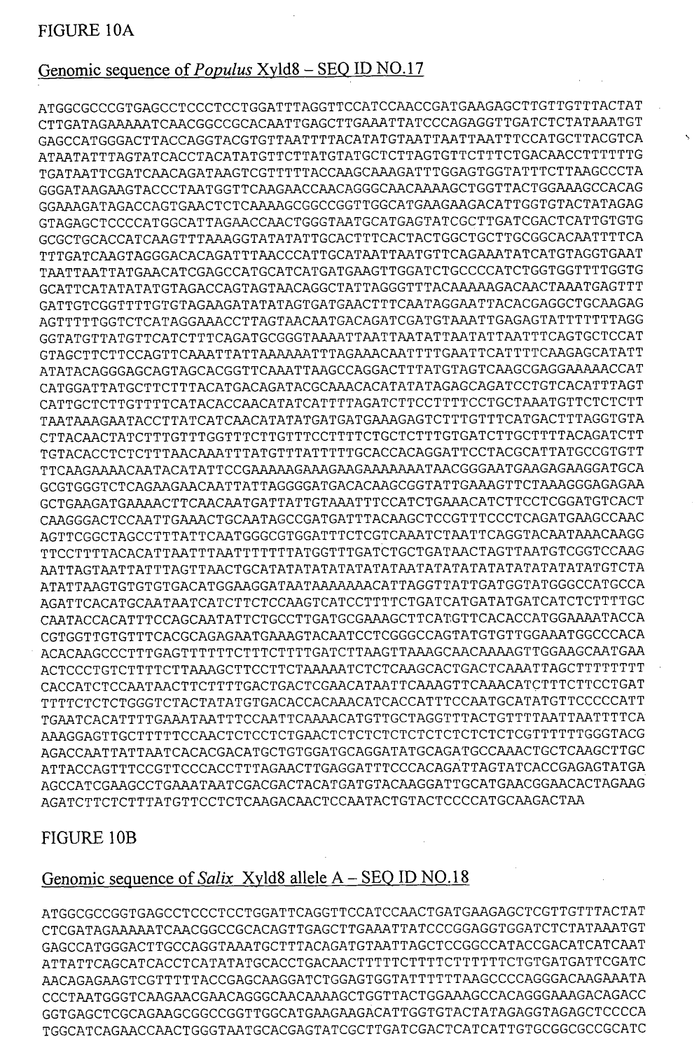

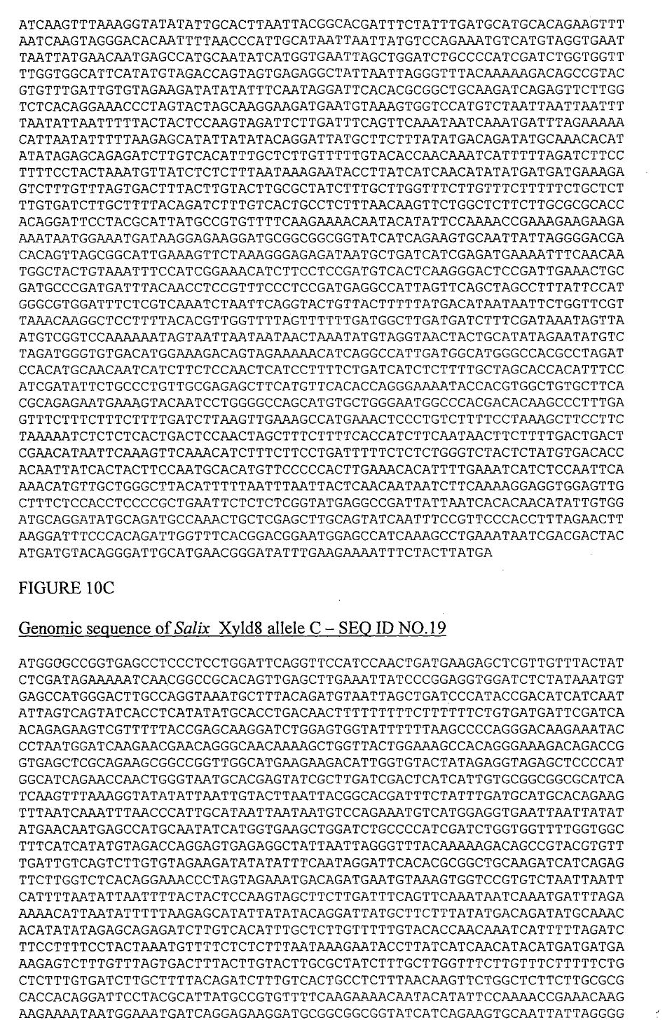

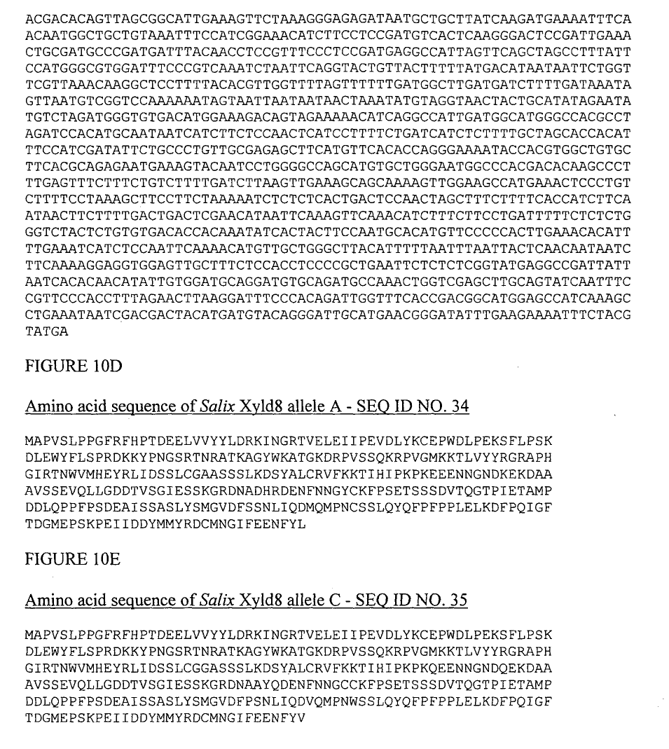

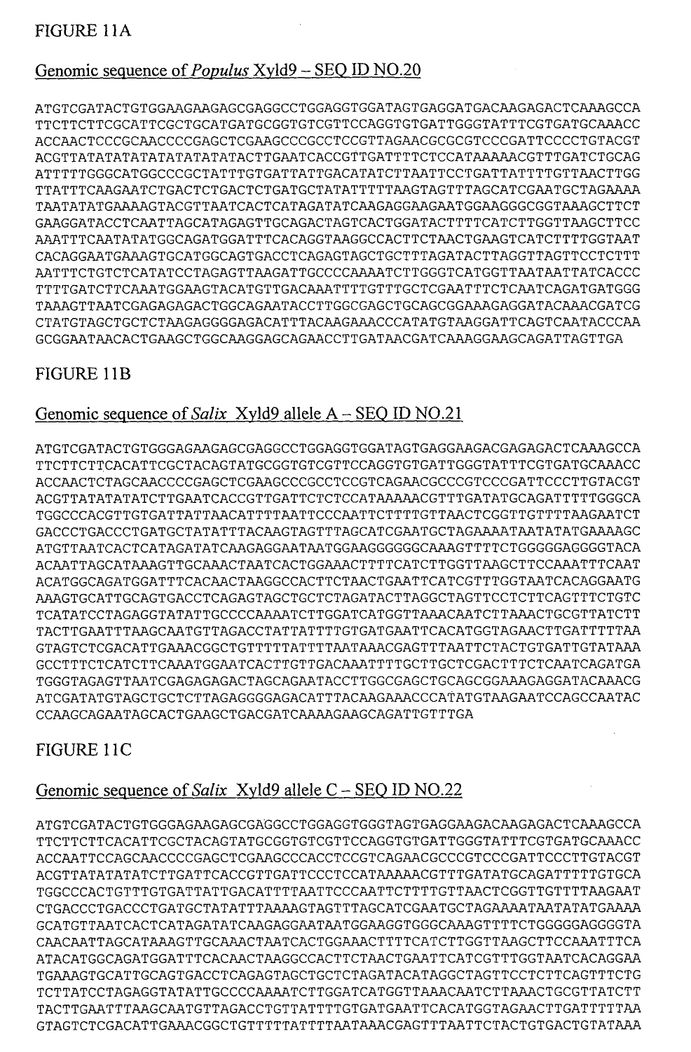

FIG. 10A: shows the nucleotide sequence of the Xyld8 polynucleotide of Populus (SEQ ID NO 17). SEQ ID NO 17 is located within the QTL region shown in FIG. 1.

FIG. 10B: shows the nucleotide sequence of the Xyld8 allele A polynucleotide of Salix (SEQ ID NO 18).

FIG. 10C: shows the nucleotide sequence of the Xyld8 allele C polynucleotide of Salix (SEQ ID NO 19).

FIG. 10D: shows the amino acid sequence of the Xyld8 allele A polypeptide of Salix (SEQ ID NO 34).

FIG. 10E: shows the amino acid sequence of the Xyld8 allele C polypeptide of Salix (SEQ ID NO 35).

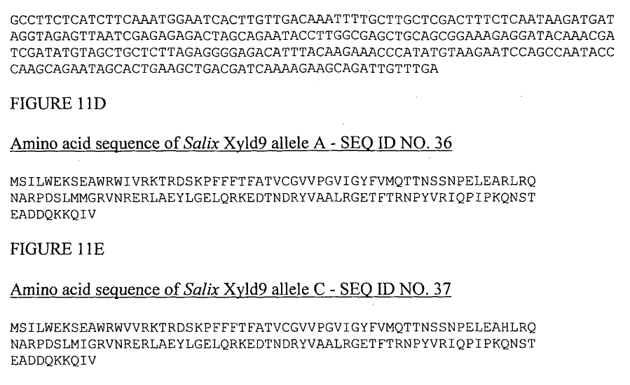

FIG. 11A: shows the nucleotide sequence of the Xyld9 polynucleotide of Populus (SEQ ID NO 20). SEQ ID NO 20 is located within the QTL region shown in FIG. 1.

FIG. 11B: shows the nucleotide sequence of the Xyld9 allele A polynucleotide of Salix (SEQ ID NO 21).

FIG. 11C: shows the nucleotide sequence of the Xyld9 allele C polynucleotide of Salix (SEQ ID NO 22).

FIG. 11D: shows the amino acid sequence of the Xyld9 allele A polypeptide of Salix (SEQ ID NO 36).

FIG. 11E: shows the amino acid sequence of the Xyld9 allele C polypeptide of Salix (SEQ ID NO 37).

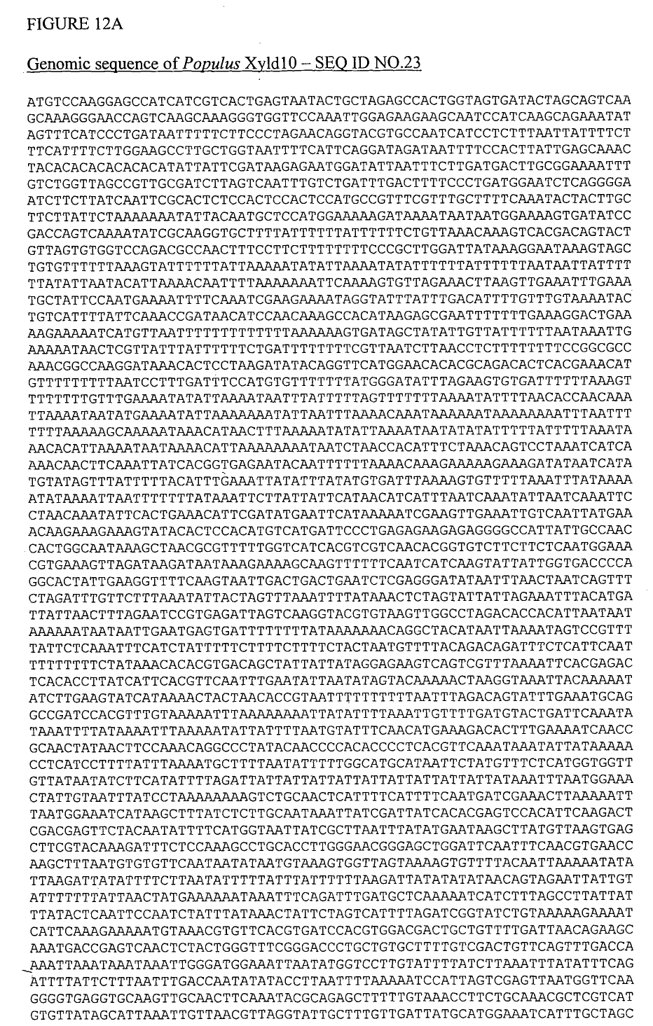

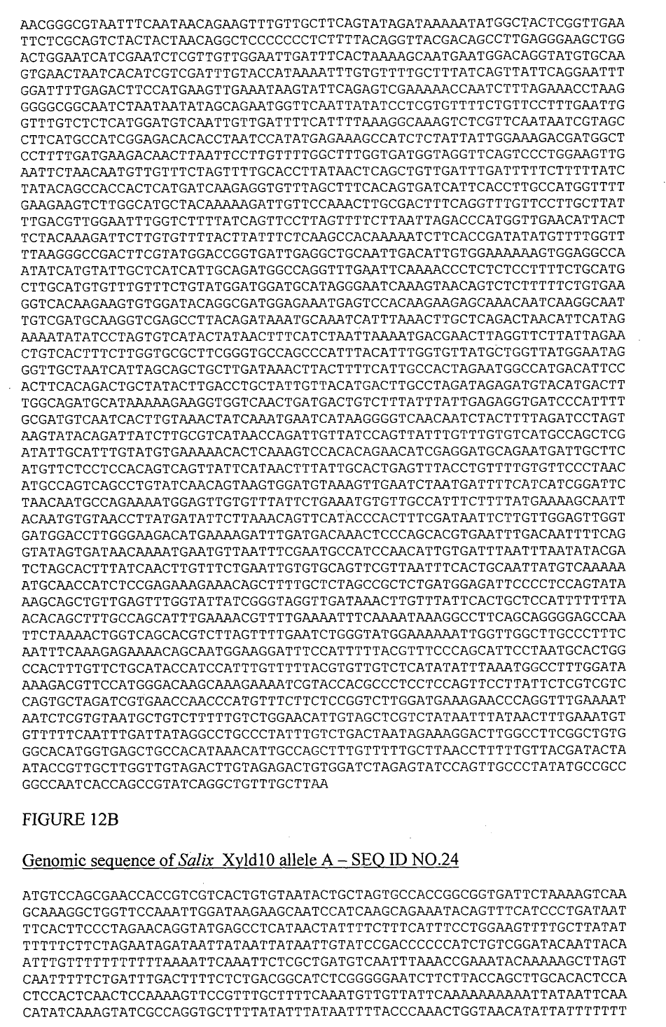

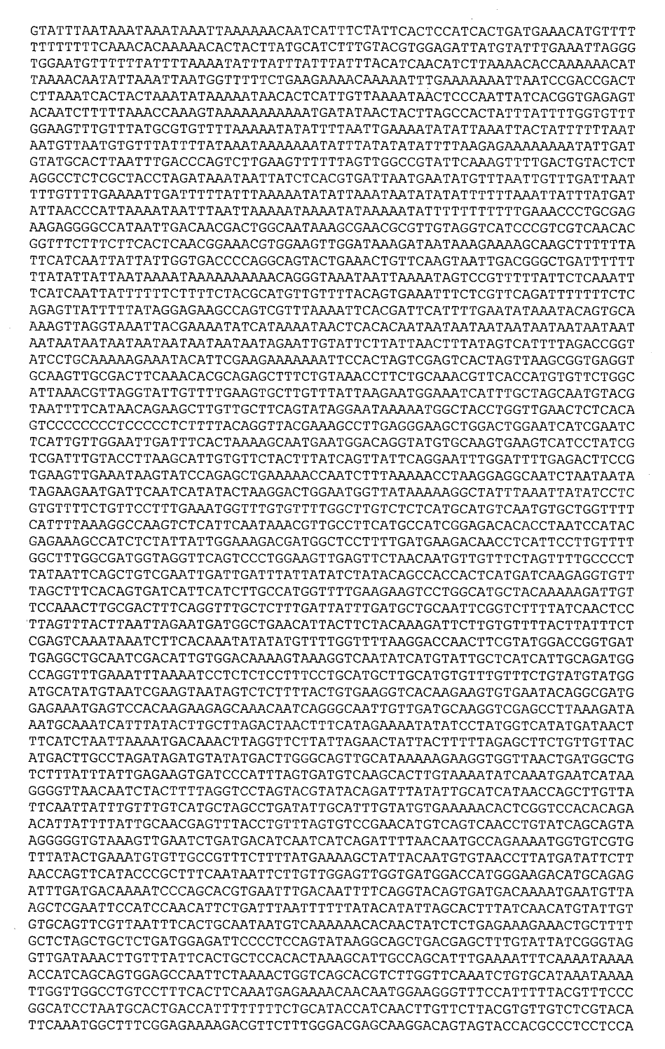

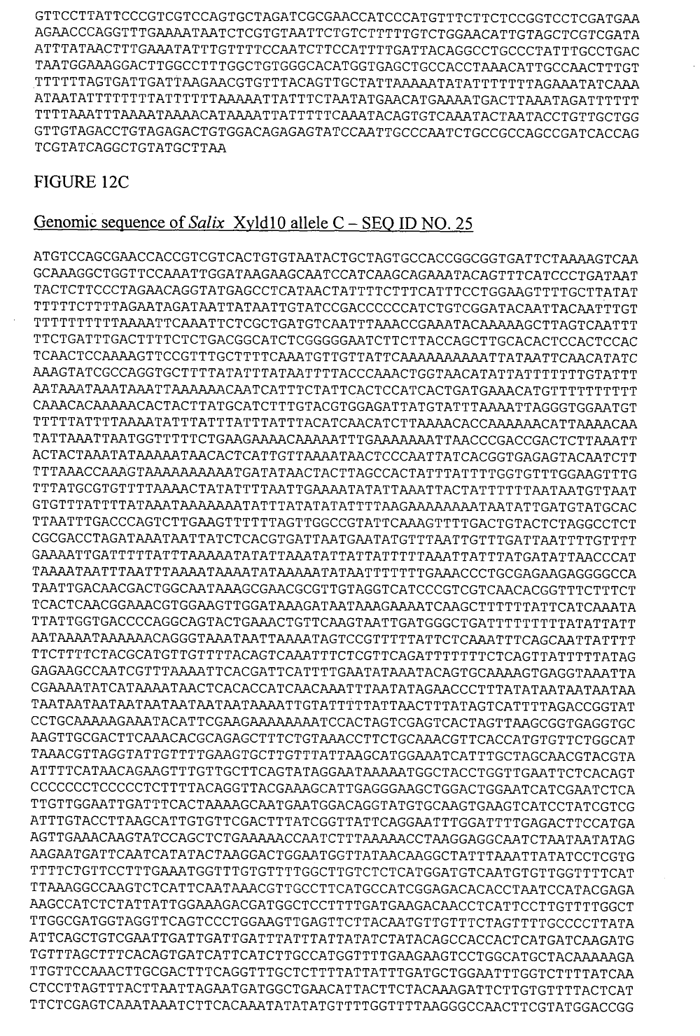

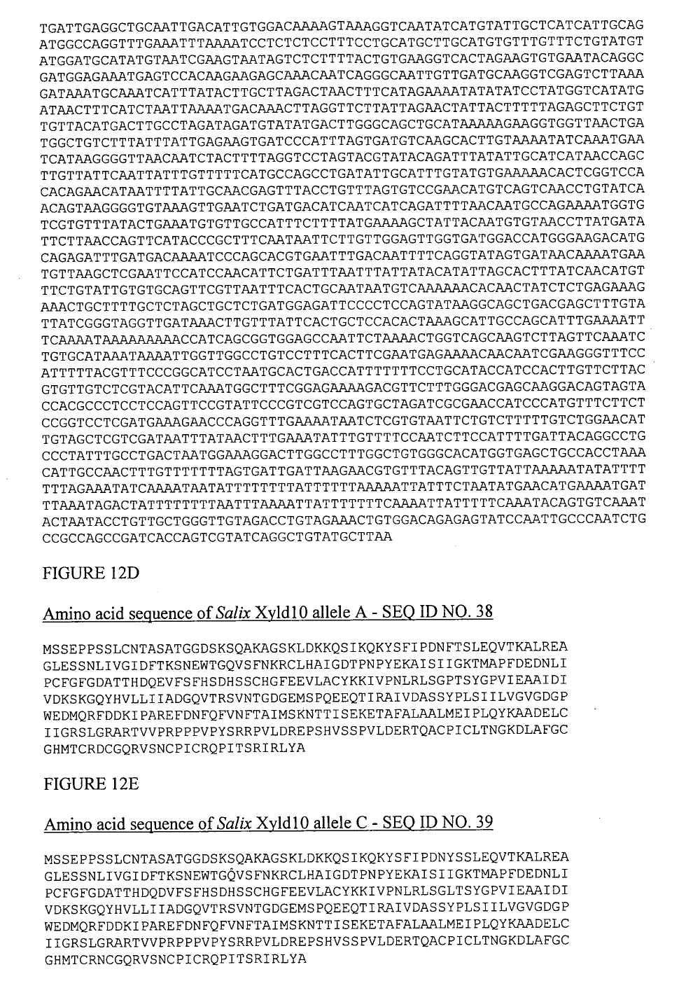

FIG. 12A: shows the nucleotide sequence of the Xyld10 polynucleotide of Populus (SEQ ID NO 23). SEQ ID NO 23 is located within the QTL region shown in FIG. 1.

FIG. 12B: shows the nucleotide sequence of the Xyld10 allele A polynucleotide of Salix (SEQ ID NO 24).

FIG. 12C: shows the nucleotide sequence of the Xyld10 allele C polynucleotide of Salix (SEQ ID NO 25).

FIG. 12D: shows the amino acid sequence of the Xyld10 allele A polypeptide of Salix (SEQ ID NO 38).

FIG. 12E: shows the amino acid sequence of the Xyld10 allele C polypeptide of Salix (SEQ ID NO 39).

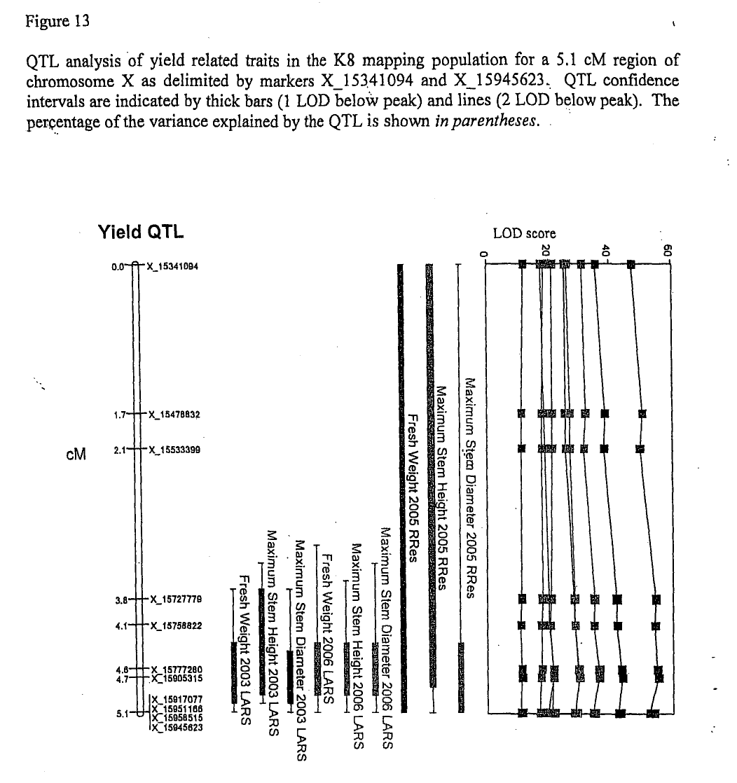

FIG. 13: shows QTL analysis of yield related traits in the K8 mapping population for a 5.1 cM region of chromosome X as delimited by markers X_{—}15341094 and X_{—}15945623. QTL confidence intervals are indicated by thick bars (1 LOD below peak) and lines (2 LOD below peak). The percentage of the variance explained by the QTL is shown in parentheses.

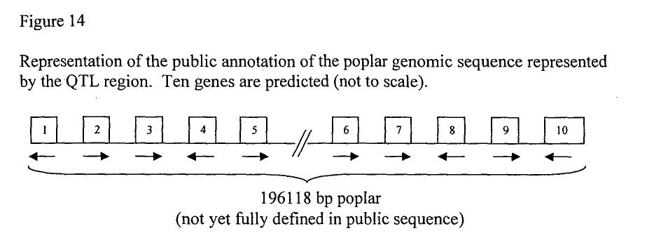

FIG. 14 shows representation of the public annotation of the poplar genomic sequence represented by the QTL region. Ten genes are predicted (not to scale).

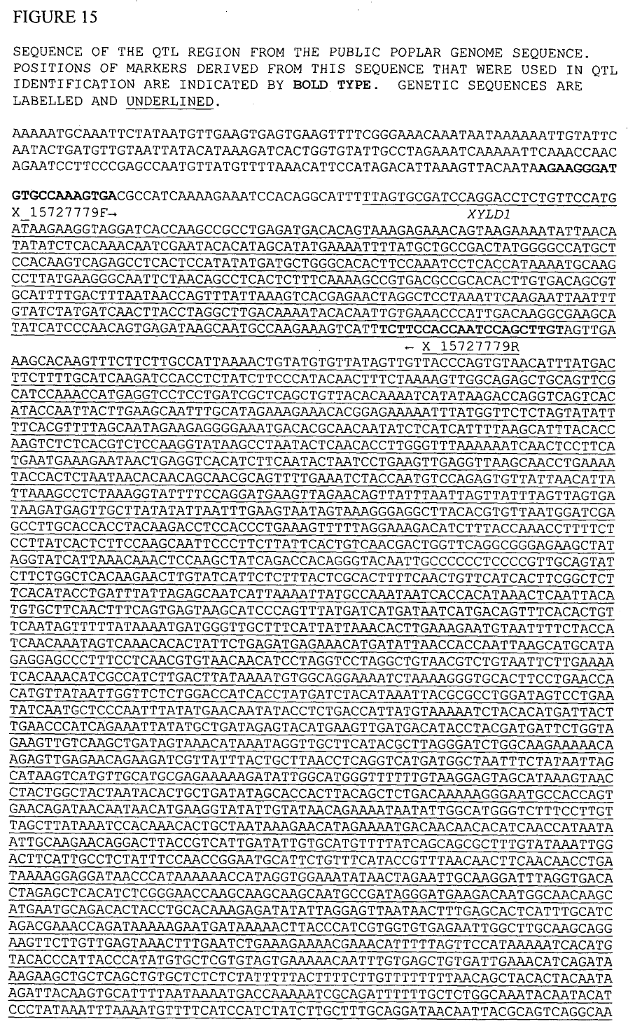

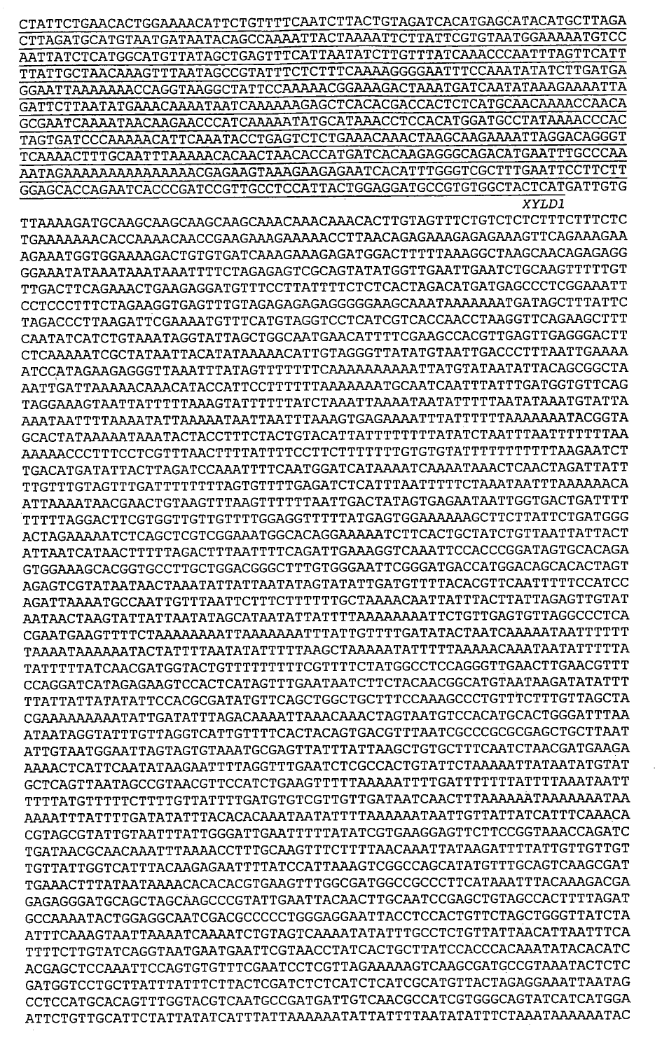

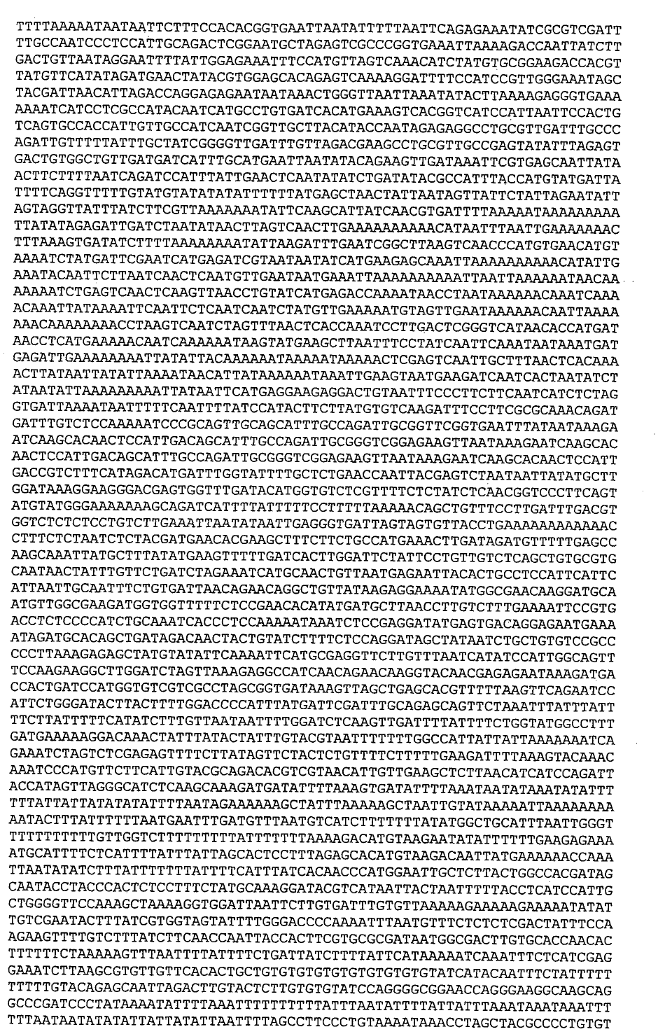

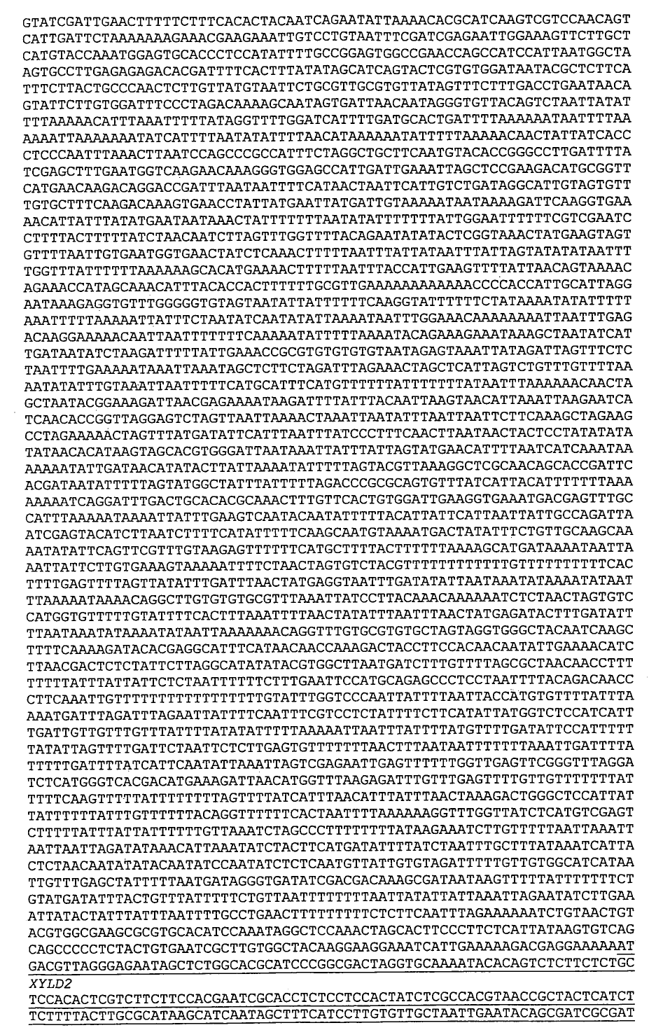

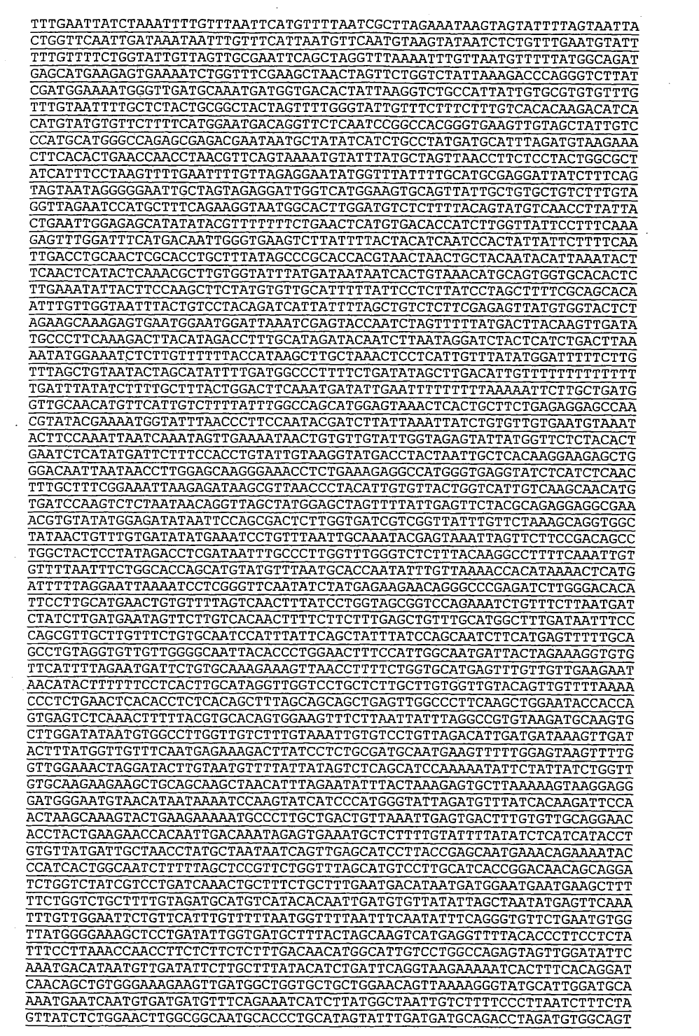

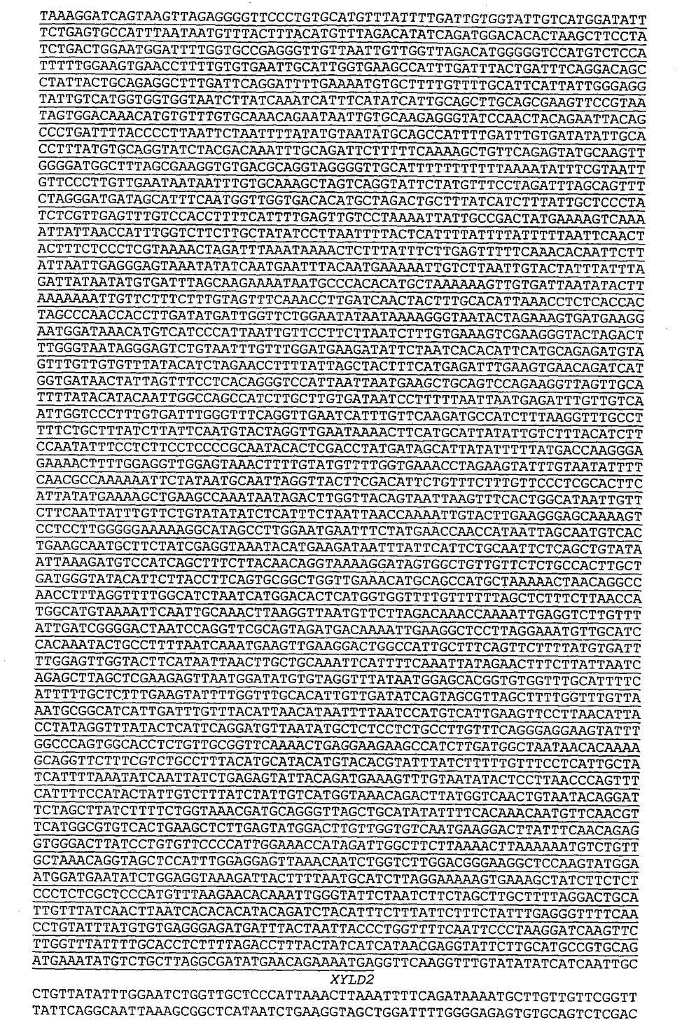

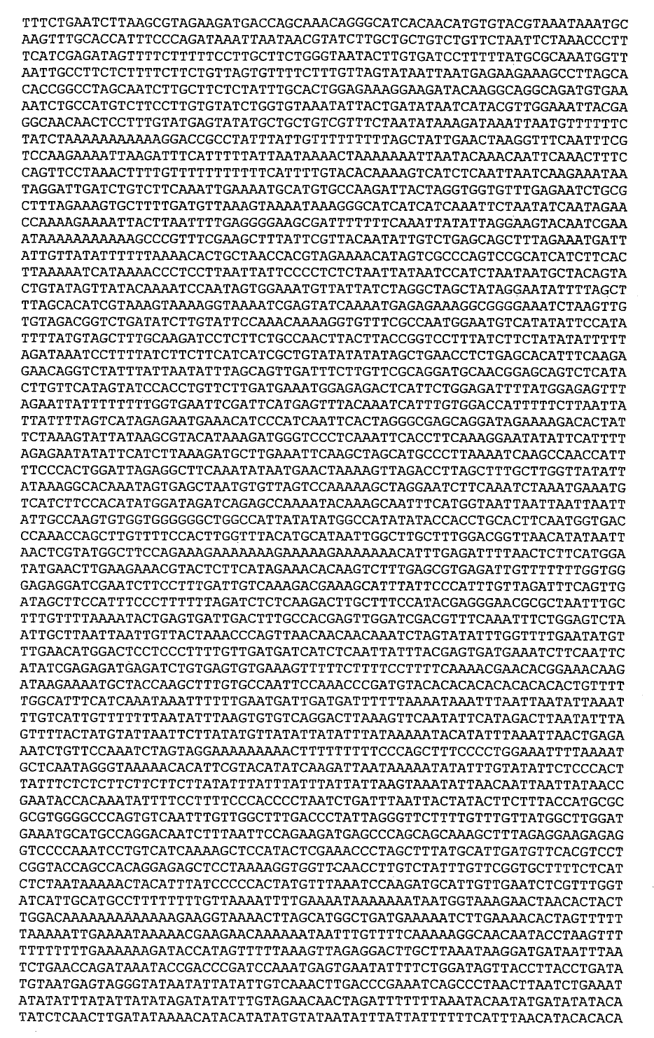

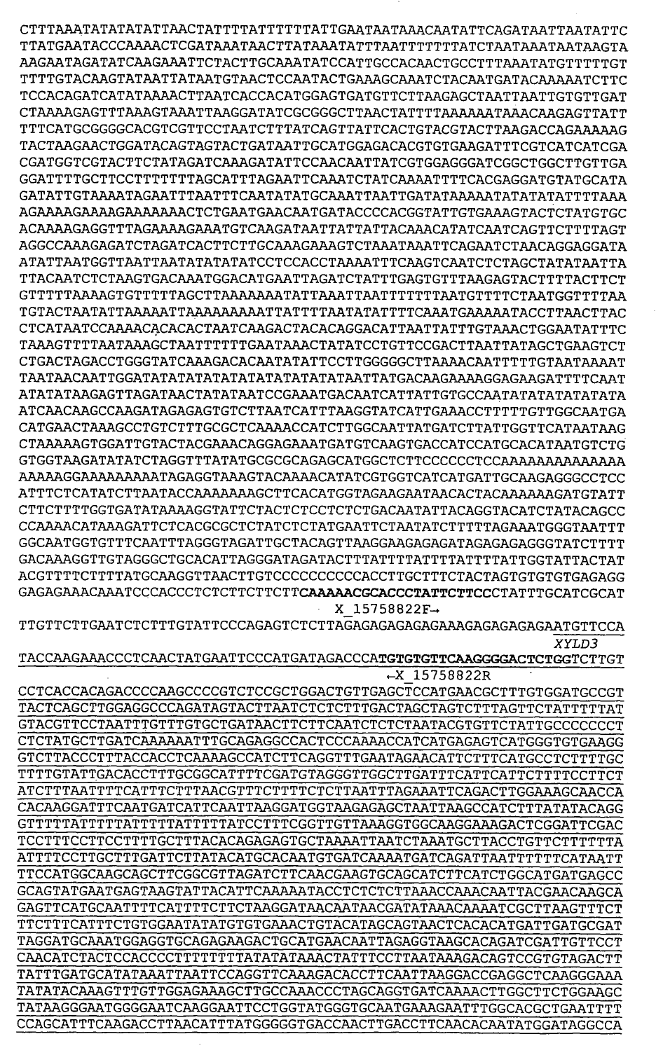

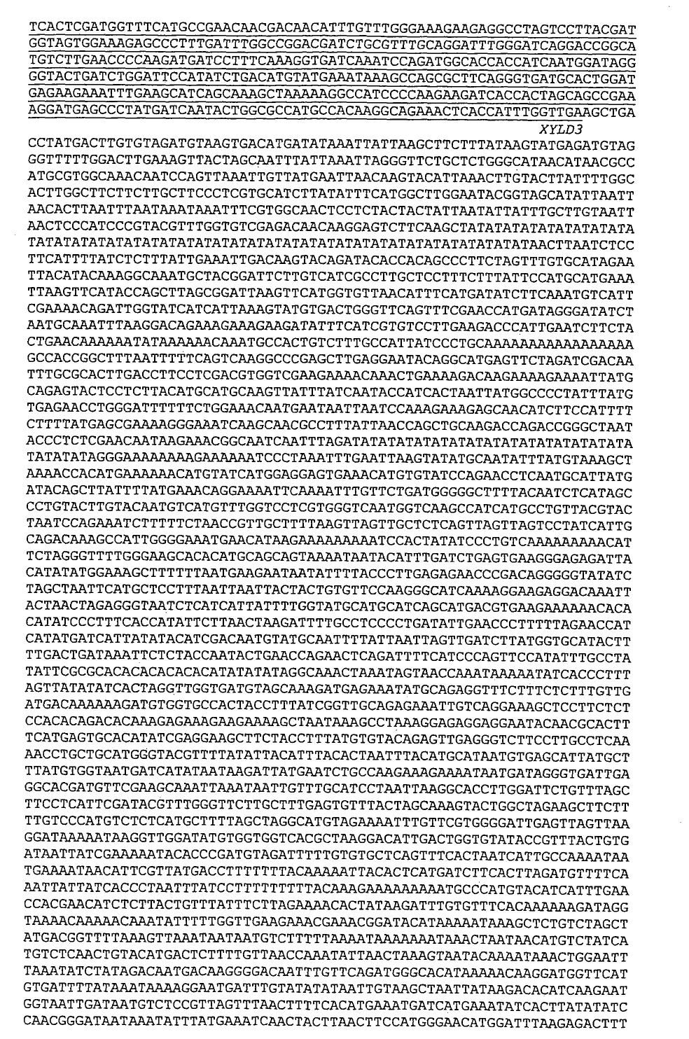

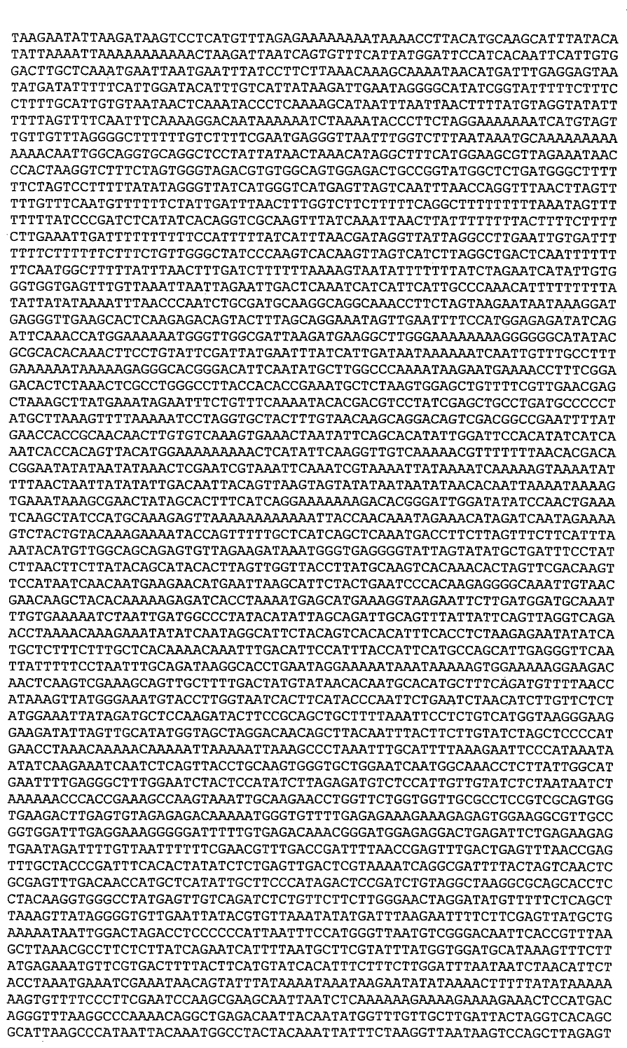

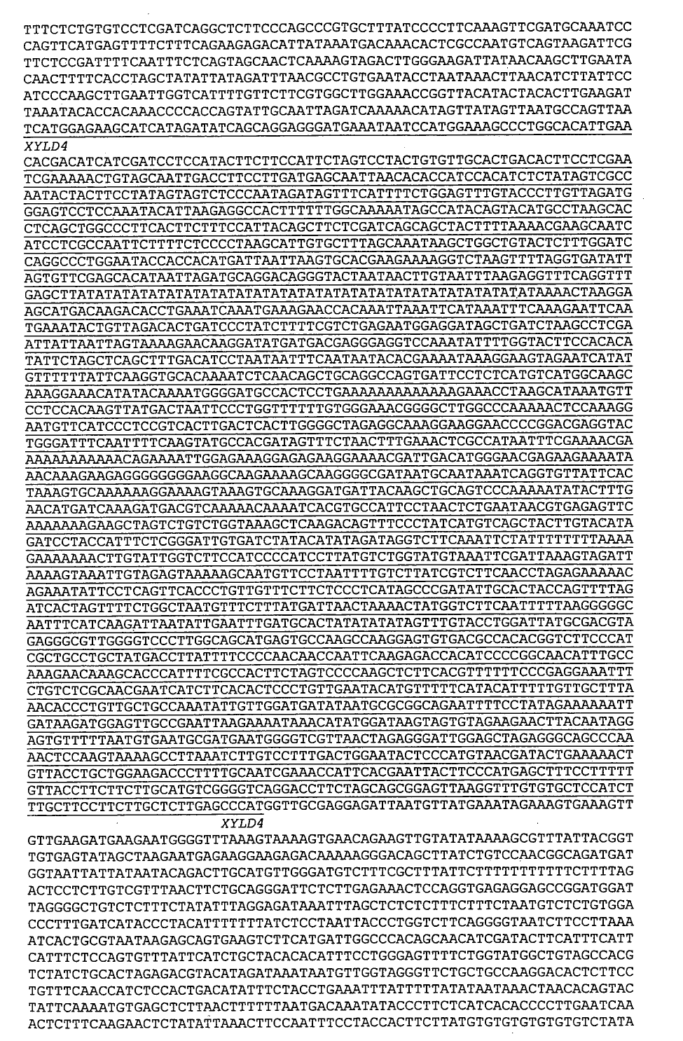

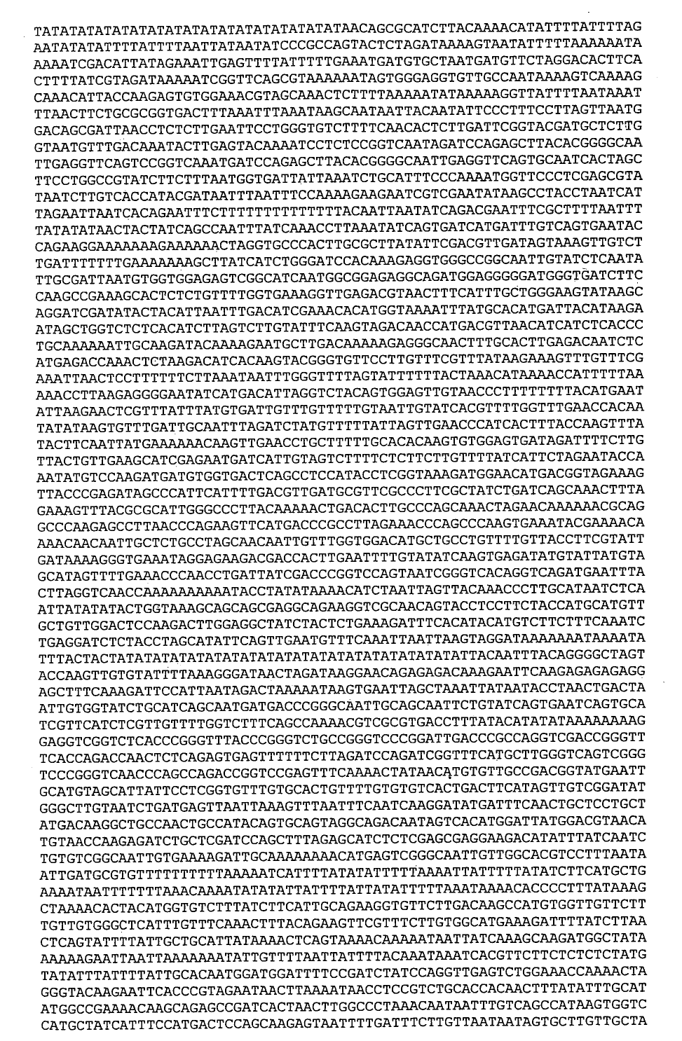

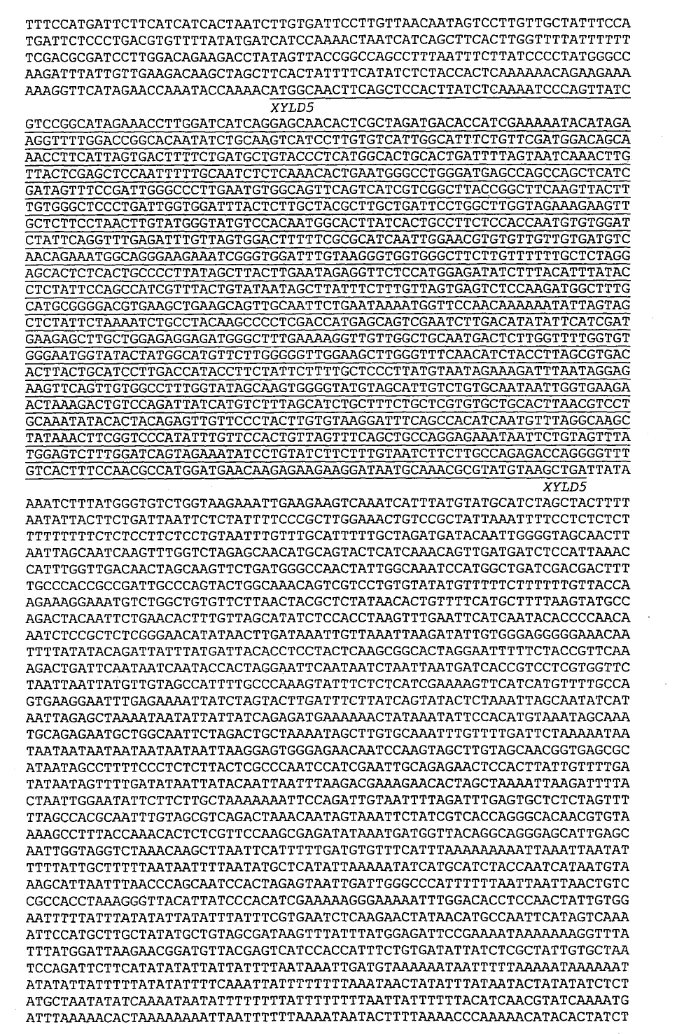

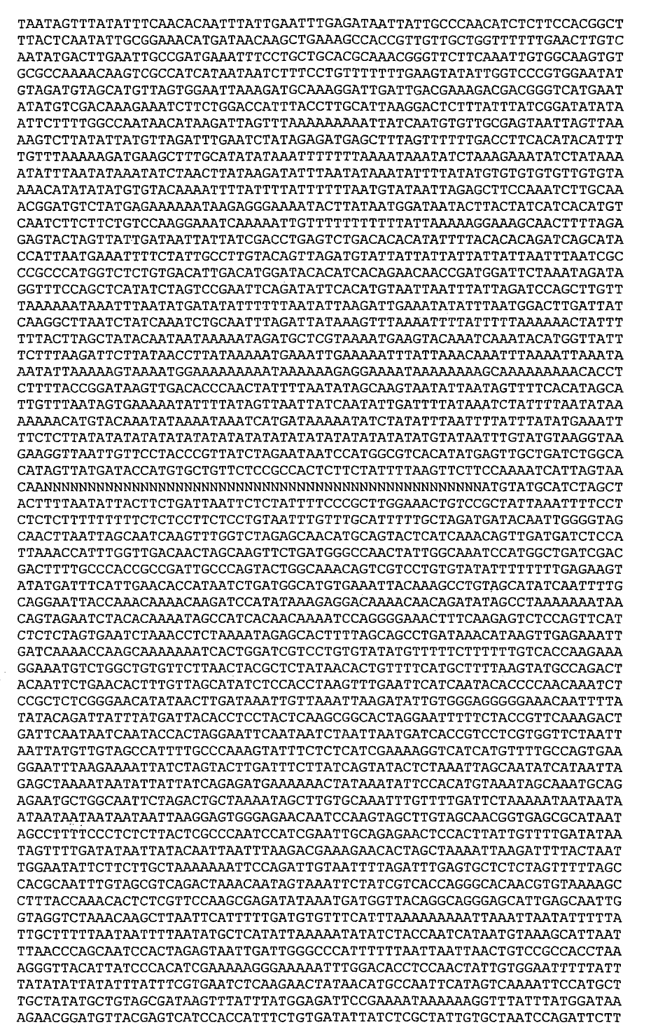

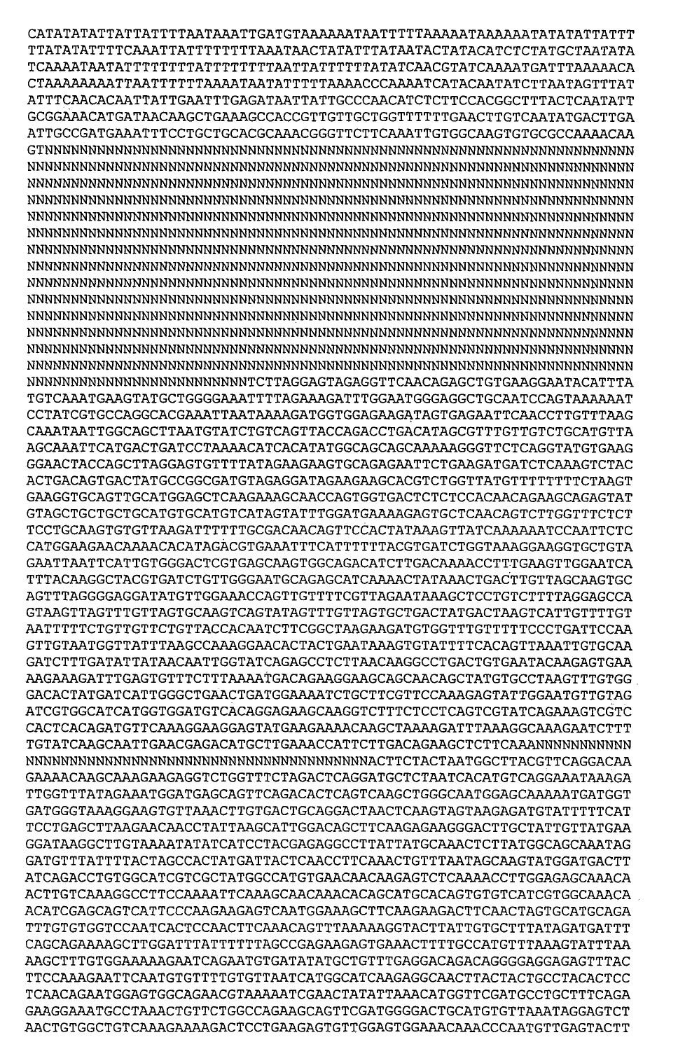

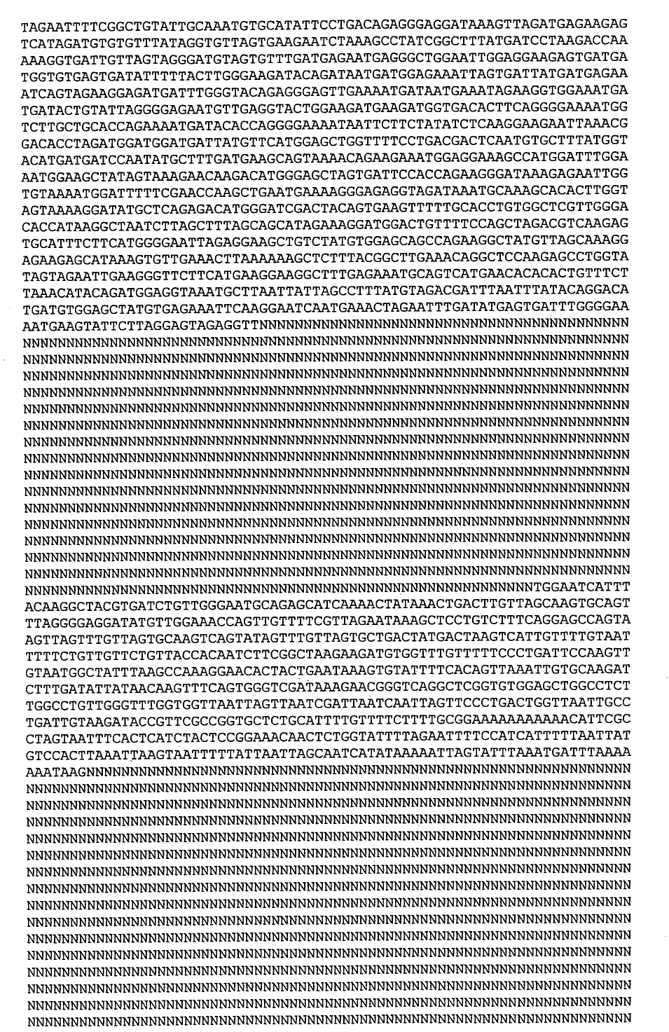

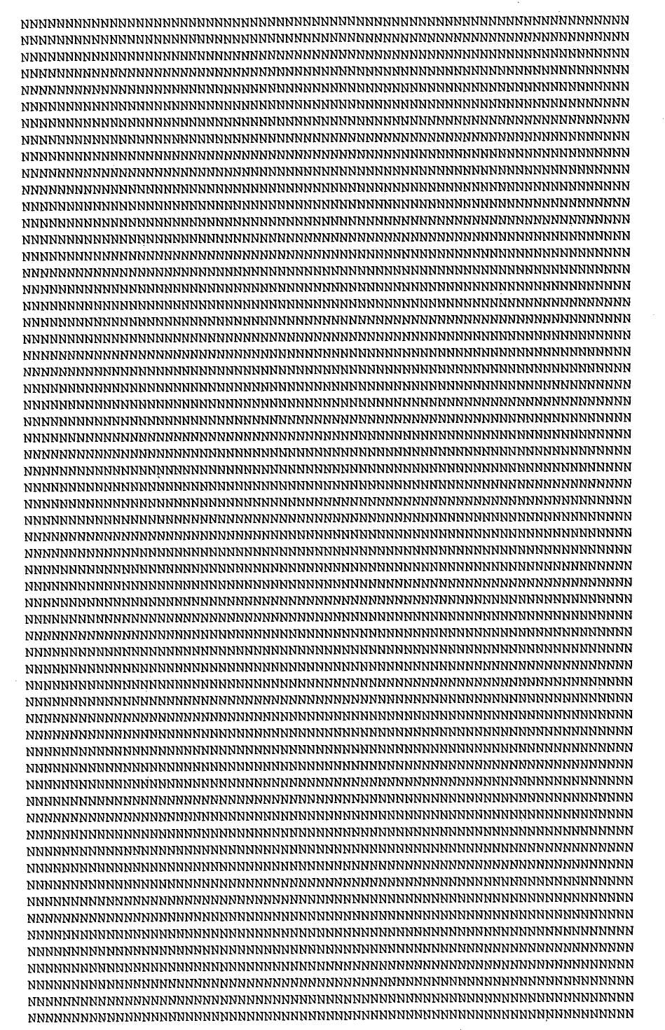

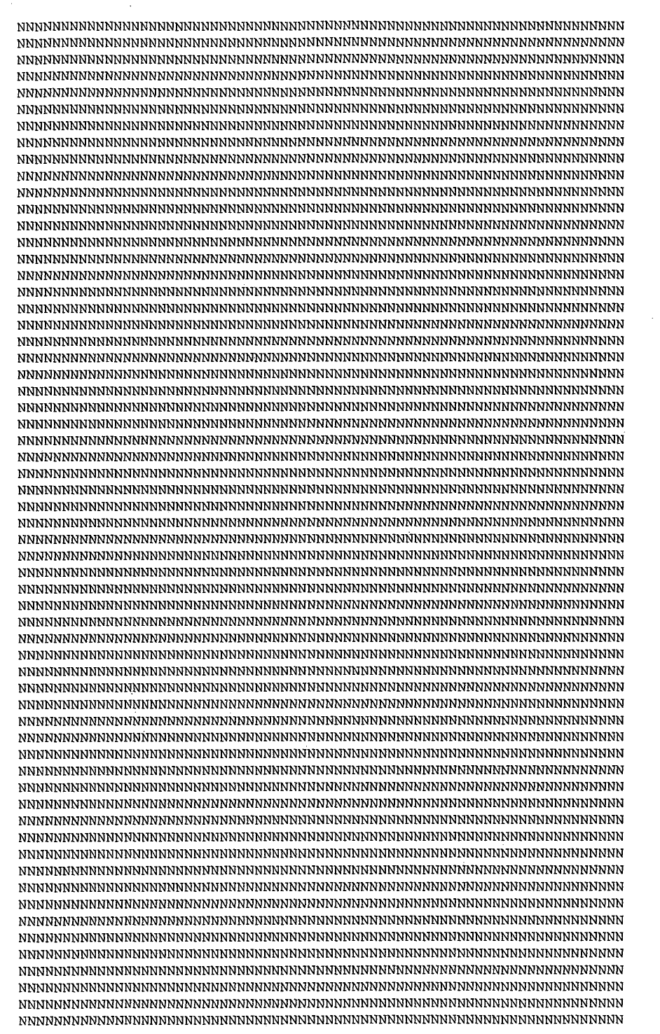

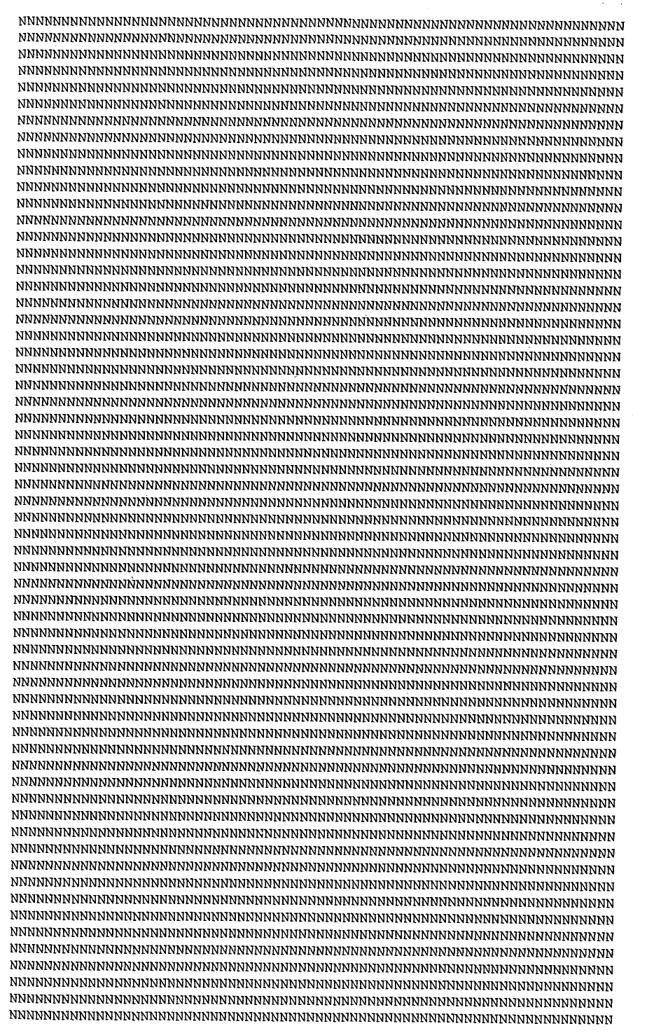

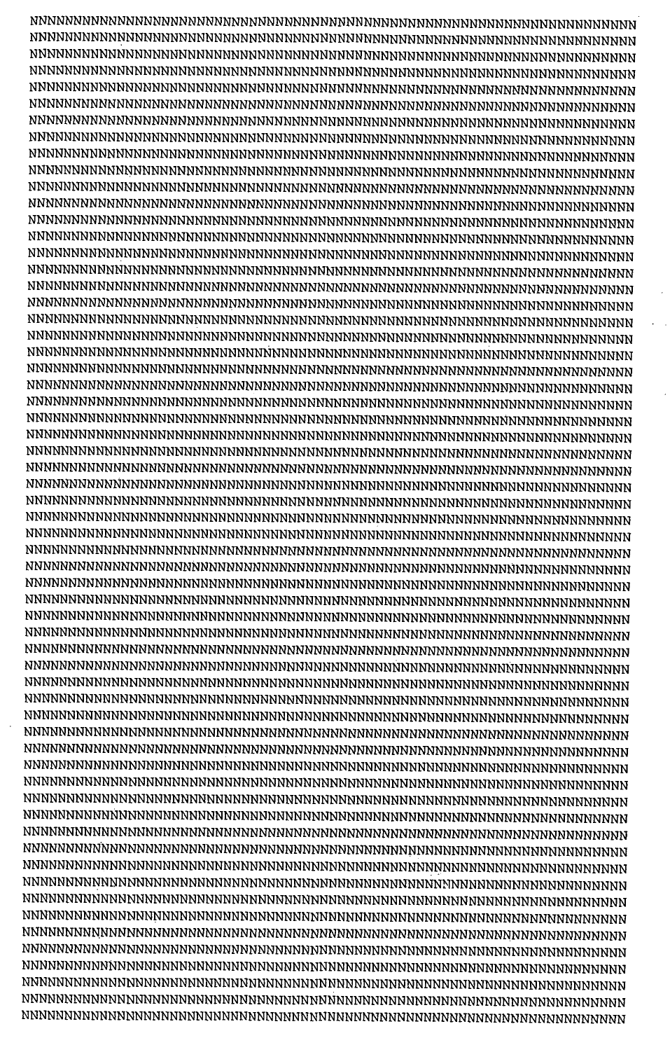

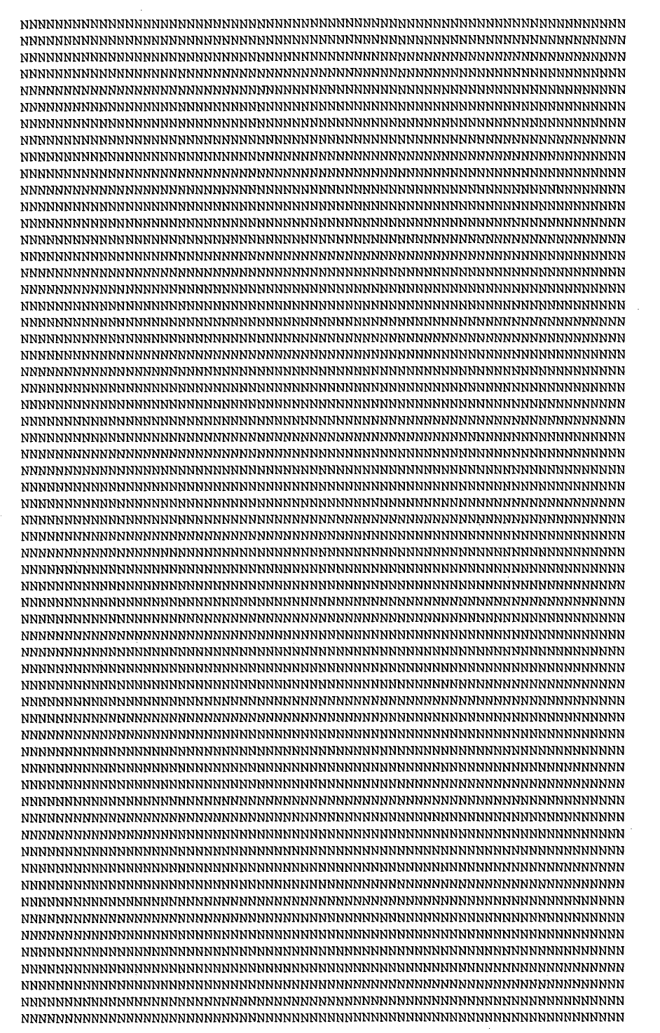

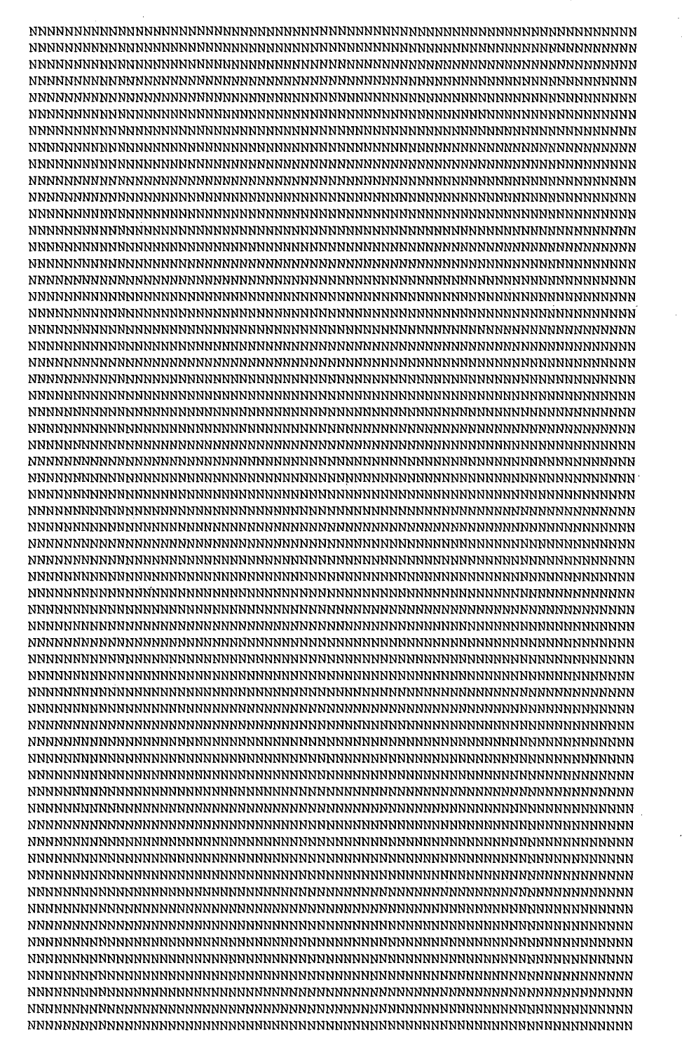

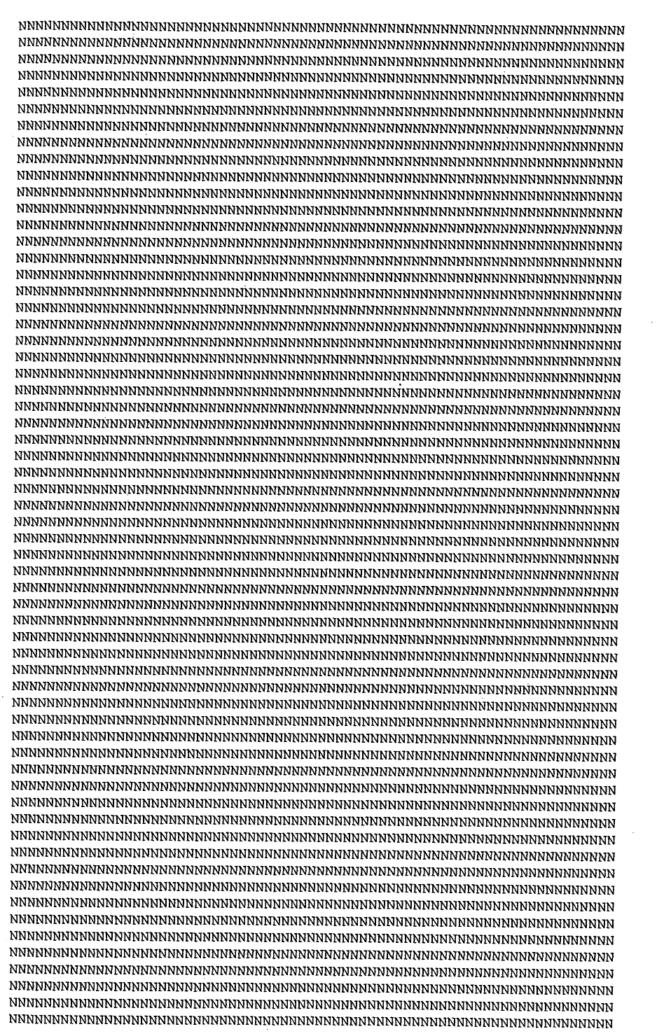

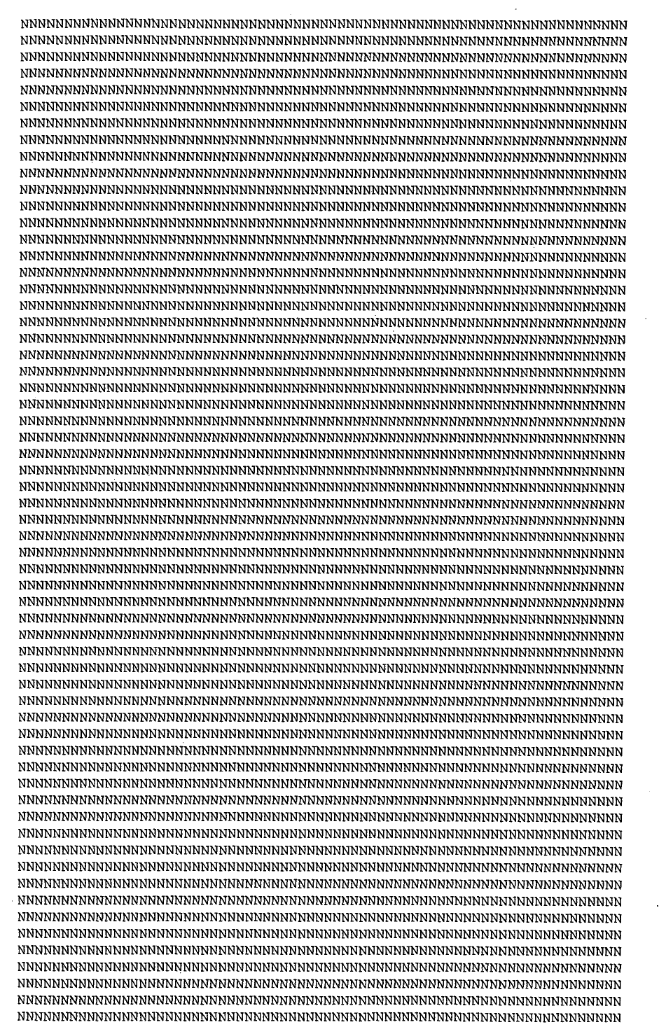

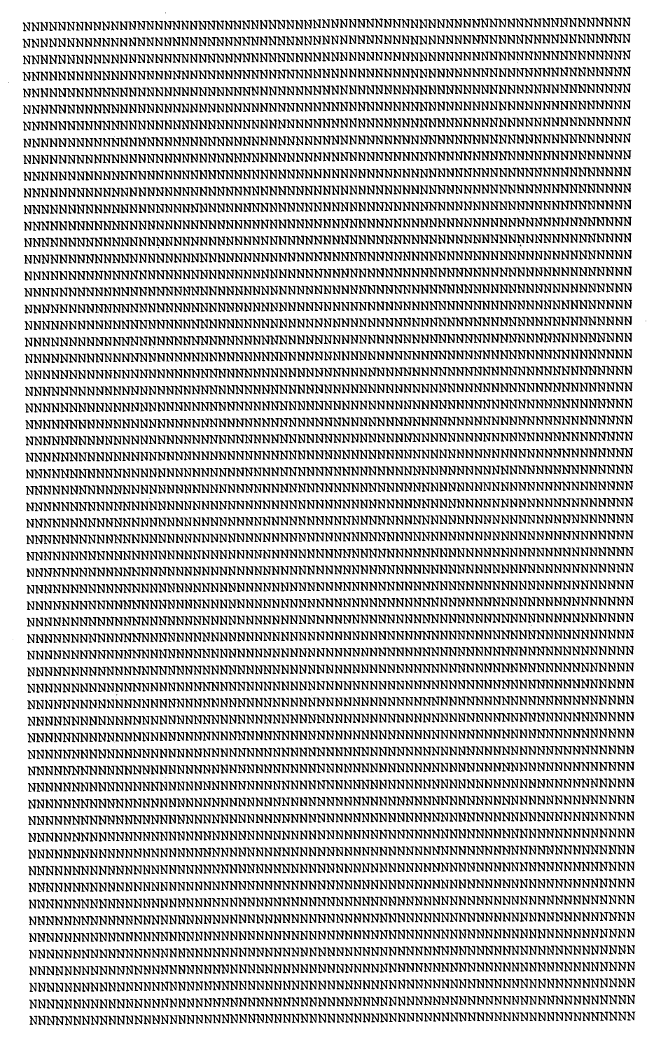

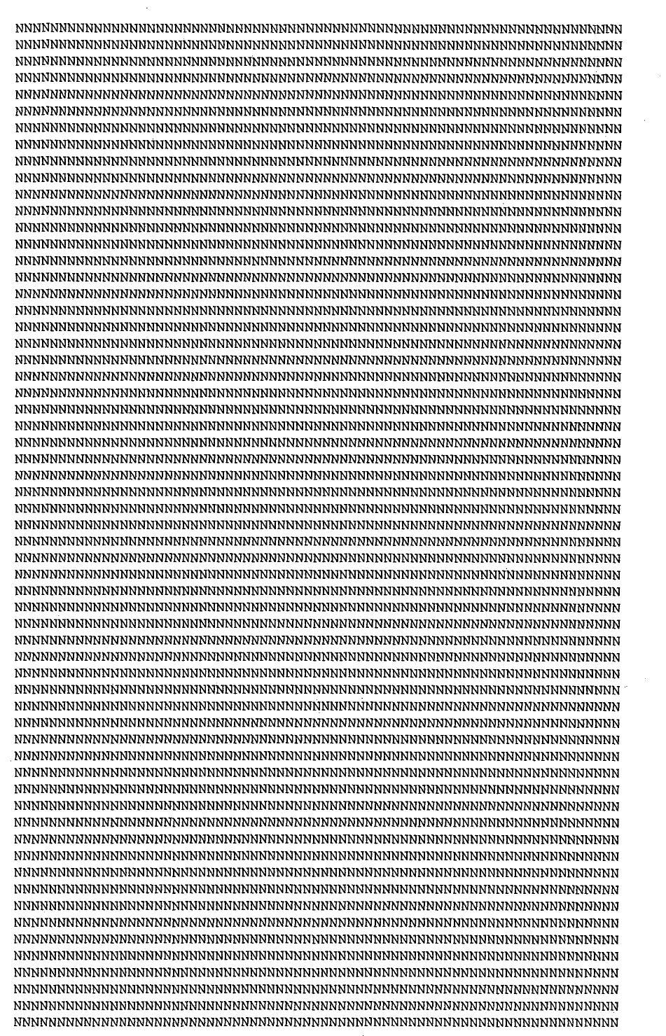

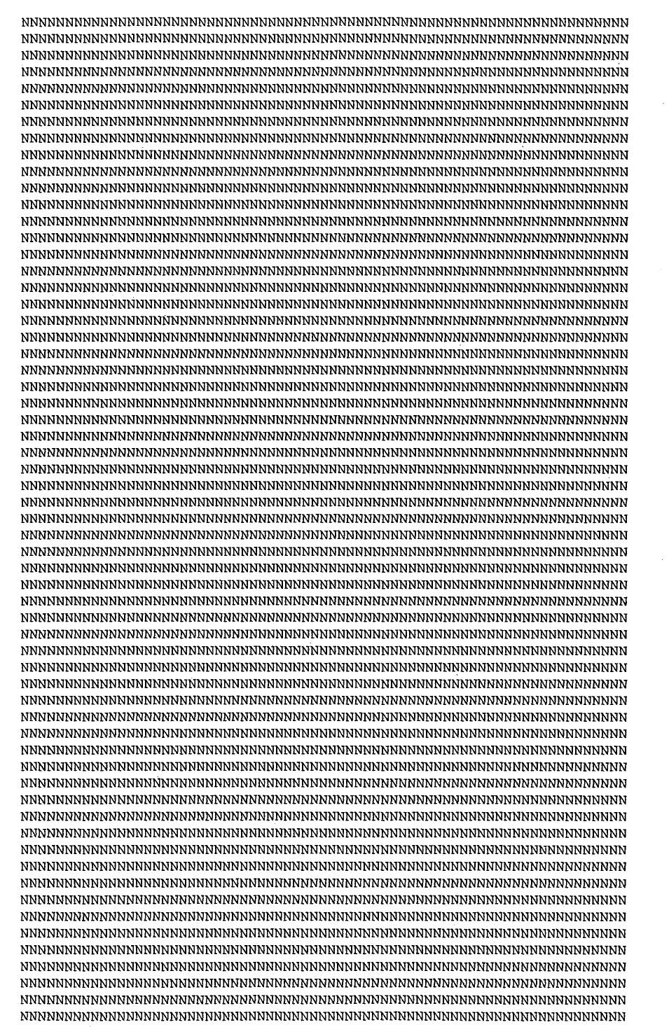

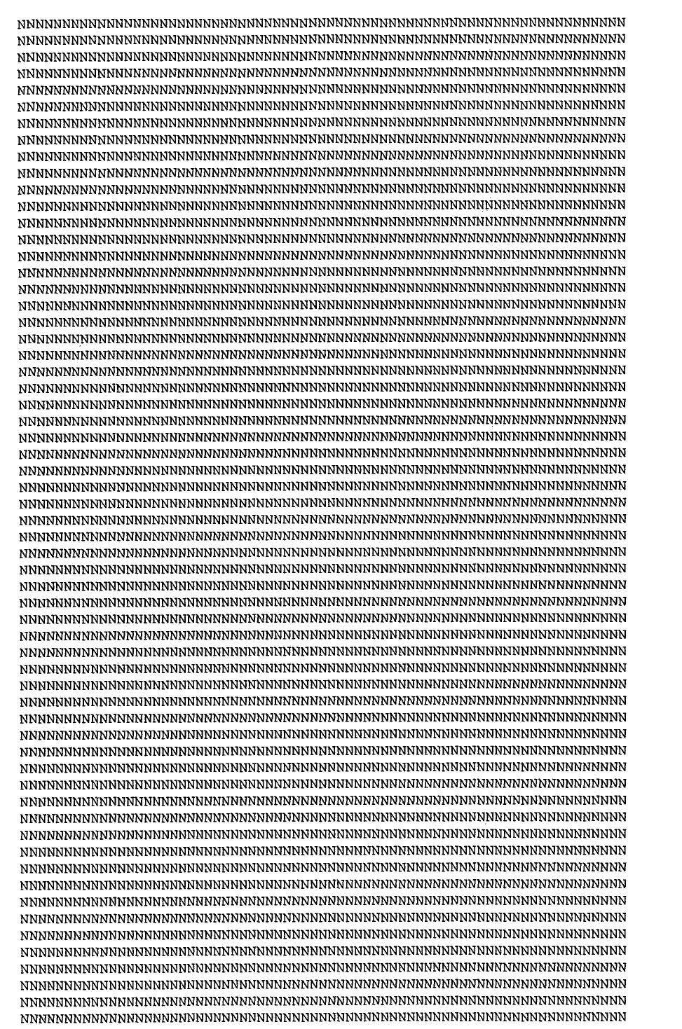

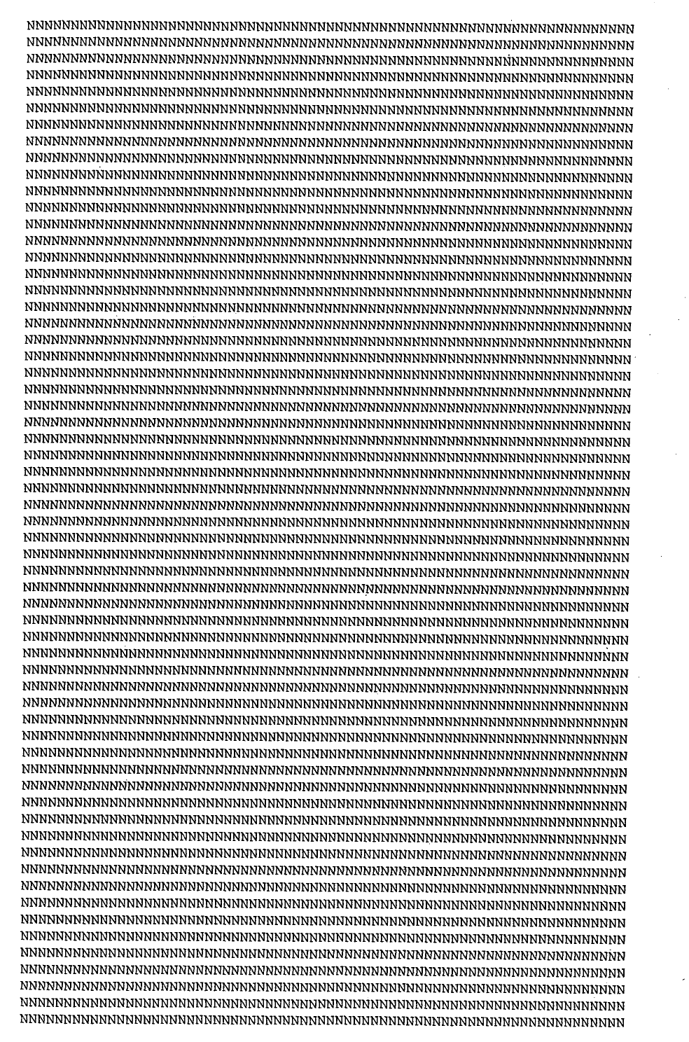

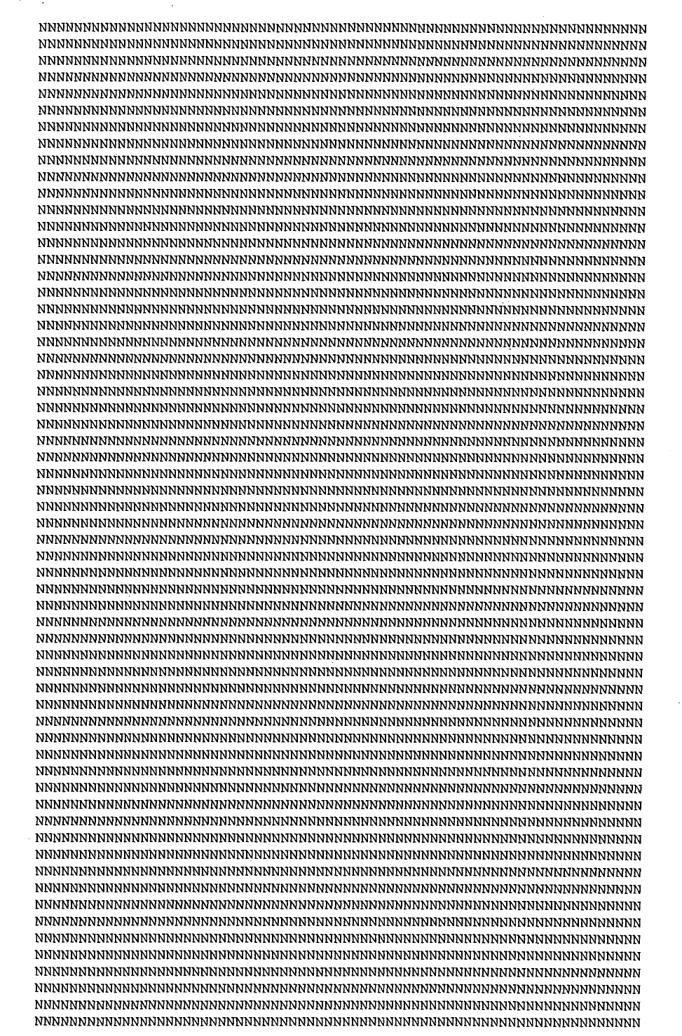

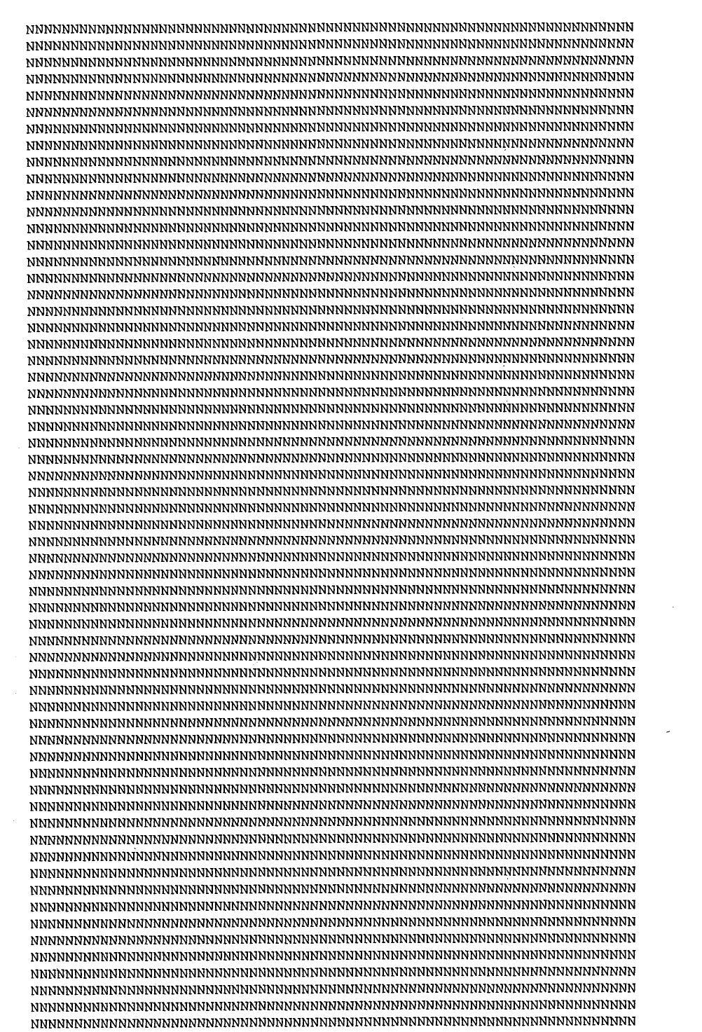

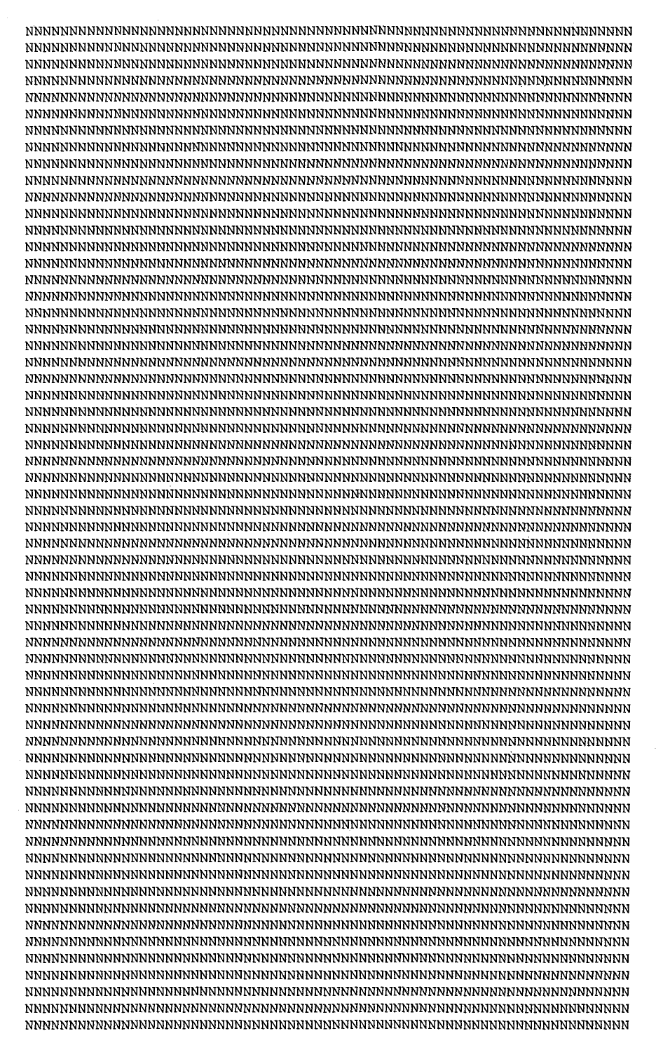

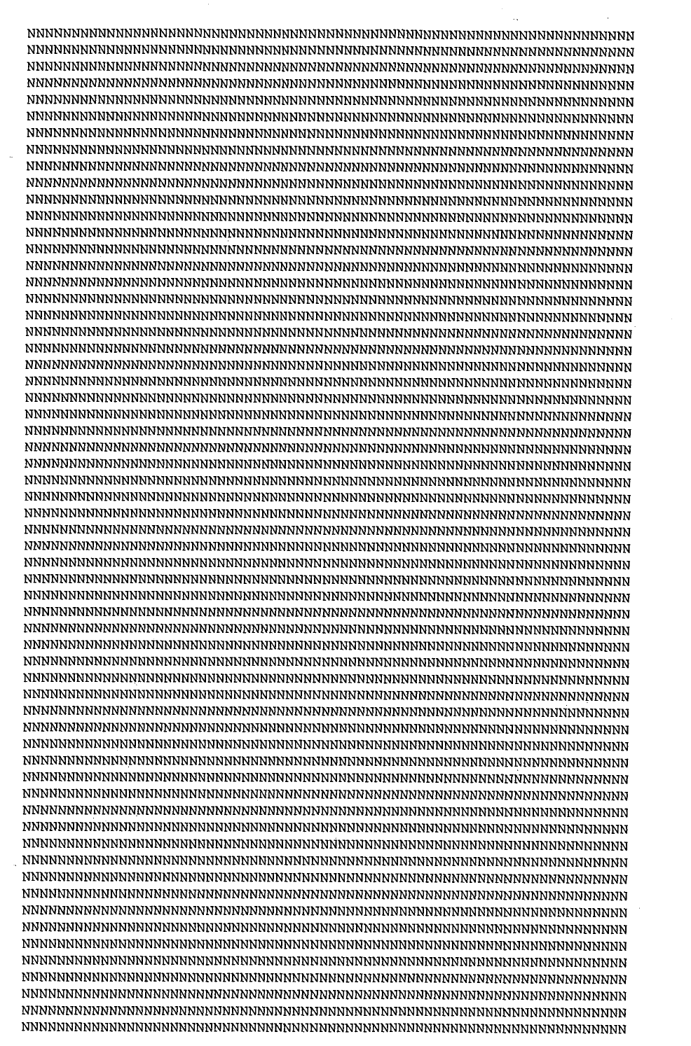

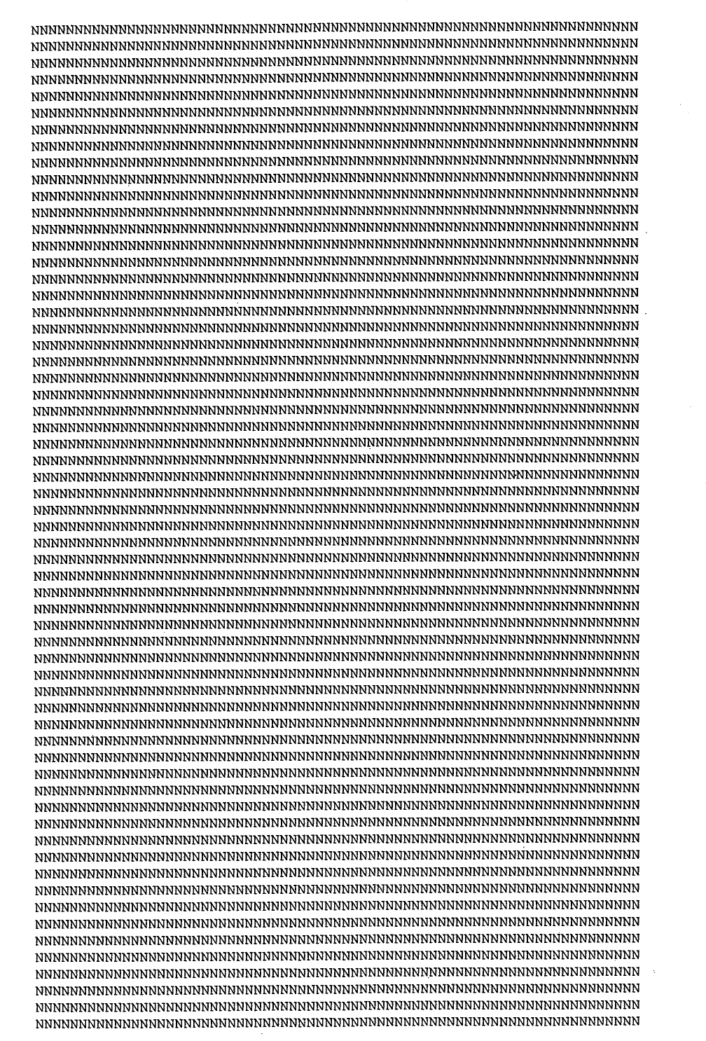

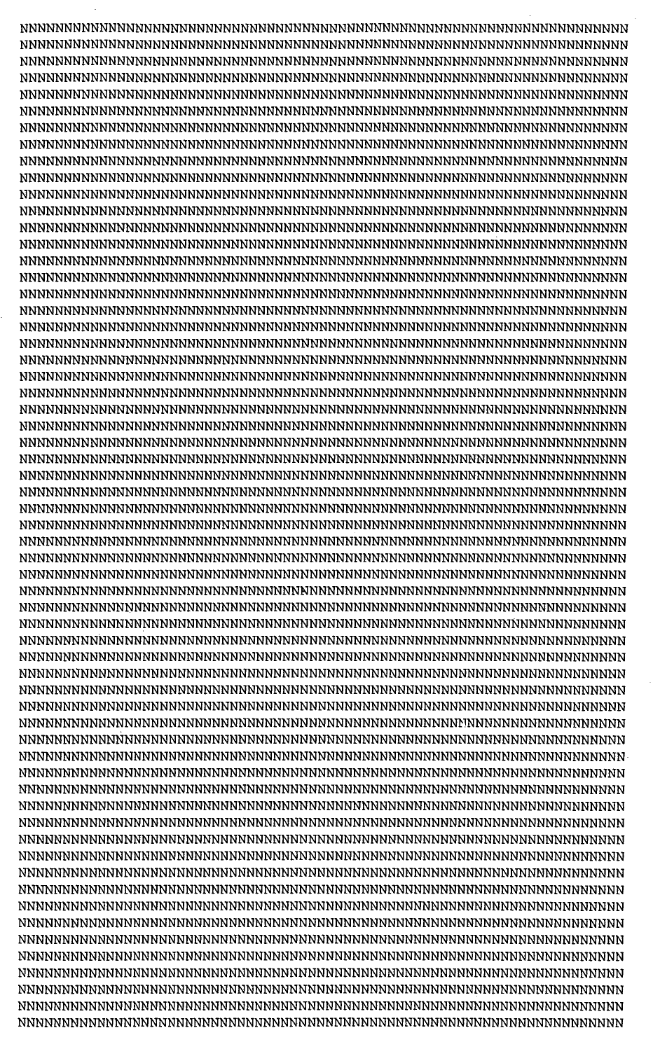

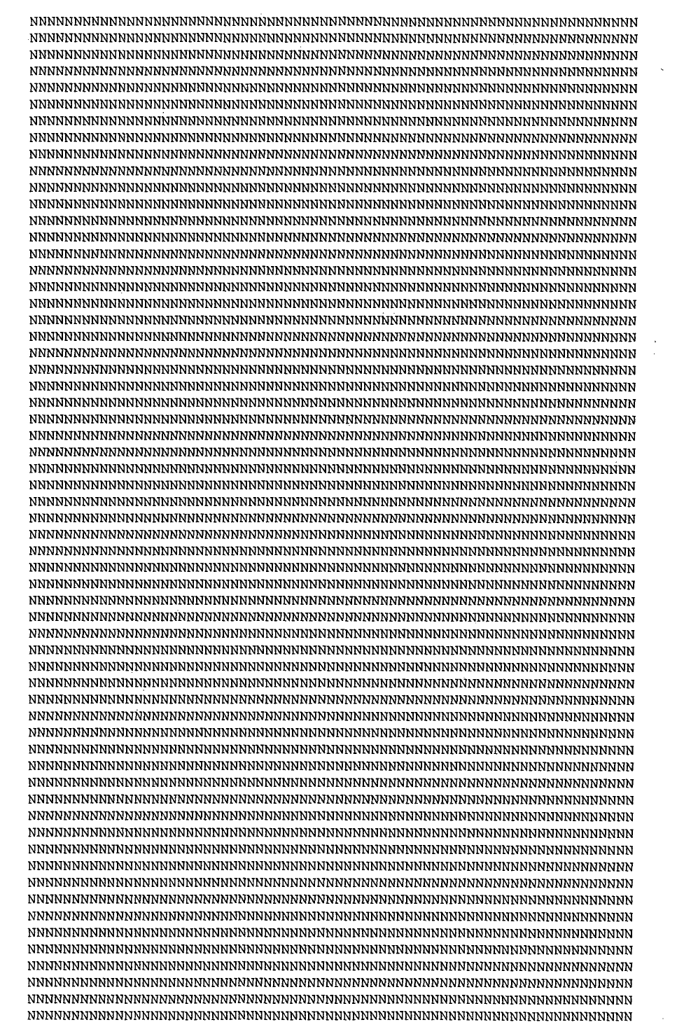

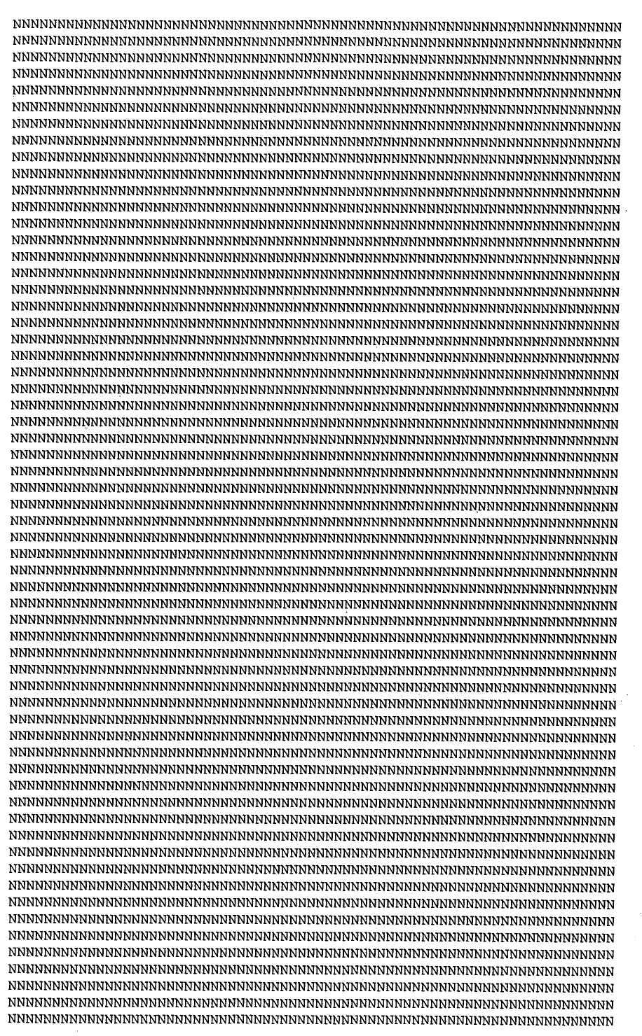

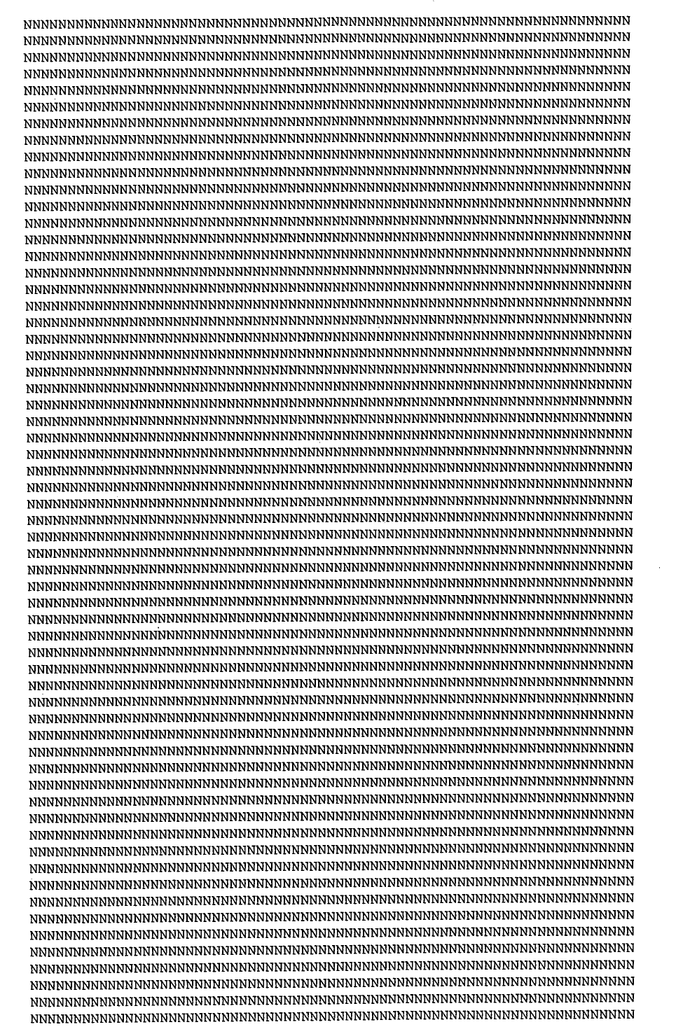

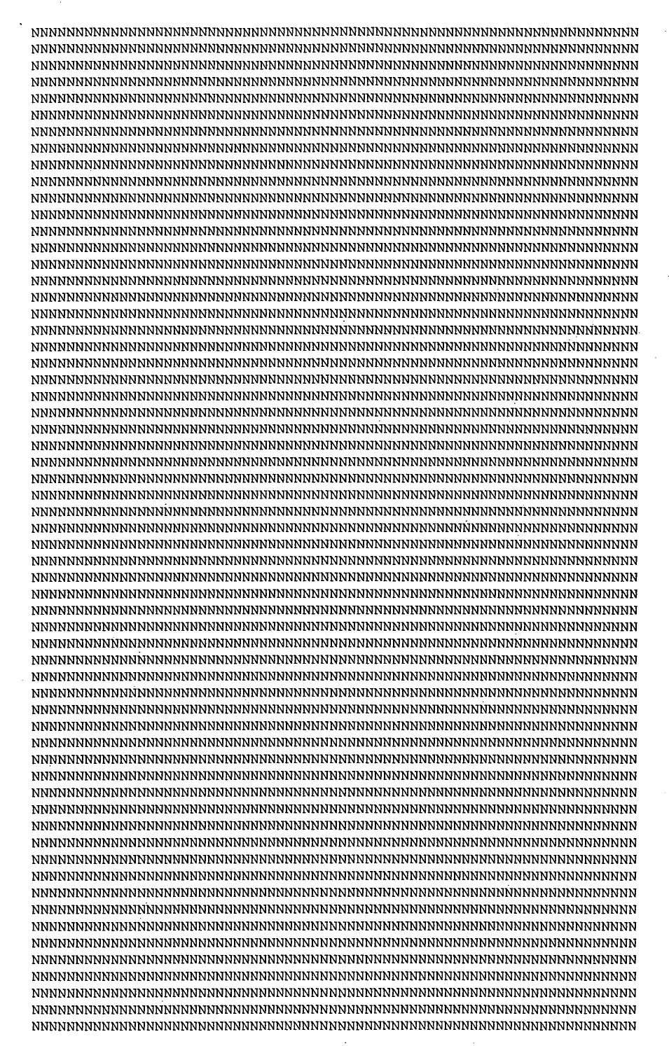

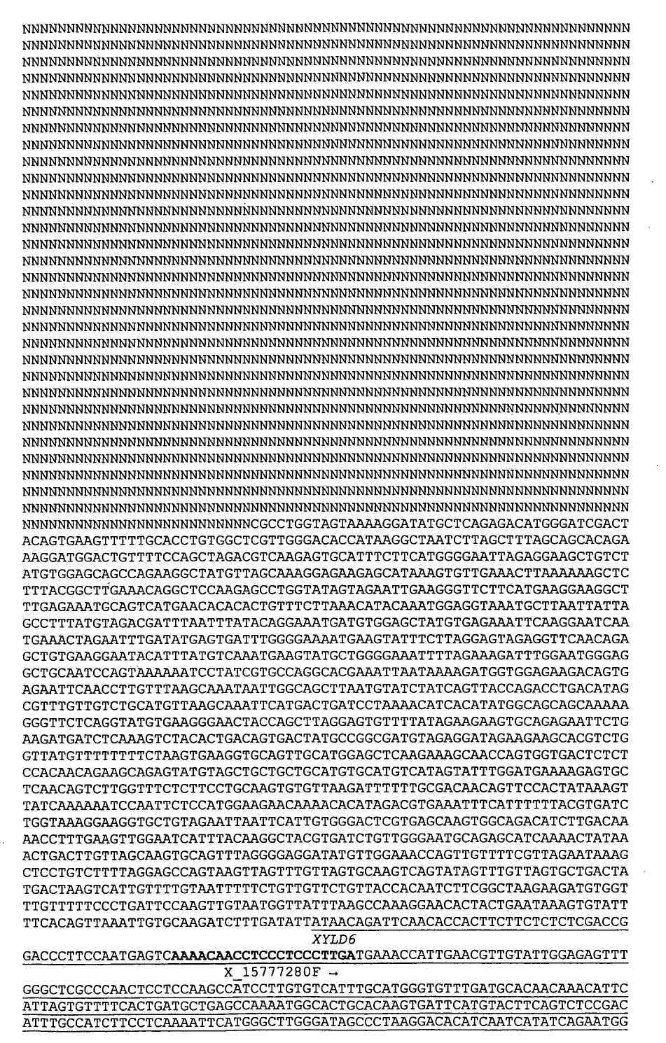

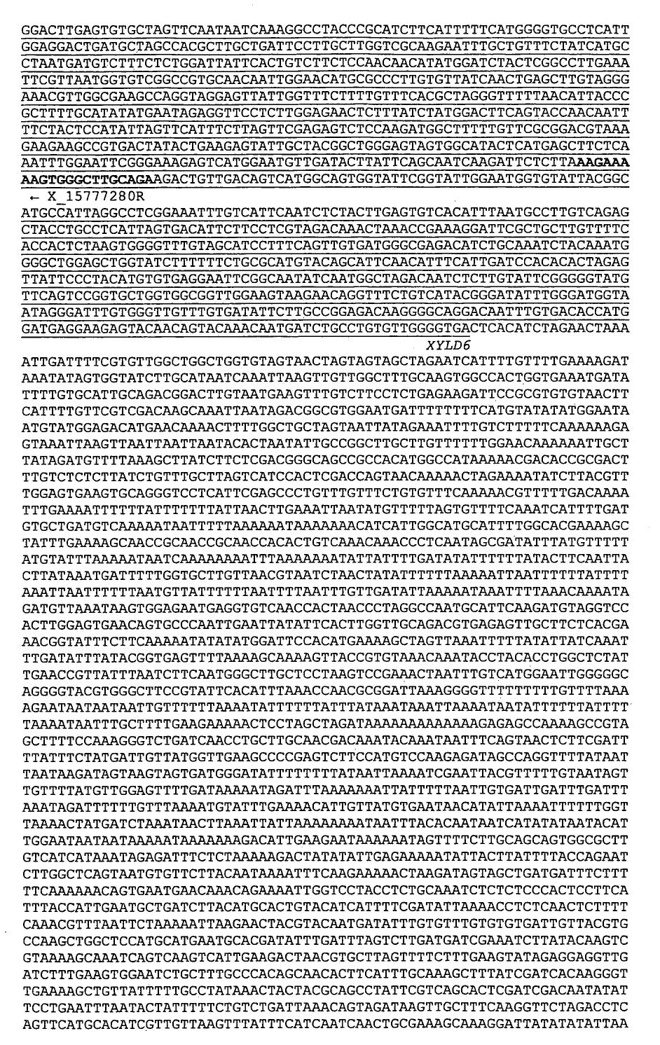

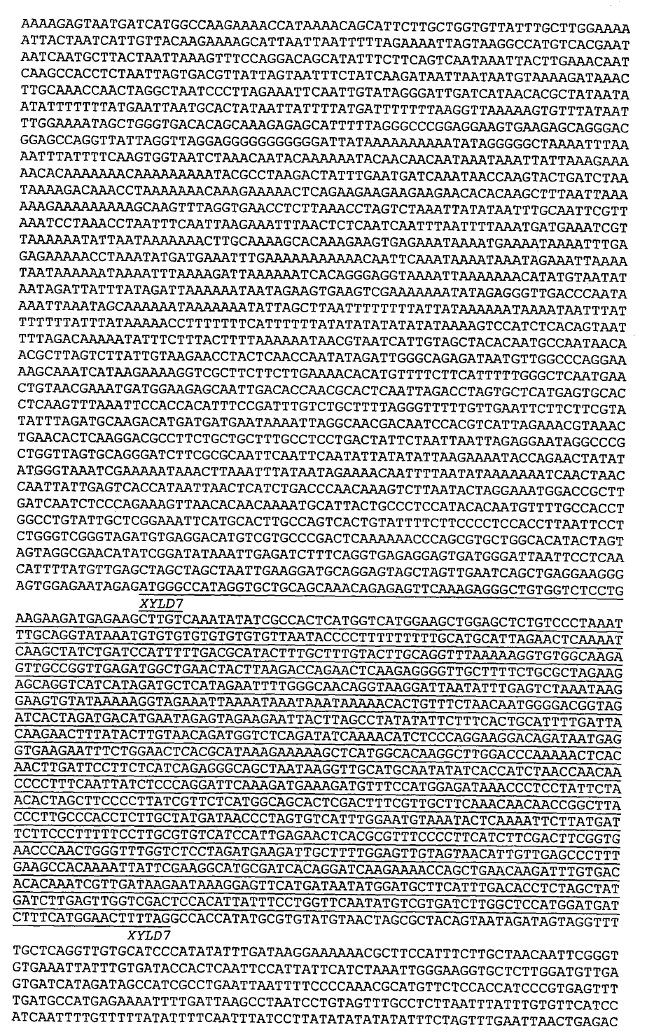

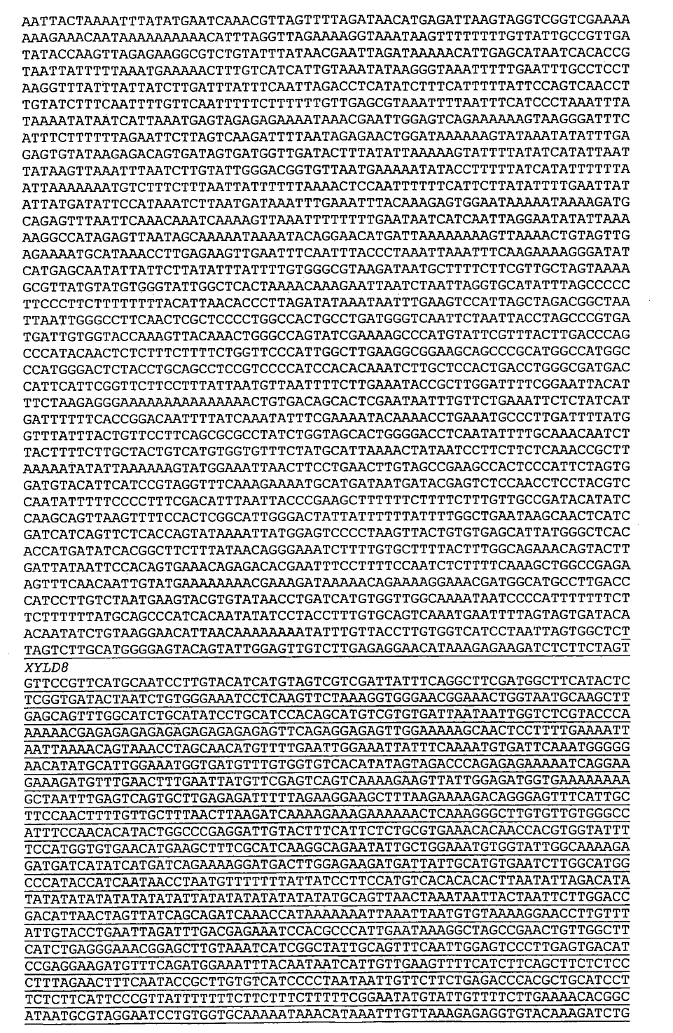

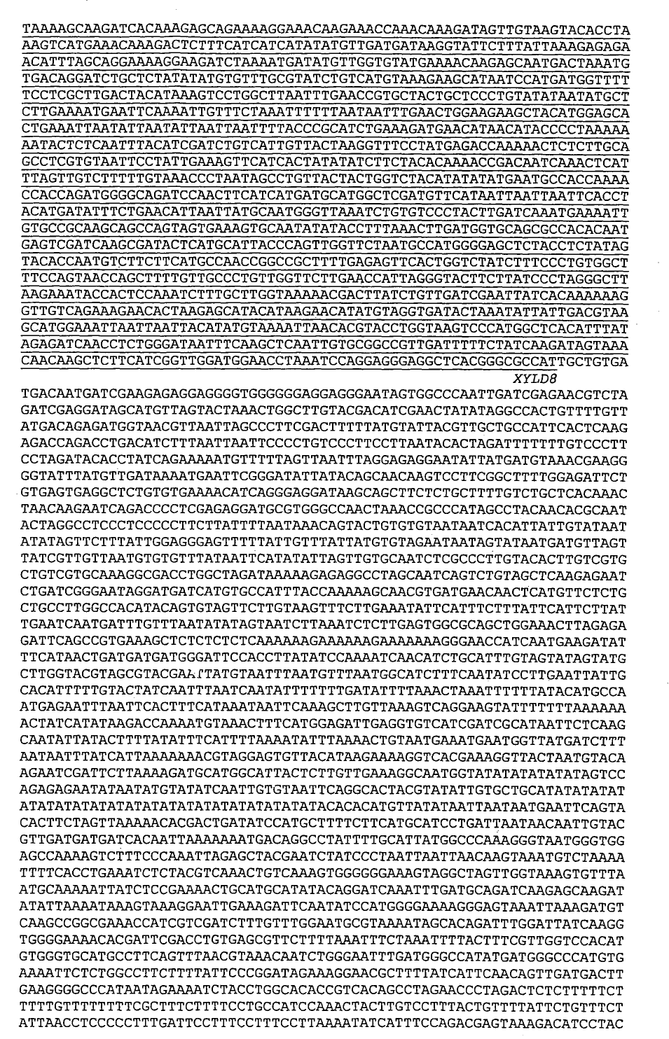

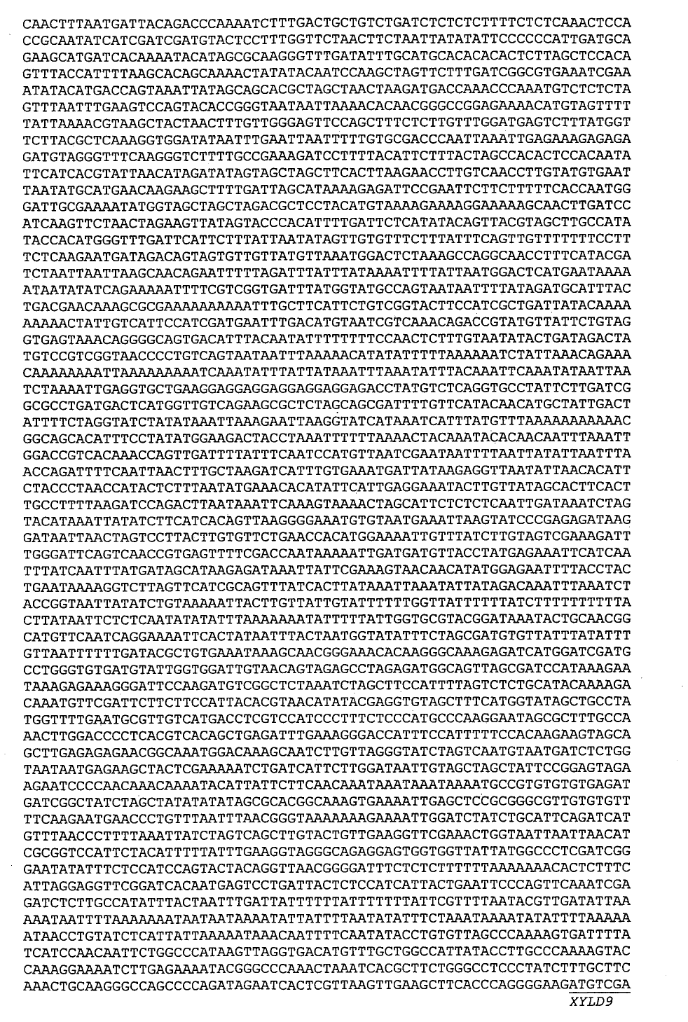

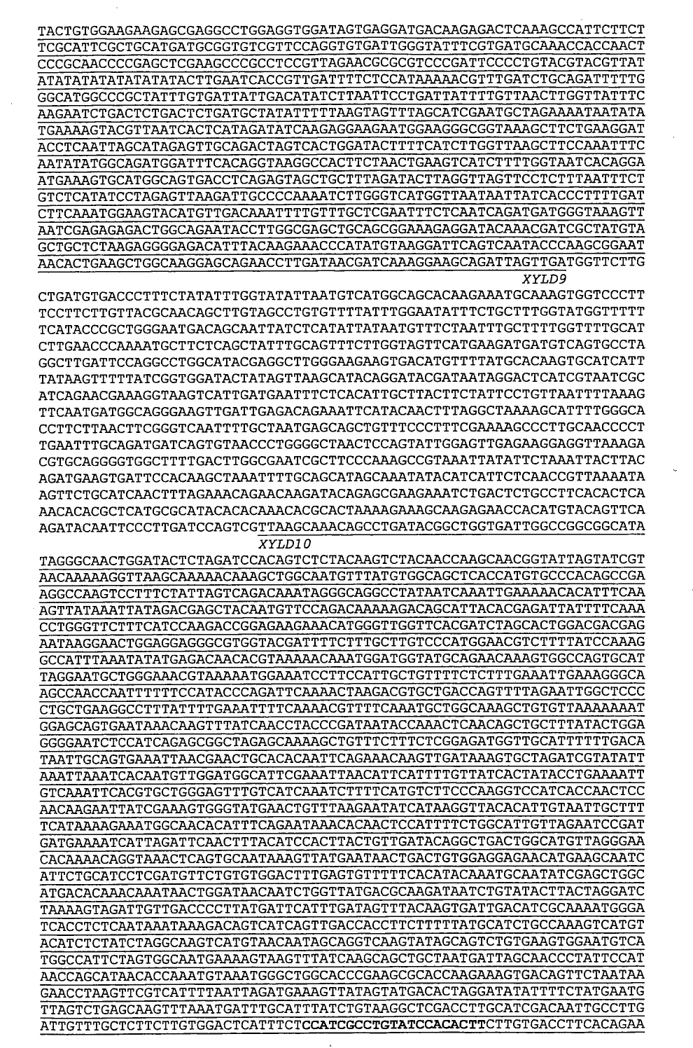

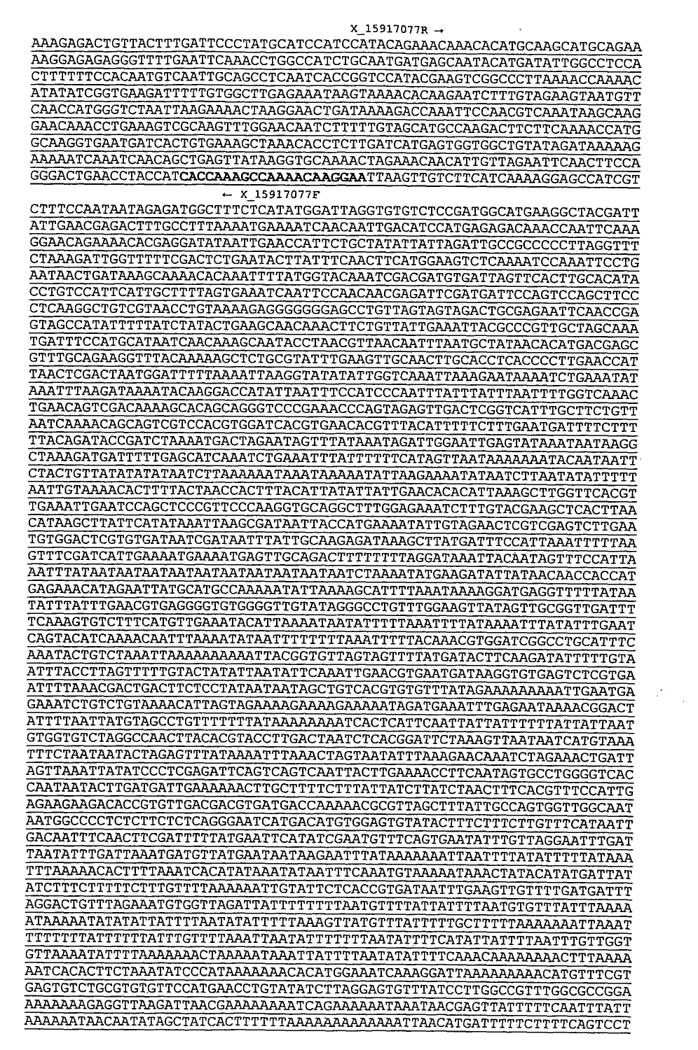

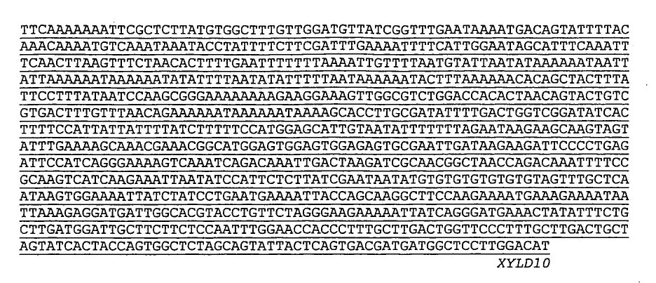

FIG. 15 shows the QTL region of FIG. 1 wherein markers derived from the sequence that we used in QTL identification are indicated by bold type. Gene sequences are labelled and underlined.

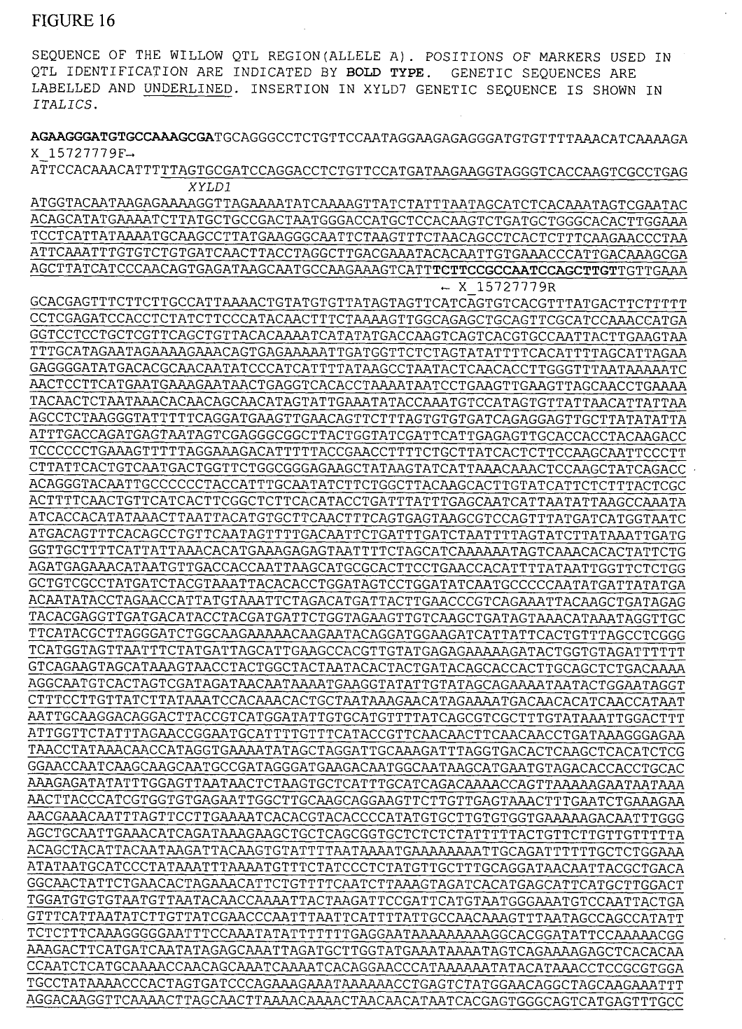

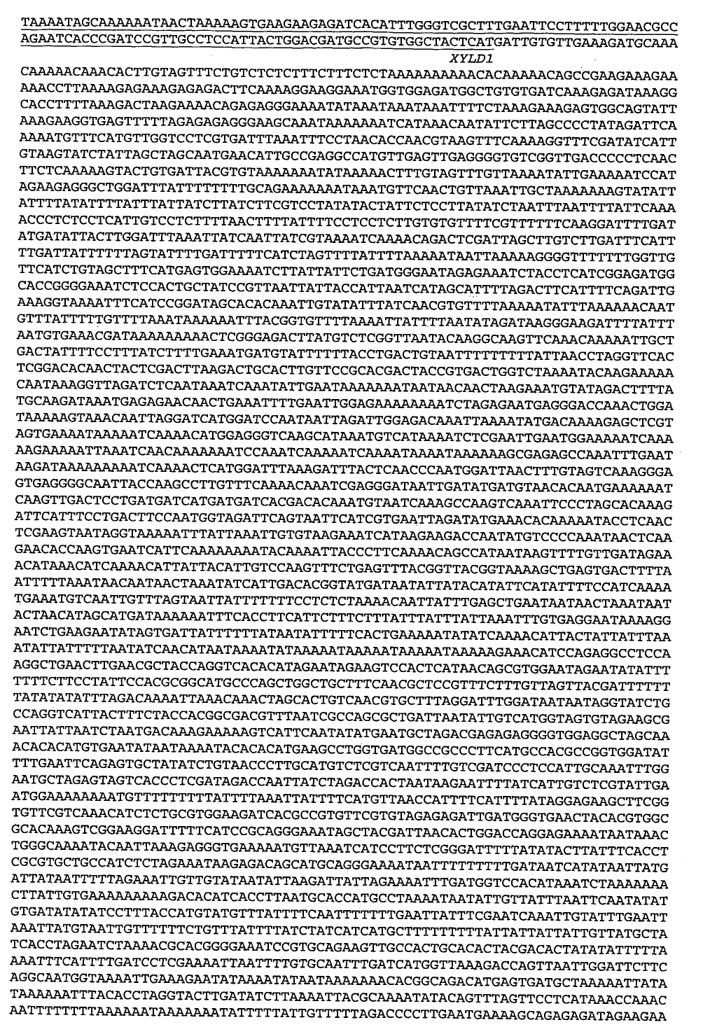

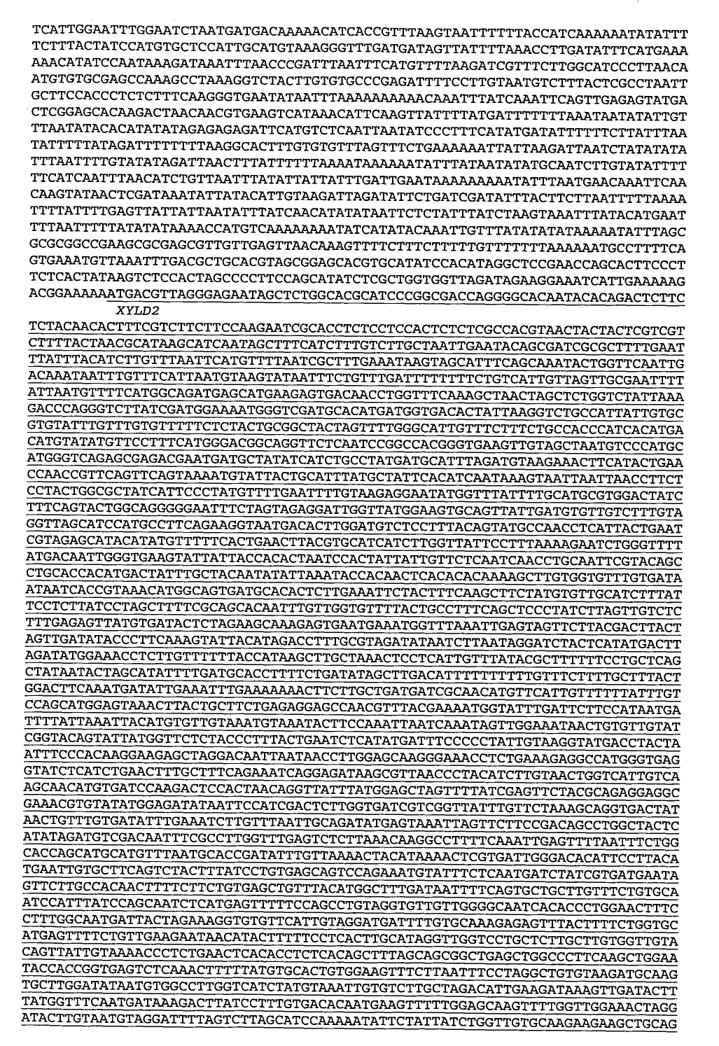

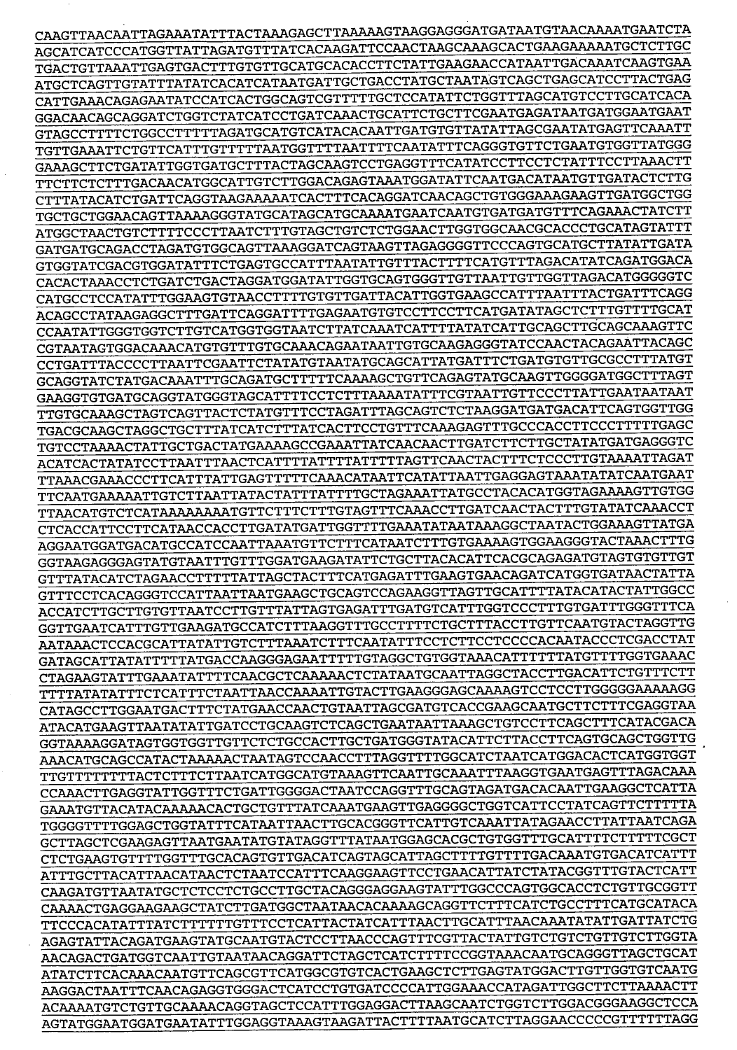

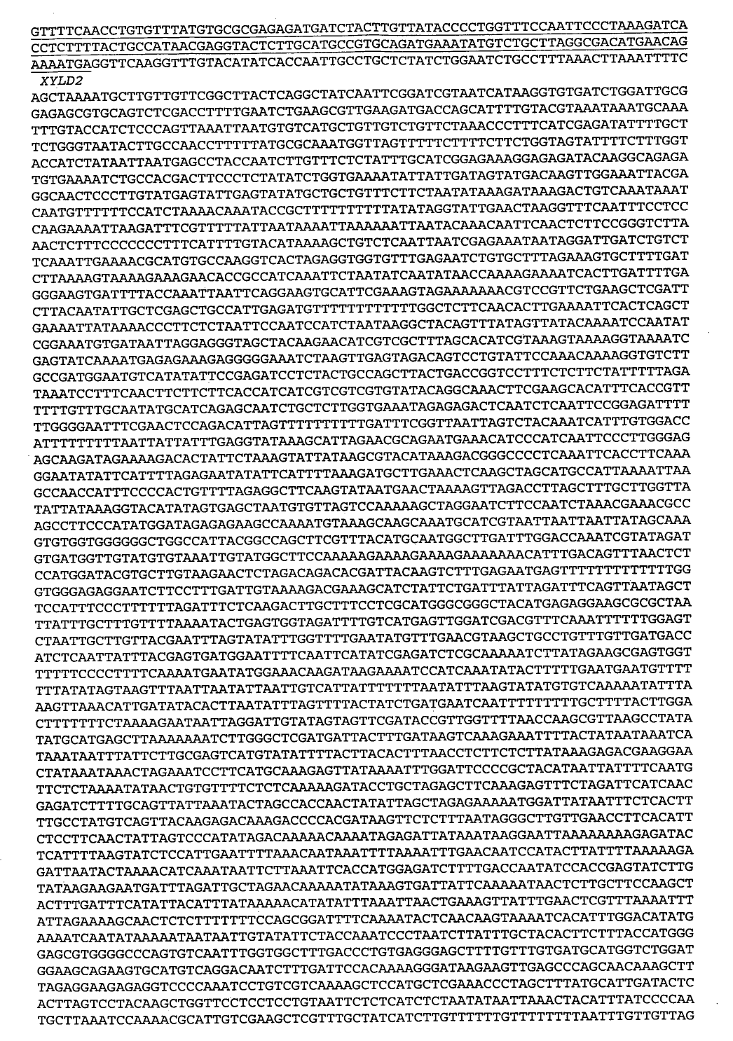

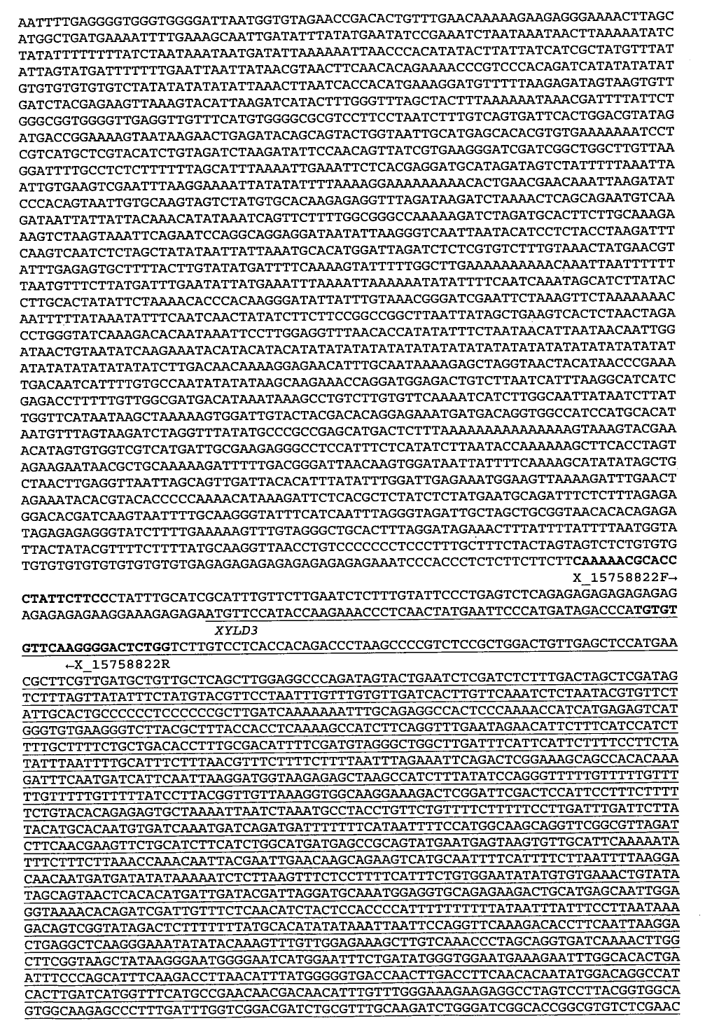

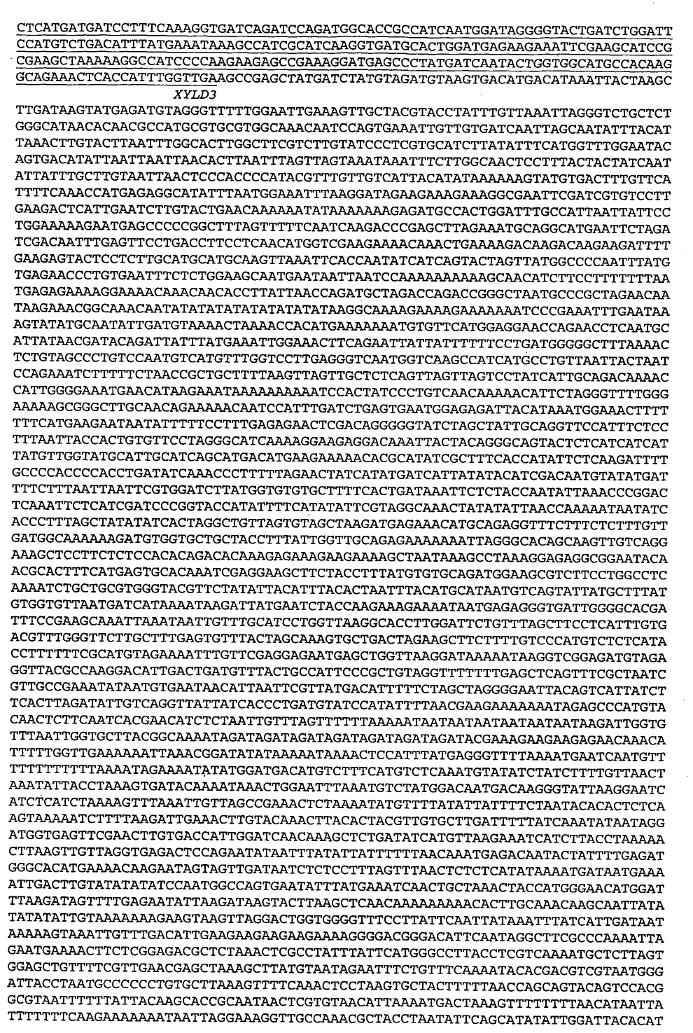

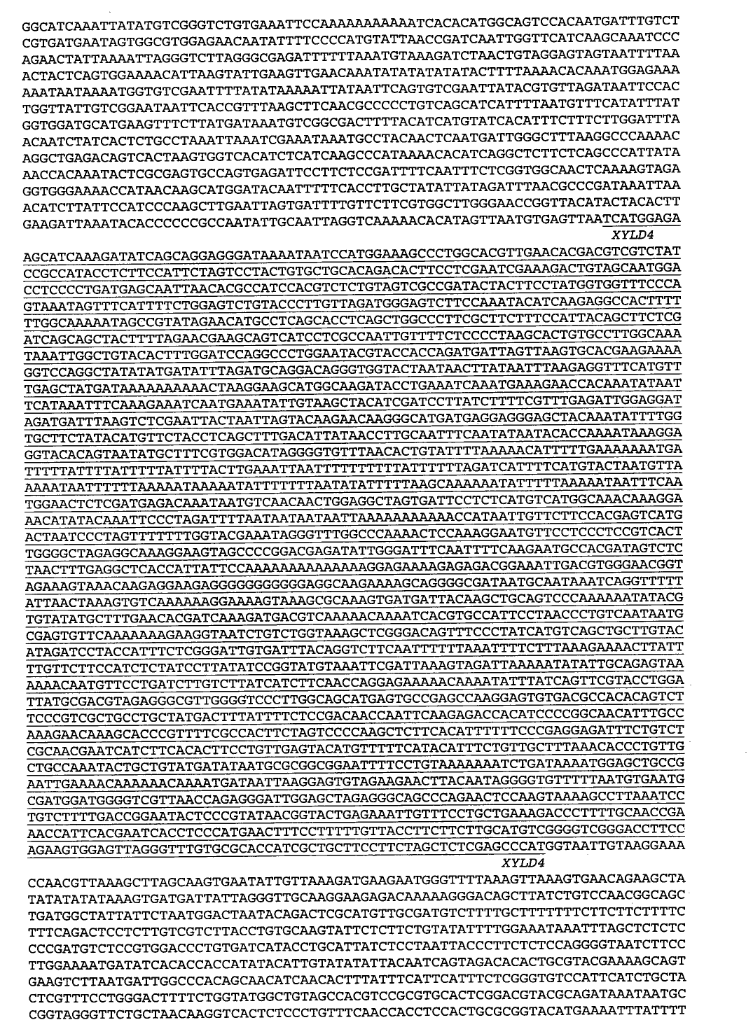

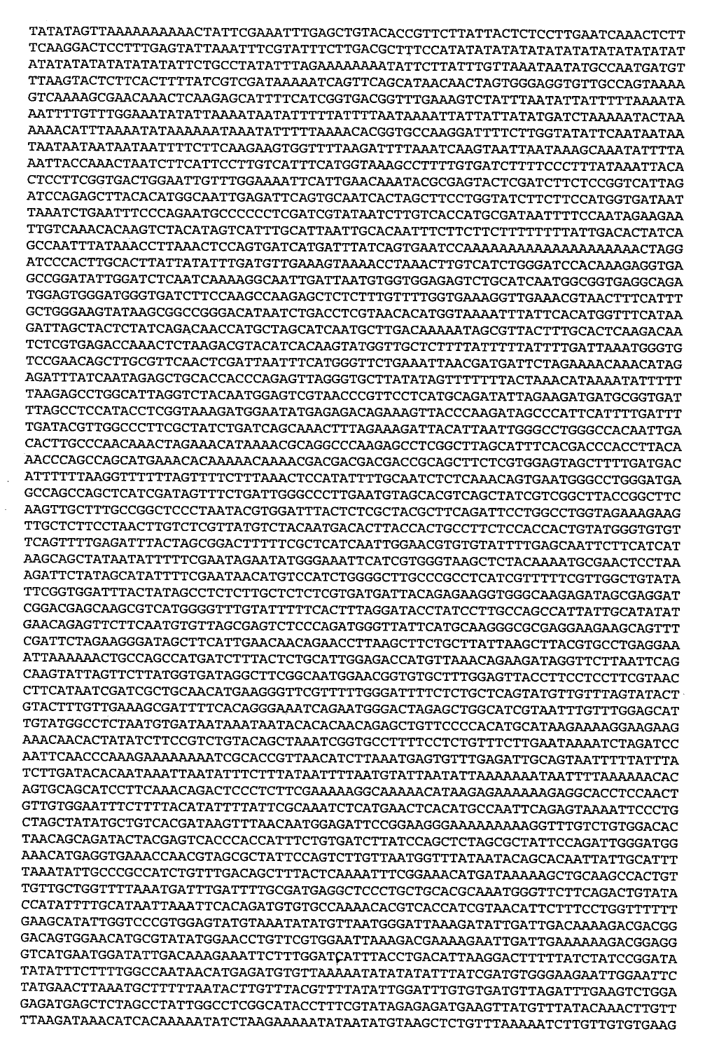

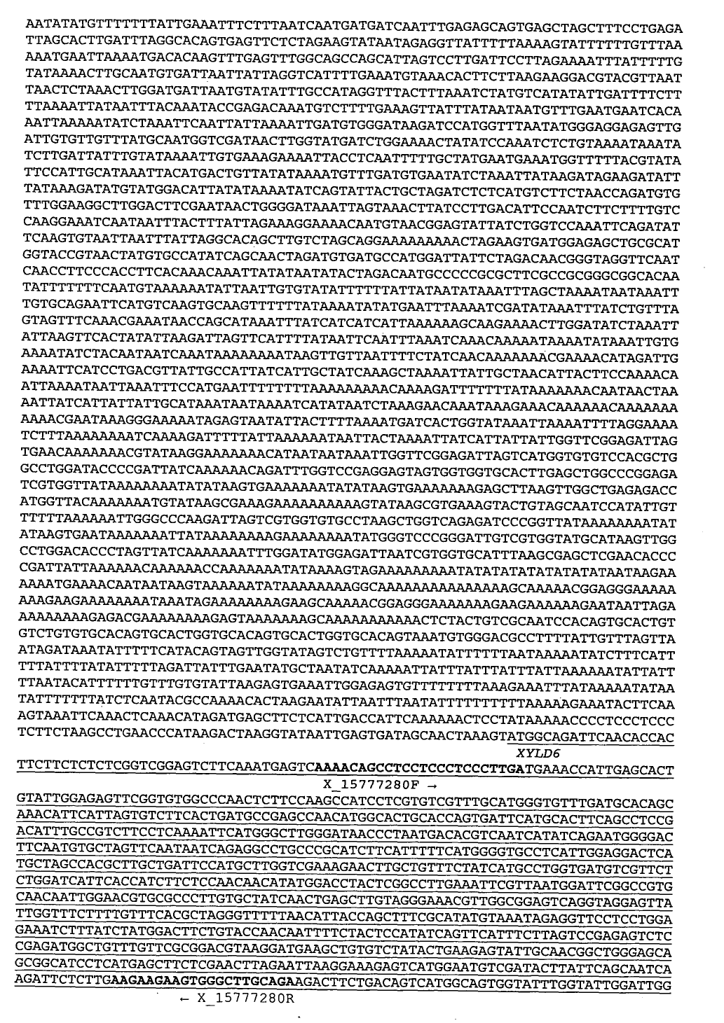

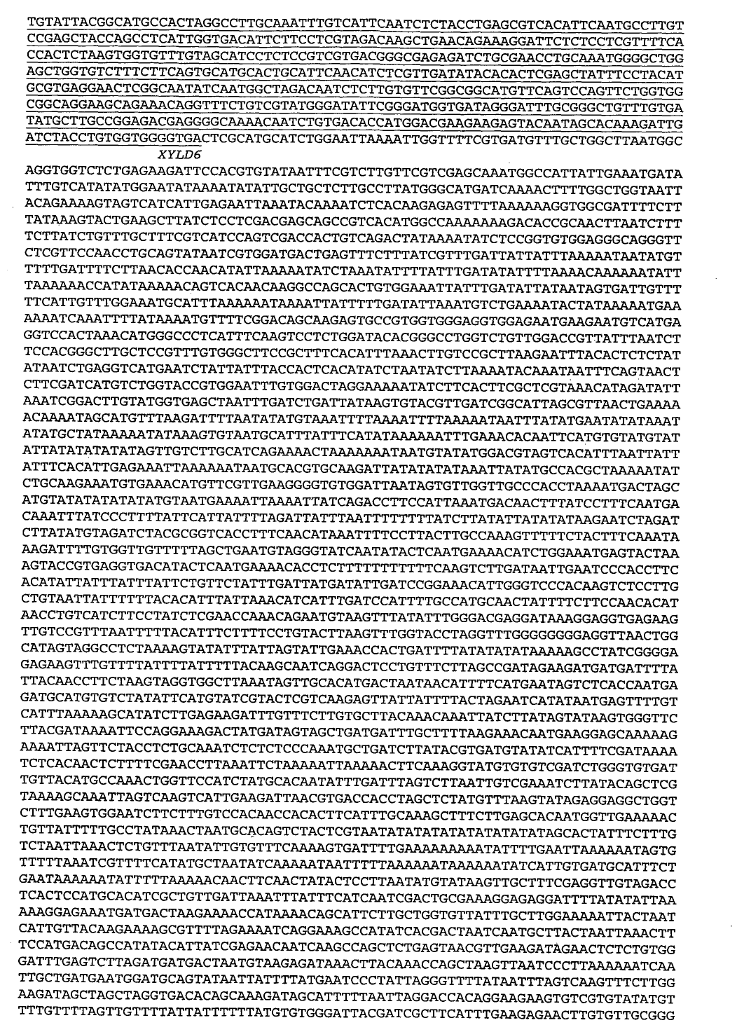

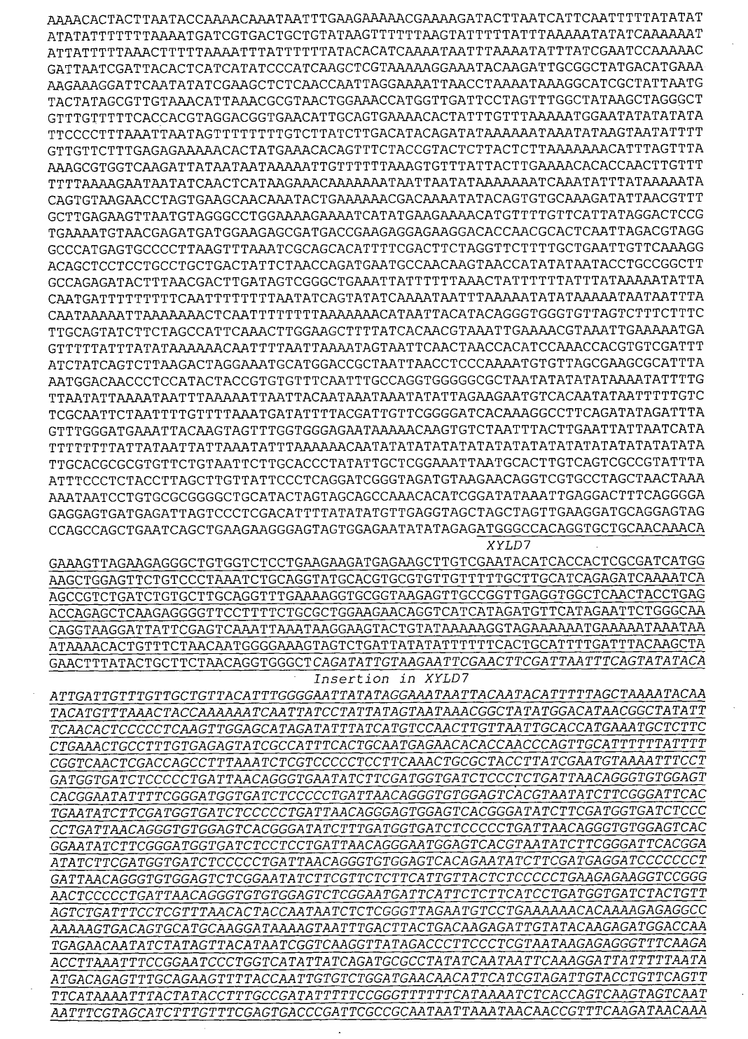

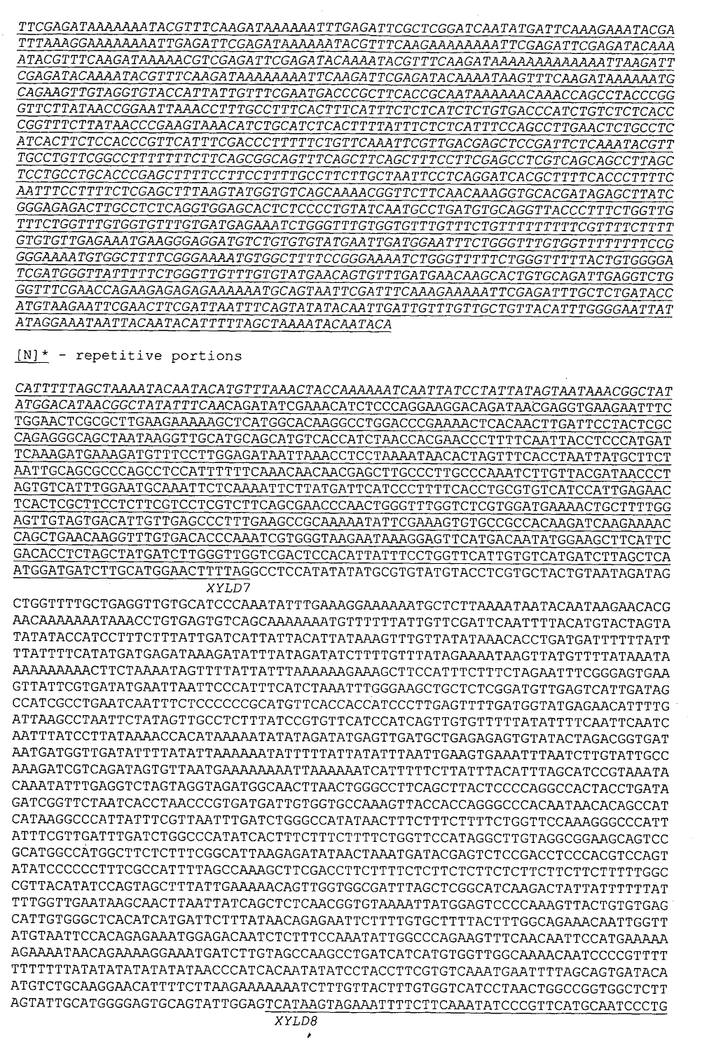

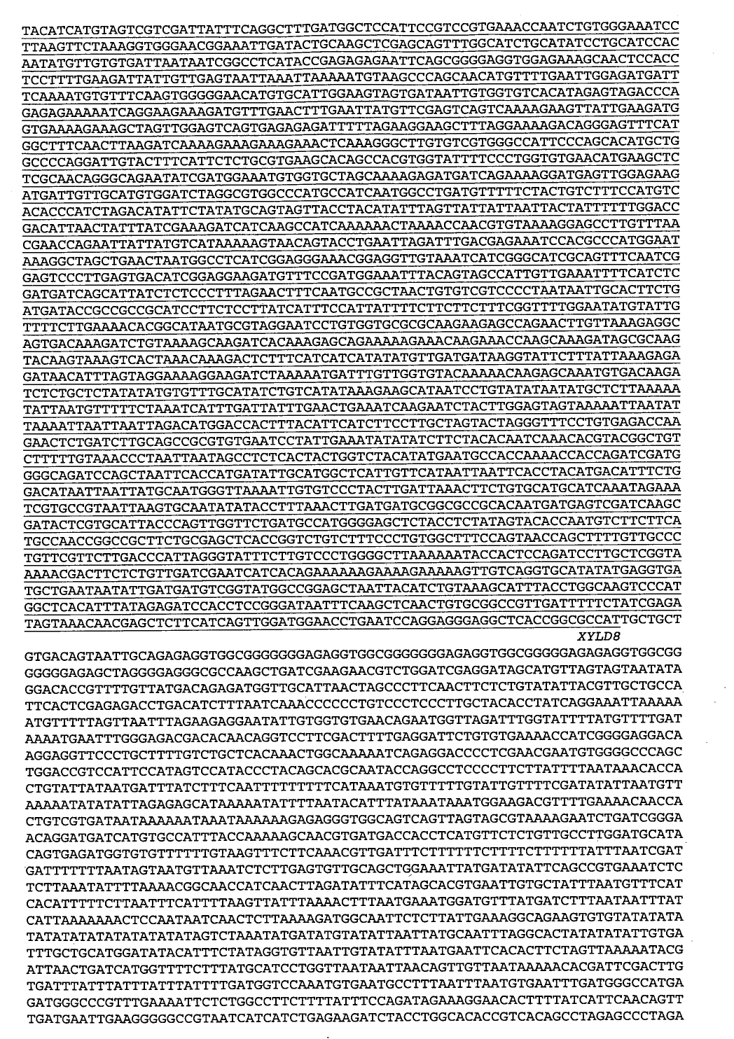

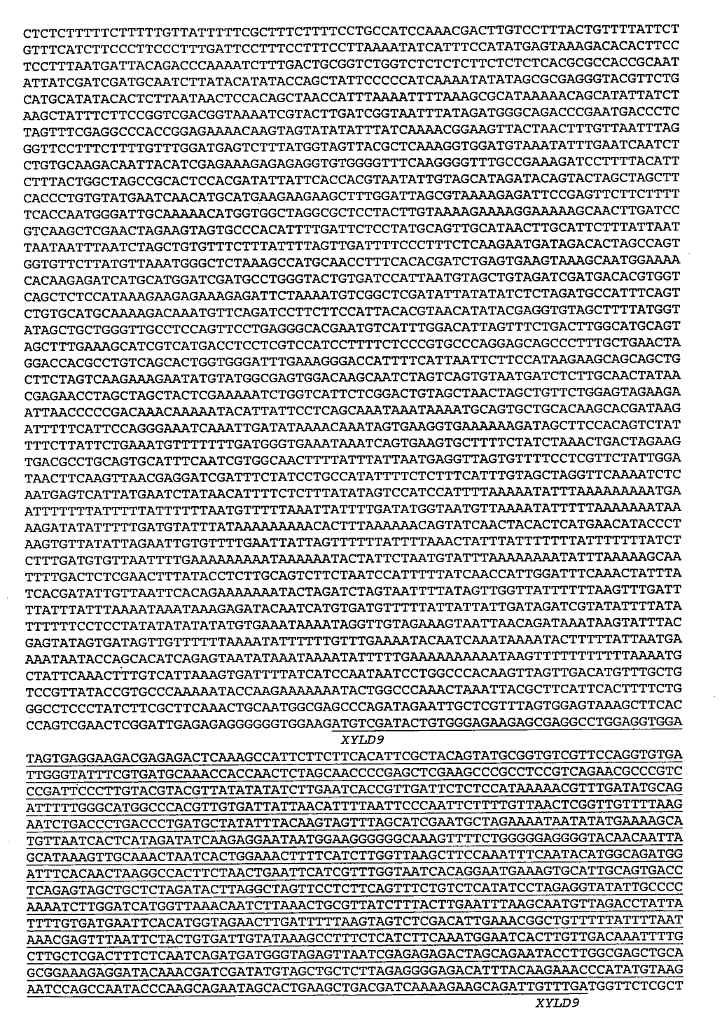

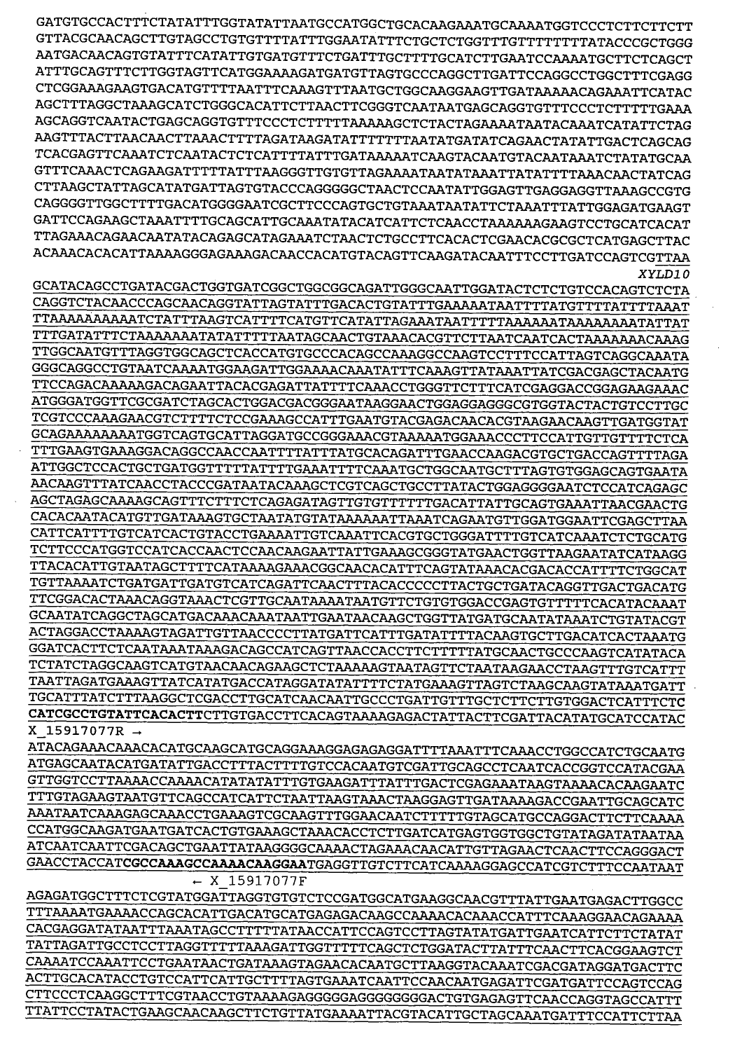

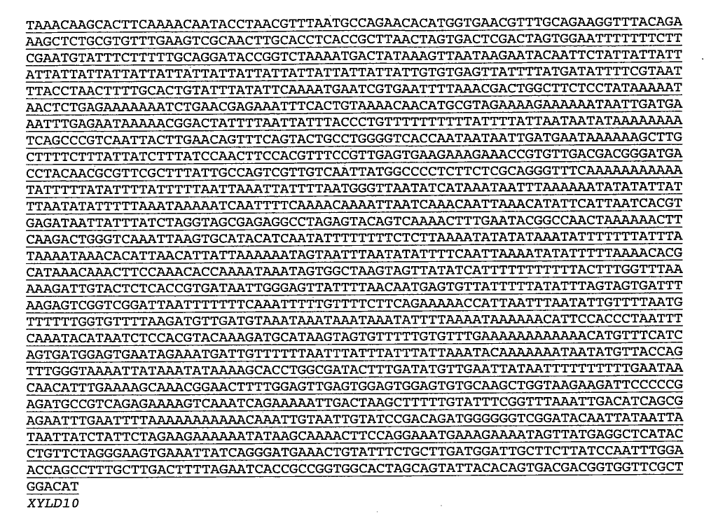

FIG. 16 shows the QTL region of FIG. 2 wherein markers derived from the sequence that we used in QTL identification are indicated by bold type. Gene sequences are labelled and underlined.

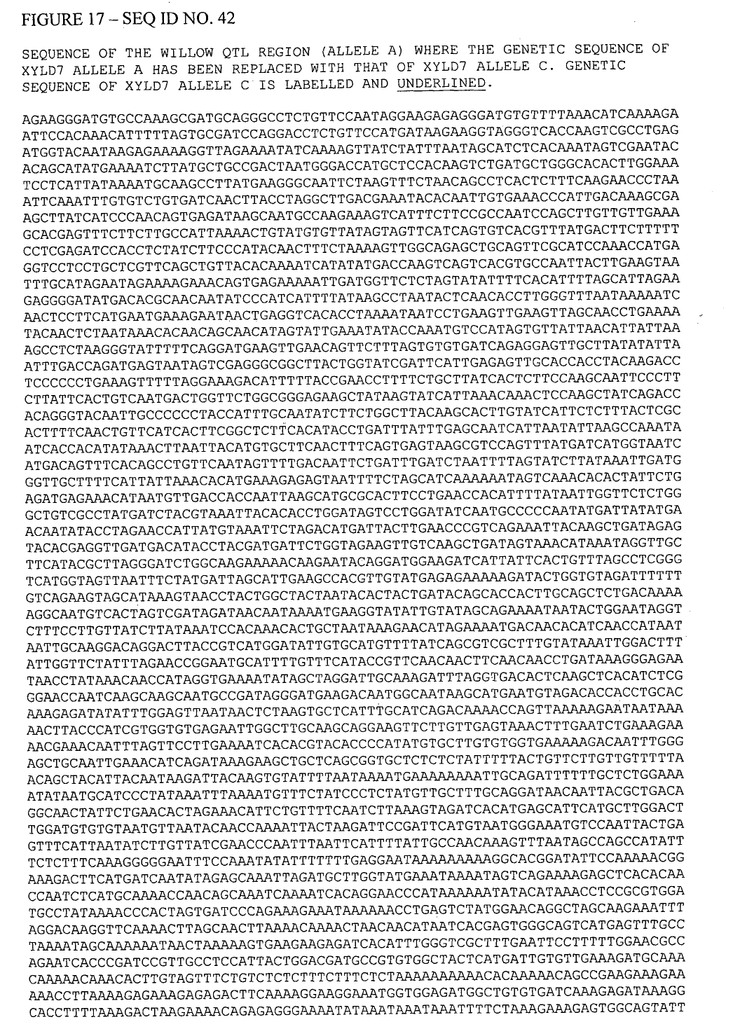

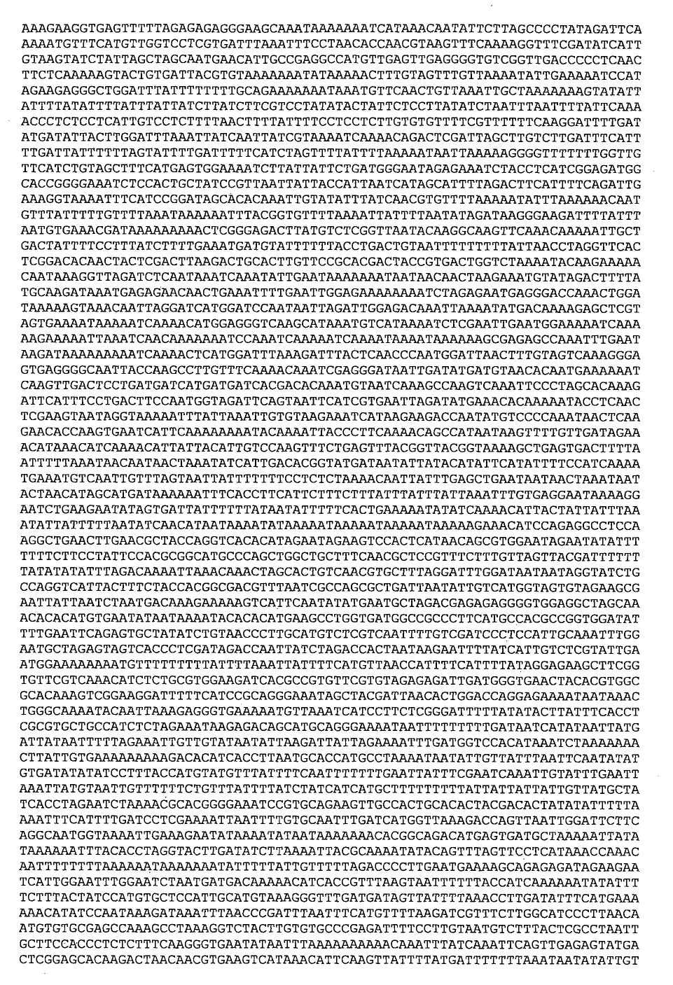

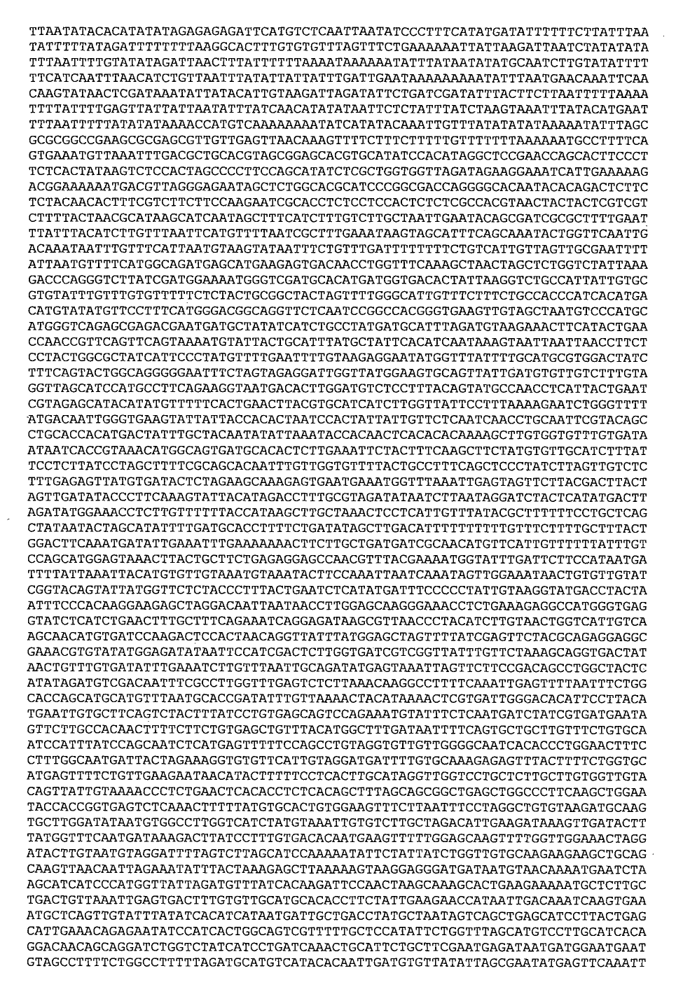

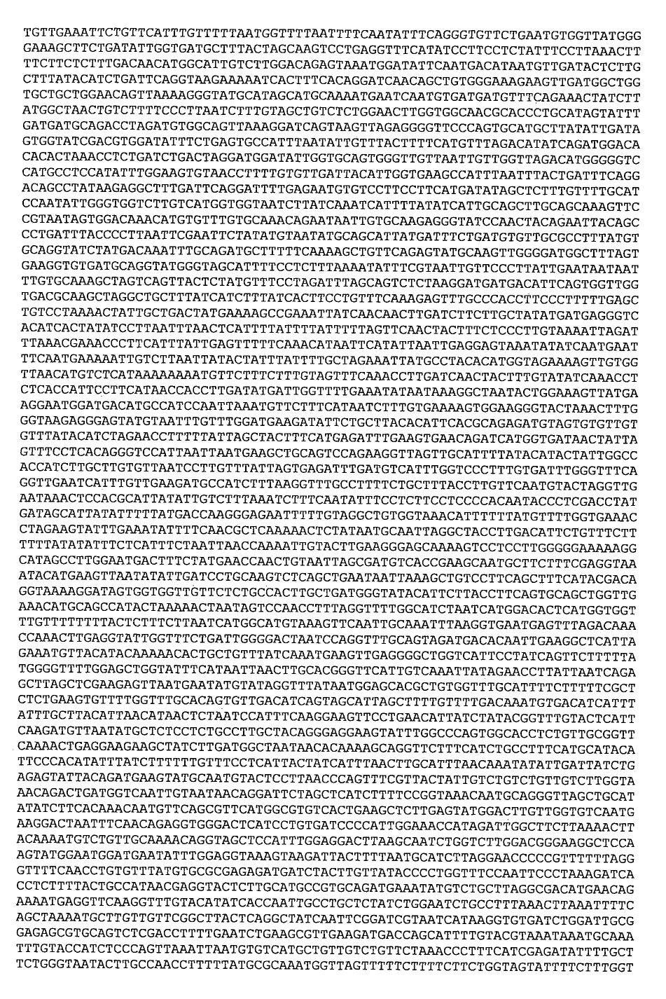

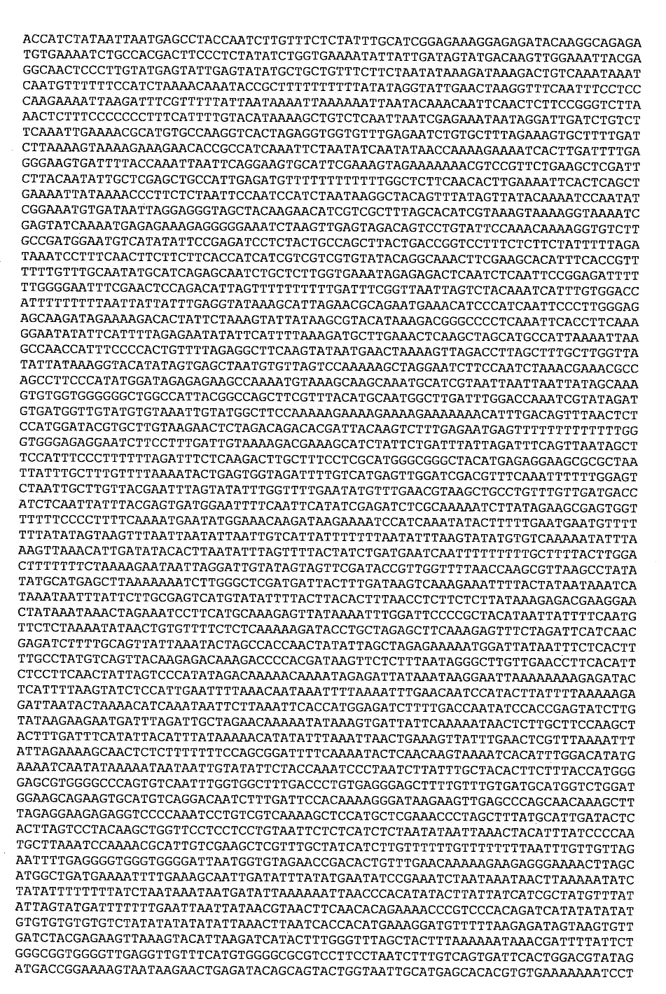

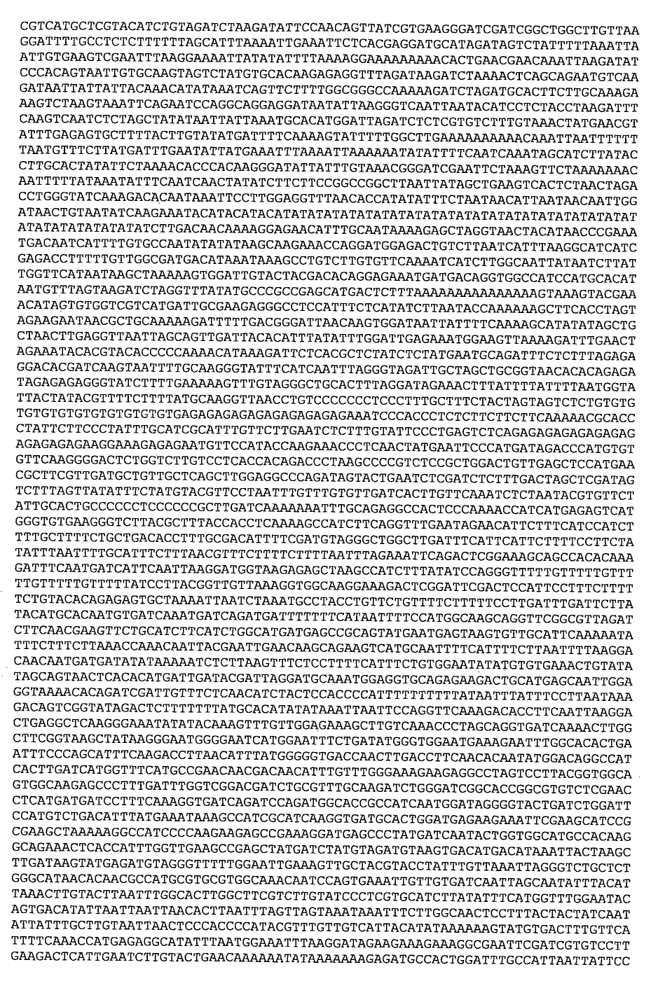

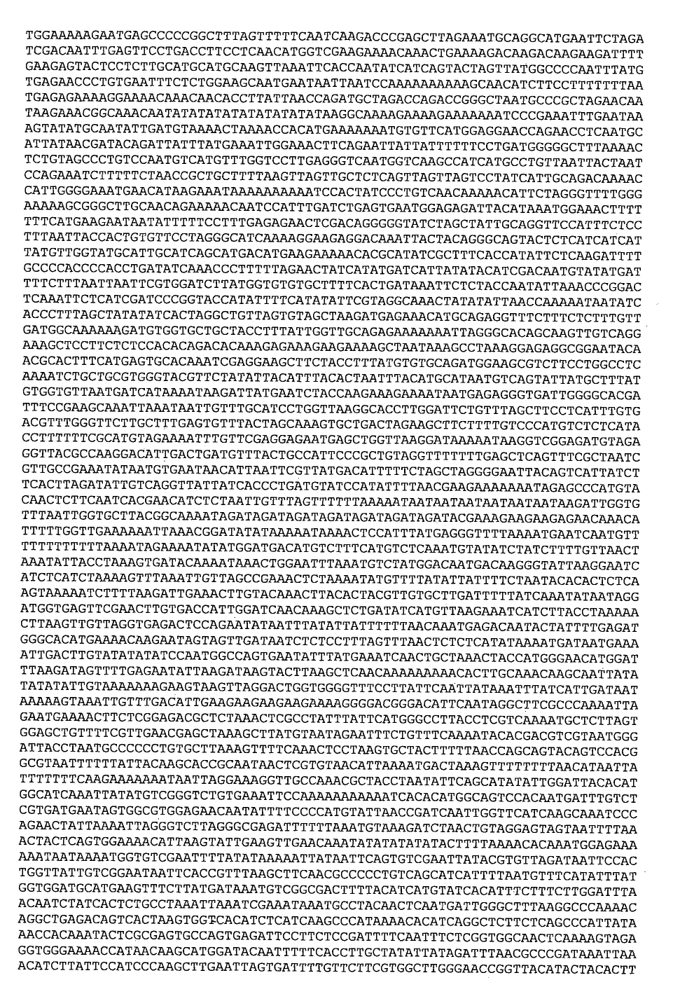

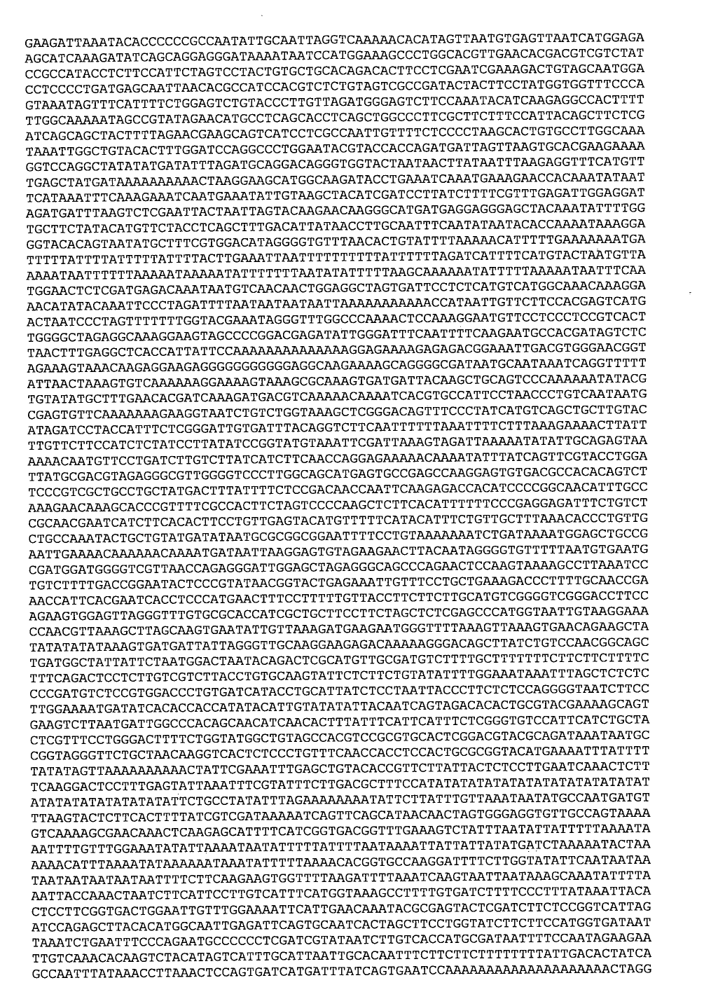

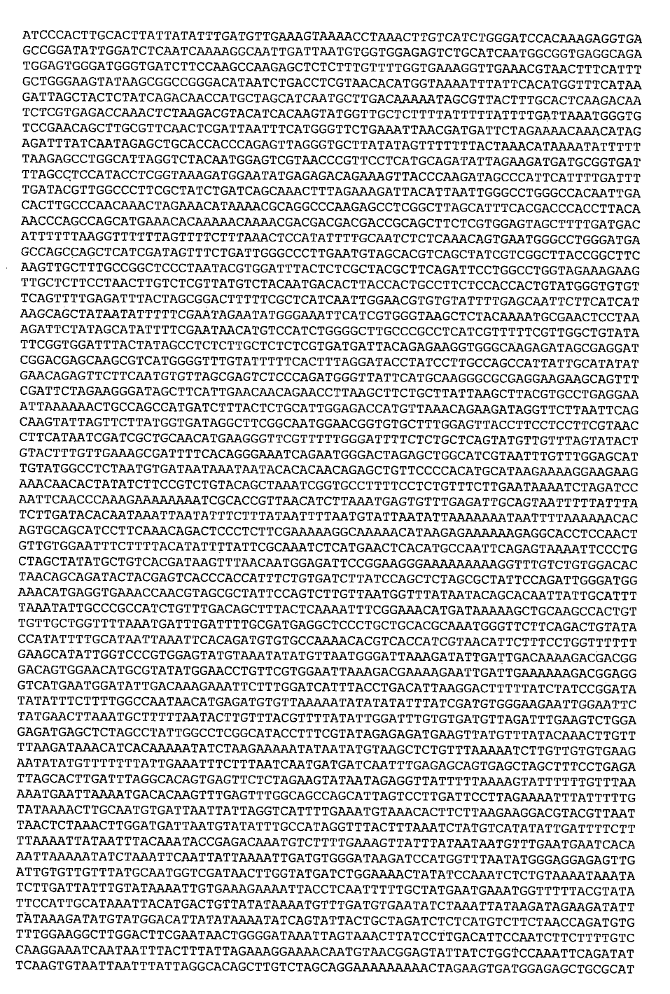

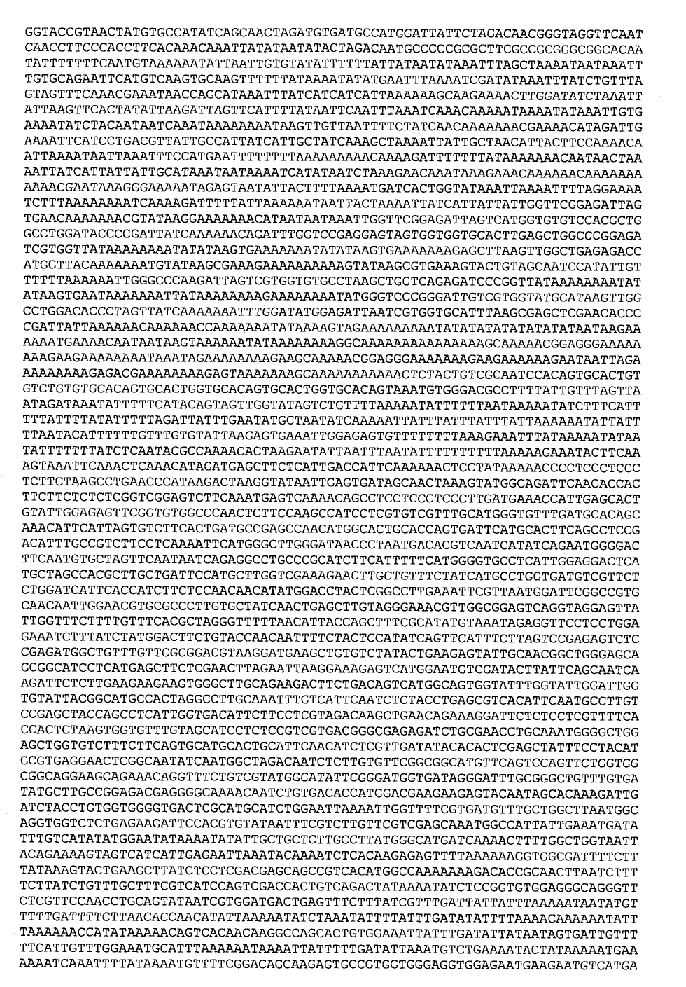

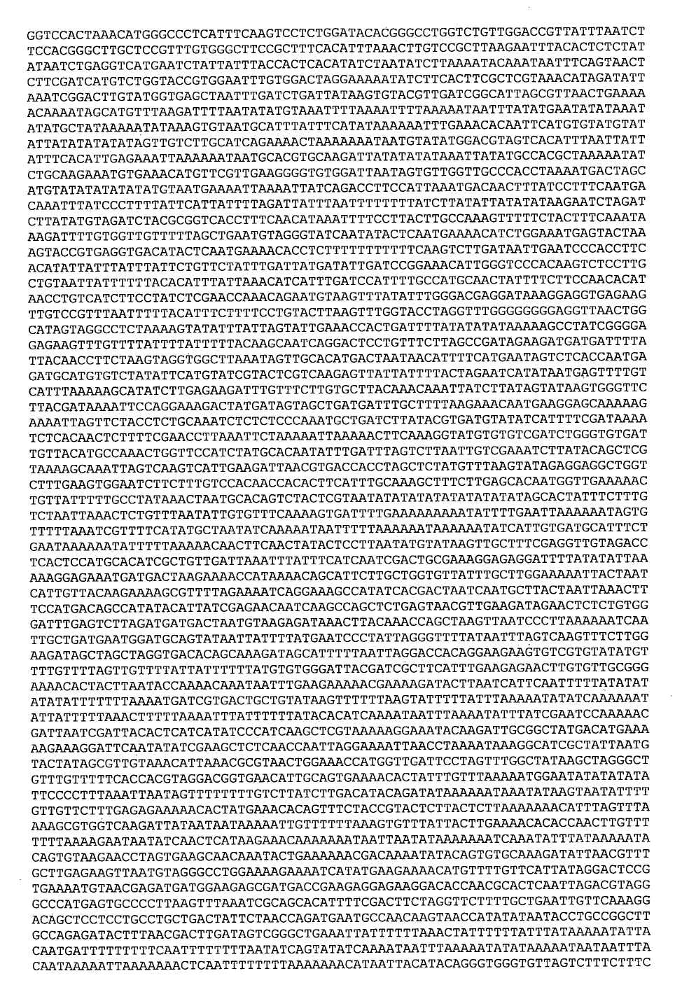

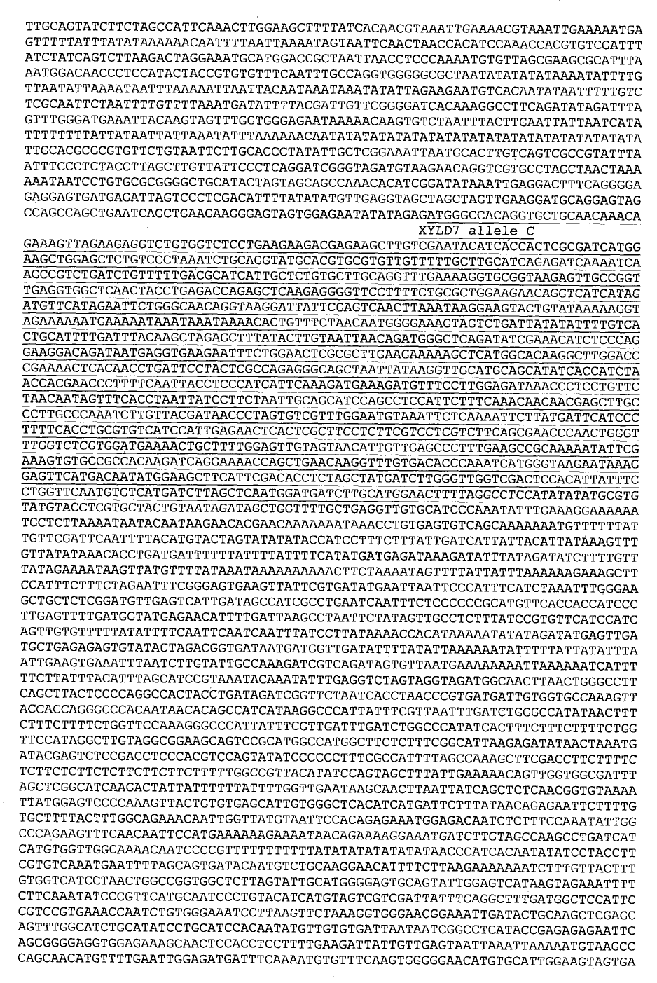

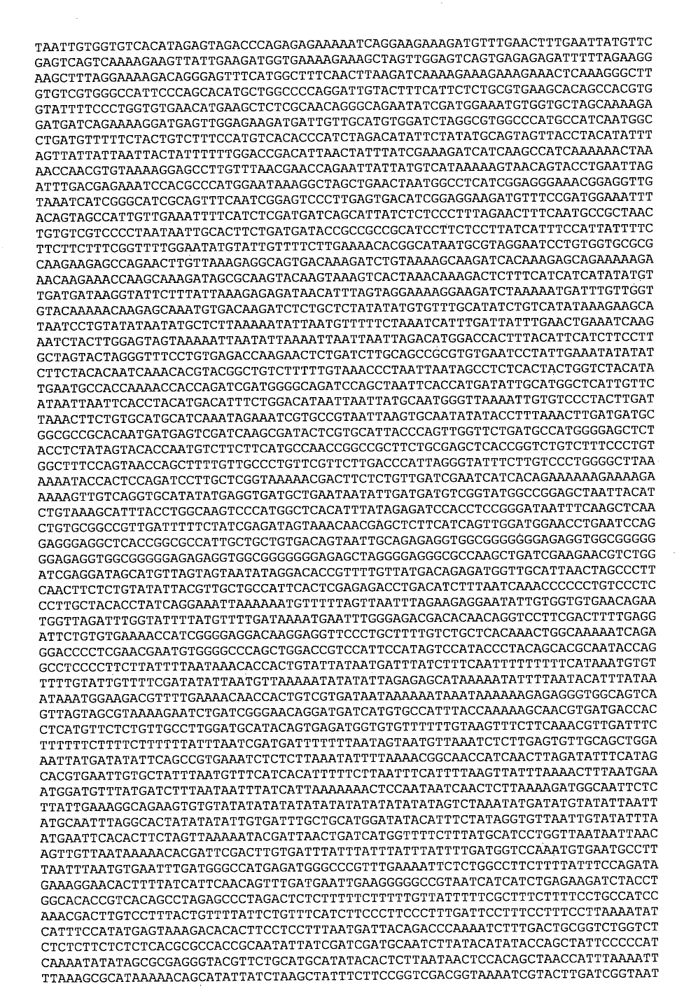

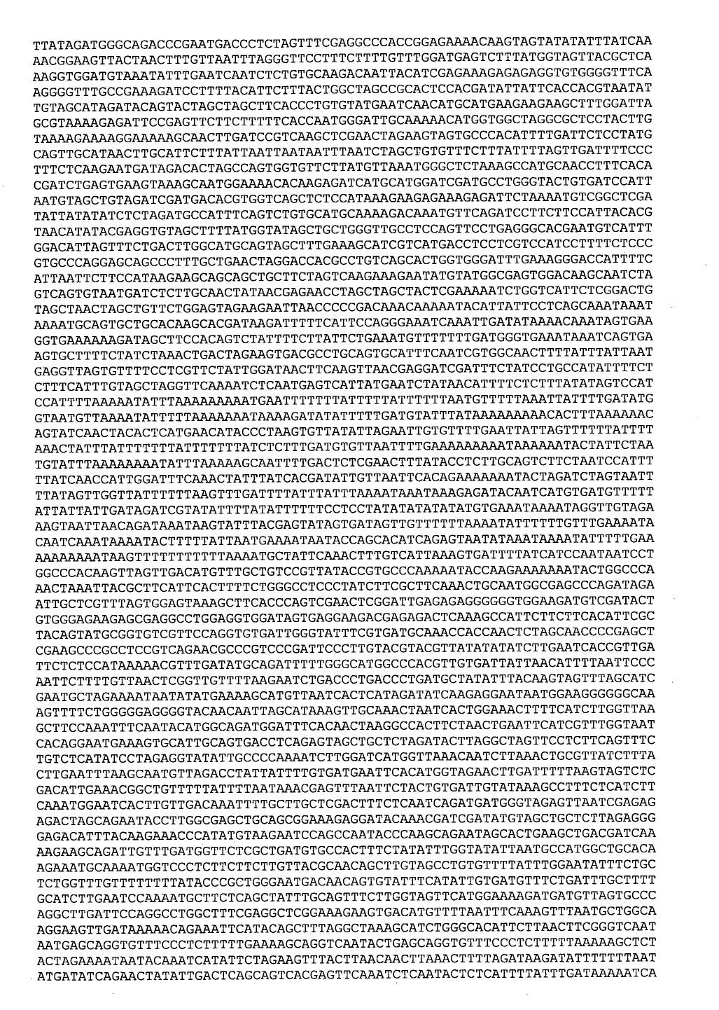

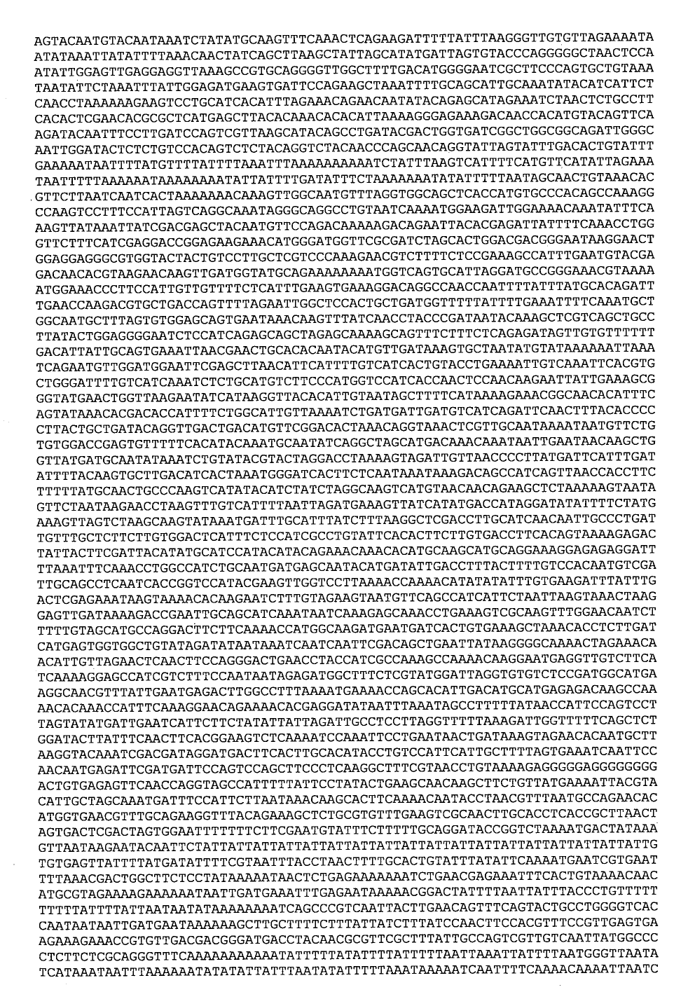

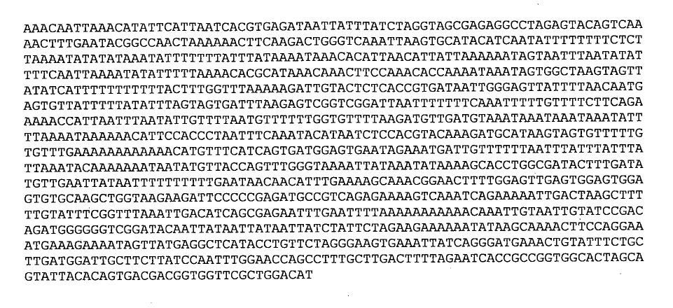

FIG. 17 shows the QTL region of FIG. 2 wherein the sequence of Xyld7 allele A has been replaced with Xyld7 allele C.

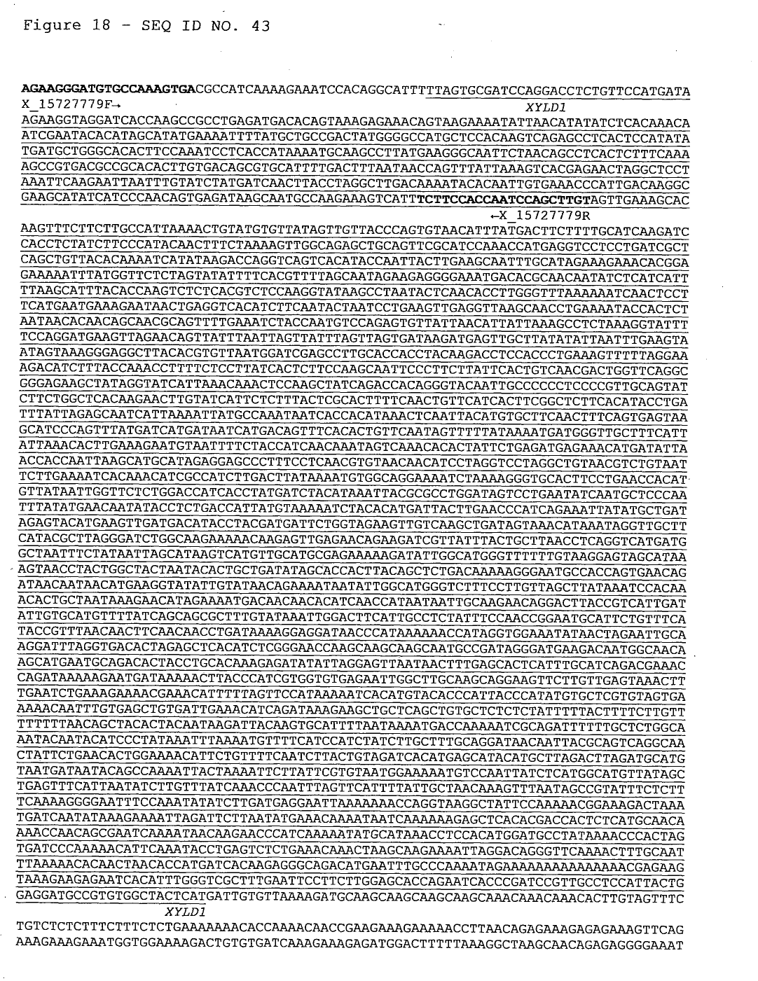

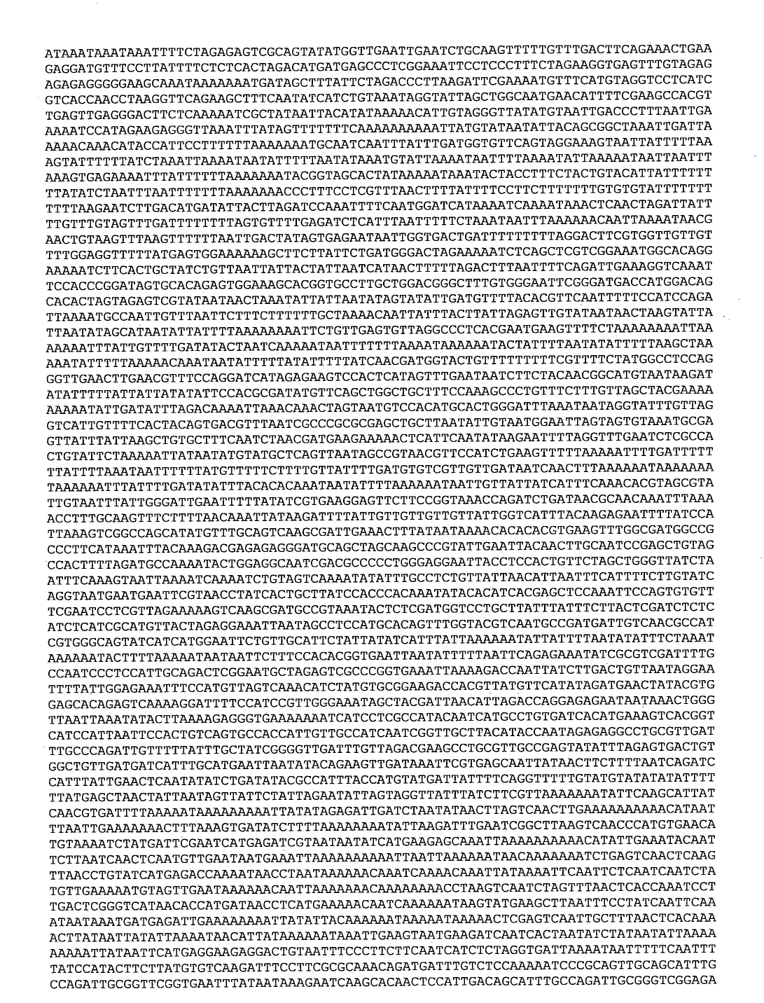

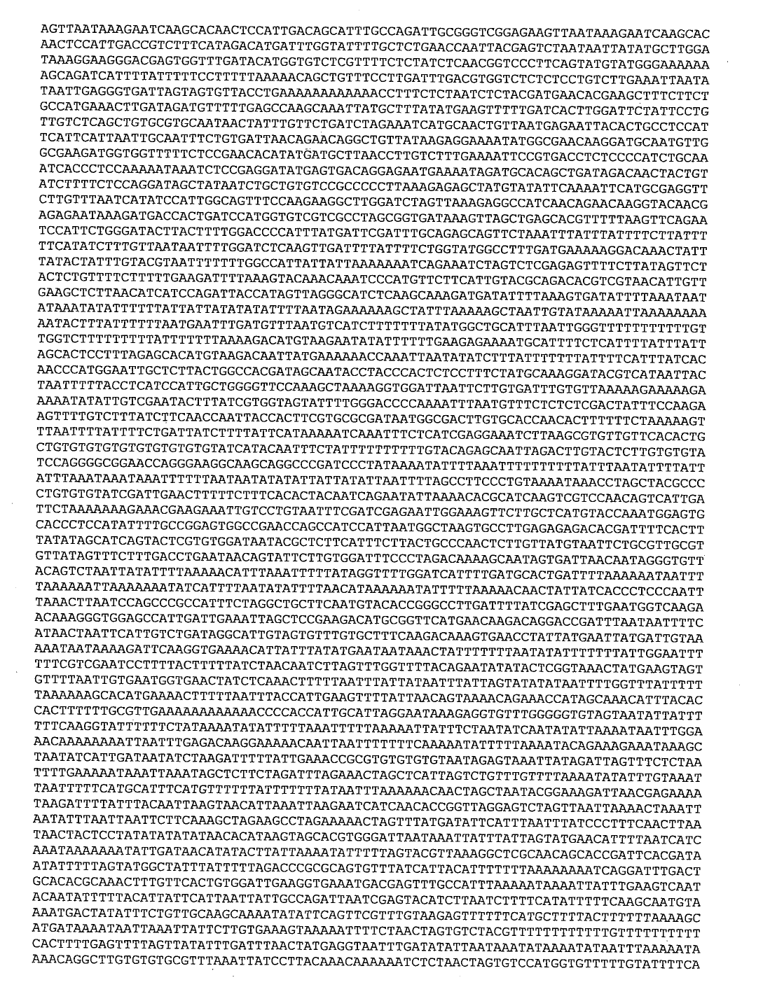

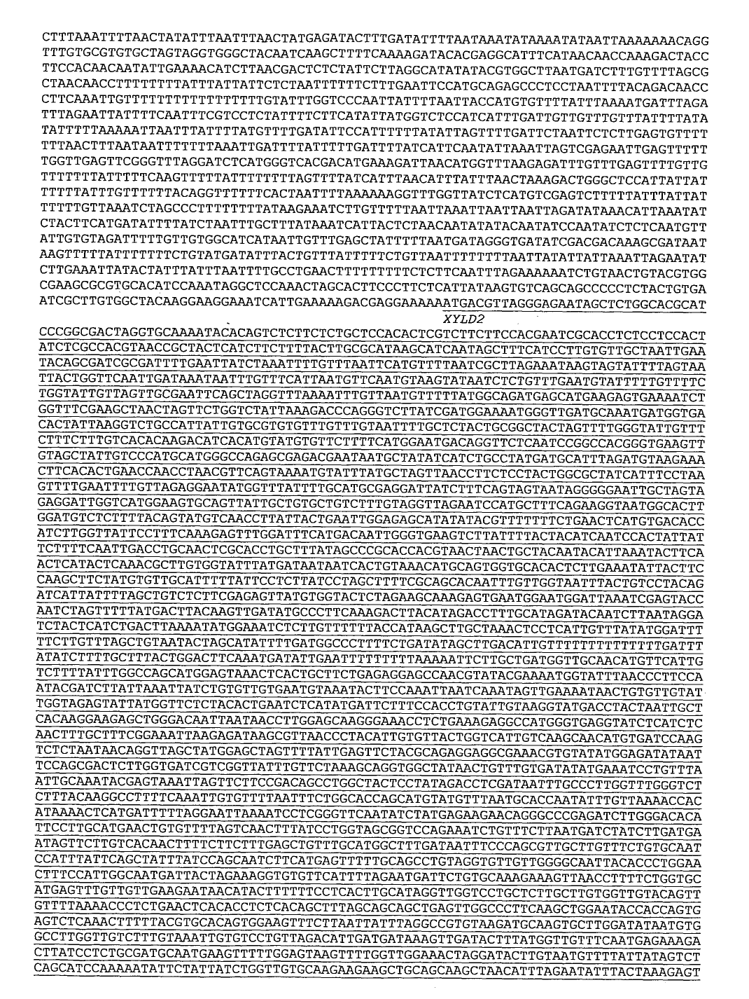

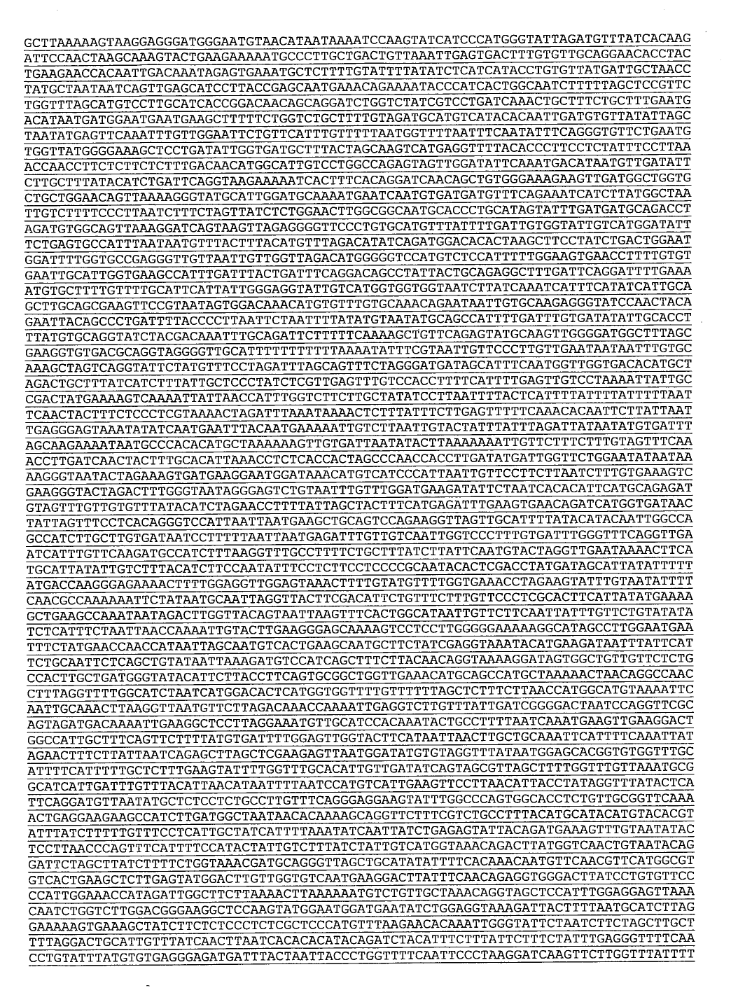

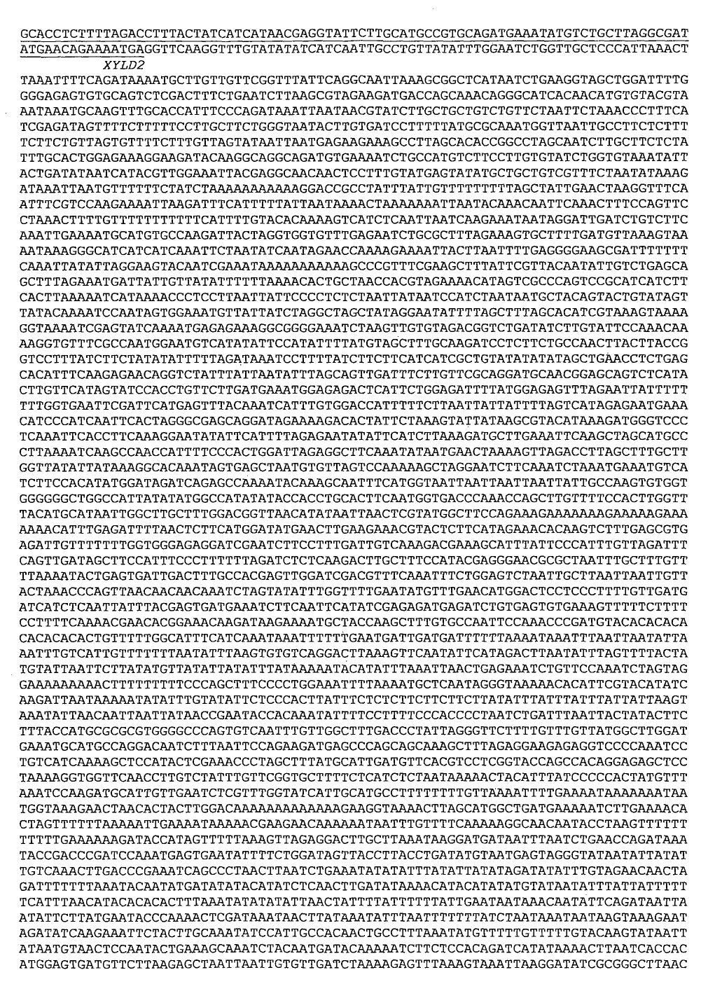

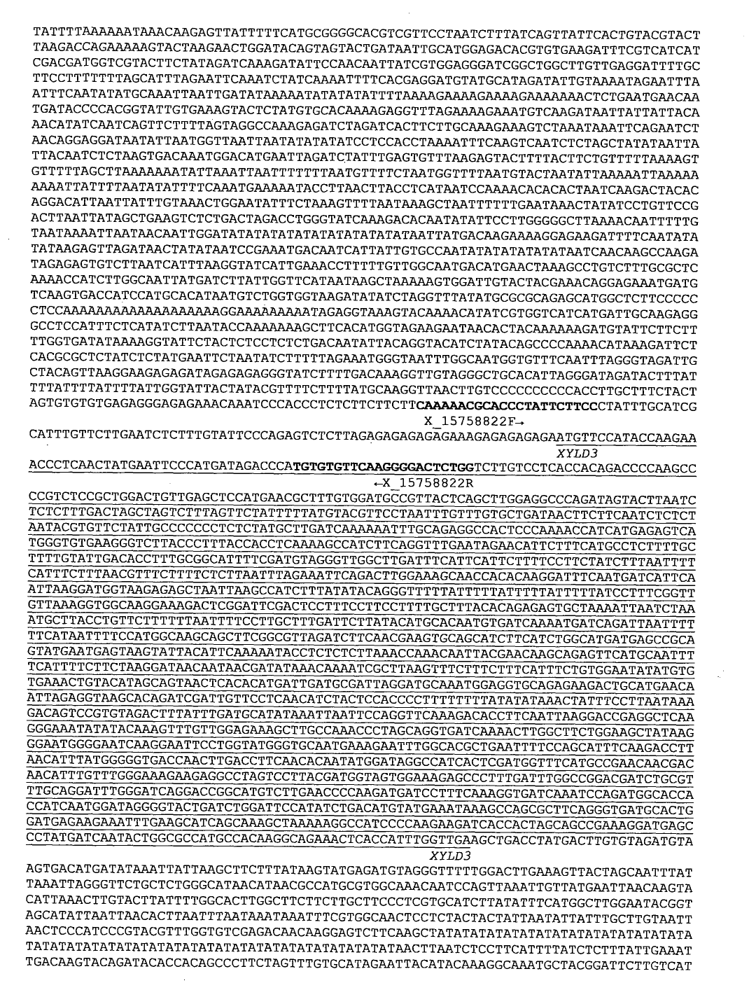

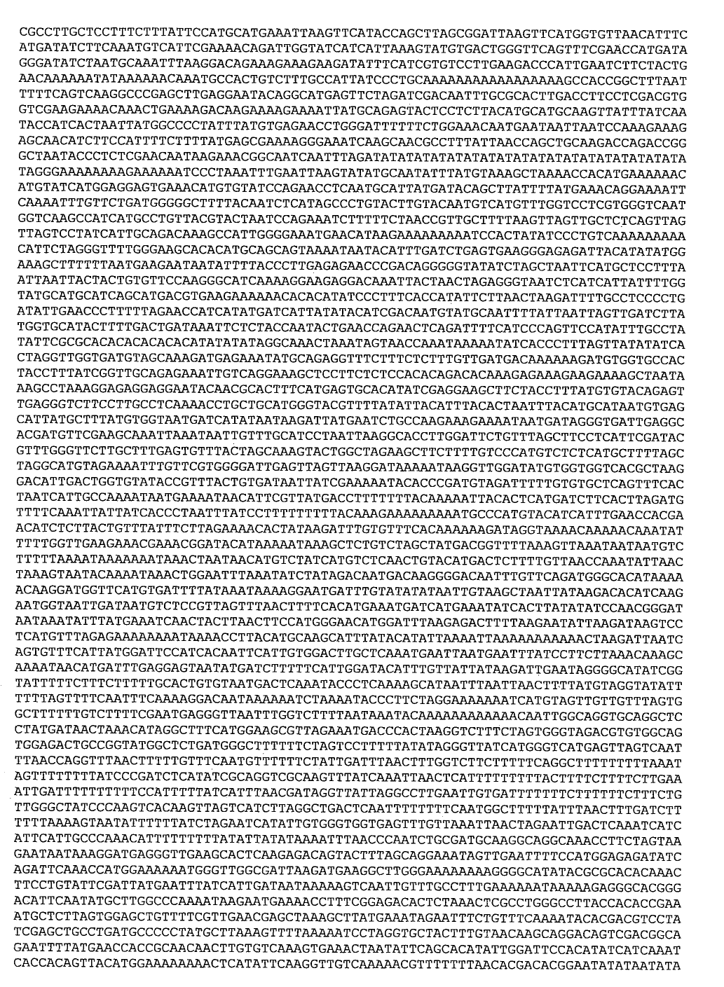

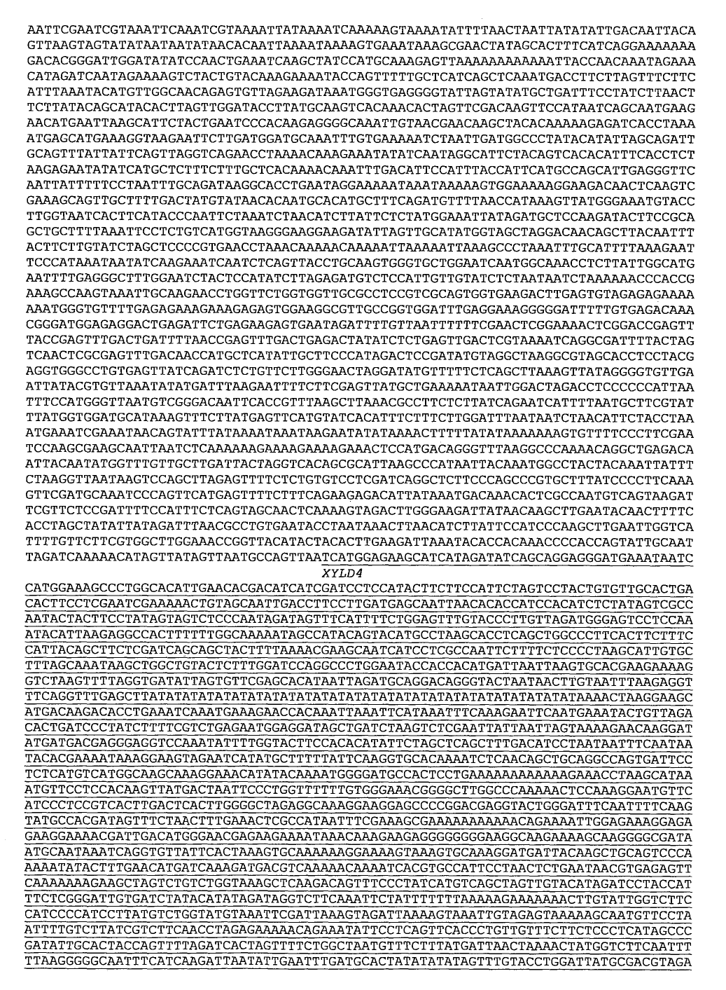

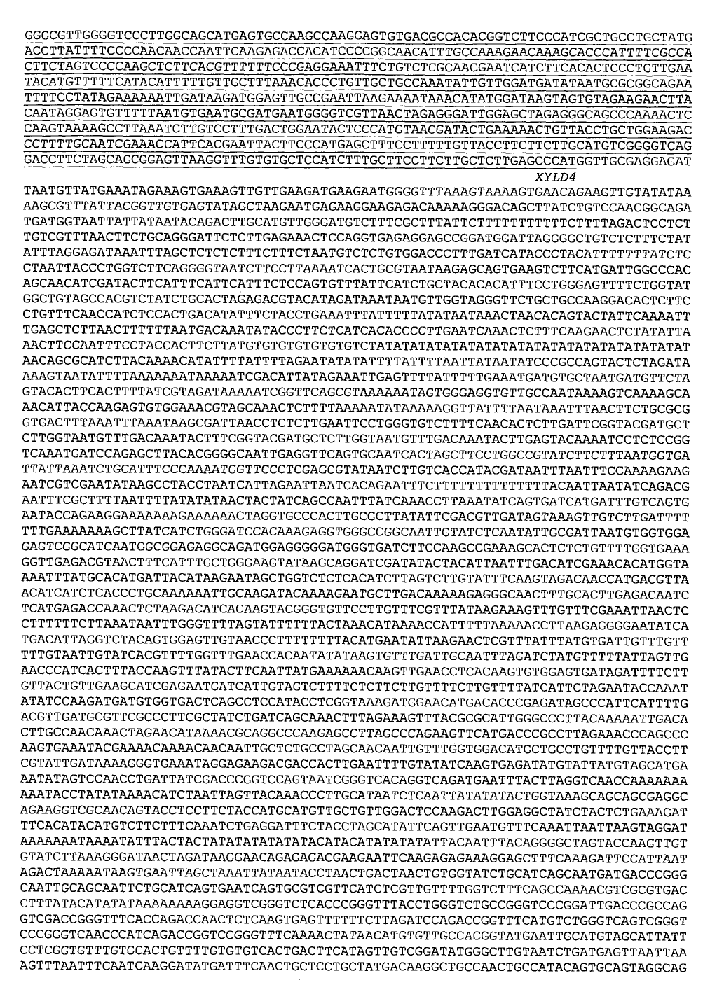

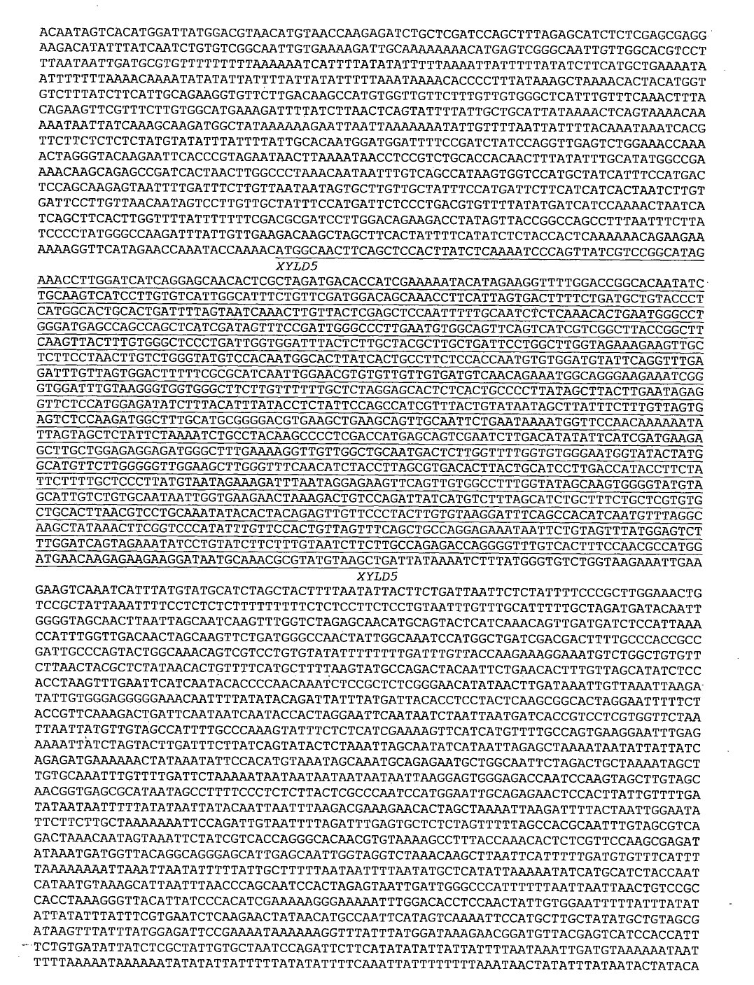

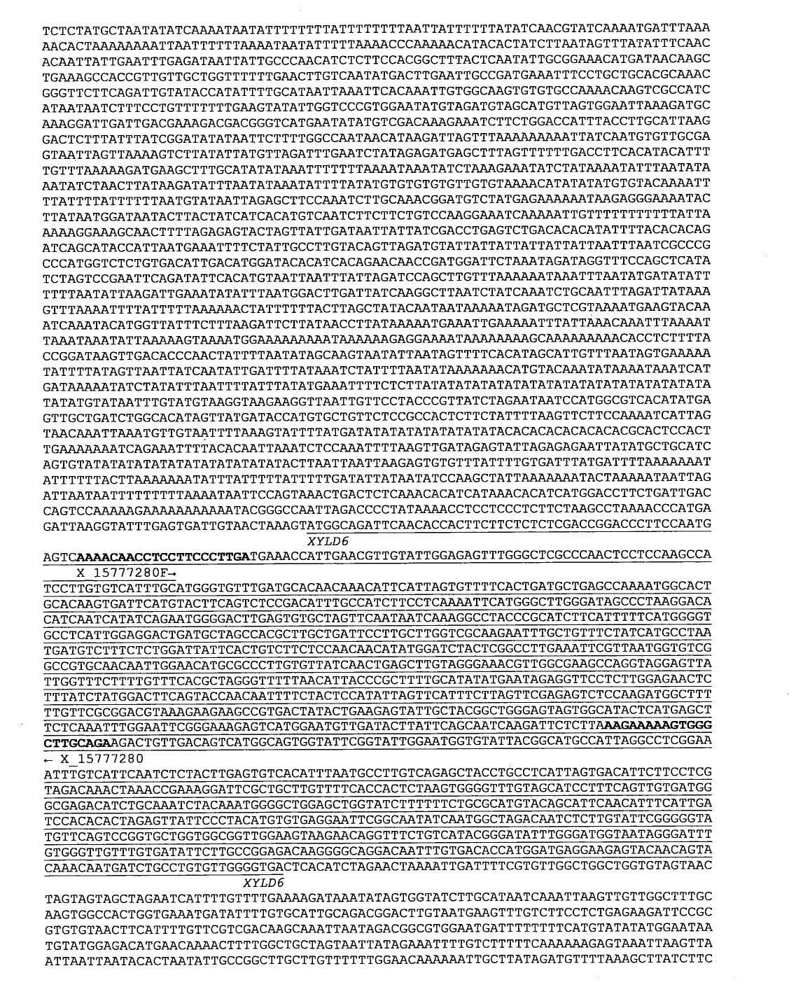

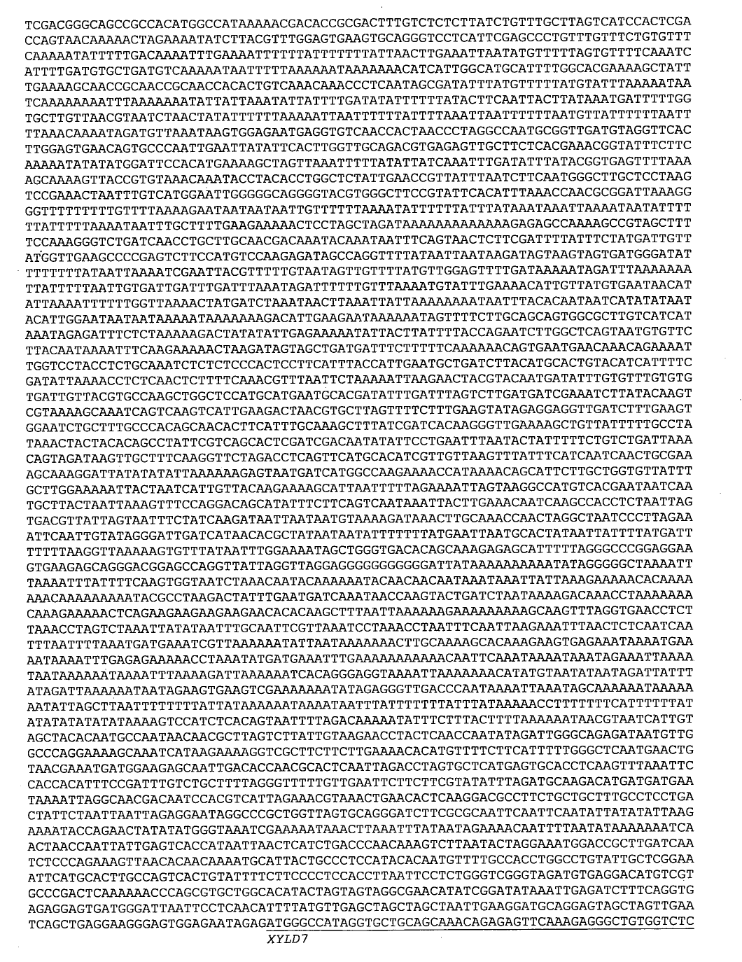

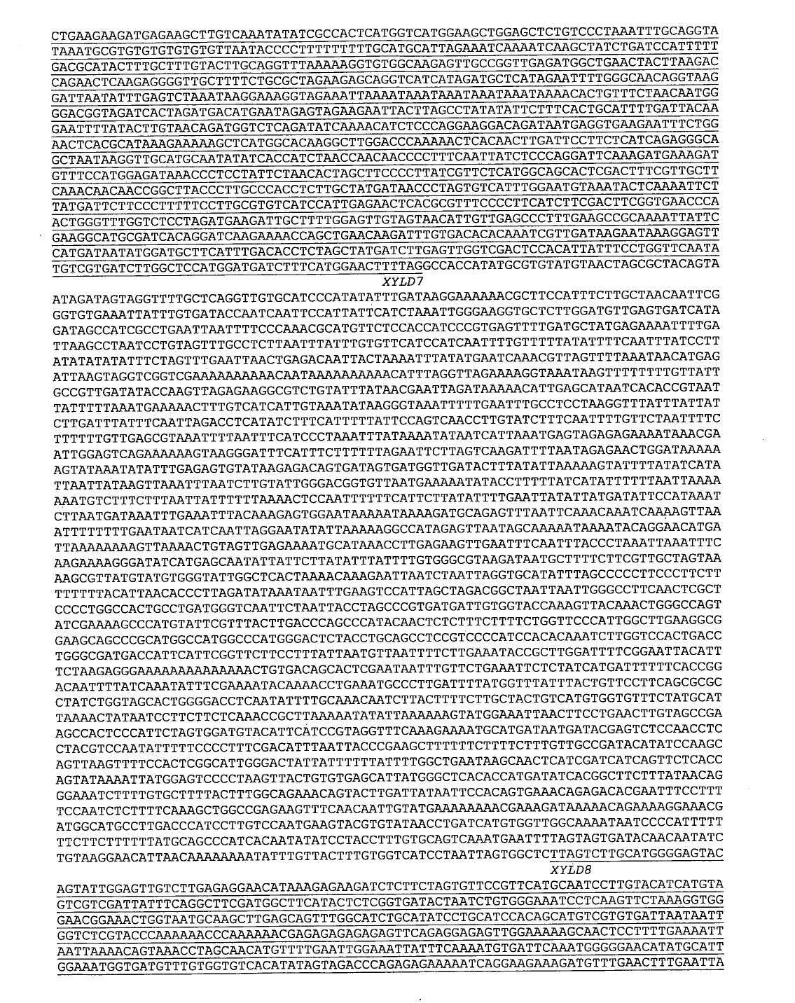

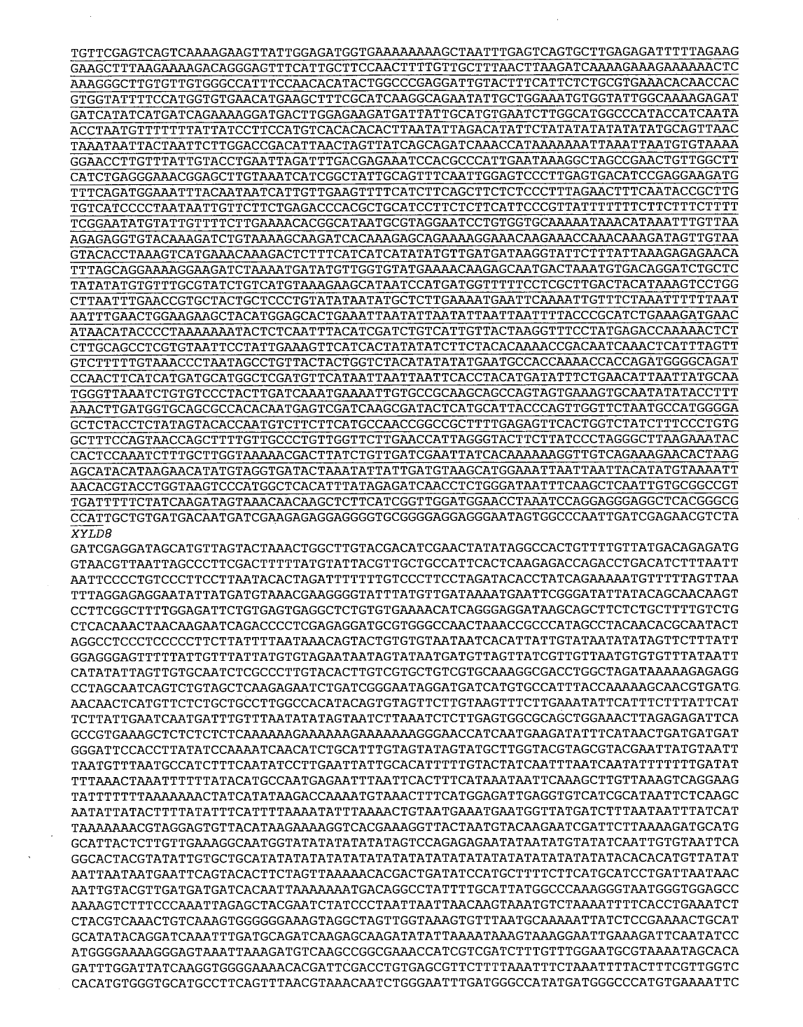

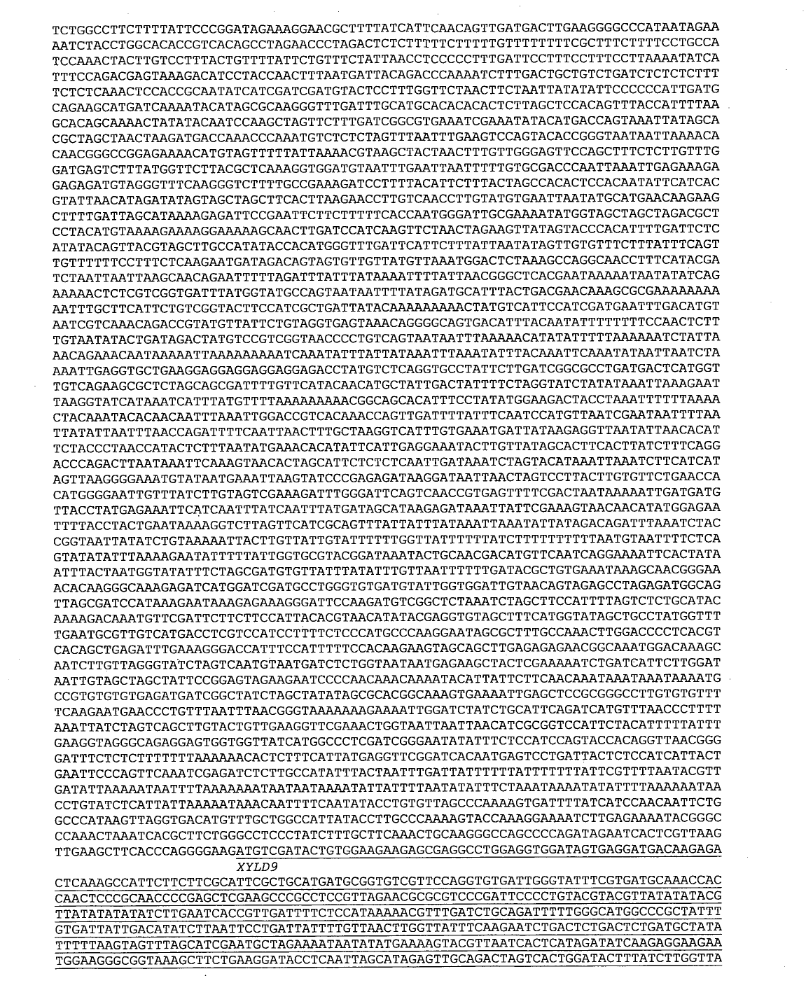

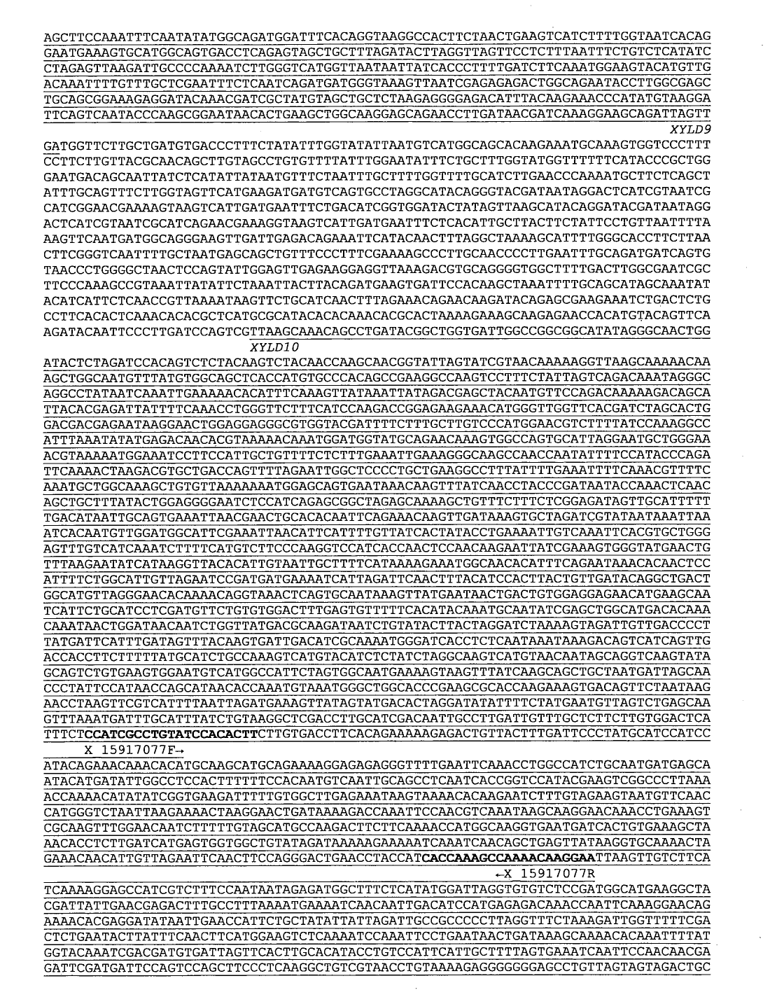

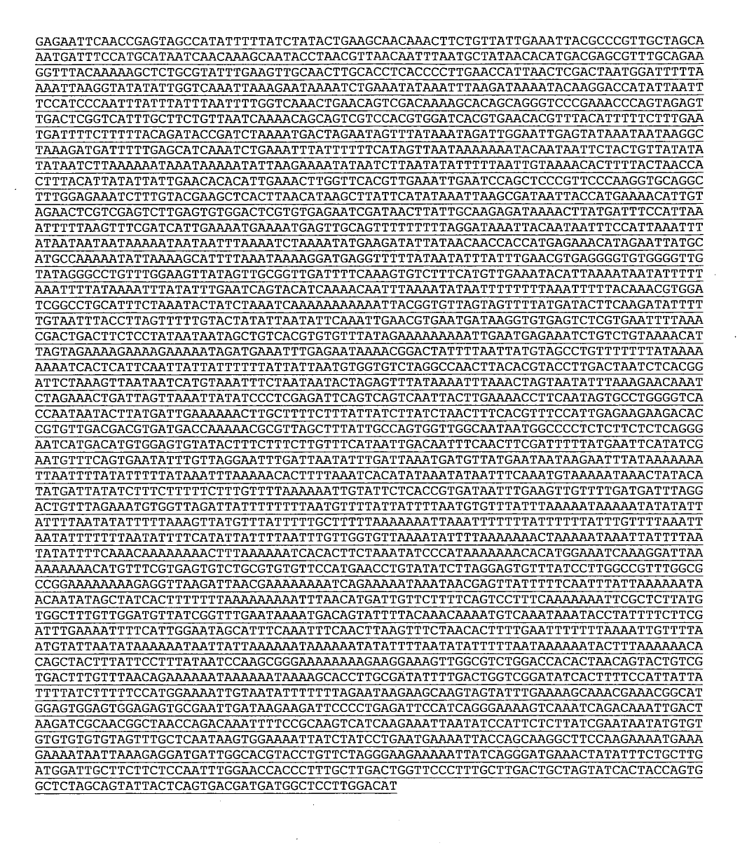

FIG. 18 shows the sequence of a QTL region in Populus associated with improved yield wherein the poplar sequence is derived from the public sequence annotation of the poplar genome (www.phytozome.net.).

### DETAILED DESCRIPTION

The present invention relates to Xyld1, Xyld2, Xyld3, Xyld4, Xyld5, Xyld6, Xyld7, Xyld8, Xyld9 and Xyld10 polynucleotides and polypeptides and homologues thereof.

In preferred embodiments of the present invention, the polynucleotide comprises a nucleotide sequence which encodes a Salix allele C polypeptide selected from the group consisting of Xyld1, Xyld2, Xyld3, Xyld4, Xyld5, Xyld6, Xyld7, Xyld8, Xyld9 and Xyld10, or a homologue of said polynucleotide.

In preferred embodiments of the present invention, the polypeptide is a Salix allele C polypeptide selected from the group consisting of Xyld1, Xyld2, Xyld3, Xyld4, Xyld5, Xyld6, Xyld7, Xyld8, Xyld9 and Xyld10, or a homologue of said polypeptide.

The Xyld1 polynucleotide is shown in SEQ ID NO 4 and SEQ ID NO 5. SEQ ID NO 4 (as shown in FIG. 3A) shows a sequence of the gene in Populus and SEQ ID NO 5 (as shown in FIG. 3B) shows a sequence of the gene (allele A) in Salix. SEQ ID NO 27 (as shown in FIG. 3C) shows the Salix Xyld1 allele A polypeptide sequence.

The Xyld2 polynucleotide is shown in SEQ ID NO 6 and SEQ ID NO 7. SEQ ID NO 6 (as shown in FIG. 4A) shows a sequence of the gene in Populus and SEQ ID NO 7 (as shown in FIG. 4B) shows a sequence of the gene (allele A) in Salix. SEQ ID NO 28 (as shown in FIG. 4C) shows the Salix Xyld2 allele A in Salix polypeptide sequence.

The Xyld3 polynucleotide is shown in SEQ ID NO 8 and SEQ ID NO 9 and homologues thereof. SEQ ID NO 8 (as shown in FIG. 5A) shows a sequence of the gene in Populus and SEQ ID NO 9 (as shown in FIG. 5B) shows a sequence of the gene (allele A) in Salix. SEQ ID NO 29 (as shown in FIG. 5C) shows the Salix Xyld3 allele A polypeptide sequence.

The Xyld4 polynucleotide is shown in SEQ ID NO 10, SEQ ID NO 11 and SEQ ID NO 12. SEQ ID NO 10 (as shown in FIG. 6A) shows a sequence of the gene in Populus. SEQ ID NO 11 (as shown in FIG. 6B) shows a sequence of the gene (allele A) in Salix. SEQ ID NO 12 (as shown in FIG. 6C) shows a sequence of the gene (allele C) in Salix. SEQ ID NO 30 (as shown in FIG. 6D) shows the Salix Xyld4 allele A polypeptide sequence. SEQ ID NO 31 (as shown in FIG. 6E) shows the Salix Xyld4 allele C polypeptide sequence.

The Xyld5 polynucleotide is shown in SEQ ID NO 13. SEQ ID NO 13 (as shown in FIG. 7) shows a sequence of the gene in Populus.

The Xyld6 polynucleotide is shown in SEQ ID NO 14, SEQ ID NO 15 and SEQ ID NO 16. SEQ ID NO 14 (as shown in FIG. 8A) shows a sequence of the gene in Populus. SEQ ID NO 15 (as shown in FIG. 8B) shows a sequence of the gene (allele A) in Salix. SEQ ID NO 16 (as shown in FIG. 8C) shows a sequence of the gene (allele C) in Salix. SEQ ID NO 32 (as shown in FIG. 8D) shows the Salix Xyld6 allele A polypeptide sequence. SEQ ID NO 33 (as shown in FIG. 8E) shows the Salix Xyld6 allele C polypeptide sequence.

The Xyld7 polynucleotide is shown in SEQ ID NO 3, SEQ ID NO 2 and SEQ ID NO 1. SEQ ID NO 3 (as shown in FIG. 9A) shows a sequence of the gene in Populus. SEQ ID NO 2 (as shown in FIG. 9B) shows a sequence of the gene (allele A) in Salix. SEQ ID NO 1 (as shown in FIG. 9C) shows a sequence of the gene (allele C) in Salix. An alignment of Xyld7 allele A (SEQ ID NO 2) sequence with the Xyld7 allele C sequence (SEQ ID NO 1) (as shown in the alignment of FIG. 9D) indicates Xyld7 allele A has an insertion region with extra nucleotides that are not present in Xyld7 allele C sequence SEQ ID NO 1. SEQ ID NO 26 (as shown in FIG. 9E) shows the Salix Xyld7 allele C polypeptide sequence.

The Xyld8 polynucleotide is shown in SEQ ID NO 17, SEQ ID NO 18 and SEQ ID NO 19. SEQ ID NO 17 (as shown in FIG. 10A) shows a sequence of the gene in Populus. SEQ ID NO 18 (as shown in FIG. 10B) shows a sequence of the gene (allele A) in Salix. SEQ ID NO 19 (as shown in FIG. 10C) shows a sequence of the gene (allele C) in Salix. SEQ ID NO 34 (as shown in FIG. 10D) shows the Salix Xyld8 allele A polypeptide sequence. SEQ ID NO 35 (as shown in FIG. 10E) shows the Salix Xyld8 allele C polypeptide sequence.

The Xyld9 polynucleotide is shown in SEQ ID NO 20, SEQ ID NO 21 and SEQ ID NO 22. SEQ ID NO 20 (as shown in FIG. 11A) shows a sequence of the gene in Populus. SEQ ID NO 21 (as shown in FIG. 11B) shows a sequence of the gene (allele A) in Salix. SEQ ID NO 22 (as shown in FIG. 11C) shows a sequence of the gene (allele C) in Salix. SEQ ID NO 36 (as shown in FIG. 11D) shows the Salix Xyld9 allele A polypeptide sequence. SEQ ID NO 37 (as shown in FIG. 11E) shows the Salix Xyld9 allele C polypeptide sequence.

The Xyld10 polynucleotide is shown in SEQ ID NO 23, SEQ ID NO 24 and SEQ ID NO 25. SEQ ID NO 23 (as shown in FIG. 12A) shows a sequence of the gene in Populus. SEQ ID NO 24 (as shown in FIG. 12B) shows a sequence of the gene (allele A) in Salix. SEQ ID NO 25 (as shown in FIG. 12C) shows a sequence of the gene (allele C) in Salix. SEQ ID NO 38 (as shown in FIG. 12D) shows the Salix Xyld10 allele A polypeptide sequence. SEQ ID NO 39 (as shown in FIG. 12E) shows the Salix Xyld10 allele C polypeptide sequence.

The importance of Xyld1, Xyld2, Xyld3, Xyld4, Xyld5, Xyld6, Xyld7, Xyld8, Xyld9 and Xyld10 in genetic improvement in crop plants was established following the identification of a QTL region in Salix associated with improved harvestable biomass yield. The corresponding QTL region in Populus is shown in FIG. 1. A comparison of this QTL region with information from the Populus trichocarpa genome database (http://genome.jgi-psf.org/Poptr1_{—}1/Poptr1_{—}1.home.html) indicated that the QTL region comprises Xyld1, Xyld2, Xyld3, Xyld4, Xyld5, Xyld6, Xyld7, Xyld8, Xyld9 and Xyld10.

The information provided on Xyld1, Xyld2, Xyld3, Xyld4, Xyld5, Xyld6, Xyld7, Xyld8, Xyld9 and Xyld10 provides a route to exploitation in crops, other cultivated plants or model plants, not directly related to Populus or Salix as the information disclosed herein enables homologous genes to Xyld1, Xyld2, Xyld3, Xyld4, Xyld5, Xyld6, Xyld7, Xyld8, Xyld9 and Xyld10 to be identified.

Details of Xyld1, Xyld2, Xyld3, Xyld4, Xyld5, Xyld6, Xyld7, Xyld8, Xyld9 and Xyld10 are detailed below:

### 1. Xyld1

Xyld1 shows best homology in Arabidopsis thaliana with Locus AT3G12740, or ALIS1 (ALA-Interacting Subunit). ALIS1 is a member of a family of phospholipid transporters (ALIS1-ALIS5) which are homologs of the Cdc50p/Lem3p family in yeast that are essential for the trafficking of yeast P4-ATPases. The Arabidopsis ALIS proteins are 27-30% identical to yeast Cdc50p and similarity ranges from 48-53%. In yeast ALIS1 shows strong affinity to ALA3. In Arabidopsis, ALA3 has been shown to be important for trans-Golgi proliferation of slime vesicles containing polysaccharides and enzymes for secretion. In yeast, ALA3 function requires interaction with the ALIS1. In Arabidopsis plants, ALIS1, like ALA3, is localised to membranes of Golgi-like structures and is expressed in root peripheral columella cells. It has been proposed that the ALIS1 protein is a β-sub-unit of ALA3 in Arabidopsis and that this protein is important part of the Golgi machinery in plants required for secretory processes during development.

### Relevant Publications

Poulsen L R, López-Marqués R L, McDowell S C, Okkeri J, Licht D, Schulz A, Pomorski T, Harper J F, Palmgren M G. 2008 The Arabidopsis P4-ATPase ALA3 localizes to the golgi and requires a beta-subunit to function in lipid translocation and secretory vesicle formation. Plant Cell. 3:658-76.

Bosco C D, Lezhneva L, Biehl A, Leister D, Strotmann H, Wanner G, Meurer J. 2004 Inactivation of the chloroplast ATP synthase gamma subunit results in high non-photochemical fluorescence quenching and altered nuclear gene expression in Arabidopsis thaliana. J Biol Chem. 279(2):1060-9.

### 2. Xyld 2

Xyld 2 shows strongest homology to Arabidopsis thaliana gene ALDH5F1 (Locus AT1G79440; previous nomenclature SSADH; EC 1.2.1.24) which is a member of the aldehyde dehydrogenases (ALDHs) protein superfamily of NAD(P)C-dependent enzymes that oxidize a wide range of endogenous and exogenous aliphatic and aromatic aldehydes. The Arabidopsis genome contains 14 unique ALDH sequences encoding members of nine ALDH families, including eight known families and one novel family (ALDH22) that is currently known only in plants. Of these, there is one succinic semialdehyde dehydrogenase gene, ALDH5F1, which encodes a protein of 528 amino acids. ALDH5F1 is the only confirmed identified member of the succinic semialdehyde family in plants. The Arabidopsis protein is localized to mitochondria and a kinetic analysis showed that the recombinant enzyme was specific for succinic semialdehyde and regulated by adenine nucleotides. T-DNA knockout mutants of ALDH5F1 result in dwarfed plants with necrotic lesions and are sensitive to both ultraviolet-B light and heat stress. Plants with ssadh mutations accumulate elevated levels of H₂O₂, suggesting a role for this gene in stress regulation detoxification pathway plant, providing defense against environmental stress by preventing the accumulation of reactive oxygen species.

### Relevant Publications

Hueser, A F, UI L. 2008 Analysis of GABA-shunt metabolites in Arabidopsis thaliana 19th International Conference on Arabidopsis Research

Ludewig F, Hüser A, Fromm H, Beauclair L, Bouché N. 2008 Mutants of GABA transaminase (POP2) suppress the severe phenotype of succinic semialdehyde dehydrogenase (ssadh) mutants in Arabidopsis. PLoS ONE 3(10):e3383

Zybailov B, Rutschow H, Friso G, Rudella A, Emanuelsson O, Sun Q, van Wijk K J. 2008 Sorting signals, N-terminal modifications and abundance of the chloroplast proteome. PLoS ONE 3(4):e1994

Fait A, Yellin A, Fromm H. 2005 GABA shunt deficiencies and accumulation of reactive oxygen intermediates: insight from Arabidopsis mutants. FEBS Lett. 579(2):415-20

Kirch H H, Bartels D, Wei Y, Schnable P S, Wood A J. 2004 The ALDH gene superfamily of Arabidopsis. Trends Plant Sci. 9(8):371-7

Breitkreuz K E, Allan W L, Van Cauwenberghe O R, Jakobs C, Talibi D, Andre B, Shelp B J. 2003 A novel gamma-hydroxybutyrate dehydrogenase: identification and expression of an Arabidopsis cDNA and potential role under oxygen deficiency. J Biol Chem. 278(42):41552-6

### 3. Xyld3

Xyld3 shows strongest homology with Arabidopsis thaliana ALTERED PHLOEM DEVELOPMENT (APL) gene (Locus AT1G79430), which encodes a MYB coiled-coil-type transcription factor that is required for phloem identity in Arabidopsis. APL has been proposed to have a dual role both in promoting phloem differentiation and in repressing xylem differentiation during vascular development.

### Relevant Publications

Truernit E, Bauby H, Dubreucq B, Grandjean O, Runions J, Barthelemy J, Palauqui J C. 2008 High-resolution whole-mount imaging of three-dimensional tissue organization and gene expression enables the study of Phloem development and structure in Arabidopsis. Plant Cell. 20(6):1494-503

Lehesranta S, Lindgren O, Taehtiharju S, Carlsbecker A, Helariutta Y 2008 The role of APL as a transcriptional regulator in specifying vascular identity 19th International Conference on Arabidopsis Research

Carlsbecker A, Lindgren O, Bonke M, Thitamadee S, Tähtiharju S, Helariutta Y 2004 Genetic analysis of procambial development in the Arabidopsis root 15th International Conference on Arabidopsis Research

Bonke M, Hauser M-T, Helariutta Y 2002 The APL locus is required for phloem development in Arabidopsis roots. 13th International Conference on Arabidopsis Research

### 4. Xyld4

Xyld4 show strongest homology in Arabidopsis thaliana to Locus AT1G79420. Function not yet described.

### 5. Xyld5

Xyld5 shows strongest homology with AtOCT2 in Arabidopsis thaliana (Locus AT1G79360). ATOCT2 is one of six Arabidopsis organic cation/carnitine transporter (OCT)-like proteins, named AtOCT1-AtOCT6 (loci At1g73220, At1g79360, At1g16390, At3g20660, At1g79410 and At1g16370, respectively) that have been identified. These proteins cluster in a small subfamily within the ‘organic solute cotransporters’ included in the large sugar transporter family of the major facilitator superfamily (MFS). AtOCT1 shares features of organic cation/camitine transporters (OCTs). In animals, mammalian plasma membrane OCTs are involved in homeostasis and distribution of various small endogenous amines (e.g. camitine, choline) and detoxification of xenobiotics such as nicotine. AtOCT1 is able to transport carnitine in yeast and is likely to be involved in the transport of camitine or related molecules across the plasma membrane in plants.

The orthologous gene sequence has not yet been identified in willow.

### Related Publication

Tohge T, Nishiyama Y, Hirai M Y, Yano M, Nakajima J, Awazuhara M, Inoue E, Takahashi H, Goodenowe D B, Kitayama M, Noji M, Yamazaki M, Saito K. 2005 Functional genomics by integrated analysis of metabolome and transcriptome of Arabidopsis plants over-expressing an MYB transcription factor. Plant J. 42(2):218-35

### 6. Xyld6

Xyld6 shows best fit with ATOCT3 Arabidopsis ORGANIC CATION/CARNITINE TRANSPORTER2). ATOCT3 is one of six Arabidopsis organic cation/carnitine transporter (OCT)-like proteins, named AtOCT1-AtOCT6 (loci At1g73220, At1g79360, At1g16390, At3g20660, At1g79410 and At1g16370, respectively) referred to above. These proteins cluster in a small subfamily within the ‘organic solute cotransporters’ included in the large sugar transporter family of the major facilitator superfamily (MFS).

### Relevant Publications

Lelandais-Brière C, Jovanovic M, Torres G A, Penin Y, Lemoine R, Corre-Menguy F, Hartmann C. 2007 Disruption of AtOCT1, an organic cation transporter gene, affects root development and carnitine-related responses in Arabidopsis. Plant J. 51(2):154-64

Price J, Laxmi A, St Martin S K, Jang J C. 2004 Global transcription profiling reveals multiple sugar signal transduction mechanisms in Arabidopsis. Plant Cell.16(8):2128-50

### 7. Xyld7

Xyld7 shows homology with members of the R2R3-type MYB gene family in Arabidopsis. Although no functional data are available for most of the 125 R2R3-type AtMYB genes, a number of functions have been assigned concerning many aspects of plant secondary metabolism, as well as the identity and fate of plant cells. This includes regulation of phenylpropanoid metabolism, control of development and determination of cell fate and identity, plant responses to environmental factors and mediating hormone actions.

### Relevant Publications

Stracke R, Werber M, Weisshaar B. 2001 The R2R3-MYB gene family in Arabidopsis thaliana. Curr Opin Plant Biol. 4(5):447-56

Riechmann J L, Heard J, Martin G, Reuber L, Jiang C, Keddie J, Adam L, Pineda O, Ratcliffe O J, Samaha R R, Creelman R, Pilgrim M, Broun P, Zhang J Z, Ghandehari D, Sherman B K, Yu G. 2000 Arabidopsis transcription factors: genome-wide comparative analysis among eukaryotes. Science. 290(5499):2105-10

### 8. Xyld8

Xyld8 shows best fit with ANAC028, Arabidopsis NAC domain containing protein (Locus AT1G65910). NAC (NAM, ATAF, and CUC) is a plant-specific gene family. NAC family transcription factors are involved in maintaining organ or tissue boundaries regulating the transition from growth by cell division to growth by cell expansion. Most NAC proteins contain a highly conserved N-terminal DNA-binding domain, a nuclear localization signal sequence, and a variable C-terminal domain. 75 and 105 NAC genes were predicted in the Oryza sativa and Arabidopsis genomes, respectively. The functions of only some of these have been described. The first reported NAC genes were NAM from petunia and CUC2 from Arabidopsis that participate in shoot apical meristem development. CUC1, CUC2 and nam are expressed at the boundaries between cotyledonary primordial and between floral organs and are specifically involved in shoot apical meristem formation and separation of cotyledons and floral organs. Other development-related NAC genes have been suggested with roles in controlling cell expansion of specific flower organs e.g. NAP or auxindependent formation of the lateral root system e.g. NAC1. Some of NAC genes, such as ATAF1 and ATAF2 genes from Arabidopsis and the StNAC gene from potato, are induced by pathogen attack and wounding. More recently, a few NAC genes, such as AtNAC072 (RD26), AtNAC019, AtNAC055 from Arabidopsis, and BnNAC from Brassica (31), were found to be involved in responses to environmental stress. Seven members of NAC family At2g18060, At4g36160, At5g66300, At1g12260, At1g62700, At5g62380, and At1g71930 have been designated as VASCULAR-RELATED NAC-DOMAIN PROTEIN 1 (VND1 to VND7). Members of these could induce transdifferentiation of various cells into metaxylem- and protoxylem-like vessel elements, respectively, in Arabidopsis and poplar. Similarly ANAC012 and ANAC073 also appear to have a role in xylem development and secondary wall thickening in Arabidopis.

### Relevant Publications

Ooka H, Satoh K, Doi K, Nagata T, Otomo Y, Murakami K, Matsubara K, Osato N, Kawai J, Carninci P, Hayashizaki Y, Suzuki K, Kojima K, Takahara Y, Yamamoto K, Kikuchi S. 2003 Comprehensive analysis of NAC family genes in Oryza sativa and Arabidopsis thaliana. DNA Res. 10(6):239-47

Riechmann J L, Heard J, Martin G, Reuber L, Jiang C, Keddie J, Adam L, Pineda O, Ratcliffe O J, Samaha R R, Creelman R, Pilgrim M, Broun P, Zhang J Z, Ghandehari D, Sherman B K, Yu G. 2000 Arabidopsis transcription factors: genome-wide comparative analysis among eukaryotes. Science 290(5499):2105-10

### 9. Xyld9

Xyld9 show strongest homology in Arabidopsis thaliana to Locus AT1G79390. The function of this expressed protein has not yet been described

### 10. Xyld10

Xyld10 shows homology to the RGLG2 (RING DOMAIN LIGASE2) locus of Arabidopsis thaliana (Locus AT1G79380). In functional terms, the RING domain can basically be considered a protein-interaction domain. RING-finger proteins have been implicated in a range of diverse biological processes and biochemical activities, from transcriptional and translational regulation to targeted protein degradation.

### Relevant Publications

Kosarev P, Mayer K F, Hardtke C S. 2002 Evaluation and classification of RING-finger domains encoded by the Arabidopsis genome. Genome Biol. 3(4):RESEARCH 0016.1

Further homologous genes to Xyld1, Gene Xyld2, Gene Xyld3, Gene Xyld4, Gene Xyld6, Gene Xyld7, Gene Xyld8, Gene Xyld9 and Gene Xyld10 can be identified, for example, through in silico sequence similarity searches for crops/cultivated or model plants for which such sequence resources exist. Where such resources are lacking, standard molecular biology methods can be employed to clone homologous genes. As examples, degenerate primers can designed to amino acid sequences and used in PCR to amplify and clone target genes, or alternatively, sequences can be used in hybridisation approaches if sufficient similarity is expected.

Once homologous genes are identified by any such approach, and the crop/plant specific sequence is determined, polymorphisms within a given gene can identified through sequencing or restriction analysis, as examples.

1. Direct application in genetic improvement.

The gene defined here facilitates direct use for selection of high yielding plants in crop breeding programmes. Several laboratories have collections of polymorphic markers for general use in mapping studies or for assessing genetic diversity. Now that the gene has been identified here and a sequence provided, if markers linked to the gene described here are available in these laboratories they could be directly employed in selection programmes for improving yield.

The efficiency of the use of QTL-associated marker in marker-assisted selection strategies will be dependent on the degree of genetic linkage that exists between the marker to be used and the causal polymorphism that underlies the QTL. To maximise the efficiency of marker-assisted selections based on a QTL, such as that described here, markers that are tightly linked to the region would be required to minimise the likelihood that linkage between the marker and the causal polymorphism will breakdown through recombination. The information described here provides a route to efficient achievement of the identification of markers whose linkage to the causal polymorphism will not be broken easily by recombination. Although anonymous markers such as Amplified Fragment Length Polymorphism (AFLP) and Random Amplified Polymorphism (RAPD) classes for example, could be screened in large numbers to identify those that may fall into regions of the genome linked to the QTL by chance, more efficient methods based on the sequence information provided here can be used in more direct approaches.

Using knowledge of the underlying sequence information that is publicly available in Populus (http://genome.jgi-psf.org/Poptr1_{—}1/Poptr1_{—}1.home.html) or that which is provided here for willow, specific markers can be developed that are targeted directly at this region or to a region that is closely linked in genetic terms. Markers of this class could include, as examples, microsatellite markers, Restriction Length Fragment Length Polymoprhisms (RFLP), Cleaved Amplified Polymorphisms (CAPS), Single Nucleotide Polymorphisms (SNPS) and INSertion/DELetion (INDELs). For microsatellite markers, primer pairs that amplify potentially highly polymorphic simple sequence repeat units could be designed from Salix or Populus sequence in this region. These could be specific to either genus or could be directly transferable from one genus to the other, if nucleotide sequence is sufficiently conserved at the priming sites. This is often true if priming sites are selected within coding regions (Hanley, S. J., Mallott, M. D. & Karp A. (2006) Tree Genetics and Genomes, 3, 35-48) (Hanley et al, 2006). Microsatellite primer sets would then be tested for their ability to detect polymoprhisms in the germplasm under study, and those that distinguish between alleles could be used in marker-assisted selections. Similarly, for the development of other markers types (SNP, CAPS, INDEL) sequence information for the QTL region could be used to design primer sets to generate amplicons that could then be examined for polymorphisms in the germplasm under study, either from sequencing or restriction digestion analysis.

2. Application in transgenic genetic improvement strategies.

The sequences supplied provide a route to crop improvement through genetic manipulation via transgenic approaches. The sequences provided could be used directly to generate constructs for testing in transformation experiments. Such experiments may involve overexpression, gene-silencing or introduction of a beneficial allele into any recipient genotype. Such experiments may utilise the Salix or Populus sequences provided here or be based on homologous genes derived from any plant of interest.

This disclosure relates to representative markers, and alleles thereof, that correspond to and identify a locus that is associated with harvestable yield.

The methods, markers, and alleles of the present invention provide a simple, inexpensive and reliable means of identifying the haplotype associated with the harvestable biomass yield locus. By identifying the chromosome haplotype in this region, it is possible to predict whether the harvestable biomass yield associated QTL contributes to small or large yield of plant.

Thus, one aspect of this disclosure concerns markers (and alleles thereof) genetically linked to a polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, which is associated with a harvestable biomass yield associated QTL that provides a contribution to harvestable biomass yield in willow.

Another aspect of this disclosure concerns markers (and alleles thereof) genetically linked to a polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, which is associated with a harvestable biomass yield associated QTL that provides a contribution to harvestable biomass yield in willow.

Kits including probes that detect the markers described herein are also a feature of this disclosure.

Another aspect of this disclosure concerns a method for predicting harvestable biomass yield in a crop plant. The method can include genotyping a sample obtained from a subject crop plant for one or more markers genetically linked to a polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25. The markers are chosen to individually or collectively identify a haplotype associated with harvestable biomass yield. The haplotype is correlated with harvestable biomass yield providing a prediction of the harvestable biomass yield of the subject plant.

A further aspect of this disclosure concerns a method for predicting harvestable biomass yield in a crop plant. The method can include genotyping a sample obtained from a subject crop plant for one or more markers genetically linked to a polynucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39. The markers are chosen to individually or collectively identify a haplotype associated with harvestable biomass yield. The haplotype is correlated with harvestable biomass yield providing a prediction of the harvestable biomass yield of the subject plant.

In certain embodiments, the haplotype is correlated with harvestable biomass yield by comparing the haplotype to an index of average harvestable biomass yield by plant variety.

### Definitions

The poplar and willow chromosomes are referred to as ‘linkage groups’. This is because there are more sequence contigs than chromosomes in the poplar assembly.

An “allele” is understood within the scope of the invention to refer to a given form of a gene, or of any kind of identifiable genetic element such as a marker, that occupies a specific position or locus on a chromosome. Variant forms of genes occurring at the same locus are said to be alleles of one another. In a diploid cell or organism, the two alleles of a given gene (or marker) typically occupy corresponding loci on a pair of homologous chromosomes.

An allele associated with a quantitative trait may comprise a single gene or multiple genes or even a gene encoding a genetic factor contributing to the phenotype represented by said QTL.

The term “breeding”, and grammatical variants thereof, refer to any process that generates a progeny individual. Breedings can be sexual or asexual, or any combination thereof. Exemplary non-limiting types of breedings include crossings, selfings, doubled haploid derivative generation, and combinations thereof.

By “exogenous gene/polynucleotide” it is meant that the gene/polynucleotide is transformed into the unmodified plant from an external source. The exogenous nucleotide may, for example, be derived from a genomic DNA or cDNA sequence. Typically the exogenous gene is derived from a different source and has a sequence different to the endogenous gene. Alternatively, introduction of an exogenous gene having a sequence identical to the endogenous gene may be used to increase the number of copies of the endogenous gene sequence present in the plant.

The term “Homozygous” refers to like alleles at one or more corresponding loci on homologous chromosomes.

The term “Heterozygous refers to unlike alleles at one or more corresponding loci on homologous chromosomes.

The term “Gene” refers to a unit of DNA which performs one function. Usually, this is equated with the production of one RNA or one protein. A gene may contain coding regions, introns, untranslated regions and control regions.

As used herein, the phrase “genetic marker” refers to a feature of an individual's genome (e.g., a nucleotide or a polynucleotide sequence that is present in an individual's genome) that is associated with one or more loci of interest. Typically, a genetic marker is polymorphic and the variant forms (or llel. Genetic markers include, for example, single nucleotide polymorphisms (SNPs), indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), Microsatellites or Simple sequence repeat (SSRs) among many other examples.

Genetic markers can, for example, be used to locate genetic loci containing alleles that contribute to variability in expression of phenotypic traits on a chromosome.

A genetic marker can be physically located in a position on a chromosome that is within or outside of to the genetic locus with which it is associated (i.e., is intragenic or extragenic, respectively). Stated another way, whereas genetic markers are typically employed when the location on a chromosome of the gene that corresponds to the locus of interest has not been identified and there is a non-zero rate of recombination between the genetic marker and the locus of interest, the presently disclosed subject matter can also employ genetic markers that are physically within the boundaries of a genetic locus (e.g., inside a genomic sequence that corresponds to a gene such as, but not limited to a polymorphism within an intron or an exon of a gene). In some embodiments of the presently disclosed subject matter, the one or more genetic markers comprise between one and ten markers, and in some embodiments the one or more genetic markers comprise more than ten genetic markers.

The term “genotype” refers to the set of alleles present in a subject at one or more loci under investigation. At any one autosomal locus a geneotype will be either homozygous (with two identical alleles) or heterozygous (with two different alleles).

The term “haplotype” refers to the set of alleles an individual inherited from one parent. A diploid individual thus has two haplotypes. The term “haplotype” can be used in a more limited sense to refer to physically linked and/or unlinked genetic markers (e.g., sequence polymorphisms) associated with a phenotypic trait. The phrase “haplotype block” (sometimes also referred to in the literature simply as a haplotype) refers to a group of two or more genetic markers that are physically linked on a single chromosome (or a portion thereof). Typically, each block has a few common haplotypes, and a subset of the genetic markers (i.e., a “haplotype tag”) can be chosen that uniquely identifies each of these haplotypes.

As used herein, the terms “hybrid”, “hybrid plant,” and “hybrid progeny” refers to an individual produced from genetically different parents (e.g., a genetically heterozygous or mostly heterozygous individual).

If two individuals possess the same allele at a particular locus, the alleles are termed “identical by descent” if the alleles were inherited from one common ancestor (i.e., the alleles are copies of the same parental allele). The alternative is that the alleles are “identical by state” (i.e., the alleles appear the same but are derived from two different copies of the allele). Identity by descent information is useful for linkage studies; both identity by descent and identity by state information can be used in association studies such as those described herein, although identity by descent information can be particularly useful.

The term “linkage”/“genetic linkage”, and grammatical variants thereof, refers to the association of two or more (and/or traits) at positions on the same chromosome, preferably such that recombination between the two loci is reduced to a proportion significantly less than 50%. The term linkage can also be used in reference to the association between one or more loci and a trait if an allele (or alleles) and the trait, or absence thereof, are observed together in significantly greater than 50% of occurrences. A linkage group is a set of loci, in which all members are linked either directly or indirectly to all other members of the set.

“linkage disequilibrium” (also called “allelic association”) refers to a phenomenon wherein particular alleles at two or more loci tend to remain together in linkage groups when segregating from parents to offspring with a greater frequency than expected from their individual frequencies in a given population. For example, a genetic marker allele and a QTL allele can show linkage disequilibrium when they occur together with frequencies greater than those predicted from the individual allele frequencies. Linkage disequilibrium can occur for several reasons including, but not limited to the alleles being in close proximity on a chromosome

“Locus” refers to a region on a chromosome, which comprises a gene or a genetic marker or the like.

As used herein, the phrase “nucleic acid” refers to any physical string of monomer units that can be corresponded to a string of nucleotides, including a polymer of nucleotides (e.g., a typical DNA, cDNA or RNA polymer), modified oligonucleotides (e.g., oligonucleotides comprising bases that are not typical to biological RNA or DNA, such as 2′-O-methylated oligonucleotides), and the like. In some embodiments, a nucleic acid can be single-stranded, double-stranded, multi-stranded, or combinations thereof. Unless otherwise indicated, a particular nucleic acid sequence of the presently disclosed subject matter optionally comprises or encodes complementary sequences, in addition to any sequence explicitly indicated.

The term “protein” includes single-chain polypeptide molecules as well as multiple-polypeptide complexes where individual constituent polypeptides are linked by covalent or non-covalent means.

The phrase “phenotypic trait” refers to the appearance or other detectable characteristic of an individual, resulting from the interaction of its genome with the environment.

“The term Microsatellite or SSRs (Simple sequence repeats) (Marker)” refers to a type of genetic marker that consists of numerous repeats of short sequences of DNA bases, which are found at loci throughout the plant's DNA and have a likelihood of being highly polymorphic.

“Polymorphism” refers to the presence in a population of two or more different forms of a gene, genetic marker, or inherited trait.

The term “quantitative trait locus” (QTL) refers to an association between a genetic marker and a chromosomal region and/or gene that affects the phenotype of a trait of interest. Typically, this is determined statistically; e.g., based on one or more methods published in the literature. A QTL can be a chromosomal region and/or a genetic locus with at least two alleles that differentially affect the expression of a phenotypic trait (either a quantitative trait or a qualitative trait).

“Sequence Homology or Sequence identity” is used herein interchangeably. The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. If two sequences which are to be compared with each other differ in length, sequence identity preferably relates to the percentage of the nucleotide residues of the shorter sequence which are identical with the nucleotide residues of the longer sequence. Sequence identity can be determined conventionally with the use of computer programs such as the Bestfit program (Wisconsin Sequence Analysis Package, Version 8 for Unix, Genetics Computer Group, University Research Park, 575 Science Drive Madison, Wis. 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W. R. Pearson (1990), Methods in Enzymology 183, 63-98, appended examples and http://workbench.sdsc.edu/). For this purpose, the “default” parameter settings may be used.

Preferably, reference to a sequence which has a percent identity to any one of SEQ ID NOs: 1-43 as detailed herein refers to a sequence which has the stated percent identity over the entire length of the SEQ ID NO referred to.

Another indication that two nucleic acid sequences are substantially identical is that the two molecules hybridize to each other under stringent conditions.

In general, unless otherwise specified, when referring to a “plant” it is intended to cover a plant at any stage of development, including sing cells and seeds. Thus, in particular embodiments, the present invention provides a plant cell.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

“Harvestable biomass yield” is calculated according to the plants parts that constitute relevant harvestable product. In one embodiment, a harvestable biomass yield corresponds to the total of the above ground biomass being the harvestable product. Preferred examples, where the harvestable product of the crop may be the above ground biomass are trees such as, for example (but not limited to), Salex or Popular. In another embodiment, a harvestable biomass yield corresponds to only one part of the plant being the harvestable product. Preferred examples, where the harvestable product of the crop may be a part of the plant are parts of food crops such as, for example (but not limited to), the kernel in maize or the grain in rice.

The genomic DNA can be assayed to determine which markers are present using any method known in the art. For example, single-strand conformation polymorphism (SSCP) analysis, base excision sequence scanning (BESS), restriction fragment length polymorphism (RFLP) analysis, heteroduplex analysis, denaturing gradient gel electrophoresis (DGGE), temperature gradient electrophoresis, allelic polymerase chain reaction (PCR), ligase chain reaction direct sequencing, mini sequencing, nucleic acid hybridization, or micro-array-type detection can be used to identify the polymorphisms present in the sample.

The methods described herein include genotyping a sample of genetic material obtained from a subject plant for one or more markers to determine the allele present at the marker locus.

### Detection of Alleles

The nucleic acids obtained from the sample can be genotyped to identify the particular allele present for a marker locus. A sample of sufficient quantity to permit direct detection of marker alleles from the sample can be obtained from the plant.

Alternatively, a smaller sample is obtained from the subject and the nucleic acids are amplified prior to detection. Optionally, the nucleic acid sample is purified (or partially purified) prior to detection of the marker alleles. Any target nucleic that is informative for a chromosome haplotype in the interval corresponding to the sequence located between reference nucleotide position A and reference nucleotide position B can be detected. The target nucleic acid may correspond to a marker locus localized in this interval. Any method of detecting a nucleic acid molecule can be used such as hybridization and/or sequencing assays.

### Hybridization

Hybridization is the binding of complementary strands of DNA, DNA/RNA, or RNA. Hybridization can occur when primers or probes bind to target sequences such as target sequences within willow genomic DNA. Probes and primers that are useful generally include nucleic acid sequences that hybridize (for example under high stringency conditions) with at least 10, 12, 14, 16, 18, or 20 to the sequences provided. Physical methods of detecting hybridization or binding of complementary strands of nucleic acid molecules, include but are not limited to, such methods as DNase I or chemical footprinting, gel shift and affinity cleavage assays, Southern and Northern blotting, dot blotting and light absorption detection procedures. The binding between a nucleic acid primer or probe and its 26 target nucleic acid is frequently characterized by the temperature (Tm) at which 50% of the nucleic acid probe is melted from its target. A higher (Tm) means a stronger or more stable complex relative to a complex with a lower (Tm).

More generally, complementary nucleic acids form a stable duplex or triplex when the strands bind, (hybridize), to each other by forming Watson-Crick, Hoogsteen or reverse Hoogsteen base pairs. Stable binding occurs when an oligonucleotide molecule remains detectably bound to a target nucleic acid sequence under the required conditions.

Complementarity is the degree to which bases in one nucleic acid strand base pair with the bases in a second nucleic acid strand. Complementarity is conveniently described by percentage, that is, the proportion of nucleotides that form base pairs between two strands or within a specific region or domain of two strands.

For example, if 10 nucleotides of a 15-nucleotide oligonucleotide form base pairs with a targeted region of a DNA molecule, that oligonucleotide is said to have 66.67% complementarity to the region of DNA targeted.

‘Sufficient complementarity’ means that a sufficient number of base pairs exist between an oligonucleotide molecule and a target nucleic acid sequence to achieve detectable binding. When expressed or measured by percentage of base pairs formed, the percentage complementarity that fulfills this goal can range from as little as about 50% complementarity to full (100%) complementary. In general, sufficient complementarity is at least about 50%, for example at least about 75% complementarity, at least about 90% complementarity, at least about 95% complementarity, at least about 98% complementarity, or even at least about 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), 27 Molecular Cloning: A Laboratory Manual, 2nd ed., vol. 1-3, Cold Spring Harbor Laboratory Press, Cold Spring Harbor, N.Y., 1989.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning: a laboratory manual, second edition, Cold Spring Harbor Laboratory, Plainview, N.Y. (chapters 9 and I 1).

The following is an exemplary set of hybridization conditions and is not limiting.

Very High Stringency (Detects Sequences that Share at Least 90% Complementarity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Complementarity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 50% Complementarity)

Hybridization: 6×SSC at RT to 55° C. for 16-20 hours

Wash at least twice: 2×-3×SSC at RT to 55° C. for 20-30 minutes each.

Methods for labeling nucleic acid molecules so they can be detected are well known. Examples of such labels include non-radiolabels and radiolabels. Non-radiolabels include, but are not limited to an enzyme, chemiluminescent compound, fluorescent compound (such as FITC, Cy3, and Cy5), metal complex, hapten, enzyme, colorimetric agent, a dye, or combinations thereof. Radiolabels include, but are not limited to, ¹²⁵I and ³⁵S. For example, radioactive and fluorescent labeling methods, as well as other methods known in the art, are suitable for use with the present disclosure. In one example, primers used to amplify the subject's nucleic acids are labeled (such as with biotin, a radiolabel, or a fluorophore). In another example, amplified target nucleic acid samples are end-labeled to form labeled 28 amplified material. For example, amplified nucleic acid molecules can be labeled by including labeled nucleotides in the amplification reactions.

Nucleic acid molecules associated corresponding to one or more marker loci can also be detected by hybridization procedures using a labeled nucleic acid probe, such as a probe that detects only one alternative allele at a marker locus. Most commonly, the target nucleic acid (or amplified target nucleic acid) is separated based on size or charge and transferred to a solid support. The solid support (such as membrane made of nylon or nitrocellulose) is contacted with a labeled nucleic acid probe, which hybridizes to it complementary target under suitable hybridization conditions to form a hybridization complex.

Hybridization conditions for a given combination of array and target material can be optimized routinely in an empirical manner close to the Tm of the expected duplexes, thereby maximizing the discriminating power of the method. For example, the hybridization conditions can be selected to permit discrimination between matched and mismatched oligonucleotides. Hybridization conditions can be chosen to correspond to those known to be suitable in standard procedures for hybridization to filters (and optionally for hybridization to arrays). In particular, temperature is controlled to substantially eliminate formation of duplexes between sequences other than an exactly complementary allele of the selected marker. A variety of known hybridization solvents can be employed, the choice being dependent on considerations known to one of skill in the art (see U.S. Pat. No. 5,981,185).

Once the target nucleic acid molecules have been hybridized with the labeled probes, the presence of the hybridization complex can be analyzed, for example by detecting the complexes.

Methods for detecting hybridized nucleic acid complexes are well known in the art. In one example, detection includes detecting one or more labels present on the oligonucleotides, the target (e.g., amplified) sequences, or both. Detection can include treating the hybridized complex with a buffer and/or a conjugating solution to effect conjugation or coupling of the hybridized complex with the detection label, and treating the conjugated, hybridized complex with a detection reagent. In one example, the conjugating solution includes streptavidin alkaline phosphatase, avidin alkaline phosphatase, or horseradish peroxidase. Specific, non-limiting examples of conjugating solutions include streptavidin alkaline phosphatase, avidin alkaline phosphatase, or horseradish peroxidase. The conjugated, hybridized complex can be treated with a detection reagent. In one example, the detection reagent includes enzyme-labeled fluorescence reagents or calorimetric reagents. In one specific non-limiting example, the detection reagent is enzyme-labeled fluorescence reagent (ELF) from Molecular Probes, Inc. (Eugene, Oreg.). The hybridized complex can then be placed on a detection device, such as an ultraviolet (UV) transilluminator (manufactured by UVP, Inc. of Upland, Calif.). The signal is developed and the increased signal intensity can be recorded with a recording device, such as a charge coupled device (CCD) camera (manufactured by Photometrics, Inc. of Tucson, Ariz.).

In particular examples, these steps are not performed when radiolabels are used.

In particular examples, the method further includes quantification, for instance by determining the amount of hybridization.

### Allele Specific PCR

Allele-specific PCR differentiates between target regions differing in the presence of absence of a variation or polymorphism. PCR amplification primers are chosen based upon their complementarity to the target sequence, such as a sequence disclosed herein. The primers bind only to certain alleles of the target sequence. This method is described by Gibbs, Nucleic Acid Res. 17:12427 2448, 1989.

### Allele Specific Oligonucleotide Screening Methods

Further screening methods employ the allele-specific oligonucleotide (ASO) screening methods (e.g. see Saiki et al., Nature 324:163-166, 1986).

Oligonucleotides with one or more base pair mismatches are generated for any particular allele. ASO screening methods detect mismatches between one allele in the target genomic or PCR amplified DNA and the other allele, showing decreased binding of the oligonucleotide relative to the second allele (i.e. the other allele) oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wildtype allele.

### Ligase Mediated Allele Detection Method

Ligase can also be used to detect point mutations, such as the SNPs in Table 3 in a ligation amplification reaction (e.g. as described in Wu et al., Genomics 4:560-569, 1989). The ligation amplification reaction (LAR) utilizes amplification of specific DNA sequence using sequential rounds of template dependent ligation (e.g. as described in Wu, supra, and Barany, Proc. Nat. Acad. Sci. 88:189-193, 1990).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation.

Each melting domain melts cooperatively at a distinct, base-specific melting temperature (Tm). Melting domains are at least 20 base pairs in length, and can be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W. H. Freeman and Co., New York (1992).

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527, 1986, and Myers et al., in Genomic Analysis, A Practical Approach, K. Davies Ed. IRL Press Limited, Oxford, pp. 95 139, 1988. The electrophoresis system is maintained at a temperature slightly below the Tm of the melting domains of the target sequences.

In an alternative method of denaturing gradient gel electrophoresis, the target sequences can be initially attached to a stretch of GC nucleotides, termed a GC clamp, as described in Chapter 7 of Erlich, supra. In one example, at least 80% of the nucleotides in the GC clamp are either guanine or cytosine. In another example, the GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which can be visualized by ethidium bromide staining.

### Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

### Single-Strand Conformation Polymorphism Analysis

Target sequences or alleles can be differentiated using single-strand conformation polymorphism analysis, which identifies base differences by alteration in electrophoretic migration of single stranded PCR products, for example as described in Orita et al., Proc. Nat. Acad. Sci. 85:2766-2770, 1989. Amplified PCR products can be generated as described above, and heated or otherwise denatured, to form single stranded amplification products. Single-stranded nucleic acids can refold or form secondary structures which are partially dependent on the base sequence. Thus, electrophoretic mobility of single-stranded amplification products can detect base-sequence difference between alleles or target sequences.

### Chemical or Enzymatic Cleavage of Mismatches

Differences between target sequences can also be detected by differential chemical cleavage of mismatched base pairs, for example as described in Grompe et al., Am. J. Hum. Genet. 48:212-222, 1991. In another method, differences between target sequences can be detected by enzymatic cleavage of mismatched base pairs, as described in Nelson et al., Nature Genetics 4:11-18, 1993. Briefly, genetic material from an animal and an affected family member can be used to generate mismatch free heterohybrid DNA duplexes. As used herein, ‘heterohybrid’ means a DNA duplex strand comprising one strand of DNA from one animal, and a second DNA strand from another animal, usually an animal differing in the phenotype for the trait of interest.

### Non-Gel Systems

Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e. there is a mismatch of some form, the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

### Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only).

Generally these programs help in the design of PCR primers by searching for bits of known repeated-sequence elements and then optimizing the Tm by analyzing the length and GC content of a putative primer. Commercial software is also available 35 and primer selection procedures are rapidly being included in most general sequence analysis packages.

Designing oligonucleotides for use as either sequencing or PCR primers to detect requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding programs.

If a possible stem structure is observed, the sequence of the primer can be shifted a few nucleotides in either direction to minimize the predicted secondary structure.

When the amplified sequence is intended for subsequence cloning, the sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed.

Embodiments of the present invention involve transformation of plants with a polynucleotide according to the present invention. The polynucleotide may, for example, be recovered from the cells of a natural host, or it may be synthesized directly in vitro. Extraction from the natural host enables the isolation de novo of novel sequences, whereas in vitro DNA synthesis generally requires pre-existing sequence information. Direct chemical in vitro synthesis can be achieved by sequential manual synthesis or by automated procedures. DNA sequences may also be constructed by standard techniques of annealing and ligating fragments, or by other methods known in the art. Examples of such cloning procedures are given in Sambrook et al. (1989).

The polynucleotide may be isolated by direct cloning of segments of plant genomic DNA. Suitable segments of genomic DNA may be obtained by fragmentation using restriction endonucleases, sonication, physical shearing, or other methods known in the art. A DNA sequence may be obtained by identification of a sequence which is known to be expressed in a different organism, and then isolating the homologous coding sequence from an organism of choice. A coding sequence may be obtained by the isolation of messenger RNA (mRNA or polyA+ RNA) from plant tissue or isolation of a protein and performing “back-translation” of its sequence. The tissue used for RNA isolation is selected on the basis that suitable gene coding sequences are believed to be expressed in that tissue at optimal levels for isolation.

Various methods for isolating mRNA from plant tissue are well known to those skilled in the art, including for example using an oligo-dT oligonucleotide immobilised on an inert matrix. The isolated mRNA may be used to produce its complementary DNA sequence (cDNA) by use of the enzyme reverse transcriptase (RT) or other enzymes having reverse trancriptase activity. Isolation of an individual cDNA sequence from a pool of cDNAs may be achieved by cloning into bacterial or viral vectors, or by employing the polymerase chain reaction (PCR) with selected oligonucleotide primers. The production and isolation of a specific cDNA from mRNA may be achieved by a combination of the reverse transcription and PCR steps in a process known as RT-PCR.

Various methods may be employed to improve the efficiency of isolation of the desired sequence through enrichment or selection methods including the isolation and comparison of mRNA (or the resulting single or double-stranded cDNA) from more than one source in order to identify those sequences expressed predominantly in the tissue of choice. Numerous methods of differential screening, hybridisation, or cloning are known to those skilled in the art including cDNA-AFLP, cascade hybridisation, and commercial kits for selective or differential cloning.

The selected cDNA may then be used to evaluate the genomic features of its gene of origin, by use as a hybridisation probe in a Southern blot of plant genomic DNA to reveal the complexity of the genome with respect to that sequence. Alternatively, sequence information from the cDNA may be used to devise oligonucleotides and these can be used in the same way as hybridisation probes; for PCR primers to produce hybridisation probes, or for PCR primers to be used in direct genome analysis.

Similarly the selected cDNA may be used to evaluate the expression profile of its gene of origin, by use as a hybridisation probe in a Northern blot of RNA extracted from various plant tissues, or from a developmental or temporal series. Again sequence information from the cDNA may be used to devise oligonucleotides which can be used as hybridisation probes, to produce hybridisation probes, or directly for RT-PCR. The selected cDNA, or derived oligonucleotides, may then be used as a hybridisation probe to challenge a library of cloned genomic DNA fragments and identify overlapping DNA sequences.

In embodiments of the present invention, the polynucleotide according to the present invention may be coupled to a promoter which directs expression of SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA in the transgenic plant. The term “promoter” may be used to refer to a region of DNA sequence located upstream of (i.e. 5′ to) the gene coding sequence which is recognised by and bound by RNA polymerase in order for transcription to be initiated.

In further embodiments of the present invention, the polynucleotide according to the present invention may be coupled to a promoter which directs expression of a nucleotide sequence encoding the polypeptide of SEQ ID NO 26, 27, 28, 29, 30, 31, 32, 33, 34, 35, 36, 37, 38 or 39 in the transgenic plant.

There are, broadly speaking, four types of promoters found in plant tissues; constitutive, tissue-specific, developmentally-regulated, and inducible/repressible, although it should be understood that these types are not necessarily mutually exclusive.

A constitutive promoter directs the expression of a gene throughout the various parts of a plant continuously during plant development, although the gene may not be expressed at the same level in all cell types. Examples of known constitutive promoters include those associated with the cauliflower mosaic virus 35S transcript (Odell et al, 1985), the rice actin 1 gene (Zhang et al, 1991) and the maize ubiquitin 1 gene (Cornejo et al., 1993). Constitutive promoters such as the Carnation Etched Ring Virus (CERV) promoter (Hull et al., 1986) are particularly preferred in the present invention.

A tissue-specific promoter is one which directs the expression of a gene in one (or a few) parts of a plant, usually throughout the lifetime of those plant parts. The category of tissue-specific promoter commonly also includes promoters whose specificity is not absolute, i.e. they may also direct expression at a lower level in tissues other than the preferred tissue. Examples of tissue-specific promoters known in the art include those associated with the patatin gene expressed in potato tuber and the high molecular weight glutenin gene expressed in wheat, barley or maize endosperm.

A developmentally-regulated promoter directs a change in the expression of a gene in one or more parts of a plant at a specific time during plant development. The gene may be expressed in that plant part at other times at a different (usually lower) level, and may also be expressed in other plant parts.

An inducible promoter is capable of directing the expression of a gene in response to an inducer. In the absence of the inducer the gene will not be expressed. The inducer may act directly upon the promoter sequence, or may act by counteracting the effect of a repressor molecule. The inducer may be a chemical agent such as a metabolite, a protein, a growth regulator, or a toxic element, a physiological stress such as heat, wounding, or osmotic pressure, or an indirect consequence of the action of a pathogen or pest. A developmentally-regulated promoter might be described as a specific type of inducible promoter responding to an endogenous inducer produced by the plant or to an environmental stimulus at a particular point in the life cycle of the plant. Examples of known inducible promoters include those associated with wound response, such as described by Warner et al (1993), temperature response as disclosed by Benfey & Chua (1989), and chemically induced, as described by Gatz (1995).

In certain embodiments of the present invention, the polynucleotide may be transformed into plant cells leading to controlled expression under the direction of a promoter. The promoters may be obtained from different sources including animals, plants, fungi, bacteria, and viruses, and different promoters may work with different efficiencies in different tissues. Promoters may also be constructed synthetically.

Exogenous genes/polynucleotides may be introduced into plants according to the present invention by means of suitable plant transformation vectors. A plant transformation vector may comprise an expression cassette comprising 5′-3′ in the direction of transcription, a promoter sequence, a coding sequence comprising SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA and, optionally a 3′ untranslated, terminator sequence including a stop signal for RNA polymerase and a polyadenylation signal for polyadenylase. Preferably the vector comprises a coding sequence comprising a nucleotide sequence encoding the polypeptide of SEQ ID NO 26, 27, 28, 29, 30, 31, 32, 33, 34, 35, 36, 37, 38 or 39. The promoter sequence may be present in one or more copies, and such copies may be identical or variants of a promoter sequence as described above. The terminator sequence may be obtained from plant, bacterial or viral genes. Suitable terminator sequences are the pea rbcS E9 terminator sequence, the nos terminator sequence derived from the nopaline synthase gene of Agrobacterium tumefaciens and the 35S terminator sequence from cauliflower mosaic virus, for example. A person skilled in the art will be readily aware of other suitable terminator sequences.

The expression cassette may also comprise a gene expression enhancing mechanism to increase the strength of the promoter. An example of such an enhancer element is that derived from a portion of the promoter of the pea plastocyanin gene, and which is the subject of International Patent Application No. WO 97/20056. These regulatory regions may be derived from the same gene as the promoter DNA sequence or may be derived from different genes, from Selex schwerinii, Selex viminalis or Populus trichocarpa or other organisms, for example from a plant of the family Solanaceae, or from the subfamily Cestroideae. All of the regulatory regions should be capable of operating in cells of the tissue to be transformed.

The promoter DNA sequence may be derived from the same gene as SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA used in the present invention or may be derived from a different gene.

The promoter DNA sequence may be derived from the same gene which comprises the nucleotide sequence encoding the polypeptide of SEQ ID NO 26, 27, 28, 29, 30, 31, 32, 33, 34, 35, 36, 37, 38 or 39 used in the present invention or may be derived from a different gene.

The expression cassette may be incorporated into a basic plant transformation vector, such as pBIN 19 Plus, pBI 101, or other suitable plant transformation vectors known in the art. In addition to the expression cassette, the plant transformation vector will contain such sequences as are necessary for the transformation process. These may include the Agrobacterium vir genes, one or more T-DNA border sequences, and a selectable marker or other means of identifying transgenic plant cells.

The term “plant transformation vector” means a construct capable of in vivo or in vitro expression. Preferably, the expression vector is incorporated in the genome of the organism. The term “incorporated” preferably covers stable incorporation into the genome.

Techniques for transforming plants are well known within the art and include Agrobacterium-mediated transformation, for example. The basic principle in the construction of genetically modified plants is to insert genetic information in the plant genome so as to obtain a stable maintenance of the inserted genetic material. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Typically, in Agrobacterium-mediated transformation a binary vector carrying a foreign DNA of interest, i.e. a chimaeric gene, is transferred from an appropriate Agrobacterium strain to a target plant by the co-cultivation of the Agrobacterium with explants from the target plant. Transformed plant tissue is then regenerated on selection media, which selection media comprises a selectable marker and plant growth hormones. An alternative is the floral dip method (Clough & Bent, 1998) whereby floral buds of an intact plant are brought into contact with a suspension of the Agrobacterium strain containing the chimeric gene, and following seed set, transformed individuals are germinated and identified by growth on selective media. Direct infection of plant tissues by Agrobacterium is a simple technique which has been widely employed and which is described in Butcher D. N. et al., (1980), Tissue Culture Methods for Plant Pathologists, eds.: D. S. Ingrams and J. P. Helgeson, 203-208.

Further suitable transformation methods include direct gene transfer into protoplasts using polyethylene glycol or electroporation techniques, particle bombardment, micro-injection and the use of silicon carbide fibres for example.

Transforming plants using ballistic transformation, including the silicon carbide whisker technique are taught in Frame B R, Drayton P R, Bagnaall S V, Lewnau C J, Bullock W P, Wilson H M, Dunwell J M, Thompson J A & Wang K (1994). Production of fertile transgenic maize plants by silicon carbide whisker-mediated transformation is taught in The Plant Journal 6: 941-948) and viral transformation techniques is taught in for example Meyer P, Heidmann I & Niedenhof I (1992). The use of cassava mosaic virus as a vector system for plants is taught in Gene 110: 213-217. Further teachings on plant transformation may be found in EP-A-0449375.

In a further aspect, the present invention relates to a vector system which carries a nucleotide sequence according to the present invention and introducing it into the genome of an organism, such as a plant. The vector system may comprise one vector, but it may comprise two vectors. In the case of two vectors, the vector system is normally referred to as a binary vector system. Binary vector systems are described in further detail in Gynheung An et al., (1980), Binary Vectors, Plant Molecular Biology Manual A3, 1-19.

One extensively employed system for transformation of plant cells uses the Ti plasmid from Agrobacterium tumefaciens or a Ri plasmid from Agrobacterium rhizogenes An et al., (1986), Plant Physiol. 81, 301-305 and Butcher D. N. et al., (1980), Tissue Culture Methods for Plant Pathologists, eds.: D. S. Ingrams and J. P. Helgeson, 203-208. After each introduction method of the desired exogenous gene according to the present invention in the plants, the presence and/or insertion of further DNA sequences may be necessary. If, for example, for the transformation the Ti- or Ri-plasmid of the plant cells is used, at least the right boundary and often however the right and the left boundary of the Ti- and Ri-plasmid T-DNA, as flanking areas of the introduced genes, can be connected. The use of T-DNA for the transformation of plant cells has been intensively studied and is described in EP-A-120516; Hoekema, in: The Binary Plant Vector System Offset-drukkerij Kanters B. B., Alblasserdam, 1985, Chapter V; Fraley, et al., Crit. Rev. Plant Sci., 4:1-46; and An et al., EMBO J. (1985) 4:277-284.

Plant cells transformed with nucleotides of the present invention may be grown and maintained in accordance with well-known tissue culturing methods such as by culturing the cells in a suitable culture medium supplied with the necessary growth factors such as amino acids, plant hormones, vitamins, etc.

The “transgenic plant” in relation to the present invention may include any plant that comprises an exogenous polynucleotide/gene according to the present invention or any plant has been modified to up or down regulate expression of the endogenous gene/polynucleotide. Preferably the exogenous gene/polynucleotide is incorporated in the genome of the plant.

In one aspect, a nucleic acid sequence, plant transformation vector or plant cell according to the present invention is in an isolated form. The term “isolated” means that the sequence is at least substantially free from at least one other component with which the sequence is naturally associated in nature and as found in nature.

In one aspect, a nucleic acid sequence, plant transformation vector or plant cell according to the invention is in a purified form. The term “purified” means in a relatively pure state—e.g. at least about 90% pure, or at least about 95% pure or at least about 98% pure.

The plants which are transformed with an exogenous gene according to the present invention include but are not limited to monocotyledonous and dicotyledonous fodder crops, forage crops, ornamental crops, fruit crops, food crops, algae, forestry trees, bioenergy crops and biofuel crops including the following species and species hybrids: Acacia spp., Acer spp., Actinidia ssp., Agave spp., Agropyron spp., Agrostis spp., Allium spp., Alnus spp., Alopecurus spp., Amaranthus spp., Ananas spp., Apium spp., Arachis spp., Areca spp., Arundo spp., Arrhenatherum spp., Asparagus spp; Avena spp., Atriplex spp., Attalea spp., Beta spp., Betula spp., Brassica spp., Bromus spp., Bouteloua spp., Camelina spp., Camellia spp., Cannabis spp., Capsicum spp., Carica spp., Carex spp., Carthamus spp., Castanea spp., Carum spp., Cinnamomum spp., Citrus spp., Cocos spp., Coffea spp., Corchorus spp., Cotoneatser spp., Cucurbita spp., Cupressus spp., Cynodon spp., Daucus spp., Dactylis spp., Eucalyptus spp., Elaeis spp., Eleusine spp., Fagus spp., Festuca spp., Ficus spp., Fraxinus spp., Geranium spp., Ginkgo spp., Glycine spp., Gossypium spp., Helianthus spp., Hemerocallis spp., Heracleum spp., Hedysarum spp., Hibiscus spp., Hordeum spp., Indigo spp., Ipomoea spp., Lettuca spp., Jatropha spp., Lotus spp., Lactuca spp., Lathyrus spp., Lens spp., Linum spp., Lolium spp., Lupinus spp., Lezula spp., Lycopersicon spp., Malus spp., Manihot spp., Medicago spp., Melilotus spp., Mentha spp., Miscanthus spp., Musa spp., Nicotiana spp., Olea spp., Onobrychis spp., Ophiopogon spp., Oryza spp., Panicum spp., Papaver spp., Petunia spp., Phaseolus spp., Pennisetum spp., Phalaris spp., Phoenix spp., Phleum spp., Phyllostachys spp., Physalis spp., Panicum spp., Picea spp., Pinus spp., Pistacia spp., Pisum spp., Poa spp., Podocarpus spp., Pogmania spp., Populus spp., Prunus spp., Quercus spp., Ribes spp., Robinia spp., Rosa spp., Raphanus spp., Rheum spp., Ricinus spp., Rubus spp., Salix spp., Sequoia spp., Sesamum spp., Setaria spp., Saccharum spp., Sambucus spp., Secale spp., Sinapis spp., Solanum spp., Sorghum spp., Trifolium spp., Triticum spp., Triticosecale spp., Trisetum spp., Tagetes spp., Theobroma spp., Triadica spp., Vicia spp., Vitis spp., Vigna spp., Viola spp., Watsonia spp., Zea spp. amongst others.

### EXAMPLES

### Example 1

### Plant Material

This study focuses on the K8 willow mapping population. This population comprises 947 full-sib individuals and was produced at Long Ashton Research Station (LARS), in 1999. The pedigree of the population is shown in Table 1.

The population was established in a field experiment at LARS in 2000 and later at Rothamsted Research (RRes), Harpenden, Herts, UK in 2003. Six clonal replicates of each K8 genotype were planted as single plots, each in a 2×3 arrangement within the field experiment. Plots were arranged in a 52×23 plot row by column design. To facilitate identification of any environmental inconsistencies across the trial site, and to allow subsequent adjustment of trait values prior to QTL analyses, a reference willow variety was planted at 64 pre-selected plot positions throughout the site. The biomass cultivar, S. viminalis var. Jorr, was selected for this role at the LARS site and the cultivar Bowles Hybrid was used at RRes. These control genotypes were also used to surround the entire site to minimise any edge effects and also to form internal tramline columns after every fourth (RRes) or fifth (LARS) column of K8 progeny. Progeny were arranged in random order in the design. For additional details, see Hanley S J (2003) Genetic mapping of important agronomic traits in biomass willow. PhD thesis, University of Bristol, UK (Hanley, 2003).

Both plantations were established from 15 cm stem cuttings, allowed to grow for one year, after which the plants were coppiced during the winter by removing the first year's growth from the stool. Plants were then allowed to grow for a further two years before a second cutback. Plants were then coppiced after each period of three seasons of growth.

### Trait Measurements

Trait measurements were made according to Table 2 below.

TABLE 2

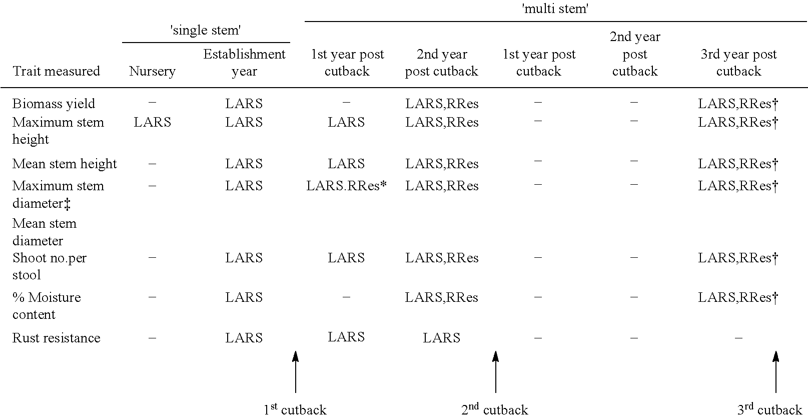

*: trait measured on 480 progeny only
†: RRes data available Spring 2008
‡: stem diameters measured at 55 cm from the stool

Trait data was first analysed for spatial inconsistencies across the trial site and data adjusted to account for this. The method of Residual Maximum Likelihood (REML) (Patterson and Thompson 1971; Robinson et al. 1982) was used to fit mixed (involving fixed and random effects) models (Searle et al. 1992) to the trait data, employing GenStat software (©Sixth Edition, Lawes Agricultural Trust, Rothamsted Experimental Station, 2002). Using theory developed by Gleeson and Cullis (1987), Cullis and Gleeson (1991) and Cullis et al. (1998), the most appropriate model to correctly describe the effects of spatial trends, defined as autoregressive components for rows and or columns, for data from each assessment was identified. This utilised the trait information provided by a reference genotypes (Jorr or Bowles Hybrid). Changes in model deviance (Genstat Committee 1993) were used to assess the significance (P<0.05) of any extra (spatial) terms in models, these changes being asymptotically distributed as chi-squared on degrees of freedom equal to the number of extra parameters.

Adjusted trait scores were then utilised in QTL analysis according to standard methodologies as included in the software package MapQTL (Kyazma).

### Identification and High Resolution Mapping of the Yield QTL

The yield QTL was first identified following an initial QTL screen based on K8 progeny numbers 1-480 only. The K8 linkage map comprised amplified fragment length polymorphism (AFLP) and microsatellite markers. In addition, a genome-wide set of Single Nucleotide Polymorphism (SNP) markers was developed and included in analysis for aligning the K8 willow map to the publicly-available poplar genome sequence. Further details of this approach are available in Hanley, S. J., Mallott, M. D. & Karp A. (2006) Tree Genetics and Genomes, 3, 35-48

Once the approximate position of the QTL was determined (on Linkage Group X; Linkage group nomenclature is a provided for the poplar genome sequence; http://genome.jgi-psf.org/Poptr1_{—}1/Poptr1_{—}1.home.html) through the initial QTL screen, an additional 11 SNP markers were developed to target this region to increase mapping resolution and further delimit the locus. The SNP markers were derived from sequencing willow orthologues of genes in this region of the poplar genome sequence. Full details of the method developed for identifying SNP markers are described in Hanley, S. J., Mallott, M. D. & Karp A. (2006) Tree Genetics and Genomes, 3, 35-48.

| | | Forward PCR | Reverse PCR | | |
|---|---|---|---|---|---|
| Marker | Class | primer(5′→3′) | primer (5′→3′) | SNapShot primer | Type |
| X_15341094* | SNP | GGGAAACAGATAGTGGGCAGTC | GCCTCCTTCTCCTGTAAGCAC | ACCTTAACCTGCAGCTCTTACCITAA | ACxAC |
| X_15478832 | SNP | TGATGCCTCCAAAGGTTTCTC | TCCTGGCGTGTTCATAGAGGT | GATGGGAAGTAAAAATTATCCGAGCAAGAT | ACxAC |
| X_15533399 | SNP | GTGGCTCTTCTCCATTGCTGT | GTGCTTTTTGCTCCACCTTTG | AATAGCAAATATGGGGGCTT | ATxAT |
| x_15727779 | SNP | AGAAGGGATGTGCCAAAGTGA | ACAAGCTGGATTGGTGGAAGA | ACTTTTGATATTTTCTAACCTTTTCTCTTA | CTxCT |
| | | | | TTGTA | |
| X_15758822 | SSR | CAAAAACGCACCCTATTCTTCC | CCAGAGTCCCCTTGAACACAC | — | abxac |
| X_15777280 | SNP | AAAACAACCTCCCTCCCTTGA | TCTGCAAGCCCACTTTTTCTT | TTTGAGGAAGACGGCAAATG | TGxTG |
| X_15905315 | SNP | CAACATATTGTGGATGCAGga | CAGTGATACAATGTCTGCAAGGA | AGGATTTCCCACAGATTGGTTTCAC | CGxCG |
| X_15917077 | SNP | TTCCTTGTTTTGGCTTTGGTG | CCATCGCCTGTATCCACACTT | ATTCAGCTGTCGAATTGATTGATT | TGxTG |
| X:15951166 | SNP | TGGTGAGCGAGAGTACGTGAA | AATCTTCCTGGCCCTCAAAAC | GGGTATGCTCAGCCTGCC | ACxAC |
| X:15945623 | SNP | ATTGGAATCTCTTGGGGCTTT | CACCTGCTCCATAATCCCTCT | TCATTGATAACTGCTATTGTTCCCCAGA | AGxAG |
| X:15958515 | SNP | CAGAGACCCAAATGGACTGGA | AACGACCTAATCCCCTGGAAA | TCAATGCATGACGGTGTTCTTGTGGTGACA | AGxAG |
| | | | | GT | |

*It should be noted that the marker numbers do not necessarily refer to the most up date position available in the poplar genome and this may change due to ongoing annotation and assembly.

All of these SNP markers were heterozygous in both mapping population parents (S3 & R13) and segregated according to the expected 1:2:1 (AA:AB:BB) ratio in the progeny. All 11 markers were used to genotype the 947 individuals of the mapping population. Forty three individuals were not included in subsequent analysis as genotyping failed in some instances and some plants had died in the field and DNA for screening was no longer available. A fine-scale linkage map was then calculated based on the 11 markers. The order of markers on the willow map is co-linear with the poplar genome sequence.

The resulting linkage map spanned 5.1 cM. This map was used in conjunction with the genotype and trait data in a second round of QTL analysis. Results of interval mapping are shown in FIG. 13 for total fresh weight for two harvest years at the LARS site (2003 & 2006) and for the RRes site in 2005. QTL for maximum stem diameter and maximum stem height are also shown for both sites for equivalent years. These traits are highly correlated with total harvestable yield in this population (Hanley S J (2003) Genetic mapping of important agronomic traits in biomass willow. PhD thesis, University of Bristol, UK).

The sequences for willow markers X_{—}15341094, X_{—}15758822, X_{—}15905315 and X_{—}15958515 also yielded SNPs that were specific to each parent indicating that there are three haplotypes segregating in this region in the K8 population. Due to the nature of the cross that generated the K8 population, there is a maximum of three alleles segregating at any given locus in this population. As explained in Example 2, the female parent of the cross, cultivar ‘S3’ was found to produce two alleles of different length (A & B). The male parent, cultivar ‘R13’ was found to contain two alleles (A & C) where A is a common allele that is present in S3. The diploid K8 mapping population can therefore inherit the following combinations of alleles: AA, AB, AC, BC. As indicated in Example 2, allele C is associated with increased harvestable biomass yield when compared to the contribution of allele A to harvestable biomass yield.

Sequence analysis of the QTL region based on the poplar genome.

QTL indicates that the most likely position of the QTL is between markers X_{—}15727779 and X_{—}15917077. The position of these markers in the poplar genome was determined by BLASTN homology searches using the willow sequence used to derive the SNP markers.

The homologous genomic region in poplar is predicted to contain 10 genes. These are referred to as Xyld1, Xyld2, Xyld3, Xyld4, Xyld5, Xyld6, Xyld7, Xyld8, Xyld9 and Xyld10. The physical size of this region is predicted to be 196118 base pairs in length. However, a gap in the public sequence prevents an accurate measure of the length. Eight of the genes have EST sequence to support their expression.

Two willow BAC clones have been identified that cover the region delimited by the two markers. Partial sequencing of these clones indicates that homologues to 9 of the 10 genes within the QTL region in poplar can be identified in willow plant ‘R13’. ‘R13’ contains two alleles (A and C) and FIG. 2 shows the sequence of the QTL region of allele A. Alleles A and C of the 9 willow genes were identified using routine techniques and are shown in the Figures.

The amino acid sequences of the polypeptides encoded by Alleles A and C of the 9 willow genes are shown in the Figures. These were identified using cDNA sequences that allowed exons in the gene sequences to be identified and thus the polypeptide sequence to be predicted. The cDNA sequences were predicted by full sequencing of salix transcripts that allowed intron-exon boundaries to be identified. In some cases the exons were predicted using annotation information on the public poplar genome website. These predictions are based on transcript sequencing in poplar and gene prediction algorithms. Polypeptide sequences were predicted using partially sequenced willow transcripts in conjunction with public poplar genome annotation data which is based on gene finding algorithms and poplar transcript sequence information (Tuskan et al., 2006. The Genome of Black Cottonwood, Populus trichocarpa (Torr. & Gray) Science 313 p5793.”

Details of the genes are detailed below:

### 1. Xyld1

Shows best homology in Arabidopsis thaliana with Locus AT3G12740, or ALIS1 (ALA-Interacting Subunit). ALIS1 is a member of a family of phospholipid transporters (ALIS1-ALIS5) which are homologs of the Cdc50p/Lem3p family in yeast that are essential for the trafficking of yeast P4-ATPases. The Arabidopsis ALIS proteins are 27-30% identical to yeast Cdc50p and similarity ranges from 48-53%. In yeast ALIS1 shows strong affinity to ALA3. In Arabidopsis, ALA3 has been shown to be important for trans-Golgi proliferation of slime vesicles containing polysaccharides and enzymes for secretion. In yeast, ALA3 function requires interaction with the ALIS1. In Arabidopsis plants, ALIS1, like ALA3, is localised to membranes of Golgi-like structures and is expressed in root peripheral columella cells. It has been proposed that the ALIS1 protein is a β-sub-unit of ALA3 in Arabidopsis and that this protein is important part of the Golgi machinery in plants required for secretory processes during development.

### Relevant Publications

Poulsen L R, López-Marqués R L, McDowell S C, Okkeri J, Licht D, Schulz A, Pomorski T, Harper J F, Palmgren M G. 2008 The Arabidopsis P4-ATPase ALA3 localizes to the golgi and requires a beta-subunit to function in lipid translocation and secretory vesicle formation. Plant Cell. 3:658-76.

Bosco C D, Lezhneva L, Biehl A, Leister D, Strotmann H, Wanner G, Meurer J. 2004 Inactivation of the chloroplast ATP synthase gamma subunit results in high non-photochemical fluorescence quenching and altered nuclear gene expression in Arabidopsis thaliana. J Biol Chem.279(2):1060-9.

### 2. Xyld 2

Shows strongest homology to Arabidopsis thaliana gene ALDH5F1 (Locus AT1G79440; previous nomenclature SSADH; EC 1.2.1.24) which is a member of the aldehyde dehydrogenases (ALDHs) protein superfamily of NAD(P)C-dependent enzymes that oxidize a wide range of endogenous and exogenous aliphatic and aromatic aldehydes. The Arabidopsis genome contains 14 unique ALDH sequences encoding members of nine ALDH families, including eight known families and one novel family (ALDH22) that is currently known only in plants. Of these, there is one succinic semialdehyde dehydrogenase gene, ALDH5F1, which encodes a protein of 528 amino acids. ALDH5F1 is the only confirmed identified member of the succinic semialdehyde family in plants. The Arabidopsis protein is localized to mitochondria and a kinetic analysis showed that the recombinant enzyme was specific for succinic semialdehyde and regulated by adenine nucleotides. T-DNA knockout mutants of ALDH5F1 result in dwarfed plants with necrotic lesions and are sensitive to both ultraviolet-B light and heat stress. Plants with ssadh mutations accumulate elevated levels of H₂O₂, suggesting a role for this gene in stress regulation detoxification pathway plant, providing defense against environmental stress by preventing the accumulation of reactive oxygen species.

### Relevant Publications

Hueser, A F, UI L. 2008 Analysis of GABA-shunt metabolites in Arabidopsis thaliana 19th International Conference on Arabidopsis Research

Ludewig F, Hüser A, Fromm H, Beauclair L, Bouché N. 2008 Mutants of GABA transaminase (POP2) suppress the severe phenotype of succinic semialdehyde dehydrogenase (ssadh) mutants in Arabidopsis. PLoS ONE 3(10):e3383

Zybailov B, Rutschow H, Friso G, Rudella A, Emanuelsson O, Sun Q, van Wijk K J. 2008 Sorting signals, N-terminal modifications and abundance of the chloroplast proteome. PLoS ONE 3(4):e1994

Fait A, Yellin A, Fromm H. 2005 GABA shunt deficiencies and accumulation of reactive oxygen intermediates: insight from Arabidopsis mutants. FEBS Lett. 579(2):415-20

Kirch H H, Bartels D, Wei Y, Schnable P S, Wood A J. 2004 The ALDH gene superfamily of Arabidopsis. Trends Plant Sci. 9(8):371-7

Breitkreuz K E, Allan W L, Van Cauwenberghe O R, Jakobs C, Talibi D, Andre B, Shelp B J. 2003 A novel gamma-hydroxybutyrate dehydrogenase: identification and expression of an Arabidopsis cDNA and potential role under oxygen deficiency. J Biol Chem. 278(42):41552-6.

### 3. Xyld3

Shows strongest homology with Arabidopsis thaliana ALTERED PHLOEM DEVELOPMENT (APL) gene (Locus AT1G79430), which encodes a MYB coiled-coil-type transcription factor that is required for phloem identity in Arabidopsis. APL has been proposed to have a dual role both in promoting phloem differentiation and in repressing xylem differentiation during vascular development.

### Relevant Publications

Truernit E, Bauby H, Dubreucq B, Grandjean O, Runions J, Barthélémy J, Palauqui J C. 2008 High-resolution whole-mount imaging of three-dimensional tissue organization and gene expression enables the study of Phloem development and structure in Arabidopsis. Plant Cell. 20(6):1494-503

Lehesranta S, Lindgren O, Taehtiharju S, Carlsbecker A, Helariutta Y 2008 The role of APL as a transcriptional regulator in specifying vascular identity 19th International Conference on Arabidopsis Research

Carlsbecker A, Lindgren O, Bonke M, Thitamadee S, Tähtiharju S, Helariutta Y 2004 Genetic analysis of procambial development in the Arabidopsis root 15th International Conference on Arabidopsis Research

Bonke M, Hauser M-T, Helariutta Y 2002 The APL locus is required for phloem development in Arabidopsis roots. 13th International Conference on Arabidopsis Research

### 4. Xyld4

Show strongest homology in Arabidopsis thaliana to Locus AT1G79420. Function not yet described.

### 5. Xyld5

Shows strongest homology with AtOCT2 in Arabidopsis thaliana (Locus AT1G79360). ATOCT2 is one of six Arabidopsis organic cation/carnitine transporter (OCT)-like proteins, named AtOCT1-AtOCT6 (loci At1g73220, At1g79360, At1g16390, At3g20660, At1g79410 and At1g16370, respectively) that have been identified. These proteins cluster in a small subfamily within the ‘organic solute cotransporters’ included in the large sugar transporter family of the major facilitator superfamily (MFS). AtOCT1 shares features of organic cation/carnitine transporters (OCTs). In animals, mammalian plasma membrane OCTs are involved in homeostasis and distribution of various small endogenous amines (e.g. carnitine, choline) and detoxification of xenobiotics such as nicotine. AtOCT1 is able to transport carnitine in yeast and is likely to be involved in the transport of carnitine or related molecules across the plasma membrane in plants.

The orthologous gene sequence has not yet been identified in willow.

### Related Publication

Tohge T, Nishiyama Y, Hirai M Y, Yano M, Nakajima J, Awazuhara M, Inoue E, Takahashi H, Goodenowe D B, Kitayama M, Noji M, Yamazaki M, Saito K. 2005 Functional genomics by integrated analysis of metabolome and transcriptome of Arabidopsis plants over-expressing an MYB transcription factor. Plant J. 42(2):218-35

### 6. Xyld6

Shows best fit with ATOCT3 Arabidopsis ORGANIC CATION/CARNITINE TRANSPORTER2). ATOCT3 is one of six Arabidopsis organic cation/carnitine transporter (OCT)-like proteins, named AtOCT1-AtOCT6 (loci At1g73220, At1g79360, At1g16390, At3g20660, At1g79410 and At1g16370, respectively) referred to above. These proteins cluster in a small subfamily within the ‘organic solute cotransporters’ included in the large sugar transporter family of the major facilitator superfamily (MFS).

### Relevant Publications

Lelandais-Brière C, Jovanovic M, Tones G A, Perrin Y, Lemoine R, Corre-Menguy F, Hartmann C. 2007 Disruption of AtOCT1, an organic cation transporter gene, affects root development and carnitine-related responses in Arabidopsis. Plant J. 51(2):154-64

Price J, Laxmi A, St Martin S K, Jang J C. 2004 Global transcription profiling reveals multiple sugar signal transduction mechanisms in Arabidopsis. Plant Cell.16(8):2128-50

### 7. Xyld7

Shows homology with members of the R2R3-type MYB gene family in Arabidopsis. Although no functional data are available for most of the 125 R2R3-type AtMYB genes, a number of functions have been assigned concerning many aspects of plant secondary metabolism, as well as the identity and fate of plant cells. This includes regulation of phenylpropanoid metabolism, control of development and determination of cell fate and identity, plant responses to environmental factors and mediating hormone actions.

### Relevant Publications

Stracke R, Werber M, Weisshaar B. 2001 The R2R3-MYB gene family in Arabidopsis thaliana. Curr Opin Plant Biol. 4(5):447-56

Riechmann J L, Heard J, Martin G, Reuber L, Jiang C, Keddie J, Adam L, Pineda O, Ratcliffe O J, Samaha R R, Creelman R, Pilgrim M, Broun P, Zhang J Z, Ghandehari D, Sherman B K, Yu G. 2000 Arabidopsis transcription factors: genome-wide comparative analysis among eukaryotes. Science. 290(5499):2105-10

### 8. Xyld8

Shows best fit with ANAC028, Arabidopsis NAC domain containing protein (Locus AT1G65910). NAC (NAM, ATAF, and CUC) is a plant-specific gene family. NAC family transcription factors are involved in maintaining organ or tissue boundaries regulating the transition from growth by cell division to growth by cell expansion. Most NAC proteins contain a highly conserved N-terminal DNA-binding domain, a nuclear localization signal sequence, and a variable C-terminal domain. 75 and 105 NAC genes were predicted in the Oryza sativa and Arabidopsis genomes, respectively. The functions of only some of these have been described. The first reported NAC genes were NAM from petunia and CUC2 from Arabidopsis that participate in shoot apical meristem development. CUC1, CUC2 and nam are expressed at the boundaries between cotyledonary primordial and between floral organs and are specifically involved in shoot apical meristem formation and separation of cotyledons and floral organs. Other development-related NAC genes have been suggested with roles in controlling cell expansion of specific flower organs e.g. NAP or auxindependent formation of the lateral root system e.g. NAC1. Some of NAC genes, such as ATAF1 and ATAF2 genes from Arabidopsis and the StNAC gene from potato, are induced by pathogen attack and wounding. More recently, a few NAC genes, such as AtNAC072 (RD26), AtNAC019, AtNAC055 from Arabidopsis, and BnNAC from Brassica (31), were found to be involved in responses to environmental stress. Seven members of NAC family At2g18060, At4g36160, At5g66300, At1g12260, At1g62700, At5g62380, and At1g71930 have been designated as VASCULAR-RELATED NAC-DOMAIN PROTEIN 1 (VND1 to VND7). Members of these could induce transdifferentiation of various cells into metaxylem- and protoxylem-like vessel elements, respectively, in Arabidopsis and poplar. Similarly ANAC012 and ANAC073 also appear to have a role in xylem development and secondary wall thickening in Arabidopis.

### Relevant Publications

Ooka H, Satoh K, Doi K, Nagata T, Otomo Y, Murakami K, Matsubara K, Osato N, Kawai J, Caminci P, Hayashizaki Y, Suzuki K, Kojima K, Takahara Y, Yamamoto K, Kikuchi S. 2003 Comprehensive analysis of NAC family genes in Oryza sativa and Arabidopsis thaliana. DNA Res. 10(6):239-47

Riechmann J L, Heard J, Martin G, Reuber L, Jiang C, Keddie J, Adam L, Pineda O, Ratcliffe O J, Samaha R R, Creehnan R, Pilgrim M, Broun P, Zhang J Z, Ghandehari D, Sherman B K, Yu G. 2000 Arabidopsis transcription factors: genome-wide comparative analysis among eukaryotes. Science 290(5499):2105-109.

### Xyld9

Show strongest homology in Arabidopsis thaliana to Locus AT1G79390. The function of this expressed protein has not yet been described

### 10. Xyld10

Shows homology to the RGLG2 (RING DOMAIN LIGASE2) locus of Arabidopsis thaliana (Locus AT1G79380). In functional terms, the RING domain can basically be considered a protein-interaction domain. RING-finger proteins have been implicated in a range of diverse biological processes and biochemical activities, from transcriptional and translational regulation to targeted protein degradation.

### Relevant Publications

Kosarev P, Mayer K F, Hardtke C S. 2002 Evaluation and classification of RING-finger domains encoded by the Arabidopsis genome. Genome Biol. 3(4):RESEARCH 0016.1

### Example 2

Provided below is an example of the use of a diagnostic molecular marker derived from the QTL region that can be used to select for favourable alleles within a breeding programme:

A microsatellite marker was developed to screen for the three QTL alleles segregating in members of the K8 population of Salix. The microsatellite marker is amplified by PCR using the following pair of primers:

Forward primer
5′-CAAAAACGCACCCTATTCTTCC-3′
Reverse primer
5′-CCAGAGTCCCCTTGAACACAC-3′

The sequence of the amplified region for allele A (179bp) is:

CAAAAACGCACCCTATTCTTCCCTATTTGCATCGCATTTGTTCTTGAA
TCTCTTTGTATTCCCTGAGTCTCAGAGAGAGAGAGAGAGAGAGAGAGA
AGGAAAGAGAGAATGTTCCATACCAAGAAACCCTCAACTATGAATTCC
CATGATAGACCCATGTGTGTTCAAGGGGACTCTGG

These primers generate amplicons of three different lengths in the K8 mapping population and thus are informative for the three alleles that are segregating in the yield QTL region. The female parent of the cross, cultivar ‘S3’ produces two alleles of different length (A & B). The male parent, cultivar ‘R13’ contains two alleles (A & C) where A is a common allele that is present in S3.

The diploid K8 mapping population can therefore inherit the following combinations of alleles: AA, AB, AC, BC. Table 3 shows the mean trait values for each of these classes in the population for total fresh weight harvested, maximum stem diameter and maximum stem height. Analysis is based trait data collected at Long Ashton Research Station in 2003. The non-parametric rank-sum test of Kruskal-Wallis (KW) (Lehmann, 1975) was used to determine associations between marker genotypes and trait scores.

TABLE 3
Mean trait values associated with inheritance of particular QTL alleles (A, B
and C) in the K8 mapping population as determined by the application of a
microsatellite marker.

| | | microsatellite genotype | | | | | | |
|---|---|---|---|---|---|---|---|---|
| Trait | N^{o} | AA | AB | AC | BC | KW | df | Significance |
| Total fresh biomass | 902 | 1.30 | 1.90 | 1.75 | 2.17 | 132.76 | 3 | ******* |
| harvested per stool (kg) | | | | | | | | |
| Maximum stem | 849 | 16.30 | 20.12 | 19.22 | 21.37 | 186.37 | 3 | ******* |
| diameter per stool (cm) | | | | | | | | |
| Maximum stem height | 902 | 3.16 | 3.79 | 3.69 | 3.96 | 223.95 | 3 | ******* |
| per stool (m) | | | | | | | | |

N^{o}: Number of plants included in analysis
KW: Kruskal-Wallis test statistic
df: degrees of freedom
Significance: ******* = 0.0001

In this example, plants of genotype AA often give the lowest yield and plants of genotype BC often give rise to the highest yields. Where the goal of a breeding programme is to increase harvestable biomass yield, plants of genotype BC would be preferentially selected using the marker. Similarly, potential parents of genotype AA might be excluded from a crossing programme as this allele can be associated with lower yields.

### Example 3

Disruption of Xyld7 gene sequence in QTL haplotype A

An alignment of Gene Xyld7 allele A (SEQ ID NO 2) sequence with the Gene Xyld7 allele C sequence (SEQ ID NO 1) (as shown in the alignment of FIG. 9D) indicates Gene Xyld7 allele A has an insertion region with extra nucleotides that are not present in Gene Xyld7 allele C sequence SEQ ID NO 1. SEQ ID NO 26 (as shown in FIG. 9E) shows the amino acid sequence of the Salix Xyld7 allele C polypeptide.

A comparison of Xyld7 gene sequences for both alleles of plant R13 (alleles A and C) identified an insertion in Xyld7_A allele which is not present in the Xyld_C allele sequence. To determine whether the insertion is in coding sequence, the transcript of allele C of this gene was fully sequenced which confirmed that the insertion in allele A is within exon 3 of the gene. The resulting allele A transcript, if expressed, would not be expected to encode a functional protein. Indeed, while both allele B and C transcripts have been identified, no allele A derived transcript has yet been identified in plants S3 and R13 (the K8 parents which carry either the A and B alleles or the A and C alleles, respectively). It is therefore possible that allele A of this gene is non-functional in the K8 mapping population and this may contribute to the underlying phenotypic variation that is represented by the biomass yield QTL.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the following claims.

1.-44. (canceled)

45. A method of selecting a crop by marker assisted selection of an allele associated with harvestable biomass yield, wherein said allele is an allele of a polynucleotide sequence, said polynucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25, said method comprising: determining the presence of one or more markers, which markers are genetically linked to said polynucleotide.

46. A method according to claim 45, wherein the crop plant is a monocotyledonous and dicotyledonous fodder crop plant, forage crop plant, ornamental crop plant, fruit crop plant, food crop plant, an algae, a forestry tree, a bioenergy crop plant or a biofuel crop plant, Acacia spp., Acer spp., Actinidia ssp., Agave spp., Agropyron spp., Agrostis spp., Allium spp., Alnus spp., Alopecurus spp., Amaranthus spp., Ananas spp., Apium spp., Arachis spp., Areca spp., Arundo spp., Arrhenatherum spp., Asparagus spp; Avena spp., Atriplex spp., Attalea spp., Beta spp., Betula spp., Brassica spp., Bromus spp., Bouteloua spp., Camelina spp., Camellia spp., Cannabis spp., Capsicum spp., Carica spp., Carex spp., Carthamus spp., Castanea spp., Carum spp., Cinnamomum spp., Citrus spp., Cocos spp., Coffea spp., Corchorus spp., Cotoneatser spp., Cucurbita spp., Cupressus spp., Cynodon spp., Daucus spp., Dactylis spp., Eucalyptus spp., Elaeis spp., Eleusine spp., Fagus spp., Festuca spp., Ficus spp., Fraxinus spp., Geranium spp., Ginkgo spp., Glycine spp., Gossypium spp., Helianthus spp., Hemerocallis spp., Heracleum spp., Hedysarum spp., Hibiscus spp., Hordeum spp., Indigo spp., Ipomoea spp., Lettuca spp., Jatropha spp., Lotus spp., Lactuca spp., Lathyrus spp., Lens spp., Linum spp., Lolium spp., Lupinus spp., Lezula spp., Lycopersicon spp., Malus spp., Manihot spp., Medicago spp., Melilotus spp., Mentha spp., Miscanthus spp., Musa spp., Nicotiana spp., Olea spp., Onobrychis spp., Ophiopogon spp., Oryza spp., Panicum spp., Papaver spp., Petunia spp., Phaseolus spp., Pennisetum spp., Phalaris spp., Phoenix spp., Phleum spp., Phyllostachys spp., Physalis spp., Panicum spp., Picea spp., Pinus spp., Pistacia spp., Pisum spp., Poa spp., Podocarpus spp., Pogmania spp., Populus spp., Prunus spp., Quercus spp., Ribes spp., Robinia spp., Rosa spp.,Raphanus spp., Rheum spp., Ricinus spp., Rubus spp., Salix spp., Sequoia spp., Sesamum spp., Setaria spp., Saccharum spp., Sambucus spp., Secale spp., Sinapis spp., Solanum spp., Sorghum spp., Trifolium spp., Triticum spp., Triticosecale spp., Trisetum spp., Tagetes spp., Theobroma spp., Triadica spp., Vicia spp., Vitis spp., Vigna spp., Viola spp., Watsonia spp. or Zea spp.

47. A method for predicting harvestable biomass yield according to claim 45, wherein the crop is a member of the genus Salix or Populus.

48. A method according to claim 45, wherein the marker is within an interval of less than 45, 40, 35, 30, 25, 20, 15, 10, 5, 4, 3, 2, 1 or 0 centimorgans (cM) from said polynucleotide.

49. A method for producing a transgenic crop plant of claim 53, comprising introducing into an unmodified crop plant an exogenous polynucleotide, wherein said polynucleotide comprises a nucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA sequence.

50. A method for producing a transgenic crop plant of claim 54 that expresses a recombinant polypeptide comprising an amino acid sequence having at least 50, 55, 60, 65, 70, 75, 80, 85, 90, 95, 97, 98, 99 or 100% identity to SEQ ID NO 26, 27, 28, 29, 30, 31, 32, 33, 34, 35, 36, 37, 38 or 39, comprising introducing an exogenous polynucleotide comprising a cDNA encoding said recombinant polypeptide into an unmodified crop plant.

51. A method according to claim 50, wherein the exogenous polynucleotide is associated with a promoter sequence capable of directing constitutive expression of the protein encoded by the exogenous polynucleotide in the plant.

52. A method according to claim 50, wherein a primary transgenic plant is generated by introduction of the exogenous polynucleotide, and a secondary transgenic plant is produced from the primary transgenic plant.

53. A transgenic crop plant comprising an exogenous gene, wherein said gene comprises a sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA sequence.

54. A transgenic crop plant expressing a recombinant polypeptide having at least 50, 55, 60, 65, 70, 75, 80, 85, 90, 95, 97, 98, 99 or 100% identity to SEQ ID NO 26, 27, 28, 29, 30, 31, 32, 33, 34, 35, 36, 37, 38 or 39.

55. A transgenic crop plant according to claim 54, wherein the exogenous gene is derived from a plant of the genus Salix or Populus.

56. A transgenic crop plant according to claim 54, wherein the exogenous polynucleotide is associated with a promoter sequence capable of directing constitutive expression of the protein encoded by the exogenous polynucleotide in the transgenic plant.

57. A transgenic crop plant according to claim 54, wherein the plant is a secondary or subsequent generation transgenic plant derived from propagation of a primary transgenic crop plant, the primary transgenic plant being generated by introduction of the exogenous polynucleotide into a wild type plant.

58. A transgenic crop plant according to claim 57, wherein the second or subsequent generation transgenic plant is homozygous for the exogenous polynucleotide.

59. A genetic construct comprising (a) a nucleotide sequence having at least 50, 55, 60, 65, 70, 75, 80, 85, 90, 95, 97, 98, 99 or 100% identity to SEQ ID NO 1, 2, 3, 4, 5, 6, 7, 8, 9, 10, 11, 12, 13, 14, 15, 16, 17, 18, 19, 20, 21, 22, 23, 24 or 25 or the corresponding cDNA sequence, and (b) a promoter sequence capable of directing expression of the protein encoded by the nucleotide sequence in a plant comprising the genetic construct.

60. A genetic construct comprising (a) a nucleotide sequence having at least 50, 55, 60, 65, 70, 75, 80, 85, 90, 95, 97, 98, 99 or 100% identity to a nucleotide sequence encoding the polypeptide of SEQ ID NO 26, 27, 28, 29, 30, 31, 32, 33, 34, 35, 36, 37, 38 or 39, and (b) a promoter sequence capable of directing expression of the protein encoded by the nucleotide sequence in a plant comprising the genetic construct.

61. A plant transformation vector comprising a genetic construct as defined in claim 60.

62. A plant or plant cell comprising a transformation vector as defined in claim 61.
